(19) United States
(12) Patent Application Publication
(10) Pub. No.: US 2023/0383294 A1
(43) Pub. Date: Nov. 30, 2023

(54) NOVEL RNA COMPOSITIONS AND METHODS FOR INHIBITING ANGPTL3

(71) Applicant: SANOFI, Paris (FR)

(72) Inventors: Bodo BRUNNER, Frankfurt am Main (DE); Mike HELMS, Frankfurt am Main (DE); Armin HOFMEISTER, Frankfurt am Main (DE); Kerstin JAHN-HOFMANN, Frankfurt am Main (DE); Christiane METZ-WEIDMANN, Frankfurt am Main (DE); Sabine SCHEIDLER, Frankfurt am Main (DE); Pierrick RIVAL, Paris (FR)

(21) Appl. No.: 18/248,982

(22) PCT Filed: Oct. 15, 2021
(86) PCT No.: PCT/EP21/78570

§ 371 Date: Apr. 13, 2023

(30) Foreign Application Priority Data
Oct. 16, 2020 (EP) 20306223.7

(51) Int. Cl. C12N 15/113

(52) U.S. Cl. CPC C12N 15/113; C12N 2310/14; C12N 2310/351; C12N 2310/315; C12N 2310/321

(57) ABSTRACT

The present disclosure relates to dsRNAs targeting ANGPTL3, methods of inhibiting ANGPTL3 gene expression, and methods of treating one or more conditions associated with ANGPTL3 gene expression.

### SEQUENCE LISTING

Nucleic acid sequences are disclosed in the present specification that serve as references. The same sequences are also presented in a sequence listing formatted according to standard requirements for the purpose of patent matters. In case of any sequence discrepancy with the standard sequence listing, the sequences described in the present specification shall be the reference.

### FIELD OF THE INVENTION

The present disclosure relates to dsRNAs targeting ANGPTL3, methods of inhibiting ANGPTL3 gene expression, and methods of treating one or more conditions associated with ANGPTL3 gene expression.

### BACKGROUND OF THE INVENTION

Angiopoietin-like protein 3 (ANGPTL3) is an ANGPTL family member believed to be involved in lipid and glucose metabolism and angiogenesis. ANGPTL3, also known as angiopoietin 5, ANGPT5, FHBL2, and ANL3, is a 54 kDa hepatic secretory protein regulating plasma lipid levels, including levels of plasma triglycerides (TGs), very low density lipoproteins (VLDL), low density lipoproteins (LDL), and high density lipoproteins (HDL). ANGPTL3 inhibits lipoprotein lipase and endothelial lipase mediated hydrolysis of TGs and phospholipids (Tikka et al., Endocrine (2016) 52(2):187-93). Elevated levels of plasma triglycerides (e.g., 150 mg/dL or higher) and LDL (e.g., 130 mg/dL or higher), as well as diminished levels of HDL (e.g., 60 mg/dL or lower) significantly increase the risk of cardiovascular conditions such as heart disease, heart attack, stroke, and atherosclerosis, e.g., by contributing to risk factors such as obesity, hypertension, high cholesterol levels, high blood sugar, diabetes and metabolic syndrome. Very high levels of plasma triglycerides (e.g., 500 mg/dL or higher) significantly increase the risk of pancreatitis.

WO2012/177784 discloses angiopoietin-like (ANGPTL3) RNA compositions and methods of use thereof.

Double-stranded RNA molecules (dsRNAs) have been shown to block gene expression in a highly conserved regulatory mechanism known as RNA interference (RNAi). This appears to be a different mechanism of action from that of single-stranded oligonucleotides such as antisense oligonucleotides, antimiRs, and antagomiRs. In RNA interference technology, double-stranded RNAs, such as small interfering RNAs (siRNAs), bind to the RNA-induced silencing complex (“RISC”), where one strand (the “passenger strand” or “sense strand”) is displaced and the remaining strand (the “guide strand” or “antisense strand”) cooperates with RISC to bind a complementary RNA (the target RNA). Once bound, the target RNA is cleaved by RNA endonuclease Argonaute (AGO) in RISC and then further degraded by RNA exonucleases. RNAi has now been used to develop a new class of therapeutic agents for treating disorders caused by the aberrant or unwanted expression of a gene.

Due to the importance of ANGPTL3 in regulating triglyceride and lipid metabolism, and the prevalence of diseases associated with elevated triglyceride and LDL levels, there is a continuing need to identify inhibitors of ANGPTL3 expression and to test such inhibitors for efficacy and unwanted side effects such as cytotoxicity.

### SUMMARY OF THE INVENTION

Provided herein are dsRNAs useful for inhibiting expression of an ANGPTL3 gene. The dsRNAs provided herein may reduce elevated triglyceride, VLDL and/or LDL levels into normal ranges, or maintain normal triglyceride levels, resulting in overall improved health. The RNA agents of the present disclosure may be used to treat conditions such as lipid metabolism disorders characterized in whole or in part by elevated TG and/or LDL cholesterol (LDL-c) levels (e.g., hypertriglyceridemia, and hyperlipidemia such as familial combined hyperlipidemia, familial hypercholesterolemia (e.g., homozygous familial hypercholesterolemia or HoFH), and polygenic hypercholesterolemia). The RNA agents of the present disclosure also can be used to lower cardiovascular risks (e.g., atherosclerosis, arteriosclerosis, heart disease, heart attack, and stroke) in patients who have elevated TG and LDL-c levels.

Accordingly, provided herein is a double-stranded ribonucleic acid (dsRNA) that inhibits expression of a human angiopoietin-like protein 3 (ANGPTL3) gene by targeting a target sequence on an RNA transcript of the ANGPTL3 gene, wherein the dsRNA comprises a sense strand comprising a sense sequence, and an antisense strand comprising an antisense sequence, wherein the sense sequence is at least 90% identical to the target sequence, and wherein the target sequence is nucleotides 135-153, 143-161, 143-163, 144-162, 145-163, 150-168, 151-169, 1528-1546, 1530-1548, 1532-1550, 1533-1551, 1535-1553, 1602-1620, 2612-2630, or 2773-2791 of SEQ ID NO: 1181. In some embodiments, the sense strand and antisense strand of the present dsRNA are complementary to each other over a region of 15-25 contiguous nucleotides. In some embodiments, the sense strand and the antisense strand are no more than 30 nucleotides in length.

In some embodiments, the target sequence of the present dsRNA is nucleotides 135-153, 143-161, 143-163, 144-162, 145-163, 150-168, 151-169, 1528-1546, 1530-1548, 1532-1550, 1533-1551, 1535-1553, 1602-1620, 2612-2630, or 2773-2791 of SEQ ID NO: 1181. In further embodiments, the target sequence is nucleotides 135-153, 143-161, 144-162, 145-163, 150-168, or 1535-1553 of SEQ ID NO: 1181. In further embodiments, the target sequence is nucleotides 143-161, 1535-1553 and 135-153. As used herein, a target sequence defined as the range “x-y” of SEQ ID NO: Z consists of the target sequence beginning at the nucleotide in position x and ending at the nucleotide in position y of the nucleic acid sequence of SEQ ID NO: Z. Illustratively, for the sake of clarity, the target sequence defined as the range “135-153” consists of the target sequence beginning at the nucleotide in position 135 and ending at the nucleotide in position 153 of the nucleic acid sequence of SEQ ID NO: 1181.

In some embodiments, the dsRNA comprises an antisense sequence that is at least 90% identical to a nucleotide sequence selected from the group consisting of SEQ ID NOs: 227-229, 261-265, 269, 343, 356, 379, 385, 386, and 426.

In some embodiments, the sense sequence and the antisense sequence of the present dsRNA are complementary, wherein a) the sense sequence comprises a nucleotide sequence selected from the group consisting of SEQ ID NOs: 13-15, 47-51, 55, 129, 142, 165, 171, 172, and 212; or b) the antisense sequence comprises a nucleotide sequence selected from the group consisting of SEQ ID NOs: 227-229, 261-265, 269, 343, 356, 379, 385, 386, and 426.

In some embodiments, the sense strand and antisense strand of the dsRNA respectively comprise the nucleotide sequences of: a) SEQ ID NOs: 13 (sense strand) and 227 (antisense strand); b) SEQ ID NOs: 14 and 228; c) SEQ ID NOs: 15 and 229; d) SEQ ID NOs: 47 and 261; e) SEQ ID NOs: 48 and 262; f) SEQ ID NOs: 49 and 263; g) SEQ ID NOs: 50 and 264; h) SEQ ID NOs: 51 and 265; i) SEQ ID NOs: 55 and 269; j) SEQ ID NOs: 129 and 343; k) SEQ ID NOs: 142 and 356; 1) SEQ ID NOs: 165 and 379; m) SEQ ID NOs: 171 and 385; n) SEQ ID NOs: 172 and 386; or o) SEQ ID NOs: 212 and 426. In some embodiments, the sense strand and antisense strand of the dsRNA respectively comprise the nucleotide sequences of: a) SEQ ID NOs: 13 and 227; b) SEQ ID NOs: 14 and 228; c) SEQ ID NOs: 15 and 229; d) SEQ ID NOs: 51 and 265; e) SEQ ID NOs: 165 and 379; or f) SEQ ID NOs: 171 and 385.

In some embodiments, the dsRNA comprises one or more modified nucleotides, wherein at least one of the one or more modified nucleotides is 2′-deoxy-2′-fluoro-ribonucleotide, 2′-deoxyribonucleotide, or 2′-O-methyl-ribonucleotide. In further embodiments, the dsRNA comprises two or more 2′-O-methyl-ribonucleotides and two or more 2′-deoxy-2′-fluoro-ribonucleotides (e.g., in an alternating pattern). In some embodiments, the sense sequence and the antisense sequence comprise alternating 2′-O-methyl ribonucleotides and 2′-deoxy-2′-fluoro ribonucleotides.

In some embodiments, the dsRNA comprises an inverted 2′-deoxyribonucleotide at the 3′-end of its sense or antisense strand.

In some embodiments, one or both of the sense strand and the antisense strand of the present dsRNA further comprise a) a 5′ overhang comprising one or more nucleotides; and/or b) a 3′ overhang comprising one or more nucleotides. In further embodiments, an overhang in the dsRNA comprises two or three nucleotides. In certain embodiments, an overhang in the dsRNA comprises one or more thymines.

In some embodiments, the sense strand comprises a nucleotide sequence selected from the group consisting of SEQ ID NOs: 441-443, 475-479, 483, 557, 570, 593, 599, 600, and 640; and/or the antisense strand comprises a nucleotide sequence selected from the group consisting of SEQ ID NOs: 655-657, 689-693, 697, 771, 784, 807, 813, 814, and 854. In further embodiments, the sense strand and antisense strand of the dsRNA respectively comprise the nucleotide sequences of: a) SEQ ID NOs: 441 and 655; b) SEQ ID NOs: 442 and 656; c) SEQ ID NOs: 443 and 657; d) SEQ ID NOs: 475 and 689; e) SEQ ID NOs: 476 and 690; f) SEQ ID NOs: 477 and 691; g) SEQ ID NOs: 478 and 692; h) SEQ ID NOs: 479 and 693; i) SEQ ID NOs: 483 and 697; j) SEQ ID NOs: 557 and 771; k) SEQ ID NOs: 570 and 784; 1) SEQ ID NOs: 593 and 807; m) SEQ ID NOs: 599 and 813; n) SEQ ID NOs: 600 and 814; or o) SEQ ID NOs: 640 and 854.

In some embodiments, the dsRNA is conjugated to one or more ligands with or without a linker (e.g., one or more N-acetylgalactosamine (GalNAc). In some embodiments, the ligand is N-acetylgalactosamine (GalNAc) and the dsRNA is conjugated to one or more GalNAc. In some embodiments, the dsRNA is a small interfering RNA (siRNA).

In some embodiments, one or both strands of the dsRNA comprise one or more compounds having the structure of

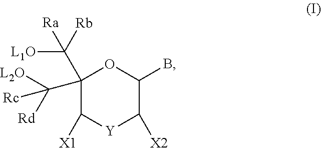

wherein:
B is a heterocyclic nucleobase,
one of L1 and L2 is an internucleoside linking group linking the compound of formula (I) to said strand(s) and the other of L1 and L2 is H, a protecting group, a phosphorus moiety or an internucleoside linking group linking the compound of formula (I) to said strand(s),
Y is O, NH, NR1 or N—C(═O)—R1, wherein R1 is:
   a (C1-C20) alkyl group, optionally substituted by one or more groups selected from an halogen atom, a (C1-C6) alkyl group, a (C3-C8) cycloalkyl group, a (C3-C14) heterocycle, a (C6-C14) aryl group, a (C5-C14) heteroaryl group, —O-Z1, —N(Z1)(Z2), —S-Z1, —CN, —C(=J)-O-Z1, —O—C(=J)-Z1, —C(=J)-N(Z1)(Z2), and —N(Z1)-C(=J)-Z2, wherein
J is O or S,
each of Z1 and Z2 is, independently, H, a (C1-C6) alkyl group, optionally substituted by one or more groups selected from a halogen atom and a (C1-C6) alkyl group,
   a (C3-C8) cycloalkyl group, optionally substituted by one or more groups selected from a halogen atom and a (C1-C6) alkyl group,
   a group —[C(═O)]m-R2-(O—CH₂—CH₂)p-R3, wherein
m is an integer meaning 0 or 1,
p is an integer ranging from 0 to 10,
R2 is a (C1-C20) alkylene group optionally substituted by a (C1-C6) alkyl group, —O-Z3, —N(Z3)(Z4), —S-Z3, —CN, —C(═K)—O—Z3, —O—C(═K)—Z3, —C(═K)—N(Z3)(Z4), or —N(Z3)-C(═K)—Z4,
wherein
K is O or S,
each of Z3 and Z4 is, independently, H, a (C1-C6) alkyl group, optionally substituted by one or more groups selected from a halogen atom and a (C1-C6) alkyl group,
and
R3 is selected from the group consisting of a hydrogen atom, a (C1-C6) alkyl group, a (C1-C6) alkoxy group, a (C3-C8) cycloalkyl group, a (C3-C14) heterocycle, a (C6-C14) aryl group or a (C5-C14) heteroaryl group,
or R3 is a cell targeting moiety,
   X1 and X2 are each, independently, a hydrogen atom, a (C1-C6) alkyl group, and
   each of Ra, Rb, Rc and Rd is, independently, H or a (C1-C6) alkyl group,
      or a pharmaceutically acceptable salt thereof.

In some embodiments, in the present dsRNA comprising one or more compounds of formula (I), Y is
a) NR1, R1 is a non-substituted (C1-C20) alkyl group;
b) NR1, R1 is a non-substituted (C1-C16) alkyl group, which includes an alkyl group selected from a group comprising methyl, isopropyl, butyl, octyl, and hexadecyl;
c) NR1, R1 is a (C3-C8) cycloalkyl group, optionally substituted by one or more groups selected from a halogen atom and a (C1-C6) alkyl group;
d) NR1, R1 is a cyclohexyl group;
e) NR1, R1 is a (C1-C20) alkyl group substituted by a (C6-C14) aryl group;
f) NR1, R1 is a methyl group substituted by a phenyl group;
g) N—C(═O)—R1, R1 is an optionally substituted (C1-C20) alkyl group; or
h) N—C(═O)—R1, R1 is methyl or pentadecyl.

In some embodiments, in the present dsRNA comprising one or more compounds of formula (I), B is selected from a group consisting of a pyrimidine, a substituted pyrimidine, a purine and a substituted purine, or a pharmaceutically acceptable salt thereof.

In some embodiments, in the present dsRNA comprising one or more compounds of formula (I), R3 is of the formula (II):

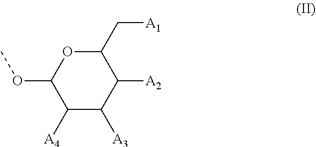

wherein A1, A2 and A3 are OH,
A4 is OH or NHC(═O)—R5, wherein R5 is a (C1-C6) alkyl group, optionally substituted by a halogen atom, or a pharmaceutically acceptable salt thereof.

In some embodiments, in the present dsRNA comprising one or more compounds of formula (I), R3 is N-acetyl-galactosamine, or a pharmaceutically acceptable salt thereof.

In some embodiments, the present dsRNA comprises one or more nucleotides from Tables A and B.

In some embodiments, the present dsRNA comprises from 2 to 10 compounds of formula (I), or a pharmaceutically acceptable salt thereof.

In some embodiments, the present dsRNA comprises 2 to 10 compounds of formula (I) on the sense strand.

In some embodiments, in the present dsRNA, the sense strand comprises two to five compounds of formula (I) at the 5′ end, and/or comprises one to three compounds of formula (I) at the 3′ end.

In some embodiments, in the present dsRNA,
a) the two to five compounds of formula (I) at the 5′ end of the sense strand comprise lgT3 and/or lgT7, optionally comprising three consecutive lgT3 nucleotides; and/or
b) the one to three compounds of formula (I) at the 3′ end of the sense strand comprise lT4 or lT3; optionally comprising two consecutive lT4.

In some embodiments, the present dsRNA comprises one or more internucleoside linking groups independently selected from the group consisting of phosphodiester, phosphotriester, phosphorothioate, phosphorodithioate, alkyl-phosphonate and phosphoramidate backbone linking groups, or a pharmaceutically acceptable salt thereof.

In some embodiments, the present dsRNA is selected from the dsRNAs in Tables 1-3.

In some embodiments, the sense strand comprises a nucleotide sequence selected from the group consisting of SEQ ID NOs: 858, 902, 907, 911, 915, 934, 970, 979, and 988; and the antisense strand comprises a nucleotide sequence selected from the group consisting of SEQ ID NOs: 1020, 1064, 1069, 1073, 1077, 1096, 1132, 1141, and 1150.

In some embodiments, the sense strand and antisense strand of the dsRNA respectively comprise the nucleotide sequences of: a) SEQ ID NOs: 858 and 1020; b) SEQ ID NOs: 902 and 1064; c) SEQ ID NOs: 907 and 1069; d) SEQ ID NOs: 911 and 1073; e) SEQ ID NOs: 915 and 1077; f) SEQ ID NOs: 934 and 1096; g) SEQ ID NOs: 970 and 1132; h) SEQ ID NOs: 979 and 1141; or i) SEQ ID NOs: 988 and 1150.

The present disclosure further provides a pharmaceutical composition comprising a dsRNA or DNA vector described herein, and a pharmaceutically acceptable excipient. The present disclosure further provides a pharmaceutical composition comprising a dsRNA as described herein, and a pharmaceutically acceptable excipient.

Also provided in this disclosure is the present dsRNA, DNA vector, or composition for use in inhibiting ANGPTL3 expression in a human in need thereof, or for use in treating or preventing an ANGPTL3-associated condition in a human in need thereof. The present disclosure also provides the dsRNA, or a composition comprising it, for use in inhibiting ANGPTL3 expression in a human in need thereof. In a particular embodiment, the expression of the ANGPTL3 gene in the liver of the human is inhibited by the dsRNA. The disclosure further provides a dsRNA, or a composition comprising it, for use in in treating or preventing an ANGPTL3-associated condition in a human in need thereof. In a particular embodiment, the ANGPTL3-associated condition is a lipid metabolism disorder. In a particular embodiment, the lipid metabolism disorder is hypertriglyceridemia.

Further provided in this disclosure is a method of inhibiting ANGPTL3 expression, or treating or preventing an ANGPTL3-associated condition, in a mammal (e.g., a human) in need thereof by administering the present dsRNA or composition to the mammal.

Further provided in this disclosure is the use of the present dsRNA in the manufacture of a medicament for inhibiting ANGPTL3 expression, or treating or preventing an ANGPTL3-associated condition, in a mammal (e.g., a human) in need thereof, as well as articles of manufacture (e.g., kits).

In some embodiments, the dsRNA inhibits the expression of the ANGPTL3 gene in the liver of the mammal (e.g., human) in the treatment methods. In certain embodiments, the ANGPTL3-associated condition is a lipid metabolism disorder, e.g., hypertriglyceridemia and associated diseases and conditions such as atherosclerosis, pancreatitis, and hyperlipidemia such as familial combined hyperlipidemia, familial hypercholesterolemia (e.g., HoFH), and polygenic hypercholesterolemia.

### BRIEF DESCRIPTION OF THE DRAWINGS

FIGS. 1A, 1B and 1C are graphs showing RT-qPCR analysis of ANGPTL3 mRNA expression in human Hep3B cell lysates following treatment with 164 test siRNAs as indicated at 0.1 or 1 nM, respectively. Expression of mRNA is represented relative to cells treated with a non-targeting siRNA control. Error bars indicate standard deviation.

FIG. 2 is a graph showing cytotoxic effects of 18 selected test siRNAs in human Hep3B cells. Cells were treated with siRNAs as indicated at 5 or 50 nM before being analyzed for viability (CellTiter-Glo assay) and toxicity (ToxiLight assay). Ratios of the resulting readings are shown relative to results for a non-targeting siRNA control. Error bars indicate standard deviation.

FIG. 3 is a graph of immune stimulation showing the amount of interferon α (IFNα) protein released into the supernatant of human peripheral blood mononuclear cells (PBMCs) isolated from three donors and transfected with selected GalNAc-conjugated siRNAs targeting ANGPTL3 or controls. Protein concentration was determined by ELISA. Error bars indicate standard deviation.

FIG. 4 is a graph showing cytotoxic effects of 11 selected GalNAc-conjugated test siRNAs in human primary hepatocytes following free uptake. Cells were treated with siRNAs as indicated at 1, 5, or 25 μM before being analyzed for viability (CellTiter-Glo assay) and toxicity (ToxiLight assay). Ratios of the resulting readings are shown relative to results for an untreated control, in comparison to toxic positive controls and a non-targeting siRNA control. Error bars indicate standard deviation.

FIG. 5 is a graph showing the amount of ANGPTL3 protein secreted into the supernatant of human primary hepatocytes treated with increasing concentrations of 11 selected GalNAc-siRNAs (free uptake) targeting ANGPTL3, as determined by ELISA. Error bars indicate standard deviation.

FIG. 6 is a graph showing the correlation between relative mRNA expression (as determined by qPCR) and protein expression (as determined by ELISA) observed in human primary hepatocytes following treatment with 11 selected GalNAc-siRNAs (plus a nucleotide control) at 10, 100, or 1000 nM, respectively (free uptake).

FIG. 7 is a graph showing serum ANGPTL3 protein levels of mice treated subcutaneously with selected GalNAc-siRNAs at 12 mg/kg at day 0. Treated mice express human ANGPTL 3 from a liver specific adeno-associated viral vector. Human ANGPTL3 levels were quantified by ELISA. Error bars indicate standard deviation.

FIG. 8 is a graph showing RT-qPCR analysis of ANGPTL3 mRNA expression in human Hep3B cell lysates following treatment with 52 additional test siRNAs as indicated at 0.1 or 1 nM, respectively. Expression of mRNA is represented relative to cells treated with a non-targeting siRNA control. Error bars indicate standard deviation.

FIG. 9 is a graph showing RT-qPCR analysis of ANGPTL3 mRNA expression in cynomolgus primary hepatocyte lysates following treatment with 52 additional test siRNAs as indicated at 0.1 and 1 nM, respectively. mRNA expression is represented relative to cells treated with a non-targeting siRNA control. Error bars indicate standard deviation.

FIG. 10 is a graph showing cytotoxic effects of 11 additional test siRNAs in human Hep3B cells. Cells were treated with siRNAs as indicated at 5 or 50 nM before being analyzed for viability (CellTiter-Glo assay) and toxicity (ToxiLight assay). Ratios of the resulting readings are shown relative to results for a non-targeting siRNA control. Error bars indicate standard deviation.

FIG. 11 is a graph of immune stimulation showing the amount of interferon α (IFNα) protein released into the supernatant of human peripheral blood mononuclear cells (PBMCs) isolated from three donors and transfected with selected GalNAc-conjugated siRNAs targeting ANGPTL3 or controls. Protein concentration was determined by ELISA. Error bars indicate standard deviation.

FIG. 12 is a graph showing cytotoxic effects of six selected GalNAc-conjugated test siRNAs in human primary hepatocytes following free uptake. Cells were treated with siRNAs as indicated at 1, 5, or 25 μM before being analyzed for viability (CellTiter-Glo assay) and toxicity (ToxiLight assay). Ratios of the resulting readings are shown relative to results for an untreated control, in comparison to toxic positive controls, a non-targeting siRNA control, and two siRNAs selected from the first round of screening. Error bars indicate standard deviation.

FIG. 13 is a graph showing the amount of ANGPTL3 protein secreted into the supernatant of human primary hepatocytes treated with increasing concentrations of 4 selected GalNAc-siRNAs (free uptake) targeting ANGPTL3, as determined by ELISA. Two siRNAs selected from the first round of screening were included as references. Error bars indicate standard deviation.

FIG. 14 is a graph showing serum ANGPTL3 protein levels of mice treated subcutaneously with selected GalNAc-siRNAs from both screening rounds at 10 mg/kg at day 0. Treated mice express human ANGPTL3 from a liver specific adeno-associated viral vector. Human ANGPTL3 levels were quantified by ELISA. Error bars indicate standard deviation.

FIGS. 15A-F are graphs showing RT-qPCR analysis of ANGPTL3 mRNA expression in primary human hepatocytes following treatment of the cells with 3×54 test siRNAs based on parent siRNA #013-c (FIGS. 15A and 15B), siRNA #051-c (FIGS. 15C and 15D), and siRNA #165-c (FIGS. 15E and 15F) at 1 nM, 10 nM, or 100 nM for 72 hours under free uptake conditions. Expression of mRNA is represented relative to cells treated with LV2, a non-targeting siRNA control. Error bars indicate standard deviation.

FIG. 16 is a graph showing immune stimulation indicated by the amount of interferon α2a (IFN-α2a) protein released into the supernatant of human peripheral blood mononuclear cells (PBMCs) isolated from three donors and transfected for 24 hours with 100 nM concentration of 24 selected modified GalNAc ANGPTL3 siRNAs together with respective parental sequences (siRNA #013-c, siRNA #051-c, and siRNA #165-c) or controls. Protein concentration was determined by ELISA. Error bars indicate standard deviation.

FIG. 17 is a graph showing cytotoxic effects of 24 selected modified GalNAc ANGPTL3 siRNAs together with respective parental sequences (siRNA #013-c, siRNA #051-c, and siRNA #165-c) in human Hep3B cells. Cells were transfected with siRNAs as indicated at 5 or 50 nM concentration for 72 hours before being analyzed for viability (CellTiter-Glo assay) and toxicity (ToxiLight assay). Ratios of the resulting readings are shown relative to results for a non-targeting siRNA control. Error bars indicate standard deviation.

FIGS. 18A, 18B, and 18C are graphs showing serum ANGPTL3 protein levels over time in mice treated once subcutaneously with selected GalNAc-siRNAs at 5 mg/kg at day 0. They show the results of 3×8 siRNAs based on parental sequences siRNA #013-c (FIG. 18A), siRNA #051-c (FIG. 18B), and siRNA #165-c (FIG. 18C). Treated mice express human ANGPTL3 from a liver-specific adeno-associated viral vector. Human ANGPTL3 levels were quantified by ELISA. Error bars indicate standard error of the mean.

### DETAILED DESCRIPTION OF THE INVENTION

The present disclosure provides novel double-stranded RNAs (dsRNAs) that inhibit expression of an angiopoietin-like protein 3 (ANGPTL3) gene. In some embodiments, the dsRNAs are small interfering RNAs (siRNAs). The dsRNAs can be used to treat conditions such as lipid metabolism disorders (e.g., dyslipidemia, mixed-dyslipidemia, hypertriglyceridemia, and associated diseases such as pancreatitis). Unless otherwise stated, “ANGPTL3” refers to human ANGPTL3 herein. An mRNA sequence of a human ANGPTL3 protein is available under NCBI Reference Sequence No. NM_014495.3 (SEQ ID NO: 1181) and its polypeptide sequence is available under NCBI Reference Sequence No. NP_055310.1 (SEQ ID NO: 1182). In certain embodiments, the present disclosure refers to cynomolgus ANGPTL3. An mRNA sequence of a cynomolgus ANGPTL3 protein is available under NCBI Reference Sequence No. XM_005543185.1 (SEQ ID NO: 1183) and its polypeptide sequence is available under NCBI Reference Sequence No. XP_005543242.1 (SEQ ID NO: 1184).

A dsRNA of the present disclosure (e.g., a dsRNA with or without a GalNAc moiety)_may have one, two, three, or all four of the following properties: (i) has a half-life of at least 24, 26, 28, 30, 32, 48, 52, 56, 60, 72, 96, or 168 hours in vitro; (ii) does not increase production of interferon α secreted from human primary PMBCs; (iii) has an IC₅₀ value of no greater than 0.001, 0.01, 0.1, 0.3, 1, 1.5, 2, 3, 4, 5, 6, 7, 8, 9, or 10 nM for inhibition of human ANGPTL3 expression in vitro (in, e.g., human Hep3B cells, human primary hepatocytes, or cynomolgus primary hepatocytes as described in the working examples below); and (iv) reduces protein levels of ANGPTL3 by at least 50%, 55%, 60%, 65%, 70%, 75%, 80%, 85%, 90%, 95%, 96%, 97%, 98%, or 99% in vivo in C57BL/6 mice expressing human ANGPTL3 (e.g., at 5, 10, 15 or more mg/kg).

In some embodiments, a dsRNA of the present disclosure comprises a GalNAc moiety and has one, two, three, or all four of the following properties: (i) has a half-life of at least 24, 48, 72, 96, or 168 hours in vitro; (ii) does not increase production of interferon α secreted from human primary PMBCs, (iii) has an IC₅₀ value of no greater than 9.68 nM for inhibition of human ANGPTL3 expression in vitro in human or cynomolgus primary hepatocytes; and (iv) reduces protein levels of human ANGPTL3 by at least 60% in vivo in C57BL/6 mice expressing human ANGPTL3 after a single subcutaneous dose of 5 mg/kg. In certain embodiments, the dsRNA has all of said properties.

It will be understood by the person skilled in the art that the dsRNAs described herein do not occur in nature (“isolated” dsRNAs).

### I. Double-Stranded RNAs

Certain aspects of the present disclosure relate to double-stranded ribonucleic acid (dsRNA) molecules targeting ANGPTL3. As used herein, the term “double-stranded RNA” or “dsRNA” refers to an oligoribonucleotide molecule comprising a duplex structure having two anti-parallel and substantially complementary nucleic acid strands. The two strands forming the duplex structure may be different portions of one larger RNA molecule, or they may be on separate RNA molecules. When the two strands are on separate RNA molecules, the dsRNA structure may function as short interfering RNA (siRNA). Where the two strands are part of one larger molecule and are connected by an uninterrupted chain of nucleotides between the 3′-end of a first strand and the 5′-end of a second strand, the connecting RNA chain is referred to as a “hairpin loop” and the RNA molecule may be termed “short hairpin RNA,” or “shRNA.” The RNA strands may have the same or a different number of nucleotides. In addition to the duplex structure, a dsRNA may comprise overhangs of one or more (e.g., 1, 2 or 3) nucleotides. A dsRNA of the present disclosure may further comprise a targeting moiety (with or without a linker) as further described below.

As used herein, the term “polynucleotide” refers to a polymeric form of nucleotides of at least 10 bases in length, either ribonucleotides or deoxyribonucleotides or a modified form of either type of nucleotide. The term includes single and double stranded forms.

A “dsRNA” may include naturally occurring ribonucleotides, and/or chemically modified analogs thereof. As used herein, “dsRNAs” are not limited to those with ribose-containing nucleotides. A dsRNA herein encompasses a double-stranded polynucleotide molecule where the ribose moiety in some or all of its nucleotides has been replaced by another moiety, so long as the resultant double-stranded molecule can inhibit the expression of a target gene by RNA interference. The dsRNA may also include one or more, but not more than 60% (e.g., not more than 50%, 40%, 30%, 20%, or 10%) deoxyribonucleotides or chemically modified analogs thereof.

A dsRNA of the present disclosure comprises a sense strand comprising a sense sequence, and an antisense strand comprising an antisense sequence, wherein the sense strand and the antisense strand are sufficiently complementary to hybridize to form a duplex structure. The term “antisense sequence” refers to a sequence that is substantially or fully complementary, and binds under physiological conditions, to a target RNA sequence in a cell. A “target sequence” refers to a nucleotide sequence on an RNA molecule (e.g., a primary RNA transcript or a messenger RNA transcript) transcribed from a target gene, e.g., an ANGPTL3 gene. The term “sense sequence” refers to a sequence that is substantially or fully complementary to the antisense sequence.

The ANGPTL3-targeting dsRNA of the present disclosure comprises a sense strand comprising a sense sequence and an antisense strand comprising an antisense sequence, wherein the sense and antisense sequences are substantially or fully complementary to each other. Unless otherwise indicated, the term “complementary” refers herein to the ability of a polynucleotide comprising a first contiguous nucleotide sequence, under certain conditions, e.g., physiological conditions, to hybridize to and form a duplex structure with another polynucleotide comprising a second contiguous nucleotide sequence. This may include base-pairing of the two polynucleotides over the entire length of the first or second contiguous nucleotide sequence; in this case, the two nucleotide sequences are considered “fully complementary” to each other. For example, in a case where a dsRNA comprises a first oligonucleotide 21 nucleotides in length and a second oligonucleotide 23 nucleotides in length, and where the two oligonucleotides form 21 contiguous base-pairs, the two oligonucleotides may be referred to as “fully complementary” to each other. Where a first polynucleotide sequence is referred to as “substantially complementary” to a second polynucleotide sequence, the two sequences may base-pair with each other over 80% or more (e.g., 90% or more) of their length of hybridization, with no more than 20% (e.g., no more than 10%) of mismatching base-pairs (e.g., for a duplex of 20 nucleotides, no more than 4 or no more than 2 mismatched base-pairs). Where two oligonucleotides are designed to form a duplex with one or more single-stranded overhangs, such overhangs shall not be regarded as mismatches for the determination of complementarity. Complementarity of two sequences may be based on Watson-Crick base-pairs and/or non-Watson-Crick base-pairs. As used herein, a polynucleotide which is “substantially complementary to at least part of” an mRNA refers to a polynucleotide which is substantially complementary to a contiguous portion of an mRNA of interest (e.g., an mRNA encoding ANGPTL3).

In some embodiments, the ANGPTL3-targeting dsRNA is an siRNA where the sense and antisense strands are not covalently linked to each other. In some embodiments, the sense and antisense strands of the ANGPTL3-targeting dsRNA are covalently linked to each other, e.g., through a hairpin loop (such as in the case of shRNA), or by means other than a hairpin loop (such as by a connecting structure referred to as a “covalent linker”).

### I.1 Lengths

In some embodiments, each of the sense sequence (in the sense strand) and the antisense sequence (in the antisense strand) is 9-30 nucleotides in length. For example, each sequence can be any of a range of nucleotide lengths having an upper limit of 21, 22, 23, 24, 25, 26, 27, 28, 29, or 30 and an independently selected lower limit of 9, 10, 11, 12, 13, 14, 15, 16, 17, 18, 19, or 20. In some embodiments, the number of nucleotides in each sequence may be 15-25, 15-30, 16-29, 17-28, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, or 19-21.

In some embodiments, each sequence is greater than 8, 9, 10, 11, 12, 13, 14, 15, 16, 17, 18, 19, 20, 21, 22, 23, 24, 25, 26, 27, 28, 29, or 30 nucleotides in length. In some embodiments, each sequence is less than 21, 22, 23, 24, 25, 26, 27, 28, 29, 30, or 31 nucleotides in length. In some embodiments, each sequence is 9, 10, 11, 12, 13, 14, 15, 16, 17, 18, 19, 20, 21, 22, 23, 24, 25, 26, 27, 28, 29, or 30 nucleotides in length.

In some embodiments, the sense and antisense sequences are each at least 15 and no greater than 25 nucleotides in length. In some embodiments, the sense and antisense sequences are each at least 19 and no greater than 25 nucleotides in length. For example, the sequences are 15, 16, 17, 18, 19, 20, 21, 22, 23, 24, or 25 nucleotides in length.

The sense sequence and antisense sequence may be of the same or different lengths. For example, the antisense sequence may have 21 nucleotides while the sense sequence may have 23 nucleotides. In another example, the antisense sequence and the sense sequence both have 19 nucleotides.

In some embodiments, the ANGPTL3-targeting dsRNA has sense and antisense strands of the same length or different lengths. For example, the sense strand may be 1, 2, 3, 4, 5, 6, or 7 nucleotides longer than the antisense strand. Alternatively, the sense strand may be 1, 2, 3, 4, 5, 6, or 7 nucleotides shorter than the antisense strand.

In some embodiments, each of the sense strand and the antisense strand is 9-36 nucleotides in length. For example, each strand can be any of a range of nucleotide lengths having an upper limit of 21, 22, 23, 24, 25, 26, 27, 28, 29, 30, 31, 32, 33, 34, 35, or 36 and an independently selected lower limit of 9, 10, 11, 12, 13, 14, 15, 16, 17, 18, 19, or 20. In some embodiments, the number of nucleotides in each strand may be 15-25, 15-30, 16-29, 17-28, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, or 19-21.

In some embodiments, each strand is greater than 8, 9, 10, 11, 12, 13, 14, 15, 16, 17, 18, 19, 20, 21, 22, 23, 24, 25, 26, 27, 28, 29, or 30 nucleotides in length. In some embodiments, each strand is less than 20, 21, 22, 23, 24, 25, 26, 27, 28, 29, 30, 31, 32, 33, 34, 35, 36, or 37 nucleotides in length. In some embodiments, each strand is 9, 10, 11, 12, 13, 14, 15, 16, 17, 18, 19, 20, 21, 22, 23, 24, 25, 26, 27, 28, 29, 30, 31, 32, 33, 34, 35, or 36 nucleotides in length.

In some embodiments, the sense and antisense strands are each at least 15 and no greater than 25 nucleotides in length. In some embodiments, the sense and antisense strands are each at least 19 and no greater than 23 nucleotides in length. For example, the strands are 19, 20, 21, 22, or 23 nucleotides in length.

In some embodiments, the sense strand may have 21, 22, 23, 24, or 25 nucleotides, including any modified nucleotides, while the antisense strand may have 21, 22, or 23 nucleotides, including any modified nucleotides. In certain embodiments, the sense strand may have a sense sequence having 19, 20, or 21 nucleotides, while the antisense strand may have an antisense sequence having 19, 20, or 21 nucleotides.

### I.2 Overhangs

In some embodiments, a dsRNA of the present disclosure comprises one or more overhangs at the 3′-end, 5′-end, or both ends of one or both of the sense and antisense strands. In some embodiments, the one or more overhangs improve the stability and/or inhibitory activity of the dsRNA.

“Overhang” refers herein to the unpaired nucleotide(s) that protrude from the duplex structure of a dsRNA when a 3′ end of a first strand of the dsRNA extends beyond the 5′ end of a second strand, or vice versa. “Blunt end” means that there are no unpaired nucleotides at that end of the dsRNA, i.e., no nucleotide overhang. A “blunt-ended” dsRNA is a dsRNA that is double-stranded over its entire length, i.e., no nucleotide overhang at either end of the duplex molecule. Chemical caps or non-nucleotide chemical moieties conjugated to the 3′ end and/or the 5′ end of a dsRNA are not considered herein in determining whether a dsRNA has an overhang or not.

In some embodiments, an overhang comprises one or more, two or more, three or more, or four or more nucleotides. For example, the overhang may comprise 1, 2, 3, or 4 nucleotides.

In some embodiments, an overhang of the present disclosure comprises one or more nucleotides (e.g., ribonucleotides or deoxyribonucleotides, naturally occurring or chemically modified analogs thereof). In some embodiments, the overhang comprises one or more thymines or chemically modified analogs thereof. In certain embodiments, the overhang comprises one or more thymines.

In some embodiments, the dsRNA comprises an overhang located at the 3′-end of the antisense strand. In some embodiments, the dsRNA comprises a blunt end at the 5′-end of the antisense strand. In some embodiments, the dsRNA comprises an overhang located at the 3′-end of the antisense strand and a blunt end at the 5′-end of the antisense strand. In some embodiments, the dsRNA comprises an overhang located at the 3′-end of the sense strand. In some embodiments, the dsRNA comprises a blunt end at the 5′-end of the sense strand. In some embodiments, the dsRNA comprises an overhang located at the 3′-end of the sense strand and a blunt end at the 5′-end of the sense strand. In some embodiments, the dsRNA comprises overhangs located at the 3′-end of both the sense and antisense strands of the dsRNA.

In some embodiments, the dsRNA comprises an overhang located at the 5′-end of the antisense strand. In some embodiments, the dsRNA comprises a blunt end at the 3′-end of the antisense strand. In some embodiments, the dsRNA comprises an overhang located at the 5′-end of the antisense strand and a blunt end at the 3′-end of the antisense strand. In some embodiments, the dsRNA comprises an overhang located at the 5′-end of the sense strand. In some embodiments, the dsRNA comprises a blunt end at the 3′-end of the sense strand. In some embodiments, the dsRNA comprises an overhang located at the 5′-end of the sense strand and a blunt end at the 3′-end of the sense strand. In some embodiments, the dsRNA comprises overhangs located at both the 5′-end of the sense and antisense strands of the dsRNA.

In some embodiments, the dsRNA comprises an overhang located at the 3′-end of the antisense strand and an overhang at the 5′-end of the antisense strand. In some embodiments, the dsRNA comprises an overhang located at the 3′-end of the sense strand and an overhang at the 5′-end of the sense strand.

In some embodiments, the dsRNA has two blunt ends.

In some embodiments, the overhang is the result of the sense strand being longer than the antisense strand. In some embodiments, the overhang is the result of the antisense strand being longer than the sense strand. In some embodiments, the overhang is the result of sense and antisense strands of the same length being staggered. In some embodiments, the overhang forms a mismatch with the target mRNA. In some embodiments, the overhang is complementary to the target mRNA.

In certain embodiments, a dsRNA of the present disclosure contains a sense strand having the sequence of 5′-CCA-[sense sequence]-invdT, and the antisense strand having the sequence of 5′-[antisense sequence]-dTdT-3′, where the trinucleotide CCA may be modified (e.g., 2′-O-Methyl-C and 2′-O-Methyl-A).

I.3 Target and dsRNA Sequences

The antisense strand of a dsRNA of the present disclosure comprises an antisense sequence that may be substantially or fully complementary to a target sequence of 12-30 nucleotides in length in an ANGPTL3 RNA (e.g., an mRNA). For example, the target sequence can be any of a range of nucleotide lengths having an upper limit of 19, 20, 21, 22, 23, 24, 25, 26, 27, 28, 29, or 30 and an independently selected lower limit of 12, 13, 14, 15, 16, 17, 18, or 19. In some embodiments, the number of nucleotides in the target sequence may be 15-25, 15-30, 16-29, 17-28, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, or 19-21.

In some embodiments, the target sequence is greater than 12, 13, 14, 15, 16, 17, 18, 19, or 20 nucleotides in length. In some embodiments, the target sequence is less than 21, 22, 23, 24, 25, 26, 27, 28, 29, or 30 nucleotides in length. In some embodiments, the target sequence is 12, 13, 14, 15, 16, 17, 18, 19, 20, 21, 22, 23, 24, 25, 26, 27, 28, 29, or 30 nucleotides in length. In certain embodiments, the target sequence is at least 15 and no greater than 25 nucleotides in length; for example, at least 19 and no greater than 23 nucleotides in length, or 15, 16, 17, 18, 19, 20, 21, 22, 23, 24, or 25 nucleotides in length.

The target sequence may be in the 5′-noncoding region, the coding region, or the 3′ noncoding region of the ANGPTL3 mRNA transcript. The target sequence may also be located at the junction of the noncoding and coding regions.

In some embodiments, the dsRNA antisense strand comprises an antisense sequence having one or more mismatch (e.g., one, two, three, or four mismatches) to the target sequence. In certain embodiments, the antisense sequence is fully complementary to the corresponding portion in the human ANGPTL3 mRNA sequence and is fully complementary or substantially complementary (e.g., comprises at least one or two mismatches) to the corresponding portion in a cynomolgus ANGPTL3 mRNA sequence. One advantage of such dsRNAs is to allow pre-clinical in vivo studies of the dsRNAs in non-human primates such as cynomolgus monkeys. In certain embodiments, the dsRNA sense strand comprises a sense sequence that is at least 70%, 75%, 80%, 85%, 90%, 95%, 96%, 97%, 98%, or 99% identical to the target sequence (e.g., in human or cynomolgus ANGPTL3 mRNA).

In some embodiments, the target sequence in a human ANGPTL3 mRNA sequence (SEQ ID NO:1181) has start and end nucleotide positions at or around (e.g., within 3 nucleotides of) the following nucleotides: 135 and 153, 143 and 161, 143 and 163, 144 and 162, 145 and 163, 150 and 168, 151 and 169, 1528 and 1546, 1530 and 1548, 1532 and 1550, 1533 and 1551, 1535 and 1553, 1602 and 1620, 2612 and 2630, and 2773 and 2791. In some embodiments, the target sequence has a start nucleotide position between 135 and 151 and an end nucleotide position between 153 and 169, or a start nucleotide position between 1528 and 1535 and an end nucleotide position between 1546 and 1553. In certain embodiments, the target sequence corresponds to nucleotide positions 135-153, 143-161, 144-162, 145-163, 150-168, or 1535-1553 of the human ANGPTL3 mRNA sequence, where the start and end positions may vary within 3 nucleotides of the numbered positions. In some embodiments, the target sequence is a sequence listed in Table 1 as a sense sequence, or a sequence that includes at least 80% nucleotides (e.g., at least 90%) of the listed sequence.

In some embodiments, a dsRNA of the present disclosure comprises a sense strand comprising a sense sequence shown in Table 1. For example, the sense strand comprises a sequence selected from SEQ ID NOs: 13-15, 47-51, 55, 129, 142, 165, 171, 172, and 212, or a sequence having at least 15, 16, 17, or 18 contiguous nucleotides derived from said selected sequence. In certain embodiments, the sense strand comprises a sequence selected from SEQ ID NOs: 13-15, 51, 165, and 171.

In some embodiments, a dsRNA of the present disclosure comprises an antisense strand comprising an antisense sequence shown in Table 1. In some embodiments, the antisense strand comprises a sequence selected from SEQ ID NOs: 227-229, 261-265, 269, 343, 356, 379, 385, 386, and 426, or a sequence having at least 15, 16, 17, or 18 contiguous nucleotides derived from said selected sequence. In certain embodiments, the antisense strand comprises a sequence selected from SEQ ID NOs: 227-229, 265, 379, and 385.

In some embodiments, a dsRNA of the present disclosure comprises a sense strand comprising a sense sequence shown in Table 1 and an antisense strand comprising an antisense sequence shown in Table 1. In some embodiments, the sense and antisense strands respectively comprise the sequences of:
SEQ ID NOs: 13 and 227;
SEQ ID NOs: 14 and 228;
SEQ ID NOs: 15 and 229;
SEQ ID NOs: 47 and 261;
SEQ ID NOs: 48 and 262;
SEQ ID NOs: 49 and 263;
SEQ ID NOs: 50 and 264;
SEQ ID NOs: 51 and 265;
SEQ ID NOs: 55 and 269;
SEQ ID NOs: 129 and 343;
SEQ ID NOs: 142 and 356;
SEQ ID NOs: 165 and 379;
SEQ ID NOs: 171 and 385;
SEQ ID NOs: 172 and 386; or
SEQ ID NOs: 212 and 426.

In certain embodiments, the sense and antisense strands respectively comprise the sequences of:
SEQ ID NOs: 13 and 227;
SEQ ID NOs: 14 and 228;
SEQ ID NOs: 15 and 229;
SEQ ID NOs: 51 and 265;
SEQ ID NOs: 165 and 379; or
SEQ ID NOs: 171 and 385.

In some embodiments, the antisense sequence is fully complementary to a sequence selected from SEQ ID NOs: 13-15, 47-51, 55, 129, 142, 165, 171, 172, and 212. In some embodiments, the antisense sequence is substantially complementary to a sequence selected from SEQ ID NOs: 13-15, 47-51, 55, 129, 142, 165, 171, 172, and 212, wherein the antisense sequence comprises at least one mismatch (e.g., one, two, three, or four mismatches) to the selected sequence.

In some embodiments, the antisense sequence is fully complementary to a sequence selected from SEQ ID NOs: 13-15, 51, 165, and 171. In some embodiments, the antisense sequence is substantially complementary to a sequence selected from SEQ ID NOs: 13-15, 51, 165, and 171, wherein the antisense sequence comprises at least one mismatch (e.g., one, two, three, or four mismatches) to the selected sequence.

In some embodiments, the antisense sequence of the ANGPTL3-targeting dsRNA comprises one or more mismatches to the target sequence (for example, due to allelic differences among individuals in a general population). For example, the antisense sequence comprises one or more mismatches (e.g., one, two, three, or four mismatches) to the target sequence. In some embodiments, the one or more mismatches are not located in the center of the region of complementarity. In some embodiments, the one or more mismatches are located within five, four, three, two, or one nucleotide of the 5′ and/or 3′ ends of the region of complementarity. For example, for a dsRNA containing a 19 nucleotide antisense sequence, in some embodiments the antisense sequence may not contain any mismatch within the central 9 nucleotides of the region of complementarity between it and its target sequence in the ANGPTL3 mRNA.

Table 1 below lists the sense and antisense sequences of exemplary siRNA constructs (CNST). The start (ST) and end (ED) nucleotide positions in NM_014495.3 (SEQ ID NO:1181) are indicated. “SEQ” denotes SEQ ID NOs.

TABLE 1
Sequences of Exemplary siRNA Constructs

| CNST | | | Sense Sequence | | Antisense Sequence | |
|---|---|---|---|---|---|---|
| # | ST | ED | (5′-3′) | SEQ | (5′-3′) | SEQ |
| 001 | 2 | 20 | UAUAUAGAGUUAAGAAGUC | 1 | GACUUCUUAACUCUAUAUA | 215 |
| 002 | 3 | 21 | AUAUAGAGUUAAGAAGUCU | 2 | AGACUUCUUAACUCUAUAU | 216 |
| 003 | 4 | 22 | UAUAGAGUUAAGAAGUCUA | 3 | UAGACUUCUUAACUCUAUA | 217 |
| 004 | 9 | 27 | AGUUAAGAAGUCUAGGUCU | 4 | AGACCUAGACUUCUUAACU | 218 |
| 005 | 10 | 28 | GUUAAGAAGUCUAGGUCUG | 5 | CAGACCUAGACUUCUUAAC | 219 |
| 006 | 11 | 29 | UUAAGAAGUCUAGGUCUGC | 6 | GCAGACCUAGACUUCUUAA | 220 |
| 007 | 12 | 30 | UAAGAAGUCUAGGUCUGCU | 7 | AGCAGACCUAGACUUCUUA | 221 |
| 008 | 13 | 31 | AAGAAGUCUAGGUCUGCUU | 8 | AAGCAGACCUAGACUUCUU | 222 |
| 009 | 14 | 32 | AGAAGUCUAGGUCUGCUUC | 9 | GAAGCAGACCUAGACUUCU | 223 |
| 010 | 15 | 33 | GAAGUCUAGGUCUGCUUCC | 10 | GGAAGCAGACCUAGACUUC | 224 |
| 011 | 16 | 34 | AAGUCUAGGUCUGCUUCCA | 11 | UGGAAGCAGACCUAGACUU | 225 |
| 012 | 141 | 159 | CAAGACAAUUCAUCAUUUG | 12 | CAAAUGAUGAAUUGUCUUG | 226 |
| 013 | 143 | 161 | AGACAAUUCAUCAUUUGAU | 13 | AUCAAAUGAUGAAUUGUCU | 227 |
| 014 | 144 | 162 | GACAAUUCAUCAUUUGAUU | 14 | AAUCAAAUGAUGAAUUGUC | 228 |
| 015 | 145 | 163 | ACAAUUCAUCAUUUGAUUC | 15 | GAAUCAAAUGAUGAAUUGU | 229 |
| 016 | 601 | 619 | GCAUCAAAGACCUUCUCCA | 16 | UGGAGAAGGUCUUUGAUGC | 230 |
| 017 | 720 | 738 | AUUUCUCUAUCUUCCAAGC | 17 | GCUUGGAAGAUAGAGAAAU | 231 |
| 018 | 723 | 741 | UCUCUAUCUUCCAAGCCAA | 18 | UUGGCUUGGAAGAUAGAGA | 232 |
| 019 | 724 | 742 | CUCUAUCUUCCAAGCCAAG | 19 | CUUGGCUUGGAAGAUAGAG | 233 |
| 020 | 725 | 743 | UCUAUCUUCCAAGCCAAGA | 20 | UCUUGGCUUGGAAGAUAGA | 234 |
| 021 | 726 | 744 | CUAUCUUCCAAGCCAAGAG | 21 | CUCUUGGCUUGGAAGAUAG | 235 |
| 022 | 748 | 766 | CAAGAACUACUCCCUUUCU | 22 | AGAAAGGGAGUAGUUCUUG | 236 |
| 023 | 750 | 768 | AGAACUACUCCCUUUCUUC | 23 | GAAGAAAGGGAGUAGUUCU | 237 |
| 024 | 751 | 769 | GAACUACUCCCUUUCUUCA | 24 | UGAAGAAAGGGAGUAGUUC | 238 |
| 025 | 752 | 770 | AACUACUCCCUUUCUUCAG | 25 | CUGAAGAAAGGGAGUAGUU | 239 |
| 026 | 785 | 803 | AAAUGUAAAACAUGAUGGC | 26 | GCCAUCAUGUUUUACAUUU | 240 |
| 027 | 786 | 804 | AAUGUAAAACAUGAUGGCA | 27 | UGCCAUCAUGUUUUACAUU | 241 |
| 028 | 790 | 808 | UAAAACAUGAUGGCAUUCC | 28 | GGAAUGCCAUCAUGUUUUA | 242 |
| 029 | 887 | 905 | UUUUCAUGUCUACUGUGAU | 29 | AUCACAGUAGACAUGAAAA | 243 |
| 030 | 888 | 906 | UUUCAUGUCUACUGUGAUG | 30 | CAUCACAGUAGACAUGAAA | 244 |
| 031 | 890 | 908 | UCAUGUCUACUGUGAUGUU | 31 | AACAUCACAGUAGACAUGA | 245 |
| 032 | 1068 | 1086 | GUUUUACGAAUUGAGUUGG | 32 | CCAACUCAAUUCGUAAAAC | 246 |
| 033 | 1069 | 1087 | UUUUACGAAUUGAGUUGGA | 33 | UCCAACUCAAUUCGUAAAA | 247 |
| 034 | 1142 | 1160 | CGAAACCAACUAUACGCUA | 34 | UAGCGUAUAGUUGGUUUCG | 248 |
| 035 | 1143 | 1161 | GAAACCAACUAUACGCUAC | 35 | GUAGCGUAUAGUUGGUUUC | 249 |
| 036 | 1144 | 1162 | AAACCAACUAUACGCUACA | 36 | UGUAGCGUAUAGUUGGUUU | 250 |
| 037 | 1145 | 1163 | AACCAACUAUACGCUACAU | 37 | AUGUAGCGUAUAGUUGGUU | 251 |
| 038 | 1235 | 1253 | UCACAAAGCAAAAGGACAC | 38 | GUGUCCUUUUGCUUUGUGA | 252 |
| 039 | 1270 | 1288 | GUUAUUCAGGAGGCUGGUG | 39 | CACCAGCCUCCUGAAUAAC | 253 |
| 040 | 1271 | 1289 | UUAUUCAGGAGGCUGGUGG | 40 | CCACCAGCCUCCUGAAUAA | 254 |
| 041 | 1272 | 1290 | UAUUCAGGAGGCUGGUGGU | 41 | ACCACCAGCCUCCUGAAUA | 255 |
| 042 | 1503 | 1521 | CUCAUUCCAAGUUAAUGUG | 42 | CACAUUAACUUGGAAUGAG | 256 |
| 043 | 1504 | 1522 | UCAUUCCAAGUUAAUGUGG | 43 | CCACAUUAACUUGGAAUGA | 257 |
| 044 | 1505 | 1523 | CAUUCCAAGUUAAUGUGGU | 44 | ACCACAUUAACUUGGAAUG | 258 |
| 045 | 1525 | 1543 | UAAUAAUCUGGUAUUAAAU | 45 | AUUUAAUACCAGAUUAUUA | 259 |
| 046 | 1526 | 1544 | AAUAAUCUGGUAUUAAAUC | 46 | GAUUUAAUACCAGAUUAUU | 260 |
| 047 | 1528 | 1546 | UAAUCUGGUAUUAAAUCCU | 47 | AGGAUUUAAUACCAGAUUA | 261 |
| 048 | 1530 | 1548 | AUCUGGUAUUAAAUCCUUA | 48 | UAAGGAUUUAAUACCAGAU | 262 |
| 049 | 1532 | 1550 | CUGGUAUUAAAUCCUUAAG | 49 | CUUAAGGAUUUAAUACCAG | 263 |
| 050 | 1533 | 1551 | UGGUAUUAAAUCCUUAAGA | 50 | UCUUAAGGAUUUAAUACCA | 264 |
| 051 | 1535 | 1553 | GUAUUAAAUCCUUAAGAGA | 51 | UCUCUUAAGGAUUUAAUAC | 265 |
| 052 | 1596 | 1614 | AUUUAAGAUUAAACAUACA | 52 | UGUAUGUUUAAUCUUAAAU | 266 |
| 053 | 1600 | 1618 | AAGAUUAAACAUACAAUCA | 53 | UGAUUGUAUGUUUAAUCUU | 267 |
| 054 | 1601 | 1619 | AGAUUAAACAUACAAUCAC | 54 | GUGAUUGUAUGUUUAAUCU | 268 |
| 055 | 1602 | 1620 | GAUUAAACAUACAAUCACA | 55 | UGUGAUUGUAUGUUUAAUC | 269 |
| 056 | 1606 | 1624 | AAACAUACAAUCACAUAAC | 56 | GUUAUGUGAUUGUAUGUUU | 270 |
| 057 | 1607 | 1625 | AACAUACAAUCACAUAACC | 57 | GGUUAUGUGAUUGUAUGUU | 271 |
| 058 | 1608 | 1626 | ACAUACAAUCACAUAACCU | 58 | AGGUUAUGUGAUUGUAUGU | 272 |
| 059 | 1610 | 1628 | AUACAAUCACAUAACCUUA | 59 | UAAGGUUAUGUGAUUGUAU | 273 |
| 060 | 1612 | 1630 | ACAAUCACAUAACCUUAAA | 60 | UUUAAGGUUAUGUGAUUGU | 274 |
| 061 | 1613 | 1631 | CAAUCACAUAACCUUAAAG | 61 | CUUUAAGGUUAUGUGAUUG | 275 |
| 062 | 1614 | 1632 | AAUCACAUAACCUUAAAGA | 62 | UCUUUAAGGUUAUGUGAUU | 276 |
| 063 | 1615 | 1633 | AUCACAUAACCUUAAAGAA | 63 | UUCUUUAAGGUUAUGUGAU | 277 |
| 064 | 1616 | 1634 | UCACAUAACCUUAAAGAAU | 64 | AUUCUUUAAGGUUAUGUGA | 278 |
| 065 | 1617 | 1635 | CACAUAACCUUAAAGAAUA | 65 | UAUUCUUUAAGGUUAUGUG | 279 |
| 066 | 1618 | 1636 | ACAUAACCUUAAAGAAUAC | 66 | GUAUUCUUUAAGGUUAUGU | 280 |
| 067 | 1619 | 1637 | CAUAACCUUAAAGAAUACC | 67 | GGUAUUCUUUAAGGUUAUG | 281 |
| 068 | 1643 | 1661 | CAUUUCUCAAUCAAAAUUC | 68 | GAAUUUUGAUUGAGAAAUG | 282 |
| 069 | 1646 | 1664 | UUCUCAAUCAAAAUUCUUA | 69 | UAAGAAUUUUGAUUGAGAA | 283 |
| 070 | 1683 | 1701 | AUUUUGUGAUGUGGGAAUC | 70 | GAUUCCCACAUCACAAAAU | 284 |
| 071 | 1684 | 1702 | UUUUGUGAUGUGGGAAUCA | 71 | UGAUUCCCACAUCACAAAA | 285 |
| 072 | 1685 | 1703 | UUUGUGAUGUGGGAAUCAA | 72 | UUGAUUCCCACAUCACAAA | 286 |
| 073 | 1686 | 1704 | UUGUGAUGUGGGAAUCAAU | 73 | AUUGAUUCCCACAUCACAA | 287 |
| 074 | 1687 | 1705 | UGUGAUGUGGGAAUCAAUU | 74 | AAUUGAUUCCCACAUCACA | 288 |
| 075 | 1688 | 1706 | GUGAUGUGGGAAUCAAUUU | 75 | AAAUUGAUUCCCACAUCAC | 289 |
| 076 | 1689 | 1707 | UGAUGUGGGAAUCAAUUUU | 76 | AAAAUUGAUUCCCACAUCA | 290 |
| 077 | 1690 | 1708 | GAUGUGGGAAUCAAUUUUA | 77 | UAAAAUUGAUUCCCACAUC | 291 |
| 078 | 1691 | 1709 | AUGUGGGAAUCAAUUUUAG | 78 | CUAAAAUUGAUUCCCACAU | 292 |
| 079 | 1692 | 1710 | UGUGGGAAUCAAUUUUAGA | 79 | UCUAAAAUUGAUUCCCACA | 293 |
| 080 | 1693 | 1711 | GUGGGAAUCAAUUUUAGAU | 80 | AUCUAAAAUUGAUUCCCAC | 294 |
| 081 | 1694 | 1712 | UGGGAAUCAAUUUUAGAUG | 81 | CAUCUAAAAUUGAUUCCCA | 295 |
| 082 | 1695 | 1713 | GGGAAUCAAUUUUAGAUGG | 82 | CCAUCUAAAAUUGAUUCCC | 296 |
| 083 | 1696 | 1714 | GGAAUCAAUUUUAGAUGGU | 83 | ACCAUCUAAAAUUGAUUCC | 297 |
| 084 | 1697 | 1715 | GAAUCAAUUUUAGAUGGUC | 84 | GACCAUCUAAAAUUGAUUC | 298 |
| 085 | 1797 | 1815 | CAUCAUAUGAGCUAAUAUC | 85 | GAUAUUAGCUCAUAUGAUG | 299 |
| 086 | 1798 | 1816 | AUCAUAUGAGCUAAUAUCA | 86 | UGAUAUUAGCUCAUAUGAU | 300 |
| 087 | 1799 | 1817 | UCAUAUGAGCUAAUAUCAC | 87 | GUGAUAUUAGCUCAUAUGA | 301 |
| 088 | 1800 | 1818 | CAUAUGAGCUAAUAUCACA | 88 | UGUGAUAUUAGCUCAUAUG | 302 |
| 089 | 1801 | 1819 | AUAUGAGCUAAUAUCACAA | 89 | UUGUGAUAUUAGCUCAUAU | 303 |
| 090 | 1824 | 1842 | CCCAGUUUAAAAAACUAGU | 90 | ACUAGUUUUUUAAACUGGG | 304 |
| 091 | 1851 | 1869 | UAAAACUCUAAACUUGACU | 91 | AGUCAAGUUUAGAGUUUUA | 305 |
| 092 | 1852 | 1870 | AAAACUCUAAACUUGACUA | 92 | UAGUCAAGUUUAGAGUUUU | 306 |
| 093 | 1853 | 1871 | AAACUCUAAACUUGACUAA | 93 | UUAGUCAAGUUUAGAGUUU | 307 |
| 094 | 1855 | 1873 | ACUCUAAACUUGACUAAAU | 94 | AUUUAGUCAAGUUUAGAGU | 308 |
| 095 | 1856 | 1874 | CUCUAAACUUGACUAAAUA | 95 | UAUUUAGUCAAGUUUAGAG | 309 |
| 096 | 1936 | 1954 | GUCAGCACAGAGUAUGUGU | 96 | ACACAUACUCUGUGCUGAC | 310 |
| 097 | 2029 | 2047 | GAUUUAUUUAUGAAACCUA | 97 | UAGGUUUCAUAAAUAAAUC | 311 |
| 098 | 2034 | 2052 | AUUUAUGAAACCUAAUGAA | 98 | UUCAUUAGGUUUCAUAAAU | 312 |
| 099 | 2035 | 2053 | UUUAUGAAACCUAAUGAAG | 99 | CUUCAUUAGGUUUCAUAAA | 313 |
| 100 | 2037 | 2055 | UAUGAAACCUAAUGAAGCA | 100 | UGCUUCAUUAGGUUUCAUA | 314 |
| 101 | 2038 | 2056 | AUGAAACCUAAUGAAGCAG | 101 | CUGCUUCAUUAGGUUUCAU | 315 |
| 102 | 2110 | 2128 | UACUAAGUCACAUUGACUU | 102 | AAGUCAAUGUGACUUAGUA | 316 |
| 103 | 2111 | 2129 | ACUAAGUCACAUUGACUUU | 103 | AAAGUCAAUGUGACUUAGU | 317 |
| 104 | 2112 | 2130 | CUAAGUCACAUUGACUUUA | 104 | UAAAGUCAAUGUGACUUAG | 318 |
| 105 | 2113 | 2131 | UAAGUCACAUUGACUUUAA | 105 | UUAAAGUCAAUGUGACUUA | 319 |
| 106 | 2135 | 2153 | GAGGUAUCACUAUACCUUA | 106 | UAAGGUAUAGUGAUACCUC | 320 |
| 107 | 2196 | 2214 | CUUAAUACUAUGAAAACAA | 107 | UUGUUUUCAUAGUAUUAAG | 321 |
| 108 | 2267 | 2285 | CAUCGAGUUAAAGUUUAUA | 108 | UAUAAACUUUAACUCGAUG | 322 |
| 109 | 2268 | 2286 | AUCGAGUUAAAGUUUAUAU | 109 | AUAUAAACUUUAACUCGAU | 323 |
| 110 | 2269 | 2287 | UCGAGUUAAAGUUUAUAUU | 110 | AAUAUAAACUUUAACUCGA | 324 |
| 111 | 2270 | 2288 | CGAGUUAAAGUUUAUAUUU | 111 | AAAUAUAAACUUUAACUCG | 325 |
| 112 | 2277 | 2295 | AAGUUUAUAUUUCCCCUAA | 112 | UUAGGGGAAAUAUAAACUU | 326 |
| 113 | 2278 | 2296 | AGUUUAUAUUUCCCCUAAA | 113 | UUUAGGGGAAAUAUAAACU | 327 |
| 114 | 2285 | 2303 | AUUUCCCCUAAAUAUGCUG | 114 | CAGCAUAUUUAGGGGAAAU | 328 |
| 115 | 2489 | 2507 | CCCUAAAUCCCUAAAGAUU | 115 | AAUCUUUAGGGAUUUAGGG | 329 |
| 116 | 2490 | 2508 | CCUAAAUCCCUAAAGAUUA | 116 | UAAUCUUUAGGGAUUUAGG | 330 |
| 117 | 2491 | 2509 | CUAAAUCCCUAAAGAUUAG | 117 | CUAAUCUUUAGGGAUUUAG | 331 |
| 118 | 2492 | 2510 | UAAAUCCCUAAAGAUUAGA | 118 | UCUAAUCUUUAGGGAUUUA | 332 |
| 119 | 2493 | 2511 | AAAUCCCUAAAGAUUAGAU | 119 | AUCUAAUCUUUAGGGAUUU | 333 |
| 120 | 2593 | 2611 | UUAAACCCAUUUGUUAAAG | 120 | CUUUAACAAAUGGGUUUAA | 334 |
| 121 | 2602 | 2620 | UUUGUUAAAGGAUAUAGUG | 121 | CACUAUAUCCUUUAACAAA | 335 |
| 122 | 2603 | 2621 | UUGUUAAAGGAUAUAGUGC | 122 | GCACUAUAUCCUUUAACAA | 336 |
| 123 | 2604 | 2622 | UGUUAAAGGAUAUAGUGCC | 123 | GGCACUAUAUCCUUUAACA | 337 |
| 124 | 2605 | 2623 | GUUAAAGGAUAUAGUGCCC | 124 | GGGCACUAUAUCCUUUAAC | 338 |
| 125 | 2606 | 2624 | UUAAAGGAUAUAGUGCCCA | 125 | UGGGCACUAUAUCCUUUAA | 339 |
| 126 | 2607 | 2625 | UAAAGGAUAUAGUGCCCAA | 126 | UUGGGCACUAUAUCCUUUA | 340 |
| 127 | 2610 | 2628 | AGGAUAUAGUGCCCAAGUU | 127 | AACUUGGGCACUAUAUCCU | 341 |
| 128 | 2611 | 2629 | GGAUAUAGUGCCCAAGUUA | 128 | UAACUUGGGCACUAUAUCC | 342 |
| 129 | 2612 | 2630 | GAUAUAGUGCCCAAGUUAU | 129 | AUAACUUGGGCACUAUAUC | 343 |
| 130 | 2613 | 2631 | AUAUAGUGCCCAAGUUAUA | 130 | UAUAACUUGGGCACUAUAU | 344 |
| 131 | 2633 | 2651 | GGUGACCUACCUUUGUCAA | 131 | UUGACAAAGGUAGGUCACC | 345 |
| 132 | 2634 | 2652 | GUGACCUACCUUUGUCAAU | 132 | AUUGACAAAGGUAGGUCAC | 346 |
| 133 | 2635 | 2653 | UGACCUACCUUUGUCAAUA | 133 | UAUUGACAAAGGUAGGUCA | 347 |
| 134 | 2663 | 2681 | AUGUAUUUCAAAUUAUCCA | 134 | UGGAUAAUUUGAAAUACAU | 348 |
| 135 | 2669 | 2687 | UUCAAAUUAUCCAAUAUAC | 135 | GUAUAUUGGAUAAUUUGAA | 349 |
| 136 | 2670 | 2688 | UCAAAUUAUCCAAUAUACA | 136 | UGUAUAUUGGAUAAUUUGA | 350 |
| 137 | 2674 | 2692 | AUUAUCCAAUAUACAUGUC | 137 | GACAUGUAUAUUGGAUAAU | 351 |
| 138 | 2675 | 2693 | UUAUCCAAUAUACAUGUCA | 138 | UGACAUGUAUAUUGGAUAA | 352 |
| 139 | 2676 | 2694 | UAUCCAAUAUACAUGUCAU | 139 | AUGACAUGUAUAUUGGAUA | 353 |
| 140 | 2687 | 2705 | CAUGUCAUAUAUAUUUUUA | 140 | UAAAAAUAUAUAUGACAUG | 354 |
| 141 | 2772 | 2790 | AGUACAAAAUAAUAAAGGU | 141 | ACCUUUAUUAUUUUGUACU | 355 |
| 142 | 2773 | 2791 | GUACAAAAUAAUAAAGGUA | 142 | UACCUUUAUUAUUUUGUAC | 356 |
| 143 | 2802 | 2820 | AUAAUUUUCAGGACCACAG | 143 | CUGUGGUCCUGAAAAUUAU | 357 |
| 144 | 2804 | 2822 | AAUUUUCAGGACCACAGAC | 144 | GUCUGUGGUCCUGAAAAUU | 358 |
| 145 | 2806 | 2824 | UUUUCAGGACCACAGACUA | 145 | UAGUCUGUGGUCCUGAAAA | 359 |
| 146 | 2807 | 2825 | UUUCAGGACCACAGACUAA | 146 | UUAGUCUGUGGUCCUGAAA | 360 |
| 147 | 2808 | 2826 | UUCAGGACCACAGACUAAG | 147 | CUUAGUCUGUGGUCCUGAA | 361 |
| 148 | 2809 | 2827 | UCAGGACCACAGACUAAGC | 148 | GCUUAGUCUGUGGUCCUGA | 362 |
| 149 | 2811 | 2829 | AGGACCACAGACUAAGCUG | 149 | CAGCUUAGUCUGUGGUCCU | 363 |
| 150 | 2812 | 2830 | GGACCACAGACUAAGCUGU | 150 | ACAGCUUAGUCUGUGGUCC | 364 |
| 151 | 2813 | 2831 | GACCACAGACUAAGCUGUC | 151 | GACAGCUUAGUCUGUGGUC | 365 |
| 152 | 2847 | 2865 | UUUUUUAGGGCCAGAAUAC | 152 | GUAUUCUGGCCCUAAAAAA | 366 |
| 153 | 2848 | 2866 | UUUUUAGGGCCAGAAUACC | 153 | GGUAUUCUGGCCCUAAAAA | 367 |
| 154 | 2849 | 2867 | UUUUAGGGCCAGAAUACCA | 154 | UGGUAUUCUGGCCCUAAAA | 368 |
| 155 | 2850 | 2868 | UUUAGGGCCAGAAUACCAA | 155 | UUGGUAUUCUGGCCCUAAA | 369 |
| 156 | 2851 | 2869 | UUAGGGCCAGAAUACCAAA | 156 | UUUGGUAUUCUGGCCCUAA | 370 |
| 157 | 2852 | 2870 | UAGGGCCAGAAUACCAAAA | 157 | UUUUGGUAUUCUGGCCCUA | 371 |
| 158 | 2853 | 2871 | AGGGCCAGAAUACCAAAAU | 158 | AUUUUGGUAUUCUGGCCCU | 372 |
| 159 | 2890 | 2908 | AAAUUGGACAAUUUCAAAU | 159 | AUUUGAAAUUGUCCAAUUU | 373 |
| 160 | 2892 | 2910 | AUUGGACAAUUUCAAAUGC | 160 | GCAUUUGAAAUUGUCCAAU | 374 |
| 161 | 2893 | 2911 | UUGGACAAUUUCAAAUGCA | 161 | UGCAUUUGAAAUUGUCCAA | 375 |
| 162 | 2926 | 2944 | UUAAUAUAUGAGUUGCUUC | 162 | GAAGCAACUCAUAUAUUAA | 376 |
| 163 | 133 | 151 | GAAUUGAUCAAGACAAUUC | 163 | GAAUUGUCUUGAUCAAUUC | 377 |
| 164 | 134 | 152 | AAUUGAUCAAGACAAUUCA | 164 | UGAAUUGUCUUGAUCAAUU | 378 |
| 165 | 135 | 153 | AUUGAUCAAGACAAUUCAU | 165 | AUGAAUUGUCUUGAUCAAU | 379 |
| 166 | 136 | 154 | UUGAUCAAGACAAUUCAUC | 166 | GAUGAAUUGUCUUGAUCAA | 380 |
| 167 | 137 | 155 | UGAUCAAGACAAUUCAUCA | 167 | UGAUGAAUUGUCUUGAUCA | 381 |
| 168 | 138 | 156 | GAUCAAGACAAUUCAUCAU | 168 | AUGAUGAAUUGUCUUGAUC | 382 |
| 169 | 139 | 157 | AUCAAGACAAUUCAUCAUU | 169 | AAUGAUGAAUUGUCUUGAU | 383 |
| 170 | 140 | 158 | UCAAGACAAUUCAUCAUUU | 170 | AAAUGAUGAAUUGUCUUGA | 384 |
| 171 | 150 | 168 | UCAUCAUUUGAUUCUCUAU | 171 | AUAGAGAAUCAAAUGAUGA | 385 |
| 172 | 151 | 169 | CAUCAUUUGAUUCUCUAUC | 172 | GAUAGAGAAUCAAAUGAUG | 386 |
| 173 | 152 | 170 | AUCAUUUGAUUCUCUAUCU | 173 | AGAUAGAGAAUCAAAUGAU | 387 |
| 174 | 676 | 694 | UAGAAAAUCAGCUCAGAAG | 174 | CUUCUGAGCUGAUUUUCUA | 388 |
| 175 | 678 | 696 | GAAAAUCAGCUCAGAAGGA | 175 | UCCUUCUGAGCUGAUUUUC | 389 |
| 176 | 679 | 697 | AAAAUCAGCUCAGAAGGAC | 176 | GUCCUUCUGAGCUGAUUUU | 390 |
| 177 | 680 | 698 | AAAUCAGCUCAGAAGGACU | 177 | AGUCCUUCUGAGCUGAUUU | 391 |
| 178 | 681 | 699 | AAUCAGCUCAGAAGGACUA | 178 | UAGUCCUUCUGAGCUGAUU | 392 |
| 179 | 798 | 816 | GAUGGCAUUCCUGCUGAAU | 179 | AUUCAGCAGGAAUGCCAUC | 393 |
| 180 | 803 | 821 | CAUUCCUGCUGAAUGUACC | 180 | GGUACAUUCAGCAGGAAUG | 394 |
| 181 | 806 | 824 | UCCUGCUGAAUGUACCACC | 181 | GGUGGUACAUUCAGCAGGA | 395 |
| 182 | 808 | 826 | CUGCUGAAUGUACCACCAU | 182 | AUGGUGGUACAUUCAGCAG | 396 |
| 183 | 809 | 827 | UGCUGAAUGUACCACCAUU | 183 | AAUGGUGGUACAUUCAGCA | 397 |
| 184 | 810 | 828 | GCUGAAUGUACCACCAUUU | 184 | AAAUGGUGGUACAUUCAGC | 398 |
| 185 | 811 | 829 | CUGAAUGUACCACCAUUUA | 185 | UAAAUGGUGGUACAUUCAG | 399 |
| 186 | 834 | 852 | AGAGGUGAACAUACAAGUG | 186 | CACUUGUAUGUUCACCUCU | 400 |
| 187 | 835 | 853 | GAGGUGAACAUACAAGUGG | 187 | CCACUUGUAUGUUCACCUC | 401 |
| 188 | 836 | 854 | AGGUGAACAUACAAGUGGC | 188 | GCCACUUGUAUGUUCACCU | 402 |
| 189 | 837 | 855 | GGUGAACAUACAAGUGGCA | 189 | UGCCACUUGUAUGUUCACC | 403 |
| 190 | 848 | 866 | AAGUGGCAUGUAUGCCAUC | 190 | GAUGGCAUACAUGCCACUU | 404 |
| 191 | 849 | 867 | AGUGGCAUGUAUGCCAUCA | 191 | UGAUGGCAUACAUGCCACU | 405 |
| 192 | 850 | 868 | GUGGCAUGUAUGCCAUCAG | 192 | CUGAUGGCAUACAUGCCAC | 406 |
| 193 | 851 | 869 | UGGCAUGUAUGCCAUCAGA | 193 | UCUGAUGGCAUACAUGCCA | 407 |
| 194 | 1151 | 1169 | CUAUACGCUACAUCUAGUU | 194 | AACUAGAUGUAGCGUAUAG | 408 |
| 195 | 1243 | 1261 | CAAAAGGACACUUCAACUG | 195 | CAGUUGAAGUGUCCUUUUG | 409 |
| 196 | 1244 | 1262 | AAAAGGACACUUCAACUGU | 196 | ACAGUUGAAGUGUCCUUUU | 410 |
| 197 | 1245 | 1263 | AAAGGACACUUCAACUGUC | 197 | GACAGUUGAAGUGUCCUUU | 411 |
| 198 | 1246 | 1264 | AAGGACACUUCAACUGUCC | 198 | GGACAGUUGAAGUGUCCUU | 412 |
| 199 | 1247 | 1265 | AGGACACUUCAACUGUCCA | 199 | UGGACAGUUGAAGUGUCCU | 413 |
| 200 | 1248 | 1266 | GGACACUUCAACUGUCCAG | 200 | CUGGACAGUUGAAGUGUCC | 414 |
| 201 | 1261 | 1279 | GUCCAGAGGGUUAUUCAGG | 201 | CCUGAAUAACCCUCUGGAC | 415 |
| 202 | 1262 | 1280 | UCCAGAGGGUUAUUCAGGA | 202 | UCCUGAAUAACCCUCUGGA | 416 |
| 203 | 1263 | 1281 | CCAGAGGGUUAUUCAGGAG | 203 | CUCCUGAAUAACCCUCUGG | 417 |
| 204 | 1264 | 1282 | CAGAGGGUUAUUCAGGAGG | 204 | CCUCCUGAAUAACCCUCUG | 418 |
| 205 | 1265 | 1283 | AGAGGGUUAUUCAGGAGGC | 205 | GCCUCCUGAAUAACCCUCU | 419 |
| 206 | 1266 | 1284 | GAGGGUUAUUCAGGAGGCU | 206 | AGCCUCCUGAAUAACCCUC | 420 |
| 207 | 1267 | 1285 | AGGGUUAUUCAGGAGGCUG | 207 | CAGCCUCCUGAAUAACCCU | 421 |
| 208 | 1269 | 1287 | GGUUAUUCAGGAGGCUGGU | 208 | ACCAGCCUCCUGAAUAACC | 422 |
| 209 | 1367 | 1385 | AAGAGGAUUAUCUUGGAAG | 209 | CUUCCAAGAUAAUCCUCUU | 423 |
| 210 | 1368 | 1386 | AGAGGAUUAUCUUGGAAGU | 210 | ACUUCCAAGAUAAUCCUCU | 424 |
| 211 | 1369 | 1387 | GAGGAUUAUCUUGGAAGUC | 211 | GACUUCCAAGAUAAUCCUC | 425 |
| 212 | 143 | 163 | AGACAAUUCAUCAUUUGAU | 212 | GAAUCAAAUGAUGAAUUGUC | 426 |
| | | | UC | | U | |
| 213 | 143 | 162 | AGACAAUUCAUCAUUUGAU | 213 | AAUCAAAUGAUGAAUUGUCU | 427 |
| | | | U | | | |
| 214 | 144 | 163 | GACAAUUCAUCAUUUGAUU | 214 | GAAUCAAAUGAUGAAUUGUC | 428 |
| | | | C | | | |

### I.4 Nucleotide Modifications

A dsRNA of the present disclosure may comprise one or more modifications, e.g., to enhance cellular uptake, affinity for the target sequence, inhibitory activity, and/or stability. Modifications may include any modification known in the art, including, for example, end modifications, base modifications, sugar modifications/replacements, and backbone modifications. End modifications may include, for example, 5′ end modifications (e.g., phosphorylation, conjugation, and inverted linkages) and 3′ end modifications (e.g., conjugation, DNA nucleotides, and inverted linkages). Base modifications may include, e.g., replacement with stabilizing bases, destabilizing bases or bases that base-pair with an expanded repertoire of partners, removal of bases (abasic modifications of nucleotides), or conjugated bases. Sugar modifications or replacements may include, e.g., modifications at the 2′ or 4′ position of the sugar moiety, or replacement of the sugar moiety. Backbone modifications may include, for example, modification or replacement of the phosphodiester linkages, e.g., with one or more phosphorothioates, phosphorodithioates, phosphotriesters, methyl and other alkyl phosphonates, phosphinates, and phosphoramidates.

As used herein, the term “nucleotide” includes naturally occurring or modified nucleotide, or a surrogate replacement moiety. A modified nucleotide is a non-naturally occurring nucleotide and is also referred to herein as a “nucleotide analog.” One of ordinary skill in the art would understand that guanine, cytosine, adenine, uracil, or thymine in a nucleotide may be replaced by other moieties without substantially altering the base-pairing properties of the modified nucleotide. For example, a nucleotide comprising inosine as its base may base-pair with nucleotides containing adenine, cytosine, or uracil. Hence, nucleotides containing uracil, guanine, or adenine may be replaced in the nucleotide sequences of the present disclosure by a nucleotide containing, for example, inosine. Sequences comprising such replacement moieties are included as embodiments of the present disclosure. A modified nucleotide may also be a nucleotide whose ribose moiety is replaced with a non-ribose moiety.

The dsRNAs of the present disclosure may include one or more modified nucleotides known in the art, including, without limitation, 2′-O-methyl modified nucleotides, 2′-fluoro modified nucleotides, 2′-deoxy modified nucleotides, 2′-O-methoxyethyl modified nucleotides, modified nucleotides comprising alternate internucleotide linkages such as thiophosphates and phosphorothioates, phosphotriester modified nucleotides, modified nucleotides terminally linked to a cholesterol derivative or lipophilic moiety, peptide nucleic acids (PNAs; see, e.g., Nielsen et al., Science (1991) 254:1497-500), constrained ethyl (cEt) modified nucleotides, inverted deoxy modified nucleotides, inverted dideoxy modified nucleotides, locked nucleic acid modified nucleotides, abasic modifications of nucleotides, 2′-amino modified nucleotides, 2′-alkyl modified nucleotides, morpholino-modified nucleotides, phosphoramidate modified nucleotides, modified nucleotides comprising modifications at other sites of the sugar or base of an oligonucleotide, and non-natural base-containing modified nucleotides. In some embodiments, at least one of the one or more modified nucleotides is a 2′-O-methyl nucleotide, a 5′-phosphorothioate nucleotide, or a terminal nucleotide linked to a cholesterol derivative, lipophilic or other targeting moiety. The incorporation of 2′-O-methyl, 2′-O-ethyl, 2′-O-propryl, 2′-O-alkyl, 2′-O-aminoalkyl, or 2′-deoxy-2′-fluoro (i.e., 2′-fluoro) groups in nucleosides of an oligonucleotide may confer enhanced hybridization properties and/or enhanced nuclease stability to the oligonucleotide. Further, oligonucleotides containing phosphorothioate backbones (e.g., phosphorothioate linkage between two neighboring nucleotides at one or more positions of the dsRNA) may have enhanced nuclease stability. In some embodiments, the dsRNA may contain nucleotides with a modified ribose, such as locked nucleic acid (LNA) units.

In some embodiments, a dsRNA of the present disclosure comprises one or more 2′-O-methyl nucleotides and one or more 2′-fluoro nucleotides. In some embodiments, the dsRNA comprises two or more 2′-O-methyl nucleotides and two or more 2′-fluoro nucleotides. In some embodiments, the dsRNA comprises two or more 2′-O-methyl nucleotides (OMe) and two or more 2′-fluoro nucleotides (F) in an alternating pattern, e.g., the pattern OMe-F-OMe-F or the pattern F-OMe-F-OMe. In some embodiments, the dsRNA comprises up to 10 contiguous nucleotides that are each a 2′-O-methyl nucleotide. In some embodiments, the dsRNA comprises up to 10 contiguous nucleotides that are each a 2′-fluoro nucleotide. In some embodiments, the dsRNA comprises two or more 2′-fluoro nucleotides at the 5′ or 3′ end of the antisense strand.

In some embodiments, a dsRNA of the present disclosure comprises one or more phosphorothioate groups. In some embodiments, a dsRNA of the present disclosure comprises two or more, three or more, four or more, five or more, six or more, seven or more, eight or more, nine or more, or 10 or more phosphorothioate groups. In some embodiments, the dsRNA does not comprise any phosphorothioate group.

In some embodiments, the dsRNA comprises one or more phosphotriester groups. In some embodiments, the dsRNA comprises two or more, three or more, four or more, five or more, six or more, seven or more, eight or more, nine or more, or 10 or more phosphotriester groups. In some embodiments, the dsRNA does not comprise any phosphotriester group.

In some embodiments, the dsRNA comprises a modified ribonucleoside such as a deoxyribonucleoside, including, for example, deoxyribonucleoside overhang(s), and one or more deoxyribonucleosides within the double-stranded portion of a dsRNA. However, it is self-evident that under no circumstances is a double-stranded DNA molecule encompassed by the term “dsRNA.”

In some embodiments, the dsRNA comprises two or more, three or more, four or more, five or more, six or more, seven or more, eight or more, nine or more, or 10 or more different modified nucleotides described herein. In some embodiments, the dsRNA comprises up to two contiguous modified nucleotides, up to three contiguous modified nucleotides, up to four contiguous modified nucleotides, up to five contiguous modified nucleotides, up to six contiguous modified nucleotides, up to seven contiguous modified nucleotides, up to eight contiguous modified nucleotides, up to nine contiguous modified nucleotides, or up to 10 contiguous modified nucleotides. In some embodiments, the contiguous modified nucleotides are the same modified nucleotide. In some embodiments, the contiguous modified nucleotides are two or more, three or more, four or more, five or more, six or more, seven or more, eight or more, nine or more, or ten or more different modified nucleotides.

Table 2 below lists the sequences of exemplary siRNA constructs (CNST) with modified nucleotides. The start (ST) and end (ED) nucleotide positions in NM_014495.3 (SEQ ID NO: 1181) are indicated. Abbreviations are as follows: SEQ=SEQ ID NO; mX=2′-O-Me nucleotide; fX=2′-F nucleotide; dX=DNA nucleotide; invdX=inverted dX; PO=phosphodiester linkage; and Hy=hydroxyl group. In these constructs, the sequences of their sense strands and antisense strands correspond to the sense and antisense sequences of the constructs in Table 1 with the same construct numbers, but for the inclusion of (1) the modified nucleotides mX and fX, (2) “Hy” at the 5′ and 3′ ends of both strands, (3) mC-C-mA at the 5′ end of the sense strand nucleotide sequence, (4) invdT at the 3′ end of the sense strand nucleotide sequence, and (5) dT-dT at the 3′ end of the antisense strand nucleotide sequence. In these constructs, a base-pair of nucleotides may be modified differently in some embodiments, e.g., one nucleotide in the base-pair is a 2′-O-Me ribonucleotide and the other is a 2′-F nucleotide. In some embodiments, the antisense strand comprises two 2′-F nucleotides at its 5′ end.

TABLE 2
Sequences of Exemplary Modified siRNA Constructs

| CNST | | | | |
|---|---|---|---|---|
| # | Sense Sequence (5′-3′) | SEQ | Antisense Sequence (5′-3′) | SEQ |
| 001 | Hy-mC-PO-mC-PO-mA-PO-fU- | 429 | Hy-fG-PO-fA-PO-mC-PO- | 643 |
| | PO-mA-PO-fU-PO-mA-PO-fU- | | fU-PO-mU-PO-fC-PO-mU- | |
| | PO-mA-PO-fG-PO-mA-PO-fG- | | PO-fU-PO-mA-PO-fA-PO- | |
| | PO-mU-PO-fU-PO-mA-PO-fA- | | mC-PO-fU-PO-mC-PO-fU- | |
| | PO-mG-PO-fA-PO-mA-PO-fG- | | PO-mA-PO-fU-PO-mA-PO- | |
| | PO-mU-PO-fC-PO-invdT-Hy | | fU-PO-mA-PO-dT-PO-dT-Hy | |
| 002 | Hy-mC-PO-mC-PO-mA-PO-fA- | 430 | Hy-fA-PO-fG-PO-mA-PO- | 644 |
| | PO-mU-PO-fA-PO-mU-PO-fA- | | fC-PO-mU-PO-fU-PO-mC- | |
| | PO-mG-PO-fA-PO-mG-PO-fU- | | PO-fU-PO-mU-PO-fA-PO- | |
| | PO-mU-PO-fA-PO-mA-PO-fG- | | mA-PO-fC-PO-mU-PO-fC- | |
| | PO-mA-PO-fA-PO-mG-PO-fU- | | PO-mU-PO-fA-PO-mU-PO- | |
| | PO-mC-PO-fU-PO-invdT-Hy | | fA-PO-mU-PO-dT-PO-dT-Hy | |
| 003 | Hy-mC-PO-mC-PO-mA-PO-fU- | 431 | Hy-fU-PO-fA-PO-mG-PO- | 645 |
| | PO-mA-PO-fU-PO-mA-PO-fG- | | fA-PO-mC-PO-fU-PO-mU- | |
| | PO-mA-PO-fG-PO-mU-PO-fU- | | PO-fC-PO-mU-PO-fU-PO- | |
| | PO-mA-PO-fA-PO-mG-PO-fA- | | mA-PO-fA-PO-mC-PO-fU- | |
| | PO-mA-PO-fG-PO-mU-PO-fC- | | PO-mC-PO-fU-PO-mA-PO- | |
| | PO-mU-PO-fA-PO-invdT-Hy | | fU-PO-mA-PO-dT-PO-dT-Hy | |
| 004 | Hy-mC-PO-mC-PO-mA-PO-fA- | 432 | Hy-fA-PO-fG-PO-mA-PO- | 646 |
| | PO-mG-PO-fU-PO-mU-PO-fA- | | fC-PO-mC-PO-fU-PO-mA- | |
| | PO-mA-PO-fG-PO-mA-PO-fA- | | PO-fG-PO-mA-PO-fC-PO- | |
| | PO-mG-PO-fU-PO-mC-PO-fU- | | mU-PO-fU-PO-mC-PO-fU- | |
| | PO-mA-PO-fG-PO-mG-PO-fU- | | PO-mU-PO-fA-PO-mA-PO- | |
| | PO-mC-PO-fU-PO-invdT-Hy | | fC-PO-mU-PO-dT-PO-dT-Hy | |
| 005 | Hy-mC-PO-mC-PO-mA-PO-fG- | 433 | Hy-fC-PO-fA-PO-mG-PO- | 647 |
| | PO-mU-PO-fU-PO-mA-PO-fA- | | fA-PO-mC-PO-fC-PO-mU- | |
| | PO-mG-PO-fA-PO-mA-PO-fG- | | PO-fA-PO-mG-PO-fA-PO- | |
| | PO-mU-PO-fC-PO-mU-PO-fA- | | mC-PO-fU-PO-mU-PO-fC- | |
| | PO-mG-PO-fG-PO-mU-PO-fC- | | PO-mU-PO-fU-PO-mA-PO- | |
| | PO-mU-PO-fG-PO-invdT-Hy | | fA-PO-mC-PO-dT-PO-dT-Hy | |
| 006 | Hy-mC-PO-mC-PO-mA-PO-fU- | 434 | Hy-fG-PO-fC-PO-mA-PO- | 648 |
| | PO-mU-PO-fA-PO-mA-PO-fG- | | fG-PO-mA-PO-fC-PO-mC- | |
| | PO-mA-PO-fA-PO-mG-PO-fU- | | PO-fU-PO-mA-PO-fG-PO- | |
| | PO-mC-PO-fU-PO-mA-PO-fG- | | mA-PO-fC-PO-mU-PO-fU- | |
| | PO-mG-PO-fU-PO-mC-PO-fU- | | PO-mC-PO-fU-PO-mU-PO- | |
| | PO-mG-PO-fC-PO-invdT-Hy | | fA-PO-mA-PO-dT-PO-dT-Hy | |
| 007 | Hy-mC-PO-mC-PO-mA-PO-fU- | 435 | Hy-fA-PO-fG-PO-mC-PO- | 649 |
| | PO-mA-PO-fA-PO-mG-PO-fA- | | fA-PO-mG-PO-fA-PO-mC- | |
| | PO-mA-PO-fG-PO-mU-PO-fC- | | PO-fC-PO-mU-PO-fA-PO- | |
| | PO-mU-PO-fA-PO-mG-PO-fG- | | mG-PO-fA-PO-mC-PO-fU- | |
| | PO-mU-PO-fC-PO-mU-PO-fG- | | PO-mU-PO-fC-PO-mU-PO- | |
| | PO-mC-PO-fU-PO-invdT-Hy | | fU-PO-mA-PO-dT-PO-dT-Hy | |
| 008 | Hy-mC-PO-mC-PO-mA-PO-fA- | 436 | Hy-fA-PO-fA-PO-mG-PO- | 650 |
| | PO-mA-PO-fG-PO-mA-PO-fA- | | fC-PO-mA-PO-fG-PO-mA- | |
| | PO-mG-PO-fU-PO-mC-PO-fU- | | PO-fC-PO-mC-PO-fU-PO- | |
| | PO-mA-PO-fG-PO-mG-PO-fU- | | mA-PO-fG-PO-mA-PO-fC- | |
| | PO-mC-PO-fU-PO-mG-PO-fC- | | PO-mU-PO-fU-PO-mC-PO- | |
| | PO-mU-PO-fU-PO-invdT-Hy | | fU-PO-mU-PO-dT-PO-dT-Hy | |
| 009 | Hy-mC-PO-mC-PO-mA-PO-fA- | 437 | Hy-fG-PO-fA-PO-mA-PO- | 651 |
| | PO-mG-PO-fA-PO-mA-PO-fG- | | fG-PO-mC-PO-fA-PO-mG- | |
| | PO-mU-PO-fC-PO-mU-PO-fA- | | PO-fA-PO-mC-PO-fC-PO- | |
| | PO-mG-PO-fG-PO-mU-PO-fC- | | mU-PO-fA-PO-mG-PO-fA- | |
| | PO-mU-PO-fG-PO-mC-PO-fU- | | PO-mC-PO-fU-PO-mU-PO- | |
| | PO-mU-PO-fC-PO-invdT-Hy | | fC-PO-mU-PO-dT-PO-dT-Hy | |
| 010 | Hy-mC-PO-mC-PO-mA-PO-fG- | 438 | Hy-fG-PO-fG-PO-mA-PO- | 652 |
| | PO-mA-PO-fA-PO-mG-PO-fU- | | fA-PO-mG-PO-fC-PO-mA- | |
| | PO-mC-PO-fU-PO-mA-PO-fG- | | PO-fG-PO-mA-PO-fC-PO- | |
| | PO-mG-PO-fU-PO-mC-PO-fU- | | mC-PO-fU-PO-mA-PO-fG- | |
| | PO-mG-PO-fC-PO-mU-PO-fU- | | PO-mA-PO-fC-PO-mU-PO- | |
| | PO-mC-PO-fC-PO-invdT-Hy | | fU-PO-mC-PO-dT-PO-dT-Hy | |
| 011 | Hy-mC-PO-mC-PO-mA-PO-fA- | 439 | Hy-fU-PO-fG-PO-mG-PO- | 653 |
| | PO-mA-PO-fG-PO-mU-PO-fC- | | fA-PO-mA-PO-fG-PO-mC- | |
| | PO-mU-PO-fA-PO-mG-PO-fG- | | PO-fA-PO-mG-PO-fA-PO- | |
| | PO-mU-PO-fC-PO-mU-PO-fG- | | mC-PO-fC-PO-mU-PO-fA- | |
| | PO-mC-PO-fU-PO-mU-PO-fC- | | PO-mG-PO-fA-PO-mC-PO- | |
| | PO-mC-PO-fA-PO-invdT-Hy | | fU-PO-mU-PO-dT-PO-dT-Hy | |
| 012 | Hy-mC-PO-mC-PO-mA-PO-fC- | 440 | Hy-fC-PO-fA-PO-mA-PO- | 654 |
| | PO-mA-PO-fA-PO-mG-PO-fA- | | fA-PO-mU-PO-fG-PO-mA- | |
| | PO-mC-PO-fA-PO-mA-PO-fU- | | PO-fU-PO-mG-PO-fA-PO- | |
| | PO-mU-PO-fC-PO-mA-PO-fU- | | mA-PO-fU-PO-mU-PO-fG- | |
| | PO-mC-PO-fA-PO-mU-PO-fU- | | PO-mU-PO-fC-PO-mU-PO- | |
| | PO-mU-PO-fG-PO-invdT-Hy | | fU-PO-mG-PO-dT-PO-dT-Hy | |
| 013 | Hy-mC-PO-mC-PO-mA-PO-fA- | 441 | Hy-fA-PO-fU-PO-mC-PO- | 655 |
| | PO-mG-PO-fA-PO-mC-PO-fA- | | fA-PO-mA-PO-fA-PO-mU- | |
| | PO-mA-PO-fU-PO-mU-PO-fC- | | PO-fG-PO-mA-PO-fU-PO- | |
| | PO-mA-PO-fU-PO-mC-PO-fA- | | mG-PO-fA-PO-mA-PO-fU- | |
| | PO-mU-PO-fU-PO-mU-PO-fG- | | PO-mU-PO-fG-PO-mU-PO- | |
| | PO-mA-PO-fU-PO-invdT-Hy | | fC-PO-mU-PO-dT-PO-dT-Hy | |
| 014 | Hy-mC-PO-mC-PO-mA-PO-fG- | 442 | Hy-fA-PO-fA-PO-mU-PO- | 656 |
| | PO-mA-PO-fC-PO-mA-PO-fA- | | fC-PO-mA-PO-fA-PO-mA- | |
| | PO-mU-PO-fU-PO-mC-PO-fA- | | PO-fU-PO-mG-PO-fA-PO- | |
| | PO-mU-PO-fC-PO-mA-PO-fU- | | mU-PO-fG-PO-mA-PO-fA- | |
| | PO-mU-PO-fU-PO-mG-PO-fA- | | PO-mU-PO-fU-PO-mG-PO- | |
| | PO-mU-PO-fU-PO-invdT-Hy | | fU-PO-mC-PO-dT-PO-dT-Hy | |
| 015 | Hy-mC-PO-mC-PO-mA-PO-fA- | 443 | Hy-fG-PO-fA-PO-mA-PO- | 657 |
| | PO-mC-PO-fA-PO-mA-PO-fU- | | fU-PO-mC-PO-fA-PO-mA- | |
| | PO-mU-PO-fC-PO-mA-PO-fU- | | PO-fA-PO-mU-PO-fG-PO- | |
| | PO-mC-PO-fA-PO-mU-PO-fU- | | mA-PO-fU-PO-mG-PO-fA- | |
| | PO-mU-PO-fG-PO-mA-PO-fU- | | PO-mA-PO-fU-PO-mU-PO- | |
| | PO-mU-PO-fC-PO-invdT-Hy | | fG-PO-mU-PO-dT-PO-dT-Hy | |
| 016 | Hy-mC-PO-mC-PO-mA-PO-fG- | 444 | Hy-fU-PO-fG-PO-mG-PO- | 658 |
| | PO-mC-PO-fA-PO-mU-PO-fC- | | fA-PO-mG-PO-fA-PO-mA- | |
| | PO-mA-PO-fA-PO-mA-PO-fG- | | PO-fG-PO-mG-PO-fU-PO- | |
| | PO-mA-PO-fC-PO-mC-PO-fU- | | mC-PO-fU-PO-mU-PO-fU- | |
| | PO-mU-PO-fC-PO-mU-PO-fC- | | PO-mG-PO-fA-PO-mU-PO- | |
| | PO-mC-PO-fA-PO-invdT-Hy | | fG-PO-mC-PO-dT-PO-dT-Hy | |
| 017 | Hy-mC-PO-mC-PO-mA-PO-fA- | 445 | Hy-fG-PO-fC-PO-mU-PO- | 659 |
| | PO-mU-PO-fU-PO-mU-PO-fC- | | fU-PO-mG-PO-fG-PO-mA- | |
| | PO-mU-PO-fC-PO-mU-PO-fA- | | PO-fA-PO-mG-PO-fA-PO- | |
| | PO-mU-PO-fC-PO-mU-PO-fU- | | mU-PO-fA-PO-mG-PO-fA- | |
| | PO-mC-PO-fC-PO-mA-PO-fA- | | PO-mG-PO-fA-PO-mA-PO- | |
| | PO-mG-PO-fC-PO-invdT-Hy | | fA-PO-mU-PO-dT-PO-dT-Hy | |
| 018 | Hy-mC-PO-mC-PO-mA-PO-fU- | 446 | Hy-fU-PO-fU-PO-mG-PO- | 660 |
| | PO-mC-PO-fU-PO-mC-PO-fU- | | fG-PO-mC-PO-fU-PO-mU- | |
| | PO-mA-PO-fU-PO-mC-PO-fU- | | PO-fG-PO-mG-PO-fA-PO- | |
| | PO-mU-PO-fC-PO-mC-PO-fA- | | mA-PO-fG-PO-mA-PO-fU- | |
| | PO-mA-PO-fG-PO-mC-PO-fC- | | PO-mA-PO-fG-PO-mA-PO- | |
| | PO-mA-PO-fA-PO-invdT-Hy | | fG-PO-mA-PO-dT-PO-dT-Hy | |
| 019 | Hy-mC-PO-mC-PO-mA-PO-fC- | 447 | Hy-fC-PO-fU-PO-mU-PO- | 661 |
| | PO-mU-PO-fC-PO-mU-PO-fA- | | fG-PO-mG-PO-fC-PO-mU- | |
| | PO-mU-PO-fC-PO-mU-PO-fU- | | PO-fU-PO-mG-PO-fG-PO- | |
| | PO-mC-PO-fC-PO-mA-PO-fA- | | mA-PO-fA-PO-mG-PO-fA- | |
| | PO-mG-PO-fC-PO-mC-PO-fA- | | PO-mU-PO-fA-PO-mG-PO- | |
| | PO-mA-PO-fG-PO-invdT-Hy | | fA-PO-mG-PO-dT-PO-dT-Hy | |
| 020 | Hy-mC-PO-mC-PO-mA-PO-fU- | 448 | Hy-fU-PO-fC-PO-mU-PO- | 662 |
| | PO-mC-PO-fU-PO-mA-PO-fU- | | fU-PO-mG-PO-fG-PO-mC- | |
| | PO-mC-PO-fU-PO-mU-PO-fC- | | PO-fU-PO-mU-PO-fG-PO- | |
| | PO-mC-PO-fA-PO-mA-PO-fG- | | mG-PO-fA-PO-mA-PO-fG- | |
| | PO-mC-PO-fC-PO-mA-PO-fA- | | PO-mA-PO-fU-PO-mA-PO- | |
| | PO-mG-PO-fA-PO-invdT-Hy | | fG-PO-mA-PO-dT-PO-dT-Hy | |
| 021 | Hy-mC-PO-mC-PO-mA-PO-fC- | 449 | Hy-fC-PO-fU-PO-mC-PO- | 663 |
| | PO-mU-PO-fA-PO-mU-PO-fC- | | fU-PO-mU-PO-fG-PO-mG- | |
| | PO-mU-PO-fU-PO-mC-PO-fC- | | PO-fC-PO-mU-PO-fU-PO- | |
| | PO-mA-PO-fA-PO-mG-PO-fC- | | mG-PO-fG-PO-mA-PO-fA- | |
| | PO-mC-PO-fA-PO-mA-PO-fG- | | PO-mG-PO-fA-PO-mU-PO- | |
| | PO-mA-PO-fG-PO-invdT-Hy | | fA-PO-mG-PO-dT-PO-dT-Hy | |
| 022 | Hy-mC-PO-mC-PO-mA-PO-fC- | 450 | Hy-fA-PO-fG-PO-mA-PO- | 664 |
| | PO-mA-PO-fA-PO-mG-PO-fA- | | fA-PO-mA-PO-fG-PO-mG- | |
| | PO-mA-PO-fC-PO-mU-PO-fA- | | PO-fG-PO-mA-PO-fG-PO- | |
| | PO-mC-PO-fU-PO-mC-PO-fC- | | mU-PO-fA-PO-mG-PO-fU- | |
| | PO-mC-PO-fU-PO-mU-PO-fU- | | PO-mU-PO-fC-PO-mU-PO- | |
| | PO-mC-PO-fU-PO-invdT-Hy | | fU-PO-mG-PO-dT-PO-dT-Hy | |
| 023 | Hy-mC-PO-mC-PO-mA-PO-fA- | 451 | Hy-fG-PO-fA-PO-mA-PO- | 665 |
| | PO-mG-PO-fA-PO-mA-PO-fC- | | fG-PO-mA-PO-fA-PO-mA- | |
| | PO-mU-PO-fA-PO-mC-PO-fU- | | PO-fG-PO-mG-PO-fG-PO- | |
| | PO-mC-PO-fC-PO-mC-PO-fU- | | mA-PO-fG-PO-mU-PO-A- | |
| | PO-mU-PO-fU-PO-mC-PO-fU- | | PO-mG-PO-fU-PO-mU-PO- | |
| | PO-mU-PO-fC-PO-invdT-Hy | | fC-PO-mU-PO-dT-PO-dT-Hy | |
| 024 | Hy-mC-PO-mC-PO-mA-PO-fG- | 452 | Hy-fU-PO-fG-PO-mA-PO- | 666 |
| | PO-mA-PO-fA-PO-mC-PO-fU- | | fA-PO-mG-PO-fA-PO-mA- | |
| | PO-mA-PO-fC-PO-mU-PO-fC- | | PO-fA-PO-mG-PO-fG-PO- | |
| | PO-mC-PO-fC-PO-mU-PO-fU- | | mG-PO-fA-PO-mG-PO-fU- | |
| | PO-mU-PO-fC-PO-mU-PO-fU- | | PO-mA-PO-fG-PO-mU-PO- | |
| | PO-mC-PO-fA-PO-invdT-Hy | | fU-PO-mC-PO-dT-PO-dT-Hy | |
| 025 | Hy-mC-PO-mC-PO-mA-PO-fA- | 453 | Hy-fC-PO-fU-PO-mG-PO- | 667 |
| | PO-mA-PO-fC-PO-mU-PO-fA- | | fA-PO-mA-PO-fG-PO-mA- | |
| | PO-mC-PO-fU-PO-mC-PO-fC- | | PO-fA-PO-mA-PO-fG-PO- | |
| | PO-mC-PO-fU-PO-mU-PO-fU- | | mG-PO-fG-PO-mA-PO-fG- | |
| | PO-mC-PO-fU-PO-mU-PO-fC- | | PO-mU-PO-fA-PO-mG-PO- | |
| | PO-mA-PO-fG-PO-invdT-Hy | | fU-PO-mU-PO-dT-PO-dT-Hy | |
| 026 | Hy-mC-PO-mC-PO-mA-PO-fA- | 454 | Hy-fG-PO-fC-PO-mC-PO- | 668 |
| | PO-mA-PO-fA-PO-mU-PO-fG- | | fA-PO-mU-PO-fC-PO-mA- | |
| | PO-mU-PO-fA-PO-mA-PO-fA- | | PO-fU-PO-mG-PO-fU-PO- | |
| | PO-mA-PO-fC-PO-mA-PO-fU- | | mU-PO-fU-PO-mU-PO-fA- | |
| | PO-mG-PO-fA-PO-mU-PO-fG- | | PO-mC-PO-fA-PO-mU-PO- | |
| | PO-mG-PO-fC-PO-invdT-Hy | | fU-PO-mU-PO-dT-PO-dT-Hy | |
| 027 | Hy-mC-PO-mC-PO-mA-PO-fA- | 455 | Hy-fU-PO-fG-PO-mC-PO- | 669 |
| | PO-mA-PO-fU-PO-mG-PO-fU- | | fC-PO-mA-PO-fU-PO-mC- | |
| | PO-mA-PO-fA-PO-mA-PO-fA- | | PO-fA-PO-mU-PO-fG-PO- | |
| | PO-mC-PO-fA-PO-mU-PO-fG- | | mU-PO-fU-PO-mU-PO-fU- | |
| | PO-mA-PO-fU-PO-mG-PO-fG- | | PO-mA-PO-fC-PO-mA-PO- | |
| | PO-mC-PO-fA-PO-invdT-Hy | | fU-PO-mU-PO-dT-PO-dT-Hy | |
| 028 | Hy-mC-PO-mC-PO-mA-PO-fU- | 456 | Hy-fG-PO-fG-PO-mA-PO- | 670 |
| | PO-mA-PO-fA-PO-mA-PO-fA- | | fA-PO-mU-PO-fG-PO-mC- | |
| | PO-mC-PO-fA-PO-mU-PO-fG- | | PO-fC-PO-mA-PO-fU-PO- | |
| | PO-mA-PO-fU-PO-mG-PO-fG- | | mC-PO-fA-PO-mU-PO-fG- | |
| | PO-mC-PO-fA-PO-mU-PO-fU- | | PO-mU-PO-fU-PO-mU-PO- | |
| | PO-mC-PO-fC-PO-invdT-Hy | | fU-PO-mA-PO-dT-PO-dT-Hy | |
| 029 | Hy-mC-PO-mC-PO-mA-PO-fU- | 457 | Hy-fA-PO-fU-PO-mC-PO- | 671 |
| | PO-mU-PO-fU-PO-mU-PO-fC- | | fA-PO-mC-PO-fA-PO-mG- | |
| | PO-mA-PO-fU-PO-mG-PO-fU- | | PO-fU-PO-mA-PO-fG-PO- | |
| | PO-mC-PO-fU-PO-mA-PO-fC- | | mA-PO-fC-PO-mA-PO-fU- | |
| | PO-mU-PO-fG-PO-mU-PO-fG- | | PO-mG-PO-fA-PO-mA-PO- | |
| | PO-mA-PO-fU-PO-invdT-Hy | | fA-PO-mA-PO-dT-PO-dT-Hy | |
| 030 | Hy-mC-PO-mC-PO-mA-PO-fU- | 458 | Hy-fC-PO-fA-PO-mU-PO- | 672 |
| | PO-mU-PO-fU-PO-mC-PO-fA- | | fC-PO-mA-PO-fC-PO-mA- | |
| | PO-mU-PO-fG-PO-mU-PO-fC- | | PO-fG-PO-mU-PO-fA-PO- | |
| | PO-mU-PO-fA-PO-mC-PO-fU- | | mG-PO-fA-PO-mC-PO-fA- | |
| | PO-mG-PO-fU-PO-mG-PO-fA- | | PO-mU-PO-fG-PO-mA-PO- | |
| | PO-mU-PO-fG-PO-invdT-Hy | | fA-PO-mA-PO-dT-PO-dT-Hy | |
| 031 | Hy-mC-PO-mC-PO-mA-PO-fU- | 459 | Hy-fA-PO-fA-PO-mC-PO- | 673 |
| | PO-mC-PO-fA-PO-mU-PO-fG- | | fA-PO-mU-PO-fC-PO-mA- | |
| | PO-mU-PO-fC-PO-mU-PO-fA- | | PO-fC-PO-mA-PO-fG-PO- | |
| | PO-mC-PO-fU-PO-mG-PO-fU- | | mU-PO-fA-PO-mG-PO-fA- | |
| | PO-mG-PO-fA-PO-mU-PO-fG- | | PO-mC-PO-fA-PO-mU-PO- | |
| | PO-mU-PO-fU-PO-invdT-Hy | | fG-PO-mA-PO-dT-PO-dT-Hy | |
| 032 | Hy-mC-PO-mC-PO-mA-PO-fG- | 460 | Hy-fC-PO-fC-PO-mA-PO- | 674 |
| | PO-mU-PO-fU-PO-mU-PO-fU- | | fA-PO-mC-PO-fU-PO-mC- | |
| | PO-mA-PO-fC-PO-mG-PO-fA- | | PO-fA-PO-mA-PO-fU-PO- | |
| | PO-mA-PO-fU-PO-mU-PO-fG- | | mU-PO-fC-PO-mG-PO-fU- | |
| | PO-mA-PO-fG-PO-mU-PO-fU- | | PO-mA-PO-fA-PO-mA-PO- | |
| | PO-mG-PO-fG-PO-invdT-Hy | | fA-PO-mC-PO-dT-PO-dT-Hy | |
| 033 | Hy-mC-PO-mC-PO-mA-PO-fU- | 461 | Hy-fU-PO-fC-PO-mC-PO- | 675 |
| | PO-mU-PO-fU-PO-mU-PO-fA- | | fA-PO-mA-PO-fC-PO-mU- | |
| | PO-mC-PO-fG-PO-mA-PO-fA- | | PO-fC-PO-mA-PO-fA-PO- | |
| | PO-mU-PO-fU-PO-mG-PO-fA- | | mU-PO-fU-PO-mC-PO-fG- | |
| | PO-mG-PO-fU-PO-mU-PO-fG- | | PO-mU-PO-fA-PO-mA-PO- | |
| | PO-mG-PO-fA-PO-invdT-Hy | | fA-PO-mA-PO-dT-PO-dT-Hy | |
| 034 | Hy-mC-PO-mC-PO-mA-PO-fC- | 462 | Hy-fU-PO-fA-PO-mG-PO- | 676 |
| | PO-mG-PO-fA-PO-mA-PO-fA- | | fC-PO-mG-PO-fU-PO-mA- | |
| | PO-mC-PO-fC-PO-mA-PO-fA- | | PO-fU-PO-mA-PO-fG-PO- | |
| | PO-mC-PO-fU-PO-mA-PO-fU- | | mU-PO-fU-PO-mG-PO-fG- | |
| | PO-mA-PO-fC-PO-mG-PO-fC- | | PO-mU-PO-fU-PO-mU-PO- | |
| | PO-mU-PO-fA-PO-invdT-Hy | | fC-PO-mG-PO-dT-PO-dT-Hy | |
| 035 | Hy-mC-PO-mC-PO-mA-PO-fG- | 463 | Hy-fG-PO-fU-PO-mA-PO- | 677 |
| | PO-mA-PO-fA-PO-mA-PO-fC- | | fG-PO-mC-PO-fG-PO-mU- | |
| | PO-mC-PO-fA-PO-mA-PO-fC- | | PO-fA-PO-mU-PO-fA-PO- | |
| | PO-mU-PO-fA-PO-mU-PO-fA- | | mG-PO-fU-PO-mU-PO-fG- | |
| | PO-mC-PO-fG-PO-mC-PO-fU- | | PO-mG-PO-fU-PO-mU-PO- | |
| | PO-mA-PO-fC-PO-invdT-Hy | | fU-PO-mC-PO-dT-PO-dT-Hy | |
| 036 | Hy-mC-PO-mC-PO-mA-PO-fA- | 464 | Hy-fU-PO-fG-PO-mU-PO- | 678 |
| | PO-mA-PO-fA-PO-mC-PO-fC- | | fA-PO-mG-PO-fC-PO-mG- | |
| | PO-mA-PO-fA-PO-mC-PO-fU- | | PO-fU-PO-mA-PO-fU-PO- | |
| | PO-mA-PO-fU-PO-mA-PO-fC- | | mA-PO-fG-PO-mU-PO-fU- | |
| | PO-mG-PO-fC-PO-mU-PO-fA- | | PO-mG-PO-fG-PO-mU-PO- | |
| | PO-mC-PO-fA-PO-invdT-Hy | | fU-PO-mU-PO-dT-PO-dT-Hy | |
| 037 | Hy-mC-PO-mC-PO-mA-PO-fA- | 465 | Hy-fA-PO-fU-PO-mG-PO- | 679 |
| | PO-mA-PO-fC-PO-mC-PO-fA- | | fU-PO-mA-PO-fG-PO-mC- | |
| | PO-mA-PO-fC-PO-mU-PO-fA- | | PO-fG-PO-mU-PO-fA-PO- | |
| | PO-mU-PO-fA-PO-mC-PO-fG- | | mU-PO-fA-PO-mG-PO-fU- | |
| | PO-mC-PO-fU-PO-mA-PO-fC- | | PO-mU-PO-fG-PO-mG-PO- | |
| | PO-mA-PO-fU-PO-invdT-Hy | | fU-PO-mU-PO-dT-PO-dT-Hy | |
| 038 | Hy-mC-PO-mC-PO-mA-PO-fU- | 466 | Hy-fG-PO-fU-PO-mG-PO- | 680 |
| | PO-mC-PO-fA-PO-mC-PO-fA- | | fU-PO-mC-PO-fC-PO-mU- | |
| | PO-mA-PO-fA-PO-mG-PO-fC- | | PO-fU-PO-mU-PO-fU-PO- | |
| | PO-mA-PO-fA-PO-mA-PO-fA- | | mG-PO-fC-PO-mU-PO-fU- | |
| | PO-mG-PO-fG-PO-mA-PO-fC- | | PO-mU-PO-fG-PO-mU-PO- | |
| | PO-mA-PO-fC-PO-invdT-Hy | | fG-PO-mA-PO-dT-PO-dT-Hy | |
| 039 | Hy-mC-PO-mC-PO-mA-PO-fG- | 467 | Hy-fC-PO-fA-PO-mC-PO- | 681 |
| | PO-mU-PO-fU-PO-mA-PO-fU- | | fC-PO-mA-PO-fG-PO-mC- | |
| | PO-mU-PO-fC-PO-mA-PO-fG- | | PO-fC-PO-mU-PO-fC-PO- | |
| | PO-mG-PO-fA-PO-mG-PO-fG- | | mC-PO-fU-PO-mG-PO-fA- | |
| | PO-mC-PO-fU-PO-mG-PO-fG- | | PO-mA-PO-fU-PO-mA-PO- | |
| | PO-mU-PO-fG-PO-invdT-Hy | | fA-PO-mC-PO-dT-PO-dT-Hy | |
| 040 | Hy-mC-PO-mC-PO-mA-PO-fU- | 468 | Hy-fC-PO-fC-PO-mA-PO- | 682 |
| | PO-mU-PO-fA-PO-mU-PO-fU- | | fC-PO-mC-PO-fA-PO-mG- | |
| | PO-mC-PO-fA-PO-mG-PO-fG- | | PO-fC-PO-mC-PO-fU-PO- | |
| | PO-mA-PO-fG-PO-mG-PO-fC- | | mC-PO-fC-PO-mU-PO-fG- | |
| | PO-mU-PO-fG-PO-mG-PO-fU- | | PO-mA-PO-fA-PO-mU-PO- | |
| | PO-mG-PO-fG-PO-invdT-Hy | | fA-PO-mA-PO-dT-PO-dT-Hy | |
| 041 | Hy-mC-PO-mC-PO-mA-PO-fU- | 469 | Hy-fA-PO-fC-PO-mC-PO- | 683 |
| | PO-mA-PO-fU-PO-mU-PO-fC- | | fA-PO-mC-PO-fC-PO-mA- | |
| | PO-mA-PO-fG-PO-mG-PO-fA- | | PO-fG-PO-mC-PO-fC-PO- | |
| | PO-mG-PO-fG-PO-mC-PO-fU- | | mU-PO-fC-PO-mC-PO-fU- | |
| | PO-mG-PO-fG-PO-mU-PO-fG- | | PO-mG-PO-fA-PO-mA-PO- | |
| | PO-mG-PO-fU-PO-invdT-Hy | | fU-PO-mA-PO-dT-PO-dT-Hy | |
| 042 | Hy-mC-PO-mC-PO-mA-PO-fC- | 470 | Hy-fC-PO-fA-PO-mC-PO- | 684 |
| | PO-mU-PO-fC-PO-mA-PO-fU- | | fA-PO-mU-PO-fU-PO-mA- | |
| | PO-mU-PO-fC-PO-mC-PO-fA- | | PO-fA-PO-mC-PO-fU-PO- | |
| | PO-mA-PO-fG-PO-mU-PO-fU- | | mU-PO-fG-PO-mG-PO-fA- | |
| | PO-mA-PO-fA-PO-mU-PO-fG- | | PO-mA-PO-fU-PO-mG-PO- | |
| | PO-mU-PO-fG-PO-invdT-Hy | | fA-PO-mG-PO-dT-PO-dT-Hy | |
| 043 | Hy-mC-PO-mC-PO-mA-PO-fU- | 471 | Hy-fC-PO-fC-PO-mA-PO- | 685 |
| | PO-mC-PO-fA-PO-mU-PO-fU- | | fC-PO-mA-PO-fU-PO-mU- | |
| | PO-mC-PO-fC-PO-mA-PO-fA- | | PO-fA-PO-mA-PO-fC-PO- | |
| | PO-mG-PO-fU-PO-mU-PO-fA- | | mU-PO-fU-PO-mG-PO-fG- | |
| | PO-mA-PO-fU-PO-mG-PO-fU- | | PO-mA-PO-fA-PO-mU-PO- | |
| | PO-mG-PO-fG-PO-invdT-Hy | | fG-PO-mA-PO-dT-PO-dT-Hy | |
| 044 | Hy-mC-PO-mC-PO-mA-PO-fC- | 472 | Hy-fA-PO-fC-PO-mC-PO- | 686 |
| | PO-mA-PO-fU-PO-mU-PO-fC- | | fA-PO-mC-PO-fA-PO-mU- | |
| | PO-mC-PO-fA-PO-mA-PO-fG- | | PO-fU-PO-mA-PO-fA-PO- | |
| | PO-mU-PO-fU-PO-mA-PO-fA- | | mC-PO-fU-PO-mU-PO-fG- | |
| | PO-mU-PO-fG-PO-mU-PO-fG- | | PO-mG-PO-fA-PO-mA-PO- | |
| | PO-mG-PO-fU-PO-invdT-Hy | | fU-PO-mG-PO-dT-PO-dT-Hy | |
| 045 | Hy-mC-PO-mC-PO-mA-PO-fU- | 473 | Hy-fA-PO-fU-PO-mU-PO- | 687 |
| | PO-mA-PO-fA-PO-mU-PO-fA- | | fU-PO-mA-PO-fA-PO-mU- | |
| | PO-mA-PO-fU-PO-mC-PO-fU- | | PO-fA-PO-mC-PO-fC-PO- | |
| | PO-mG-PO-fG-PO-mU-PO-fA- | | mA-PO-fG-PO-mA-PO-fU- | |
| | PO-mU-PO-fU-PO-mA-PO-fA- | | PO-mU-PO-fA-PO-mU-PO- | |
| | PO-mA-PO-fU-PO-invdT-Hy | | fU-PO-mA-PO-dT-PO-dT-Hy | |
| 046 | Hy-mC-PO-mC-PO-mA-PO-fA- | 474 | Hy-fG-PO-fA-PO-mU-PO- | 688 |
| | PO-mA-PO-fU-PO-mA-PO-fA- | | fU-PO-mU-PO-fA-PO-mA- | |
| | PO-mU-PO-fC-PO-mU-PO-fG- | | PO-fU-PO-mA-PO-fC-PO- | |
| | PO-mG-PO-fU-PO-mA-PO-fU- | | mC-PO-fA-PO-mG-PO-fA- | |
| | PO-mU-PO-fA-PO-mA-PO-fA- | | PO-mU-PO-fU-PO-mA-PO- | |
| | PO-mU-PO-fC-PO-invdT-Hy | | fU-PO-mU-PO-dT-PO-dT-Hy | |
| 047 | Hy-mC-PO-mC-PO-mA-PO-fU- | 475 | Hy-fA-PO-fG-PO-mG-PO- | 689 |
| | PO-mA-PO-fA-PO-mU-PO-fC- | | fA-PO-mU-PO-fU-PO-mU- | |
| | PO-mU-PO-fG-PO-mG-PO-fU- | | PO-fA-PO-mA-PO-fU-PO- | |
| | PO-mA-PO-fU-PO-mU-PO-fA- | | mA-PO-fC-PO-mC-PO-fA- | |
| | PO-mA-PO-fA-PO-mU-PO-fC- | | PO-mG-PO-fA-PO-mU-PO- | |
| | PO-mC-PO-fU-PO-invdT-Hy | | fU-PO-mA-PO-dT-PO-dT-Hy | |
| 048 | Hy-mC-PO-mC-PO-mA-PO-fA- | 476 | Hy-fU-PO-fA-PO-mA-PO- | 690 |
| | PO-mU-PO-fC-PO-mU-PO-fG- | | fG-PO-mG-PO-fA-PO-mU- | |
| | PO-mG-PO-fU-PO-mA-PO-fU- | | PO-fU-PO-mU-PO-fA-PO- | |
| | PO-mU-PO-fA-PO-mA-PO-fA- | | mA-PO-fU-PO-mA-PO-fC- | |
| | PO-mU-PO-fC-PO-mC-PO-fU- | | PO-mC-PO-fA-PO-mG-PO- | |
| | PO-mU-PO-fA-PO-invdT-Hy | | fA-PO-mU-PO-dT-PO-dT-Hy | |
| 049 | Hy-mC-PO-mC-PO-mA-PO-fC- | 477 | Hy-fC-PO-fU-PO-mU-PO- | 691 |
| | PO-mU-PO-fG-PO-mG-PO-fU- | | fA-PO-mA-PO-fG-PO-mG- | |
| | PO-mA-PO-fU-PO-mU-PO-fA- | | PO-fA-PO-mU-PO-fU-PO- | |
| | PO-mA-PO-fA-PO-mU-PO-fC- | | mU-PO-fA-PO-mA-PO-fU- | |
| | PO-mC-PO-fU-PO-mU-PO-fA- | | PO-mA-PO-fC-PO-mC-PO- | |
| | PO-mA-PO-fG-PO-invdT-Hy | | fA-PO-mG-PO-dT-PO-dT-Hy | |
| 050 | Hy-mC-PO-mC-PO-mA-PO-fU- | 478 | Hy-fU-PO-fC-PO-mU-PO- | 692 |
| | PO-mG-PO-fG-PO-mU-PO-fA- | | fU-PO-mA-PO-fA-PO-mG- | |
| | PO-mU-PO-fU-PO-mA-PO-fA- | | PO-fG-PO-mA-PO-fU-PO- | |
| | PO-mA-PO-fU-PO-mC-PO-fC- | | mU-PO-fU-PO-mA-PO-fA- | |
| | PO-mU-PO-fU-PO-mA-PO-fA- | | PO-mU-PO-fA-PO-mC-PO- | |
| | PO-mG-PO-fA-PO-invdT-Hy | | fC-PO-mA-PO-dT-PO-dT-Hy | |
| 051 | Hy-mC-PO-mC-PO-mA-PO-fG- | 479 | Hy-fU-PO-fC-PO-mU-PO- | 693 |
| | PO-mU-PO-fA-PO-mU-PO-fU- | | fC-PO-mU-PO-fU-PO-mA- | |
| | PO-mA-PO-fA-PO-mA-PO-fU- | | PO-fA-PO-mG-PO-fG-PO- | |
| | PO-mC-PO-fC-PO-mU-PO-fU- | | mA-PO-fU-PO-mU-PO-fU- | |
| | PO-mA-PO-fA-PO-mG-PO-fA- | | PO-mA-PO-fA-PO-mU-PO- | |
| | PO-mG-PO-fA-PO-invdT-Hy | | fA-PO-mC-PO-dT-PO-dT-Hy | |
| 052 | Hy-mC-PO-mC-PO-mA-PO-fA- | 480 | Hy-fU-PO-fG-PO-mU-PO- | 694 |
| | PO-mU-PO-fU-PO-mU-PO-fA- | | fA-PO-mU-PO-fG-PO-mU- | |
| | PO-mA-PO-fG-PO-mA-PO-fU- | | PO-fU-PO-mU-PO-fA-PO- | |
| | PO-mU-PO-fA-PO-mA-PO-fA- | | mA-PO-fU-PO-mC-PO-fU- | |
| | PO-mC-PO-fA-PO-mU-PO-fA- | | PO-mU-PO-fA-PO-mA-PO- | |
| | PO-mC-PO-fA-PO-invdT-Hy | | fA-PO-mU-PO-dT-PO-dT-Hy | |
| 053 | Hy-mC-PO-mC-PO-mA-PO-fA- | 481 | Hy-fU-PO-fG-PO-mA-PO- | 695 |
| | PO-mA-PO-fG-PO-mA-PO-fU- | | fU-PO-mU-PO-fG-PO-mU- | |
| | PO-mU-PO-fA-PO-mA-PO-fA- | | PO-fA-PO-mU-PO-fG-PO- | |
| | PO-mC-PO-fA-PO-mU-PO-fA- | | mU-PO-fU-PO-mU-PO-fA- | |
| | PO-mC-PO-fA-PO-mA-PO-fU- | | PO-mA-PO-fU-PO-mC-PO- | |
| | PO-mC-PO-fA-PO-invdT-Hy | | fU-PO-mU-PO-dT-PO-dT-Hy | |
| 054 | Hy-mC-PO-mC-PO-mA-PO-fA- | 482 | Hy-fG-PO-fU-PO-mG-PO- | 696 |
| | PO-mG-PO-fA-PO-mU-PO-fU- | | fA-PO-mU-PO-fU-PO-mG- | |
| | PO-mA-PO-fA-PO-mA-PO-fC- | | PO-fU-PO-mA-PO-fU-PO- | |
| | PO-mA-PO-fU-PO-mA-PO-fC- | | mG-PO-fU-PO-mU-PO-fU- | |
| | PO-mA-PO-fA-PO-mU-PO-fC- | | PO-mA-PO-fA-PO-mU-PO- | |
| | PO-mA-PO-fC-PO-invdT-Hy | | fC-PO-mU-PO-dT-PO-dT-Hy | |
| 055 | Hy-mC-PO-mC-PO-mA-PO-fG- | 483 | Hy-fU-PO-fG-PO-mU-PO- | 697 |
| | PO-mA-PO-fU-PO-mU-PO-fA- | | fG-PO-mA-PO-fU-PO-mU- | |
| | PO-mA-PO-fA-PO-mC-PO-fA- | | PO-fG-PO-mU-PO-fA-PO- | |
| | PO-mU-PO-fA-PO-mC-PO-fA- | | mU-PO-fG-PO-mU-PO-fU- | |
| | PO-mA-PO-fU-PO-mC-PO-fA- | | PO-mU-PO-fA-PO-mA-PO- | |
| | PO-mC-PO-fA-PO-invdT-Hy | | fU-PO-mC-PO-dT-PO-dT-Hy | |
| 056 | Hy-mC-PO-mC-PO-mA-PO-fA- | 484 | Hy-fG-PO-fU-PO-mU-PO- | 698 |
| | PO-mA-PO-fA-PO-mC-PO-fA- | | fA-PO-mU-PO-fG-PO-mU- | |
| | PO-mU-PO-fA-PO-mC-PO-fA- | | PO-fG-PO-mA-PO-fU-PO- | |
| | PO-mA-PO-fU-PO-mC-PO-fA- | | mU-PO-fG-PO-mU-PO-fA- | |
| | PO-mC-PO-fA-PO-mU-PO-fA- | | PO-mU-PO-fG-PO-mU-PO- | |
| | PO-mA-PO-fC-PO-invdT-Hy | | fU-PO-mU-PO-dT-PO-dT-Hy | |
| 057 | Hy-mC-PO-mC-PO-mA-PO-fA- | 485 | Hy-fG-PO-fG-PO-mU-PO- | 699 |
| | PO-mA-PO-fC-PO-mA-PO-fU- | | fU-PO-mA-PO-fU-PO-mG- | |
| | PO-mA-PO-fC-PO-mA-PO-fA- | | PO-fU-PO-mG-PO-fA-PO- | |
| | PO-mU-PO-fC-PO-mA-PO-fC- | | mU-PO-fU-PO-mG-PO-fU- | |
| | PO-mA-PO-fU-PO-mA-PO-fA- | | PO-mA-PO-fU-PO-mG-PO- | |
| | PO-mC-PO-fC-PO-invdT-Hy | | fU-PO-mU-PO-dT-PO-dT-Hy | |
| 058 | Hy-mC-PO-mC-PO-mA-PO-fA- | 486 | Hy-fA-PO-fG-PO-mG-PO- | 700 |
| | PO-mC-PO-fA-PO-mU-PO-fA- | | fU-PO-mU-PO-fA-PO-mU- | |
| | PO-mC-PO-fA-PO-mA-PO-fU- | | PO-fG-PO-mU-PO-fG-PO- | |
| | PO-mC-PO-fA-PO-mC-PO-fA- | | mA-PO-fU-PO-mU-PO-fG- | |
| | PO-mU-PO-fA-PO-mA-PO-fC- | | PO-mU-PO-fA-PO-mU-PO- | |
| | PO-mC-PO-fU-PO-invdT-Hy | | fG-PO-mU-PO-dT-PO-dT-Hy | |
| 059 | Hy-mC-PO-mC-PO-mA-PO-fA- | 487 | Hy-fU-PO-fA-PO-mA-PO- | 70 |
| | PO-mU-PO-fA-PO-mC-PO-fA- | | fG-PO-mG-PO-fU-PO-mU- | |
| | PO-mA-PO-fU-PO-mC-PO-fA- | | PO-fA-PO-mU-PO-fG-PO- | |
| | PO-mC-PO-fA-PO-mU-PO-fA- | | mU-PO-fG-PO-mA-PO-fU- | |
| | PO-mA-PO-fC-PO-mC-PO-fU- | | PO-mU-PO-fG-PO-mU-PO- | |
| | PO-mU-PO-fA-PO-invdT-Hy | | fA-PO-mU-PO-dT-PO-dT-Hy | |
| 060 | Hy-mC-PO-mC-PO-mA-PO-fA- | 488 | Hy-fU-PO-fU-PO-mU-PO- | 702 |
| | PO-mC-PO-fA-PO-mA-PO-fU- | | fA-PO-mA-PO-fG-PO-mG- | |
| | PO-mC-PO-fA-PO-mC-PO-fA- | | PO-fU-PO-mU-PO-fA-PO- | |
| | PO-mU-PO-fA-PO-mA-PO-fC- | | mU-PO-fG-PO-mU-PO-fG- | |
| | PO-mC-PO-fU-PO-mU-PO-fA- | | PO-mA-PO-fU-PO-mU-PO- | |
| | PO-mA-PO-fA-PO-invdT-Hy | | fG-PO-mU-PO-dT-PO-dT-Hy | |
| 061 | Hy-mC-PO-mC-PO-mA-PO-fC- | 489 | Hy-fC-PO-fU-PO-mU-PO- | 703 |
| | PO-mA-PO-fA-PO-mU-PO-fC- | | fU-PO-mA-PO-fA-PO-mG- | |
| | PO-mA-PO-fC-PO-mA-PO-fU- | | PO-fG-PO-mU-PO-fU-PO- | |
| | PO-mA-PO-fA-PO-mC-PO-fC- | | mA-PO-fU-PO-mG-PO-fU- | |
| | PO-mU-PO-fU-PO-mA-PO-fA- | | PO-mG-PO-fA-PO-mU-PO- | |
| | PO-mA-PO-fG-PO-invdT-Hy | | fU-PO-mG-PO-dT-PO-dT-Hy | |
| 062 | Hy-mC-PO-mC-PO-mA-PO-fA- | 490 | Hy-fU-PO-fC-PO-mU-PO- | 704 |
| | PO-mA-PO-fU-PO-mC-PO-fA- | | fU-PO-mU-PO-fA-PO-mA- | |
| | PO-mC-PO-fA-PO-mU-PO-fA- | | PO-fG-PO-mG-PO-fU-PO- | |
| | PO-mA-PO-fC-PO-mC-PO-fU- | | mU-PO-fA-PO-mU-PO-fG- | |
| | PO-mU-PO-fA-PO-mA-PO-fA- | | PO-mU-PO-fG-PO-mA-PO- | |
| | PO-mG-PO-fA-PO-invdT-Hy | | fU-PO-mU-PO-dT-PO-dT-Hy | |
| 063 | Hy-mC-PO-mC-PO-mA-PO-fA- | 491 | Hy-fU-PO-fU-PO-mC-PO- | 705 |
| | PO-mU-PO-fC-PO-mA-PO-fC- | | fU-PO-mU-PO-fU-PO-mA- | |
| | PO-mA-PO-fU-PO-mA-PO-fA- | | PO-fA-PO-mG-PO-fG-PO- | |
| | PO-mC-PO-fC-PO-mU-PO-fU- | | mU-PO-fU-PO-mA-PO-fU- | |
| | PO-mA-PO-fA-PO-mA-PO-fG- | | PO-mG-PO-fU-PO-mG-PO- | |
| | PO-mA-PO-fA-PO-invdT-Hy | | fA-PO-mU-PO-dT-PO-dT-Hy | |
| 064 | Hy-mC-PO-mC-PO-mA-PO-fU- | 492 | Hy-fA-PO-fU-PO-mU-PO- | 706 |
| | PO-mC-PO-fA-PO-mC-PO-fA- | | fC-PO-mU-PO-fU-PO-mU- | |
| | PO-mU-PO-fA-PO-mA-PO-fC- | | PO-fA-PO-mA-PO-fG-PO- | |
| | PO-mC-PO-fU-PO-mU-PO-fA- | | mG-PO-fU-PO-mU-PO-fA- | |
| | PO-mA-PO-fA-PO-mG-PO-fA- | | PO-mU-PO-fG-PO-mU-PO- | |
| | PO-mA-PO-fU-PO-invdT-Hy | | fG-PO-mA-PO-dT-PO-dT-Hy | |
| 065 | Hy-mC-PO-mC-PO-mA-PO-fC- | 493 | Hy-fU-PO-fA-PO-mU-PO- | 707 |
| | PO-mA-PO-fC-PO-mA-PO-fU- | | fU-PO-mC-PO-fU-PO-mU- | |
| | PO-mA-PO-fA-PO-mC-PO-fC- | | PO-fU-PO-mA-PO-fA-PO- | |
| | PO-mU-PO-fU-PO-mA-PO-fA- | | mG-PO-fG-PO-mU-PO-fU- | |
| | PO-mA-PO-fG-PO-mA-PO-fA- | | PO-mA-PO-fU-PO-mG-PO- | |
| | PO-mU-PO-fA-PO-invdT-Hy | | fU-PO-mG-PO-dT-PO-dT-Hy | |
| 066 | Hy-mC-PO-mC-PO-mA-PO-fA- | 494 | Hy-fG-PO-fU-PO-mA-PO- | 708 |
| | PO-mC-PO-fA-PO-mU-PO-fA- | | fU-PO-mU-PO-fC-PO-mU- | |
| | PO-mA-PO-fC-PO-mC-PO-fU- | | PO-fU-PO-mU-PO-fA-PO- | |
| | PO-mU-PO-fA-PO-mA-PO-fA- | | mA-PO-fG-PO-mG-PO-fU- | |
| | PO-mG-PO-fA-PO-mA-PO-fU- | | PO-mU-PO-fA-PO-mU-PO- | |
| | PO-mA-PO-fC-PO-invdT-Hy | | fG-PO-mU-PO-dT-PO-dT-Hy | |
| 067 | Hy-mC-PO-mC-PO-mA-PO-fC- | 495 | Hy-fG-PO-fG-PO-mU-PO- | 709 |
| | PO-mA-PO-fU-PO-mA-PO-fA- | | fA-PO-mU-PO-fU-PO-mC- | |
| | PO-mC-PO-fC-PO-mU-PO-fU- | | PO-fU-PO-mU-PO-fU-PO- | |
| | PO-mA-PO-fA-PO-mA-PO-fG- | | mA-PO-fA-PO-mG-PO-fG- | |
| | PO-mA-PO-fA-PO-mU-PO-fA- | | PO-mU-PO-fU-PO-mA-PO- | |
| | PO-mC-PO-fC-PO-invdT-Hy | | fU-PO-mG-PO-dT-PO-dT-Hy | |
| 068 | Hy-mC-PO-mC-PO-mA-PO-fC- | 496 | Hy-fG-PO-fA-PO-mA-PO- | 710 |
| | PO-mA-PO-fU-PO-mU-PO-fU- | | fU-PO-mU-PO-fU-PO-mU- | |
| | PO-mC-PO-fU-PO-mC-PO-fA- | | PO-fG-PO-mA-PO-fU-PO- | |
| | PO-mA-PO-fU-PO-mC-PO-fA- | | mU-PO-fG-PO-mA-PO-fG- | |
| | PO-mA-PO-fA-PO-mA-PO-fU- | | PO-mA-PO-fA-PO-mA-PO- | |
| | PO-mU-PO-fC-PO-invdT-Hy | | fU-PO-mG-PO-dT-PO-dT-Hy | |
| 069 | Hy-mC-PO-mC-PO-mA-PO-fU- | 497 | Hy-fU-PO-fA-PO-mA-PO- | 711 |
| | PO-mU-PO-fC-PO-mU-PO-fC- | | fG-PO-mA-PO-fA-PO-mU- | |
| | PO-mA-PO-fA-PO-mU-PO-fC- | | PO-fU-PO-mU-PO-fU-PO- | |
| | PO-mA-PO-fA-PO-mA-PO-fA- | | mG-PO-fA-PO-mU-PO-fU- | |
| | PO-mU-PO-fU-PO-mC-PO-fU- | | PO-mG-PO-fA-PO-mG-PO- | |
| | PO-mU-PO-fA-PO-invdT-Hy | | fA-PO-mA-PO-dT-PO-dT-Hy | |
| 070 | Hy-mC-PO-mC-PO-mA-PO-fA- | 498 | Hy-fG-PO-fA-PO-mU-PO- | 712 |
| | PO-mU-PO-fU-PO-mU-PO-fU- | | fU-PO-mC-PO-fC-PO-mC- | |
| | PO-mG-PO-fU-PO-mG-PO-fA- | | PO-fA-PO-mC-PO-fA-PO- | |
| | PO-mU-PO-fG-PO-mU-PO-fG- | | mU-PO-fC-PO-mA-PO-fC- | |
| | PO-mG-PO-fG-PO-mA-PO-fA- | | PO-mA-PO-fA-PO-mA-PO- | |
| | PO-mU-PO-fC-PO-invdT-Hy | | fA-PO-mU-PO-dT-PO-dT-Hy | |
| 071 | Hy-mC-PO-mC-PO-mA-PO-fU- | 499 | Hy-fU-PO-fG-PO-mA-PO- | 713 |
| | PO-mU-PO-fU-PO-mU-PO-fG- | | fU-PO-mU-PO-fC-PO-mC- | |
| | PO-mU-PO-fG-PO-mA-PO-fU- | | PO-fC-PO-mA-PO-fC-PO- | |
| | PO-mG-PO-fU-PO-mG-PO-fG- | | mA-PO-fU-PO-mC-PO-fA- | |
| | PO-mG-PO-fA-PO-mA-PO-fU- | | PO-mC-PO-fA-PO-mA-PO- | |
| | PO-mC-PO-fA-PO-invdT-Hy | | fA-PO-mA-PO-dT-PO-dT-Hy | |
| 072 | Hy-mC-PO-mC-PO-mA-PO-fU- | 500 | Hy-fU-PO-fU-PO-mG-PO- | 714 |
| | PO-mU-PO-fU-PO-mG-PO-fU- | | fA-PO-mU-PO-fU-PO-mC- | |
| | PO-mG-PO-fA-PO-mU-PO-fG- | | PO-fC-PO-mC-PO-fA-PO- | |
| | PO-mU-PO-fG-PO-mG-PO-fG- | | mC-PO-fA-PO-mU-PO-fC- | |
| | PO-mA-PO-fA-PO-mU-PO-fC- | | PO-mA-PO-fC-PO-mA-PO- | |
| | PO-mA-PO-fA-PO-invdT-Hy | | fA-PO-mA-PO-dT-PO-dT-Hy | |
| 073 | Hy-mC-PO-mC-PO-mA-PO-fU- | 501 | Hy-fA-PO-fU-PO-mU-PO- | 715 |
| | PO-mU-PO-fG-PO-mU-PO-fG- | | fG-PO-mA-PO-fU-PO-mU- | |
| | PO-mA-PO-fU-PO-mG-PO-fU- | | PO-fC-PO-mC-PO-fC-PO- | |
| | PO-mG-PO-fG-PO-mG-PO-fA- | | mA-PO-fC-PO-mA-PO-fU- | |
| | PO-mA-PO-fU-PO-mC-PO-fA- | | PO-mC-PO-fA-PO-mC-PO- | |
| | PO-mA-PO-fU-PO-invdT-Hy | | fA-PO-mA-PO-dT-PO-dT-Hy | |
| 074 | Hy-mC-PO-mC-PO-mA-PO-fU- | 502 | Hy-fA-PO-fA-PO-mU-PO- | 716 |
| | PO-mG-PO-fU-PO-mG-PO-fA- | | fU-PO-mG-PO-fA-PO-mU- | |
| | PO-mU-PO-fG-PO-mU-PO-fG- | | PO-fU-PO-mC-PO-fC-PO- | |
| | PO-mG-PO-fG-PO-mA-PO-fA- | | mC-PO-fA-PO-mC-PO-fA- | |
| | PO-mU-PO-fC-PO-mA-PO-fA- | | PO-mU-PO-fC-PO-mA-PO- | |
| | PO-mU-PO-fU-PO-invdT-Hy | | fC-PO-mA-PO-dT-PO-dT-Hy | |
| 075 | Hy-mC-PO-mC-PO-mA-PO-fG- | 503 | Hy-fA-PO-fA-PO-mA-PO- | 717 |
| | PO-mU-PO-fG-PO-mA-PO-fU- | | fU-PO-mU-PO-fG-PO-mA- | |
| | PO-mG-PO-fU-PO-mG-PO-fG- | | PO-fU-PO-mU-PO-fC-PO- | |
| | PO-mG-PO-fA-PO-mA-PO-fU- | | mC-PO-fC-PO-mA-PO-fC- | |
| | PO-mC-PO-fA-PO-mA-PO-fU- | | PO-mA-PO-fU-PO-mC-PO- | |
| | PO-mU-PO-fU-PO-invdT-Hy | | fA-PO-mC-PO-dT-PO-dT-Hy | |
| 076 | Hy-mC-PO-mC-PO-mA-PO-fU- | 504 | Hy-fA-PO-fA-PO-mA-PO- | 718 |
| | PO-mG-PO-fA-PO-mU-PO-fG- | | fA-PO-mU-PO-fU-PO-mG- | |
| | PO-mU-PO-fG-PO-mG-PO-fG- | | PO-fA-PO-mU-PO-fU-PO- | |
| | PO-mA-PO-fA-PO-mU-PO-fC- | | mC-PO-fC-PO-mC-PO-fA- | |
| | PO-mA-PO-fA-PO-mU-PO-fU- | | PO-mC-PO-fA-PO-mU-PO- | |
| | PO-mU-PO-fU-PO-invdT-Hy | | fC-PO-mA-PO-dT-PO-dT-Hy | |
| 077 | Hy-mC-PO-mC-PO-mA-PO-fG- | 505 | Hy-fU-PO-fA-PO-mA-PO- | 719 |
| | PO-mA-PO-fU-PO-mG-PO-fU- | | fA-PO-mA-PO-fU-PO-mU- | |
| | PO-mG-PO-fG-PO-mG-PO-fA- | | PO-fG-PO-mA-PO-fU-PO- | |
| | PO-mA-PO-fU-PO-mC-PO-fA- | | mU-PO-fC-PO-mC-PO-fC- | |
| | PO-mA-PO-fU-PO-mU-PO-fU- | | PO-mA-PO-fC-PO-mA-PO- | |
| | PO-mU-PO-fA-PO-invdT-Hy | | fU-PO-mC-PO-dT-PO-dT-Hy | |
| 078 | Hy-mC-PO-mC-PO-mA-PO-fA- | 506 | Hy-fC-PO-fU-PO-mA-PO- | 720 |
| | PO-mU-PO-fG-PO-mU-PO-fG- | | fA-PO-mA-PO-fA-PO-mU- | |
| | PO-mG-PO-fG-PO-mA-PO-fA- | | PO-fU-PO-mG-PO-fA-PO- | |
| | PO-mU-PO-fC-PO-mA-PO-fA- | | mU-PO-fU-PO-mC-PO-fC- | |
| | PO-mU-PO-fU-PO-mU-PO-fU- | | PO-mC-PO-fA-PO-mC-PO- | |
| | PO-mA-PO-fG-PO-invdT-Hy | | fA-PO-mU-PO-dT-PO-dT-Hy | |
| 079 | Hy-mC-PO-mC-PO-mA-PO-fU- | 507 | Hy-fU-PO-fC-PO-mU-PO- | 721 |
| | PO-mG-PO-fU-PO-mG-PO-fG- | | fA-PO-mA-PO-fA-PO-mA- | |
| | PO-mG-PO-fA-PO-mA-PO-fU- | | PO-fU-PO-mU-PO-fG-PO- | |
| | PO-mC-PO-fA-PO-mA-PO-fU- | | mA-PO-fU-PO-mU-PO-fC- | |
| | PO-mU-PO-fU-PO-mU-PO-fA- | | PO-mC-PO-fC-PO-mA-PO- | |
| | PO-mG-PO-fA-PO-invdT-Hy | | fC-PO-mA-PO-dT-PO-dT-Hy | |
| 080 | Hy-mC-PO-mC-PO-mA-PO-fG- | 508 | Hy-fA-PO-fU-PO-mC-PO- | 722 |
| | PO-mU-PO-fG-PO-mG-PO-fG- | | fU-PO-mA-PO-fA-PO-mA- | |
| | PO-mA-PO-fA-PO-mU-PO-fC- | | PO-fA-PO-mU-PO-fU-PO- | |
| | PO-mA-PO-fA-PO-mU-PO-fU- | | mG-PO-fA-PO-mU-PO-fU- | |
| | PO-mU-PO-fU-PO-mA-PO-fG- | | PO-mC-PO-fC-PO-mC-PO- | |
| | PO-mA-PO-fU-PO-invdT-Hy | | fA-PO-mC-PO-dT-PO-dT-Hy | |
| 081 | Hy-mC-PO-mC-PO-mA-PO-fU- | 509 | Hy-fC-PO-fA-PO-mU-PO- | 723 |
| | PO-mG-PO-fG-PO-mG-PO-fA- | | fC-PO-mU-PO-fA-PO-mA- | |
| | PO-mA-PO-fU-PO-mC-PO-fA- | | PO-fA-PO-mA-PO-fU-PO- | |
| | PO-mA-PO-fU-PO-mU-PO-fU- | | mU-PO-fG-PO-mA-PO-fU- | |
| | PO-mU-PO-fA-PO-mG-PO-fA- | | PO-mU-PO-fC-PO-mC-PO- | |
| | PO-mU-PO-fG-PO-invdT-Hy | | fC-PO-mA-PO-dT-PO-dT-Hy | |
| 082 | Hy-mC-PO-mC-PO-mA-PO-fG- | 510 | Hy-fC-PO-fC-PO-mA-PO- | 724 |
| | PO-mG-PO-fG-PO-mA-PO-fA- | | fU-PO-mC-PO-fU-PO-mA- | |
| | PO-mU-PO-fC-PO-mA-PO-fA- | | PO-fA-PO-mA-PO-fA-PO- | |
| | PO-mU-PO-fU-PO-mU-PO-fU- | | mU-PO-fU-PO-mG-PO-fA- | |
| | PO-mA-PO-fG-PO-mA-PO-fU- | | PO-mU-PO-fU-PO-mC-PO- | |
| | PO-mG-PO-fG-PO-invdT-Hy | | fC-PO-mC-PO-dT-PO-dT-Hy | |
| 083 | Hy-mC-PO-mC-PO-mA-PO-fG- | 511 | Hy-fA-PO-fC-PO-mC-PO- | 725 |
| | PO-mG-PO-fA-PO-mA-PO-fU- | | fA-PO-mU-PO-fC-PO-mU- | |
| | PO-mC-PO-fA-PO-mA-PO-fU- | | PO-fA-PO-mA-PO-fA-PO- | |
| | PO-mU-PO-fU-PO-mU-PO-fA- | | mA-PO-fU-PO-mU-PO-fG- | |
| | PO-mG-PO-fA-PO-mU-PO-fG- | | PO-mA-PO-fU-PO-mU-PO- | |
| | PO-mG-PO-fU-PO-invdT-Hy | | fC-PO-mC-PO-dT-PO-dT-Hy | |
| 084 | Hy-mC-PO-mC-PO-mA-PO-fG- | 512 | Hy-fG-PO-fA-PO-mC-PO- | 726 |
| | PO-mA-PO-fA-PO-mU-PO-fC- | | fC-PO-mA-PO-fU-PO-mC- | |
| | PO-mA-PO-fA-PO-mU-PO-fU- | | PO-fU-PO-mA-PO-fA-PO- | |
| | PO-mU-PO-fU-PO-mA-PO-fG- | | mA-PO-fA-PO-mU-PO-fU- | |
| | PO-mA-PO-fU-PO-mG-PO-fG- | | PO-mG-PO-fA-PO-mU-PO- | |
| | PO-mU-PO-fC-PO-invdT-Hy | | fU-PO-mC-PO-dT-PO-dT-Hy | |
| 085 | Hy-mC-PO-mC-PO-mA-PO-fC- | 513 | Hy-fG-PO-fA-PO-mU-PO- | 727 |
| | PO-mA-PO-fU-PO-mC-PO-fA- | | fA-PO-mU-PO-fU-PO-mA- | |
| | PO-mU-PO-fA-PO-mU-PO-fG- | | PO-fG-PO-mC-PO-fU-PO- | |
| | PO-mA-PO-fG-PO-mC-PO-fU- | | mC-PO-fA-PO-mU-PO-fA- | |
| | PO-mA-PO-fA-PO-mU-PO-fA- | | PO-mU-PO-fG-PO-mA-PO- | |
| | PO-mU-PO-fC-PO-invdT-Hy | | fU-PO-mG-PO-dT-PO-dT-Hy | |
| 086 | Hy-mC-PO-mC-PO-mA-PO-fA- | 514 | Hy-fU-PO-fG-PO-mA-PO- | 728 |
| | PO-mU-PO-fC-PO-mA-PO-fU- | | fU-PO-mA-PO-fU-PO-mU- | |
| | PO-mA-PO-fU-PO-mG-PO-fA- | | PO-fA-PO-mG-PO-fC-PO- | |
| | PO-mG-PO-fC-PO-mU-PO-fA- | | mU-PO-fC-PO-mA-PO-fU- | |
| | PO-mA-PO-fU-PO-mA-PO-fU- | | PO-mA-PO-fU-PO-mG-PO- | |
| | PO-mC-PO-fA-PO-invdT-Hy | | fA-PO-mU-PO-dT-PO-dT-Hy | |
| 087 | Hy-mC-PO-mC-PO-mA-PO-fU- | 515 | Hy-fG-PO-fU-PO-mG-PO- | 729 |
| | PO-mC-PO-fA-PO-mU-PO-fA- | | fA-PO-mU-PO-fA-PO-mU- | |
| | PO-mU-PO-fG-PO-mA-PO-fG- | | PO-fU-PO-mA-PO-fG-PO- | |
| | PO-mC-PO-fU-PO-mA-PO-fA- | | mC-PO-fU-PO-mC-PO-fA- | |
| | PO-mU-PO-fA-PO-mU-PO-fC- | | PO-mU-PO-fA-PO-mU-PO- | |
| | PO-mA-PO-fC-PO-invdT-Hy | | fG-PO-mA-PO-dT-PO-dT-Hy | |
| 088 | Hy-mC-PO-mC-PO-mA-PO-fC- | 516 | Hy-fU-PO-fG-PO-mU-PO- | 730 |
| | PO-mA-PO-fU-PO-mA-PO-fU- | | fG-PO-mA-PO-fU-PO-mA- | |
| | PO-mG-PO-fA-PO-mG-PO-fC- | | PO-fU-PO-mU-PO-fA-PO- | |
| | PO-mU-PO-fA-PO-mA-PO-fU- | | mG-PO-fC-PO-mU-PO-fC- | |
| | PO-mA-PO-fU-PO-mC-PO-fA- | | PO-mA-PO-fU-PO-mA-PO- | |
| | PO-mC-PO-fA-PO-invdT-Hy | | fU-PO-mG-PO-dT-PO-dT-Hy | |
| 089 | Hy-mC-PO-mC-PO-mA-PO-fA- | 517 | Hy-fU-PO-fU-PO-mG-PO- | 731 |
| | PO-mU-PO-fA-PO-mU-PO-fG- | | fU-PO-mG-PO-fA-PO-mU- | |
| | PO-mA-PO-fG-PO-mC-PO-fU- | | PO-fA-PO-mU-PO-fU-PO- | |
| | PO-mA-PO-fA-PO-mU-PO-fA- | | mA-PO-fG-PO-mC-PO-fU- | |
| | PO-mU-PO-fC-PO-mA-PO-fC- | | PO-mC-PO-fA-PO-mU-PO- | |
| | PO-mA-PO-fA-PO-invdT-Hy | | fA-PO-mU-PO-dT-PO-dT-Hy | |
| 090 | Hy-mC-PO-mC-PO-mA-PO-fC- | 518 | Hy-fA-PO-fC-PO-mU-PO- | 732 |
| | PO-mC-PO-fC-PO-mA-PO-fG- | | fA-PO-mG-PO-fU-PO-mU- | |
| | PO-mU-PO-fU-PO-mU-PO-fA- | | PO-fU-PO-mU-PO-fU-PO- | |
| | PO-mA-PO-fA-PO-mA-PO-fA- | | mU-PO-fA-PO-mA-PO-fA- | |
| | PO-mA-PO-fC-PO-mU-PO-fA- | | PO-mC-PO-fU-PO-mG-PO- | |
| | PO-mG-PO-fU-PO-invdT-Hy | | fG-PO-mG-PO-dT-PO-dT-Hy | |
| 091 | Hy-mC-PO-mC-PO-mA-PO-fU- | 519 | Hy-fA-PO-fG-PO-mU-PO- | 733 |
| | PO-mA-PO-fA-PO-mA-PO-fA- | | fC-PO-mA-PO-fA-PO-mG- | |
| | PO-mC-PO-fU-PO-mC-PO-fU- | | PO-fU-PO-mU-PO-fU-PO- | |
| | PO-mA-PO-fA-PO-mA-PO-fC- | | mA-PO-fG-PO-mA-PO-fG- | |
| | PO-mU-PO-fU-PO-mG-PO-fA- | | PO-mU-PO-fU-PO-mU-PO- | |
| | PO-mC-PO-fU-PO-invdT-Hy | | fU-PO-mA-PO-dT-PO-dT-Hy | |
| 092 | Hy-mC-PO-mC-PO-mA-PO-fA- | 520 | Hy-fU-PO-fA-PO-mG-PO- | 734 |
| | PO-mA-PO-fA-PO-mA-PO-fC- | | fU-PO-mC-PO-fA-PO-mA- | |
| | PO-mU-PO-fC-PO-mU-PO-fA- | | PO-fG-PO-mU-PO-fU-PO- | |
| | PO-mA-PO-fA-PO-mC-PO-fU- | | mU-PO-fA-PO-mG-PO-fA- | |
| | PO-mU-PO-fG-PO-mA-PO-fC- | | PO-mG-PO-fU-PO-mU-PO- | |
| | PO-mU-PO-fA-PO-invdT-Hy | | fU-PO-mU-PO-dT-PO-dT-Hy | |
| 093 | Hy-mC-PO-mC-PO-mA-PO-fA- | 521 | Hy-fU-PO-fU-PO-mA-PO- | 735 |
| | PO-mA-PO-fA-PO-mC-PO-fU- | | fG-PO-mU-PO-fC-PO-mA- | |
| | PO-mC-PO-fU-PO-mA-PO-fA- | | PO-fA-PO-mG-PO-fU-PO- | |
| | PO-mA-PO-fC-PO-mU-PO-fU- | | mU-PO-fU-PO-mA-PO-fG- | |
| | PO-mG-PO-fA-PO-mC-PO-fU- | | PO-mA-PO-fG-PO-mU-PO- | |
| | PO-mA-PO-fA-PO-invdT-Hy | | fU-PO-mU-PO-dT-PO-dT-Hy | |
| 094 | Hy-mC-PO-mC-PO-mA-PO-fA- | 522 | Hy-fA-PO-fU-PO-mU-PO- | 736 |
| | PO-mC-PO-fU-PO-mC-PO-fU- | | fU-PO-mA-PO-fG-PO-mU- | |
| | PO-mA-PO-fA-PO-mA-PO-fC- | | PO-fC-PO-mA-PO-fA-PO- | |
| | PO-mU-PO-fU-PO-mG-PO-fA- | | mG-PO-fU-PO-mU-PO-fU- | |
| | PO-mC-PO-fU-PO-mA-PO-fA- | | PO-mA-PO-fG-PO-mA-PO- | |
| | PO-mA-PO-fU-PO-invdT-Hy | | fG-PO-mU-PO-dT-PO-dT-Hy | |
| 095 | Hy-mC-PO-mC-PO-mA-PO-fC- | 523 | Hy-fU-PO-fA-PO-mU-PO- | 737 |
| | PO-mU-PO-fC-PO-mU-PO-fA- | | fU-PO-mU-PO-fA-PO-mG- | |
| | PO-mA-PO-fA-PO-mC-PO-fU- | | PO-fU-PO-mC-PO-fA-PO- | |
| | PO-mU-PO-fG-PO-mA-PO-fC- | | mA-PO-fG-PO-mU-PO-fU- | |
| | PO-mU-PO-fA-PO-mA-PO-fA- | | PO-mU-PO-fA-PO-mG-PO- | |
| | PO-mU-PO-fA-PO-invdT-Hy | | fA-PO-mG-PO-dT-PO-dT-Hy | |
| 096 | Hy-mC-PO-mC-PO-mA-PO-fG- | 524 | Hy-fA-PO-fC-PO-mA-PO- | 738 |
| | PO-mU-PO-fC-PO-mA-PO-fG- | | fC-PO-mA-PO-fU-PO-mA- | |
| | PO-mC-PO-fA-PO-mC-PO-fA- | | PO-fC-PO-mU-PO-fC-PO- | |
| | PO-mG-PO-fA-PO-mG-PO-fU- | | mU-PO-fG-PO-mU-PO-fG- | |
| | PO-mA-PO-fU-PO-mG-PO-fU- | | PO-mC-PO-fU-PO-mG-PO- | |
| | PO-mG-PO-fU-PO-invdT-Hy | | fA-PO-mC-PO-dT-PO-dT-Hy | |
| 097 | Hy-mC-PO-mC-PO-mA-PO-fG- | 525 | Hy-fU-PO-fA-PO-mG-PO- | 739 |
| | PO-mA-PO-fU-PO-mU-PO-fU- | | fG-PO-mU-PO-fU-PO-mU- | |
| | PO-mA-PO-fU-PO-mU-PO-fU- | | PO-fC-PO-mA-PO-fU-PO- | |
| | PO-mA-PO-fU-PO-mG-PO-fA- | | mA-PO-fA-PO-mA-PO-fU- | |
| | PO-mA-PO-fA-PO-mC-PO-fC- | | PO-mA-PO-fA-PO-mA-PO- | |
| | PO-mU-PO-fA-PO-invdT-Hy | | fU-PO-mC-PO-dT-PO-dT-Hy | |
| 098 | Hy-mC-PO-mC-PO-mA-PO-fA- | 526 | Hy-fU-PO-fU-PO-mC-PO- | 740 |
| | PO-mU-PO-fU-PO-mU-PO-fA- | | fA-PO-mU-PO-fU-PO-mA- | |
| | PO-mU-PO-fG-PO-mA-PO-fA- | | PO-fG-PO-mG-PO-fU-PO- | |
| | PO-mA-PO-fC-PO-mC-PO-fU- | | mU-PO-fU-PO-mC-PO-fA- | |
| | PO-mA-PO-fA-PO-mU-PO-fG- | | PO-mU-PO-fA-PO-mA-PO- | |
| | PO-mA-PO-fA-PO-invdT-Hy | | fA-PO-mU-PO-dT-PO-dT-Hy | |
| 099 | Hy-mC-PO-mC-PO-mA-PO-fU- | 527 | Hy-fC-PO-fU-PO-mU-PO- | 741 |
| | PO-mU-PO-fU-PO-mA-PO-fU- | | fC-PO-mA-PO-fU-PO-mU- | |
| | PO-mG-PO-fA-PO-mA-PO-fA- | | PO-fA-PO-mG-PO-fG-PO- | |
| | PO-mC-PO-fC-PO-mU-PO-fA- | | mU-PO-fU-PO-mU-PO-fC- | |
| | PO-mA-PO-fU-PO-mG-PO-fA- | | PO-mA-PO-fU-PO-mA-PO- | |
| | PO-mA-PO-fG-PO-invdT-Hy | | fA-PO-mA-PO-dT-PO-dT-Hy | |
| 100 | Hy-mC-PO-mC-PO-mA-PO-fU- | 528 | Hy-fU-PO-fG-PO-mC-PO- | 742 |
| | PO-mA-PO-fU-PO-mG-PO-fA- | | fU-PO-mU-PO-fC-PO-mA- | |
| | PO-mA-PO-fA-PO-mC-PO-fC- | | PO-fU-PO-mU-PO-fA-PO- | |
| | PO-mU-PO-fA-PO-mA-PO-fU- | | mG-PO-fG-PO-mU-PO-fU- | |
| | PO-mG-PO-fA-PO-mA-PO-fG- | | PO-mU-PO-fC-PO-mA-PO- | |
| | PO-mC-PO-fA-PO-invdT-Hy | | fU-PO-mA-PO-dT-PO-dT-Hy | |
| 101 | Hy-mC-PO-mC-PO-mA-PO-fA- | 529 | Hy-fC-PO-fU-PO-mG-PO- | 743 |
| | PO-mU-PO-fG-PO-mA-PO-fA- | | fC-PO-mU-PO-fU-PO-mC- | |
| | PO-mA-PO-fC-PO-mC-PO-fU- | | PO-fA-PO-mU-PO-fU-PO- | |
| | PO-mA-PO-fA-PO-mU-PO-fG- | | mA-PO-fG-PO-mG-PO-fU- | |
| | PO-mA-PO-fA-PO-mG-PO-fC- | | PO-mU-PO-fU-PO-mC-PO- | |
| | PO-mA-PO-fG-PO-invdT-Hy | | fA-PO-mU-PO-dT-PO-dT-Hy | |
| 102 | Hy-mC-PO-mC-PO-mA-PO-fU- | 530 | Hy-fA-PO-fA-PO-mG-PO- | 744 |
| | PO-mA-PO-fC-PO-mU-PO-fA- | | fU-PO-mC-PO-fA-PO-mA- | |
| | PO-mA-PO-fG-PO-mU-PO-fC- | | PO-fU-PO-mG-PO-fU-PO- | |
| | PO-mA-PO-fC-PO-mA-PO-fU- | | mG-PO-fA-PO-mC-PO-fU- | |
| | PO-mU-PO-fG-PO-mA-PO-fC- | | PO-mU-PO-fA-PO-mG-PO- | |
| | PO-mU-PO-fU-PO-invdT-Hy | | fU-PO-mA-PO-dT-PO-dT-Hy | |
| 103 | Hy-mC-PO-mC-PO-mA-PO-fA- | 531 | Hy-fA-PO-fA-PO-mA-PO- | 745 |
| | PO-mC-PO-fU-PO-mA-PO-fA- | | fG-PO-mU-PO-fC-PO-mA- | |
| | PO-mG-PO-fU-PO-mC-PO-fA- | | PO-fA-PO-mU-PO-fG-PO- | |
| | PO-mC-PO-fA-PO-mU-PO-fU- | | mU-PO-fG-PO-mA-PO-fC- | |
| | PO-mG-PO-fA-PO-mC-PO-fU- | | PO-mU-PO-fU-PO-mA-PO- | |
| | PO-mU-PO-fU-PO-invdT-Hy | | fG-PO-mU-PO-dT-PO-dT-Hy | |
| 104 | Hy-mC-PO-mC-PO-mA-PO-fC- | 532 | Hy-fU-PO-fA-PO-mA-PO- | 746 |
| | PO-mU-PO-fA-PO-mA-PO-fG- | | fA-PO-mG-PO-fU-PO-mC- | |
| | PO-mU-PO-fC-PO-mA-PO-fC- | | PO-fA-PO-mA-PO-fU-PO- | |
| | PO-mA-PO-fU-PO-mU-PO-fG- | | mG-PO-fU-PO-mG-PO-fA- | |
| | PO-mA-PO-fC-PO-mU-PO-fU- | | PO-mC-PO-fU-PO-mU-PO- | |
| | PO-mU-PO-fA-PO-invdT-Hy | | fA-PO-mG-PO-dT-PO-dT-Hy | |
| 105 | Hy-mC-PO-mC-PO-mA-PO-fU- | 533 | Hy-fU-PO-fU-PO-mA-PO- | 747 |
| | PO-mA-PO-fA-PO-mG-PO-fU- | | fA-PO-mA-PO-fG-PO-mU- | |
| | PO-mC-PO-fA-PO-mC-PO-fA- | | PO-fC-PO-mA-PO-fA-PO- | |
| | PO-mU-PO-fU-PO-mG-PO-fA- | | mU-PO-fG-PO-mU-PO-fG- | |
| | PO-mC-PO-fU-PO-mU-PO-fU- | | PO-mA-PO-fC-PO-mU-PO- | |
| | PO-mA-PO-fA-PO-invdT-Hy | | fU-PO-mA-PO-dT-PO-dT-Hy | |
| 106 | Hy-mC-PO-mC-PO-mA-PO-fG- | 534 | Hy-fU-PO-fA-PO-mA-PO- | 748 |
| | PO-mA-PO-fG-PO-mG-PO-fU- | | fG-PO-mG-PO-fU-PO-mA- | |
| | PO-mA-PO-fU-PO-mC-PO-fA- | | PO-fU-PO-mA-PO-fG-PO- | |
| | PO-mC-PO-fU-PO-mA-PO-fU- | | mU-PO-fG-PO-mA-PO-fU- | |
| | PO-mA-PO-fC-PO-mC-PO-fU- | | PO-mA-PO-fC-PO-mC-PO- | |
| | PO-mU-PO-fA-PO-invdT-Hy | | fU-PO-mC-PO-dT-PO-dT-Hy | |
| 107 | Hy-mC-PO-mC-PO-mA-PO-fC- | 535 | Hy-fU-PO-fU-PO-mG-PO- | 749 |
| | PO-mU-PO-fU-PO-mA-PO-fA- | | fU-PO-mU-PO-fU-PO-mU- | |
| | PO-mU-PO-fA-PO-mC-PO-fU- | | PO-fC-PO-mA-PO-fU-PO- | |
| | PO-mA-PO-fU-PO-mG-PO-fA- | | mA-PO-fG-PO-mU-PO-fA- | |
| | PO-mA-PO-fA-PO-mA-PO-fC- | | PO-mU-PO-fU-PO-mA-PO- | |
| | PO-mA-PO-fA-PO-invdT-Hy | | fA-PO-mG-PO-dT-PO-dT-Hy | |
| 108 | Hy-mC-PO-mC-PO-mA-PO-fC- | 536 | Hy-fU-PO-fA-PO-mU-PO- | 750 |
| | PO-mA-PO-fU-PO-mC-PO-fG- | | fA-PO-mA-PO-fA-PO-mC- | |
| | PO-mA-PO-fG-PO-mU-PO-fU- | | PO-fU-PO-mU-PO-fU-PO- | |
| | PO-mA-PO-fA-PO-mA-PO-fG- | | mA-PO-fA-PO-mC-PO-fU- | |
| | PO-mU-PO-fU-PO-mU-PO-fA- | | PO-mC-PO-fG-PO-mA-PO- | |
| | PO-mU-PO-fA-PO-invdT-Hy | | fU-PO-mG-PO-dT-PO-dT-Hy | |
| 109 | Hy-mC-PO-mC-PO-mA-PO-fA- | 537 | Hy-fA-PO-fU-PO-mA-PO- | 751 |
| | PO-mU-PO-fC-PO-mG-PO-fA- | | fU-PO-mA-PO-fA-PO-mA- | |
| | PO-mG-PO-fU-PO-mU-PO-fA- | | PO-fC-PO-mU-PO-fU-PO- | |
| | PO-mA-PO-fA-PO-mG-PO-fU- | | mU-PO-fA-PO-mA-PO-fC- | |
| | PO-mU-PO-fU-PO-mA-PO-fU- | | PO-mU-PO-fC-PO-mG-PO- | |
| | PO-mA-PO-fU-PO-invdT-Hy | | fA-PO-mU-PO-dT-PO-dT-Hy | |
| 110 | Hy-mC-PO-mC-PO-mA-PO-fU- | 538 | Hy-fA-PO-fA-PO-mU-PO- | 752 |
| | PO-mC-PO-fG-PO-mA-PO-fG- | | fA-PO-mU-PO-fA-PO-mA- | |
| | PO-mU-PO-fU-PO-mA-PO-fA- | | PO-fA-PO-mC-PO-fU-PO- | |
| | PO-mA-PO-fG-PO-mU-PO-fU- | | mU-PO-fU-PO-mA-PO-fA- | |
| | PO-mU-PO-fA-PO-mU-PO-fA- | | PO-mC-PO-fU-PO-mC-PO- | |
| | PO-mU-PO-fU-PO-invdT-Hy | | fG-PO-mA-PO-dT-PO-dT-Hy | |
| 111 | Hy-mC-PO-mC-PO-mA-PO-fC- | 539 | Hy-fA-PO-fA-PO-mA-PO- | 753 |
| | PO-mG-PO-fA-PO-mG-PO-fU- | | fU-PO-mA-PO-fU-PO-mA- | |
| | PO-mU-PO-fA-PO-mA-PO-fA- | | PO-fA-PO-mA-PO-fC-PO- | |
| | PO-mG-PO-fU-PO-mU-PO-fU- | | mU-PO-fU-PO-mU-PO-fA- | |
| | PO-mA-PO-fU-PO-mA-PO-fU- | | PO-mA-PO-fC-PO-mU-PO- | |
| | PO-mU-PO-fU-PO-invdT-Hy | | fC-PO-mG-PO-dT-PO-dT-Hy | |
| 112 | Hy-mC-PO-mC-PO-mA-PO-fA- | 540 | Hy-fU-PO-fU-PO-mA-PO- | 754 |
| | PO-mA-PO-fG-PO-mU-PO-fU- | | fG-PO-mG-PO-fG-PO-mG- | |
| | PO-mU-PO-fA-PO-mU-PO-fA- | | PO-fA-PO-mA-PO-fA-PO- | |
| | PO-mU-PO-fU-PO-mU-PO-fC- | | mU-PO-fA-PO-mU-PO-fA- | |
| | PO-mC-PO-fC-PO-mC-PO-fU- | | PO-mA-PO-fA-PO-mC-PO- | |
| | PO-mA-PO-fA-PO-invdT-Hy | | fU-PO-mU-PO-dT-PO-dT-Hy | |
| 113 | Hy-mC-PO-mC-PO-mA-PO-fA- | 541 | Hy-fU-PO-fU-PO-mU-PO- | 755 |
| | PO-mG-PO-fU-PO-mU-PO-fU- | | fA-PO-mG-PO-fG-PO-mG- | |
| | PO-mA-PO-fU-PO-mA-PO-fU- | | PO-fG-PO-mA-PO-fA-PO- | |
| | PO-mU-PO-fU-PO-mC-PO-fC- | | mA-PO-fU-PO-mA-PO-fU- | |
| | PO-mC-PO-fC-PO-mU-PO-fA- | | PO-mA-PO-fA-PO-mA-PO- | |
| | PO-mA-PO-fA-PO-invdT-Hy | | fC-PO-mU-PO-dT-PO-dT-Hy | |
| 114 | Hy-mC-PO-mC-PO-mA-PO-fA- | 542 | Hy-fC-PO-fA-PO-mG-PO- | 756 |
| | PO-mU-PO-fU-PO-mU-PO-fC- | | fC-PO-mA-PO-fU-PO-mA- | |
| | PO-mC-PO-fC-PO-mC-PO-fU- | | PO-fU-PO-mU-PO-fU-PO- | |
| | PO-mA-PO-fA-PO-mA-PO-fU- | | mA-PO-fG-PO-mG-PO-fG- | |
| | PO-mA-PO-fU-PO-mG-PO-fC- | | PO-mG-PO-fA-PO-mA-PO- | |
| | PO-mU-PO-fG-PO-invdT-Hy | | fA-PO-mU-PO-dT-PO-dT-Hy | |
| 115 | Hy-mC-PO-mC-PO-mA-PO-fC- | 543 | Hy-fA-PO-fA-PO-mU-PO- | 757 |
| | PO-mC-PO-fC-PO-mU-PO-fA- | | fC-PO-mU-PO-fU-PO-mU- | |
| | PO-mA-PO-fA-PO-mU-PO-fC- | | PO-fA-PO-mG-PO-fG-PO- | |
| | PO-mC-PO-fC-PO-mU-PO-fA- | | mG-PO-fA-PO-mU-PO-fU- | |
| | PO-mA-PO-fA-PO-mG-PO-fA- | | PO-mU-PO-fA-PO-mG-PO- | |
| | PO-mU-PO-fU-PO-invdT-Hy | | fG-PO-mG-PO-dT-PO-dT-Hy | |
| 116 | Hy-mC-PO-mC-PO-mA-PO-fC- | 544 | Hy-fU-PO-fA-PO-mA-PO- | 758 |
| | PO-mC-PO-fU-PO-mA-PO-fA- | | fU-PO-mC-PO-fU-PO-mU- | |
| | PO-mA-PO-fU-PO-mC-PO-fC- | | PO-fU-PO-mA-PO-fG-PO- | |
| | PO-mC-PO-fU-PO-mA-PO-fA- | | mG-PO-fG-PO-mA-PO-fU- | |
| | PO-mA-PO-fG-PO-mA-PO-fU- | | PO-mU-PO-fU-PO-mA-PO- | |
| | PO-mU-PO-fA-PO-invdT-Hy | | fG-PO-mG-PO-dT-PO-dT-Hy | |
| 117 | Hy-mC-PO-mC-PO-mA-PO-fC- | 545 | Hy-fC-PO-fU-PO-mA-PO- | 759 |
| | PO-mU-PO-fA-PO-mA-PO-fA- | | fA-PO-mU-PO-fC-PO-mU- | |
| | PO-mU-PO-fC-PO-mC-PO-fC- | | PO-fU-PO-mU-PO-fA-PO- | |
| | PO-mU-PO-fA-PO-mA-PO-fA- | | mG-PO-fG-PO-mG-PO-fA- | |
| | PO-mG-PO-fA-PO-mU-PO-fU- | | PO-mU-PO-fU-PO-mU-PO- | |
| | PO-mA-PO-fG-PO-invdT-Hy | | fA-PO-mG-PO-dT-PO-dT-Hy | |
| 118 | Hy-mC-PO-mC-PO-mA-PO-fU- | 546 | Hy-fU-PO-fC-PO-mU-PO- | 760 |
| | PO-mA-PO-fA-PO-mA-PO-fU- | | fA-PO-mA-PO-fU-PO-mC- | |
| | PO-mC-PO-fC-PO-mC-PO-fU- | | PO-fU-PO-mU-PO-fU-PO- | |
| | PO-mA-PO-fA-PO-mA-PO-fG- | | mA-PO-fG-PO-mG-PO-fG- | |
| | PO-mA-PO-fU-PO-mU-PO-fA- | | PO-mA-PO-fU-PO-mU-PO- | |
| | PO-mG-PO-fA-PO-invdT-Hy | | fU-PO-mA-PO-dT-PO-dT-Hy | |
| 119 | Hy-mC-PO-mC-PO-mA-PO-fA- | 547 | Hy-fA-PO-fU-PO-mC-PO- | 761 |
| | PO-mA-PO-fA-PO-mU-PO-fC- | | fU-PO-mA-PO-fA-PO-mU- | |
| | PO-mC-PO-fC-PO-mU-PO-fA- | | PO-fC-PO-mU-PO-fU-PO- | |
| | PO-mA-PO-fA-PO-mG-PO-fA- | | mU-PO-fA-PO-mG-PO-fG- | |
| | PO-mU-PO-fU-PO-mA-PO-fG- | | PO-mG-PO-fA-PO-mU-PO- | |
| | PO-mA-PO-fU-PO-invdT-Hy | | fU-PO-mU-PO-dT-PO-dT-Hy | |
| 120 | Hy-mC-PO-mC-PO-mA-PO-fU- | 548 | Hy-fC-PO-fU-PO-mU-PO- | 762 |
| | PO-mU-PO-fA-PO-mA-PO-fA- | | fU-PO-mA-PO-fA-PO-mC- | |
| | PO-mC-PO-fC-PO-mC-PO-fA- | | PO-fA-PO-mA-PO-fA-PO- | |
| | PO-mU-PO-fU-PO-mU-PO-fG- | | mU-PO-fG-PO-mG-PO-fG- | |
| | PO-mU-PO-fU-PO-mA-PO-fA- | | PO-mU-PO-fU-PO-mU-PO- | |
| | PO-mA-PO-fG-PO-invdT-Hy | | fA-PO-mA-PO-dT-PO-dT-Hy | |
| 121 | Hy-mC-PO-mC-PO-mA-PO-fU- | 549 | Hy-fC-PO-fA-PO-mC-PO- | 763 |
| | PO-mU-PO-fU-PO-mG-PO-fU- | | fU-PO-mA-PO-fU-PO-mA- | |
| | PO-mU-PO-fA-PO-mA-PO-fA- | | PO-fU-PO-mC-PO-fC-PO- | |
| | PO-mG-PO-fG-PO-mA-PO-fU- | | mU-PO-fU-PO-mU-PO-fA- | |
| | PO-mA-PO-fU-PO-mA-PO-fG- | | PO-mA-PO-fC-PO-mA-PO- | |
| | PO-mU-PO-fG-PO-invdT-Hy | | fA-PO-mA-PO-dT-PO-dT-Hy | |
| 122 | Hy-mC-PO-mC-PO-mA-PO-fU- | 550 | Hy-fG-PO-fC-PO-mA-PO- | 764 |
| | PO-mU-PO-fG-PO-mU-PO-fU- | | fC-PO-mU-PO-fA-PO-mU- | |
| | PO-mA-PO-fA-PO-mA-PO-fG- | | PO-fA-PO-mU-PO-fC-PO- | |
| | PO-mG-PO-fA-PO-mU-PO-fA- | | mC-PO-fU-PO-mU-PO-fU- | |
| | PO-mU-PO-fA-PO-mG-PO-fU- | | PO-mA-PO-fA-PO-mC-PO- | |
| | PO-mG-PO-fC-PO-invdT-Hy | | fA-PO-mA-PO-dT-PO-dT-Hy | |
| 123 | Hy-mC-PO-mC-PO-mA-PO-fU- | 551 | Hy-fG-PO-fG-PO-mC-PO- | 765 |
| | PO-mG-PO-fU-PO-mU-PO-fA- | | fA-PO-mC-PO-fU-PO-mA- | |
| | PO-mA-PO-fA-PO-mG-PO-fG- | | PO-fU-PO-mA-PO-fU-PO- | |
| | PO-mA-PO-fU-PO-mA-PO-fU- | | mC-PO-fC-PO-mU-PO-fU- | |
| | PO-mA-PO-fG-PO-mU-PO-fG- | | PO-mU-PO-fA-PO-mA-PO- | |
| | PO-mC-PO-fC-PO-invdT-Hy | | fC-PO-mA-PO-dT-PO-dT-Hy | |
| 124 | Hy-mC-PO-mC-PO-mA-PO-fG- | 552 | Hy-fG-PO-fG-PO-mG-PO- | 766 |
| | PO-mU-PO-fU-PO-mA-PO-fA- | | fC-PO-mA-PO-fC-PO-mU- | |
| | PO-mA-PO-fG-PO-mG-PO-fA- | | PO-fA-PO-mU-PO-fA-PO- | |
| | PO-mU-PO-fA-PO-mU-PO-fA- | | mU-PO-fC-PO-mC-PO-fU- | |
| | PO-mG-PO-fU-PO-mG-PO-fC- | | PO-mU-PO-fU-PO-mA-PO- | |
| | PO-mC-PO-fC-PO-invdT-Hy | | fA-PO-mC-PO-dT-PO-dT-Hy | |
| 125 | Hy-mC-PO-mC-PO-mA-PO-fU- | 553 | Hy-fU-PO-fG-PO-mG-PO- | 767 |
| | PO-mU-PO-fA-PO-mA-PO-fA- | | fG-PO-mC-PO-fA-PO-mC- | |
| | PO-mG-PO-fG-PO-mA-PO-fU- | | PO-fU-PO-mA-PO-fU-PO- | |
| | PO-mA-PO-fU-PO-mA-PO-fG- | | mA-PO-fU-PO-mC-PO-fC- | |
| | PO-mU-PO-fG-PO-mC-PO-fC- | | PO-mU-PO-fU-PO-mU-PO- | |
| | PO-mC-PO-fA-PO-invdT-Hy | | fA-PO-mA-PO-dT-PO-dT-Hy | |
| 126 | Hy-mC-PO-mC-PO-mA-PO-fU- | 554 | Hy-fU-PO-fU-PO-mG-PO- | 768 |
| | PO-mA-PO-fA-PO-mA-PO-fG- | | fG-PO-mG-PO-fC-PO-mA- | |
| | PO-mG-PO-fA-PO-mU-PO-fA- | | PO-fC-PO-mU-PO-fA-PO- | |
| | PO-mU-PO-fA-PO-mG-PO-fU- | | mU-PO-fA-PO-mU-PO-fC- | |
| | PO-mG-PO-fC-PO-mC-PO-fC- | | PO-mC-PO-fU-PO-mU-PO- | |
| | PO-mA-PO-fA-PO-invdT-Hy | | fU-PO-mA-PO-dT-PO-dT-Hy | |
| 127 | Hy-mC-PO-mC-PO-mA-PO-fA- | 555 | Hy-fA-PO-fA-PO-mC-PO- | 769 |
| | PO-mG-PO-fG-PO-mA-PO-fU- | | fU-PO-mU-PO-fG-PO-mG- | |
| | PO-mA-PO-fU-PO-mA-PO-fG- | | PO-fG-PO-mC-PO-fA-PO- | |
| | PO-mU-PO-fG-PO-mC-PO-fC- | | mC-PO-fU-PO-mA-PO-fU- | |
| | PO-mC-PO-fA-PO-mA-PO-fG- | | PO-mA-PO-fU-PO-mC-PO- | |
| | PO-mU-PO-fU-PO-invdT-Hy | | fC-PO-mU-PO-dT-PO-dT-Hy | |
| 128 | Hy-mC-PO-mC-PO-mA-PO-fG- | 556 | Hy-fU-PO-fA-PO-mA-PO- | 770 |
| | PO-mG-PO-fA-PO-mU-PO-fA- | | fC-PO-mU-PO-fU-PO-mG- | |
| | PO-mU-PO-fA-PO-mG-PO-fU- | | PO-fG-PO-mG-PO-fC-PO- | |
| | PO-mG-PO-fC-PO-mC-PO-fC- | | mA-PO-fC-PO-mU-PO-fA- | |
| | PO-mA-PO-fA-PO-mG-PO-fU- | | PO-mU-PO-fA-PO-mU-PO- | |
| | PO-mU-PO-fA-PO-invdT-Hy | | fC-PO-mC-PO-dT-PO-dT-Hy | |
| 129 | Hy-mC-PO-mC-PO-mA-PO-fG- | 557 | Hy-fA-PO-fU-PO-mA-PO- | 771 |
| | PO-mA-PO-fU-PO-mA-PO-fU- | | fA-PO-mC-PO-fU-PO-mU- | |
| | PO-mA-PO-fG-PO-mU-PO-fG- | | PO-fG-PO-mG-PO-fG-PO- | |
| | PO-mC-PO-fC-PO-mC-PO-fA- | | mC-PO-fA-PO-mC-PO-fU- | |
| | PO-mA-PO-fG-PO-mU-PO-fU- | | PO-mA-PO-fU-PO-mA-PO- | |
| | PO-mA-PO-fU-PO-invdT-Hy | | fU-PO-mC-PO-dT-PO-dT-Hy | |
| 130 | Hy-mC-PO-mC-PO-mA-PO-fA- | 558 | Hy-fU-PO-fA-PO-mU-PO- | 772 |
| | PO-mU-PO-fA-PO-mU-PO-fA- | | fA-PO-mA-PO-fC-PO-mU- | |
| | PO-mG-PO-fU-PO-mG-PO-fC- | | PO-fU-PO-mG-PO-fG-PO- | |
| | PO-mC-PO-fC-PO-mA-PO-fA- | | mG-PO-fC-PO-mA-PO-fC- | |
| | PO-mG-PO-fU-PO-mU-PO-fA- | | PO-mU-PO-fA-PO-mU-PO- | |
| | PO-mU-PO-fA-PO-invdT-Hy | | fA-PO-mU-PO-dT-PO-dT-Hy | |
| 131 | Hy-mC-PO-mC-PO-mA-PO-fG- | 559 | Hy-fU-PO-fU-PO-mG-PO- | 773 |
| | PO-mG-PO-fU-PO-mG-PO-fA- | | fA-PO-mC-PO-fA-PO-mA- | |
| | PO-mC-PO-fC-PO-mU-PO-fA- | | PO-fA-PO-mG-PO-fG-PO- | |
| | PO-mC-PO-fC-PO-mU-PO-fU- | | mU-PO-fA-PO-mG-PO-fG- | |
| | PO-mU-PO-fG-PO-mU-PO-fC- | | PO-mU-PO-fC-PO-mA-PO- | |
| | PO-mA-PO-fA-PO-invdT-Hy | | fC-PO-mC-PO-dT-PO-dT-Hy | |
| 132 | Hy-mC-PO-mC-PO-mA-PO-fG- | 560 | Hy-fA-PO-fU-PO-mU-PO- | 774 |
| | PO-mU-PO-fG-PO-mA-PO-fC- | | fG-PO-mA-PO-fC-PO-mA- | |
| | PO-mC-PO-fU-PO-mA-PO-fC- | | PO-fA-PO-mA-PO-fG-PO- | |
| | PO-mC-PO-fU-PO-mU-PO-fU- | | mG-PO-fU-PO-mA-PO-fG- | |
| | PO-mG-PO-fU-PO-mC-PO-fA- | | PO-mG-PO-fU-PO-mC-PO- | |
| | PO-mA-PO-fU-PO-invdT-Hy | | fA-PO-mC-PO-dT-PO-dT-Hy | |
| 133 | Hy-mC-PO-mC-PO-mA-PO-fU- | 561 | Hy-fU-PO-fA-PO-mU-PO- | 775 |
| | PO-mG-PO-fA-PO-mC-PO-fC- | | fU-PO-mG-PO-fA-PO-mC- | |
| | PO-mU-PO-fA-PO-mC-PO-fC- | | PO-fA-PO-mA-PO-fA-PO- | |
| | PO-mU-PO-fU-PO-mU-PO-fG- | | mG-PO-fG-PO-mU-PO-fA- | |
| | PO-mU-PO-fC-PO-mA-PO-fA- | | PO-mG-PO-fG-PO-mU-PO- | |
| | PO-mU-PO-fA-PO-invdT-Hy | | fC-PO-mA-PO-dT-PO-dT-Hy | |
| 134 | Hy-mC-PO-mC-PO-mA-PO-fA- | 562 | Hy-fU-PO-fG-PO-mG-PO- | 776 |
| | PO-mU-PO-fG-PO-mU-PO-fA- | | fA-PO-mU-PO-fA-PO-mA- | |
| | PO-mU-PO-fU-PO-mU-PO-fC- | | PO-fU-PO-mU-PO-fU-PO- | |
| | PO-mA-PO-fA-PO-mA-PO-fU- | | mG-PO-fA-PO-mA-PO-fA- | |
| | PO-mU-PO-fA-PO-mU-PO-fC- | | PO-mU-PO-fA-PO-mC-PO- | |
| | PO-mC-PO-fA-PO-invdT-Hy | | fA-PO-mU-PO-dT-PO-dT-Hy | |
| 135 | Hy-mC-PO-mC-PO-mA-PO-fU- | 563 | Hy-fG-PO-fU-PO-mA-PO- | 777 |
| | PO-mU-PO-fC-PO-mA-PO-fA- | | fU-PO-mA-PO-fU-PO-mU- | |
| | PO-mA-PO-fU-PO-mU-PO-fA- | | PO-fG-PO-mG-PO-fA-PO- | |
| | PO-mU-PO-fC-PO-mC-PO-fA- | | mU-PO-fA-PO-mA-PO-fU- | |
| | PO-mA-PO-fU-PO-mA-PO-fU- | | PO-mU-PO-fU-PO-mG-PO- | |
| | PO-mA-PO-fC-PO-invdT-Hy | | fA-PO-mA-PO-dT-PO-dT-Hy | |
| 136 | Hy-mC-PO-mC-PO-mA-PO-fU- | 564 | Hy-fU-PO-fG-PO-mU-PO- | 778 |
| | PO-mC-PO-fA-PO-mA-PO-fA- | | fA-PO-mU-PO-fA-PO-mU- | |
| | PO-mU-PO-fU-PO-mA-PO-fU- | | PO-fU-PO-mG-PO-fG-PO- | |
| | PO-mC-PO-fC-PO-mA-PO-fA- | | mA-PO-fU-PO-mA-PO-fA- | |
| | PO-mU-PO-fA-PO-mU-PO-fA- | | PO-mU-PO-fU-PO-mU-PO- | |
| | PO-mC-PO-fA-PO-invdT-Hy | | fG-PO-mA-PO-dT-PO-dT-Hy | |
| 137 | Hy-mC-PO-mC-PO-mA-PO-fA- | 565 | Hy-fG-PO-fA-PO-mC-PO- | 779 |
| | PO-mU-PO-fU-PO-mA-PO-fU- | | £A-PO-mU-PO-fG-PO-mU- | |
| | PO-mC-PO-fC-PO-mA-PO-fA- | | PO-fA-PO-mU-PO-fA-PO- | |
| | PO-mU-PO-fA-PO-mU-PO-fA- | | mU-PO-fU-PO-mG-PO-fG- | |
| | PO-mC-PO-fA-PO-mU-PO-fG- | | PO-mA-PO-fU-PO-mA-PO- | |
| | PO-mU-PO-fC-PO-invdT-Hy | | fA-PO-mU-PO-dT-PO-dT-Hy | |
| 138 | Hy-mC-PO-mC-PO-mA-PO-fU- | 566 | Hy-fU-PO-fG-PO-mA-PO- | 780 |
| | PO-mU-PO-fA-PO-mU-PO-fC- | | fC-PO-mA-PO-fU-PO-mG- | |
| | PO-mC-PO-fA-PO-mA-PO-fU- | | PO-fU-PO-mA-PO-fU-PO- | |
| | PO-mA-PO-fU-PO-mA-PO-fC- | | mA-PO-fU-PO-mU-PO-fG- | |
| | PO-mA-PO-fU-PO-mG-PO-fU- | | PO-mG-PO-fA-PO-mU-PO- | |
| | PO-mC-PO-fA-PO-invdT-Hy | | fA-PO-mA-PO-dT-PO-dT-Hy | |
| 139 | Hy-mC-PO-mC-PO-mA-PO-fU- | 567 | Hy-fA-PO-fU-PO-mG-PO- | 781 |
| | PO-mA-PO-fU-PO-mC-PO-fC- | | fA-PO-mC-PO-fA-PO-mU- | |
| | PO-mA-PO-fA-PO-mU-PO-fA- | | PO-fG-PO-mU-PO-fA-PO- | |
| | PO-mU-PO-fA-PO-mC-PO-fA- | | mU-PO-fA-PO-mU-PO-fU- | |
| | PO-mU-PO-fG-PO-mU-PO-fC- | | PO-mG-PO-fG-PO-mA-PO- | |
| | PO-mA-PO-fU-PO-invdT-Hy | | fU-PO-mA-PO-dT-PO-dT-Hy | |
| 140 | Hy-mC-PO-mC-PO-mA-PO-fC- | 568 | Hy-fU-PO-fA-PO-mA-PO- | 782 |
| | PO-mA-PO-fU-PO-mG-PO-fU- | | fA-PO-mA-PO-fA-PO-mU- | |
| | PO-mC-PO-fA-PO-mU-PO-fA- | | PO-fA-PO-mU-PO-fA-PO- | |
| | PO-mU-PO-fA-PO-mU-PO-fA- | | mU-PO-fA-PO-mU-PO-fG- | |
| | PO-mU-PO-fU-PO-mU-PO-fU- | | PO-mA-PO-fC-PO-mA-PO- | |
| | PO-mU-PO-fA-PO-invdT-Hy | | fU-PO-mG-PO-dT-PO-dT-Hy | |
| 141 | Hy-mC-PO-mC-PO-mA-PO-fA- | 569 | Hy-fA-PO-fC-PO-mC-PO- | 783 |
| | PO-mG-PO-fU-PO-mA-PO-fC- | | fU-PO-mU-PO-fU-PO-mA- | |
| | PO-mA-PO-fA-PO-mA-PO-fA- | | PO-fU-PO-mU-PO-fA-PO- | |
| | PO-mU-PO-fA-PO-mA-PO-fU- | | mU-PO-fU-PO-mU-PO-fU- | |
| | PO-mA-PO-fA-PO-mA-PO-fG- | | PO-mG-PO-fU-PO-mA-PO- | |
| | PO-mG-PO-fU-PO-invdT-Hy | | fC-PO-mU-PO-dT-PO-dT-Hy | |
| 142 | Hy-mC-PO-mC-PO-mA-PO-fG- | 570 | Hy-fU-PO-fA-PO-mC-PO- | 784 |
| | PO-mU-PO-fA-PO-mC-PO-fA- | | fC-PO-mU-PO-fU-PO-mU- | |
| | PO-mA-PO-fA-PO-mA-PO-fU- | | PO-fA-PO-mU-PO-fU-PO- | |
| | PO-mA-PO-fA-PO-mU-PO-fA- | | mA-PO-fU-PO-mU-PO-fU- | |
| | PO-mA-PO-fA-PO-mG-PO-fG- | | PO-mU-PO-fG-PO-mU-PO- | |
| | PO-mU-PO-fA-PO-invdT-Hy | | fA-PO-mC-PO-dT-PO-dT-Hy | |
| 143 | Hy-mC-PO-mC-PO-mA-PO-fA- | 571 | Hy-fC-PO-fU-PO-mG-PO- | 785 |
| | PO-mU-PO-fA-PO-mA-PO-fU- | | fU-PO-mG-PO-fG-PO-mU- | |
| | PO-mU-PO-fU-PO-mU-PO-fC- | | PO-fC-PO-mC-PO-fU-PO- | |
| | PO-mA-PO-fG-PO-mG-PO-fA- | | mG-PO-fA-PO-mA-PO-fA- | |
| | PO-mC-PO-fC-PO-mA-PO-fC- | | PO-mA-PO-fU-PO-mU-PO- | |
| | PO-mA-PO-fG-PO-invdT-Hy | | fA-PO-mU-PO-dT-PO-dT-Hy | |
| 144 | Hy-mC-PO-mC-PO-mA-PO-fA- | 572 | Hy-fG-PO-fU-PO-mC-PO- | 786 |
| | PO-mA-PO-fU-PO-mU-PO-fU- | | fU-PO-mG-PO-fU-PO-mG- | |
| | PO-mU-PO-fC-PO-mA-PO-fG- | | PO-fG-PO-mU-PO-fC-PO- | |
| | PO-mG-PO-fA-PO-mC-PO-fC- | | mC-PO-fU-PO-mG-PO-fA- | |
| | PO-mA-PO-fC-PO-mA-PO-fG- | | PO-mA-PO-fA-PO-mA-PO- | |
| | PO-mA-PO-fC-PO-invdT-Hy | | fU-PO-mU-PO-dT-PO-dT-Hy | |
| 145 | Hy-mC-PO-mC-PO-mA-PO-fU- | 573 | Hy-fU-PO-fA-PO-mG-PO- | 787 |
| | PO-mU-PO-fU-PO-mU-PO-fC- | | fU-PO-mC-PO-fU-PO-mG- | |
| | PO-mA-PO-fG-PO-mG-PO-fA- | | PO-fU-PO-mG-PO-fG-PO- | |
| | PO-mC-PO-fC-PO-mA-PO-fC- | | mU-PO-fC-PO-mC-PO-fU- | |
| | PO-mA-PO-fG-PO-mA-PO-fC- | | PO-mG-PO-fA-PO-mA-PO- | |
| | PO-mU-PO-fA-PO-invdT-Hy | | fA-PO-mA-PO-dT-PO-dT-Hy | |
| 146 | Hy-mC-PO-mC-PO-mA-PO-fU- | 574 | Hy-fU-PO-fU-PO-mA-PO- | 788 |
| | PO-mU-PO-fU-PO-mC-PO-fA- | | fG-PO-mU-PO-fC-PO-mU- | |
| | PO-mG-PO-fG-PO-mA-PO-fC- | | PO-fG-PO-mU-PO-fG-PO- | |
| | PO-mC-PO-fA-PO-mC-PO-fA- | | mG-PO-fU-PO-mC-PO-fC- | |
| | PO-mG-PO-fA-PO-mC-PO-fU- | | PO-mU-PO-fG-PO-mA-PO- | |
| | PO-mA-PO-fA-PO-invdT-Hy | | fA-PO-mA-PO-dT-PO-dT-Hy | |
| 147 | Hy-mC-PO-mC-PO-mA-PO-fU- | 575 | Hy-fC-PO-fU-PO-mU-PO- | 789 |
| | PO-mU-PO-fC-PO-mA-PO-fG- | | fA-PO-mG-PO-fU-PO-mC- | |
| | PO-mG-PO-fA-PO-mC-PO-fC- | | PO-fU-PO-mG-PO-fU-PO- | |
| | PO-mA-PO-fC-PO-mA-PO-fG- | | mG-PO-fG-PO-mU-PO-fC- | |
| | PO-mA-PO-fC-PO-mU-PO-fA- | | PO-mC-PO-fU-PO-mG-PO- | |
| | PO-mA-PO-fG-PO-invdT-Hy | | fA-PO-mA-PO-dT-PO-dT-Hy | |
| 148 | Hy-mC-PO-mC-PO-mA-PO-fU- | 576 | Hy-fG-PO-fC-PO-mU-PO- | 790 |
| | PO-mC-PO-fA-PO-mG-PO-fG- | | fU-PO-mA-PO-fG-PO-mU- | |
| | PO-mA-PO-fC-PO-mC-PO-fA- | | PO-fC-PO-mU-PO-fG-PO- | |
| | PO-mC-PO-fA-PO-mG-PO-fA- | | mU-PO-fG-PO-mG-PO-fU- | |
| | PO-mC-PO-fU-PO-mA-PO-fA- | | PO-mC-PO-fC-PO-mU-PO- | |
| | PO-mG-PO-fC-PO-invdT-Hy | | fG-PO-mA-PO-dT-PO-dT-Hy | |
| 149 | Hy-mC-PO-mC-PO-mA-PO-fA- | 577 | Hy-fC-PO-fA-PO-mG-PO- | 791 |
| | PO-mG-PO-fG-PO-mA-PO-fC- | | fC-PO-mU-PO-fU-PO-mA- | |
| | PO-mC-PO-fA-PO-mC-PO-fA- | | PO-fG-PO-mU-PO-fC-PO- | |
| | PO-mG-PO-fA-PO-mC-PO-fU- | | mU-PO-fG-PO-mU-PO-fG- | |
| | PO-mA-PO-fA-PO-mG-PO-fC- | | PO-mG-PO-fU-PO-mC-PO- | |
| | PO-mU-PO-fG-PO-invdT-Hy | | fC-PO-mU-PO-dT-PO-dT-Hy | |
| 150 | Hy-mC-PO-mC-PO-mA-PO-fG- | 578 | Hy-fA-PO-fC-PO-mA-PO- | 792 |
| | PO-mG-PO-fA-PO-mC-PO-fC- | | fG-PO-mC-PO-fU-PO-mU- | |
| | PO-mA-PO-fC-PO-mA-PO-fG- | | PO-fA-PO-mG-PO-fU-PO- | |
| | PO-mA-PO-fC-PO-mU-PO-fA- | | mC-PO-fU-PO-mG-PO-fU- | |
| | PO-mA-PO-fG-PO-mC-PO-fU- | | PO-mG-PO-fG-PO-mU-PO- | |
| | PO-mG-PO-fU-PO-invdT-Hy | | fC-PO-mC-PO-dT-PO-dT-Hy | |
| 151 | Hy-mC-PO-mC-PO-mA-PO-fG- | 579 | Hy-fG-PO-fA-PO-mC-PO- | 793 |
| | PO-mA-PO-fC-PO-mC-PO-fA- | | fA-PO-mG-PO-fC-PO-mU- | |
| | PO-mC-PO-fA-PO-mG-PO-fA- | | PO-fU-PO-mA-PO-fG-PO- | |
| | PO-mC-PO-fU-PO-mA-PO-fA- | | mU-PO-fC-PO-mU-PO-fG- | |
| | PO-mG-PO-fC-PO-mU-PO-fG- | | PO-mU-PO-fG-PO-mG-PO- | |
| | PO-mU-PO-fC-PO-invdT-Hy | | fU-PO-mC-PO-dT-PO-dT-Hy | |
| 152 | Hy-mC-PO-mC-PO-mA-PO-fU- | 580 | Hy-fG-PO-fU-PO-mA-PO- | 794 |
| | PO-mU-PO-fU-PO-mU-PO-fU- | | fU-PO-mU-PO-fC-PO-mU- | |
| | PO-mU-PO-fA-PO-mG-PO-fG- | | PO-fG-PO-mG-PO-fC-PO- | |
| | PO-mG-PO-fC-PO-mC-PO-fA- | | mC-PO-fC-PO-mU-PO-fA- | |
| | PO-mG-PO-fA-PO-mA-PO-fU- | | PO-mA-PO-fA-PO-mA-PO- | |
| | PO-mA-PO-fC-PO-invdT-Hy | | fA-PO-mA-PO-dT-PO-dT-Hy | |
| 153 | Hy-mC-PO-mC-PO-mA-PO-fU- | 581 | Hy-fG-PO-fG-PO-mU-PO- | 795 |
| | PO-mU-PO-fU-PO-mU-PO-fU- | | fA-PO-mU-PO-fU-PO-mC- | |
| | PO-mA-PO-fG-PO-mG-PO-fG- | | PO-fU-PO-mG-PO-fG-PO- | |
| | PO-mC-PO-fC-PO-mA-PO-fG- | | mC-PO-fC-PO-mC-PO-fU- | |
| | PO-mA-PO-fA-PO-mU-PO-fA- | | PO-mA-PO-fA-PO-mA-PO- | |
| | PO-mC-PO-fC-PO-invdT-Hy | | fA-PO-mA-PO-dT-PO-dT-Hy | |
| 154 | Hy-mC-PO-mC-PO-mA-PO-fU- | 582 | Hy-fU-PO-fG-PO-mG-PO- | 796 |
| | PO-mU-PO-fU-PO-mU-PO-fA- | | fU-PO-mA-PO-fU-PO-mU- | |
| | PO-mG-PO-fG-PO-mG-PO-fC- | | PO-fC-PO-mU-PO-fG-PO- | |
| | PO-mC-PO-fA-PO-mG-PO-fA- | | mG-PO-fC-PO-mC-PO-fC- | |
| | PO-mA-PO-fU-PO-mA-PO-fC- | | PO-mU-PO-fA-PO-mA-PO- | |
| | PO-mC-PO-fA-PO-invdT-Hy | | fA-PO-mA-PO-dT-PO-dT-Hy | |
| 155 | Hy-mC-PO-mC-PO-mA-PO-fU- | 583 | Hy-fU-PO-fU-PO-mG-PO- | 797 |
| | PO-mU-PO-fU-PO-mA-PO-fG- | | fG-PO-mU-PO-fA-PO-mU- | |
| | PO-mG-PO-fG-PO-mC-PO-fC- | | PO-fU-PO-mC-PO-fU-PO- | |
| | PO-mA-PO-fG-PO-mA-PO-fA- | | mG-PO-fG-PO-mC-PO-fC- | |
| | PO-mU-PO-fA-PO-mC-PO-fC- | | PO-mC-PO-fU-PO-mA-PO- | |
| | PO-mA-PO-fA-PO-invdT-Hy | | fA-PO-mA-PO-dT-PO-dT-Hy | |
| 156 | Hy-mC-PO-mC-PO-mA-PO-fU- | 584 | Hy-fU-PO-fU-PO-mU-PO- | 798 |
| | PO-mU-PO-fA-PO-mG-PO-fG- | | fG-PO-mG-PO-fU-PO-mA- | |
| | PO-mG-PO-fC-PO-mC-PO-fA- | | PO-fU-PO-mU-PO-fC-PO- | |
| | PO-mG-PO-fA-PO-mA-PO-fU- | | mU-PO-fG-PO-mG-PO-fC- | |
| | PO-mA-PO-fC-PO-mC-PO-fA- | | PO-mC-PO-fC-PO-mU-PO- | |
| | PO-mA-PO-fA-PO-invdT-Hy | | fA-PO-mA-PO-dT-PO-dT-Hy | |
| 157 | Hy-mC-PO-mC-PO-mA-PO-fU- | 585 | Hy-fU-PO-fU-PO-mU-PO- | 799 |
| | PO-mA-PO-fG-PO-mG-PO-fG- | | fU-PO-mG-PO-fG-PO-mU- | |
| | PO-mC-PO-fC-PO-mA-PO-fG- | | PO-fA-PO-mU-PO-fU-PO- | |
| | PO-mA-PO-fA-PO-mU-PO-fA- | | mC-PO-fU-PO-mG-PO-fG- | |
| | PO-mC-PO-fC-PO-mA-PO-fA- | | PO-mC-PO-fC-PO-mC-PO- | |
| | PO-mA-PO-fA-PO-invdT-Hy | | fU-PO-mA-PO-dT-PO-dT-Hy | |
| 158 | Hy-mC-PO-mC-PO-mA-PO-fA- | 586 | Hy-fA-PO-fU-PO-mU-PO- | 800 |
| | PO-mG-PO-fG-PO-mG-PO-fC- | | fU-PO-mU-PO-fG-PO-mG- | |
| | PO-mC-PO-fA-PO-mG-PO-fA- | | PO-fU-PO-mA-PO-fU-PO- | |
| | PO-mA-PO-fU-PO-mA-PO-fC- | | mU-PO-fC-PO-mU-PO-fG- | |
| | PO-mC-PO-fA-PO-mA-PO-fA- | | PO-mG-PO-fC-PO-mC-PO- | |
| | PO-mA-PO-fU-PO-invdT-Hy | | fC-PO-mU-PO-dT-PO-dT-Hy | |
| 159 | Hy-mC-PO-mC-PO-mA-PO-fA- | 587 | Hy-fA-PO-fU-PO-mU-PO- | 801 |
| | PO-mA-PO-fA-PO-mU-PO-fU- | | fU-PO-mG-PO-fA-PO-mA- | |
| | PO-mG-PO-fG-PO-mA-PO-fC- | | PO-fA-PO-mU-PO-fU-PO- | |
| | PO-mA-PO-fA-PO-mU-PO-fU- | | mG-PO-fU-PO-mC-PO-fC- | |
| | PO-mU-PO-fC-PO-mA-PO-fA- | | PO-mA-PO-fA-PO-mU-PO- | |
| | PO-mA-PO-fU-PO-invdT-Hy | | fU-PO-mU-PO-dT-PO-dT-Hy | |
| 160 | Hy-mC-PO-mC-PO-mA-PO-fA- | 588 | Hy-fG-PO-fC-PO-mA-PO- | 802 |
| | PO-mU-PO-fU-PO-mG-PO-fG- | | fU-PO-mU-PO-fU-PO-mG- | |
| | PO-mA-PO-fC-PO-mA-PO-fA- | | PO-fA-PO-mA-PO-fA-PO- | |
| | PO-mU-PO-fU-PO-mU-PO-fC- | | mU-PO-fU-PO-mG-PO-fU- | |
| | PO-mA-PO-fA-PO-mA-PO-fU- | | PO-mC-PO-fC-PO-mA-PO- | |
| | PO-mG-PO-fC-PO-invdT-Hy | | fA-PO-mU-PO-dT-PO-dT-Hy | |
| 161 | Hy-mC-PO-mC-PO-mA-PO-fU- | 589 | Hy-fU-PO-fG-PO-mC-PO- | 803 |
| | PO-mU-PO-fG-PO-mG-PO-fA- | | fA-PO-mU-PO-fU-PO-mU- | |
| | PO-mC-PO-fA-PO-mA-PO-fU- | | PO-fG-PO-mA-PO-fA-PO- | |
| | PO-mU-PO-fU-PO-mC-PO-fA- | | mA-PO-fU-PO-mU-PO-fG- | |
| | PO-mA-PO-fA-PO-mU-PO-fG- | | PO-mU-PO-fC-PO-mC-PO- | |
| | PO-mC-PO-fA-PO-invdT-Hy | | fA-PO-mA-PO-dT-PO-dT-Hy | |
| 162 | Hy-mC-PO-mC-PO-mA-PO-fU- | 590 | Hy-fG-PO-fA-PO-mA-PO- | 804 |
| | PO-mU-PO-fA-PO-mA-PO-fU- | | fG-PO-mC-PO-fA-PO-mA- | |
| | PO-mA-PO-fU-PO-mA-PO-fU- | | PO-fC-PO-mU-PO-fC-PO- | |
| | PO-mG-PO-fA-PO-mG-PO-fU- | | mA-PO-fU-PO-mA-PO-fU- | |
| | PO-mU-PO-fG-PO-mC-PO-fU- | | PO-mA-PO-fU-PO-mU-PO- | |
| | PO-mU-PO-fC-PO-invdT-Hy | | fA-PO-mA-PO-dT-PO-dT-Hy | |
| 163 | Hy-mC-PO-mC-PO-mA-PO-fG- | 591 | Hy-fG-PO-fA-PO-mA-PO- | 805 |
| | PO-mA-PO-fA-PO-mU-PO-fU- | | fU-PO-mU-PO-fG-PO-mU- | |
| | PO-mG-PO-fA-PO-mU-PO-fC- | | PO-fC-PO-mU-PO-fU-PO- | |
| | PO-mA-PO-fA-PO-mG-PO-fA- | | mG-PO-fA-PO-mU-PO-fC- | |
| | PO-mC-PO-fA-PO-mA-PO-fU- | | PO-mA-PO-fA-PO-mU-PO- | |
| | PO-mU-PO-fC-PO-invdT-Hy | | fU-PO-mC-PO-dT-PO-dT-Hy | |
| 164 | Hy-mC-PO-mC-PO-mA-PO-fA- | 592 | Hy-fU-PO-fG-PO-mA-PO- | 806 |
| | PO-mA-PO-fU-PO-mU-PO-fG- | | fA-PO-mU-PO-fU-PO-mG- | |
| | PO-mA-PO-fU-PO-mC-PO-fA- | | PO-fU-PO-mC-PO-fU-PO- | |
| | PO-mA-PO-fG-PO-mA-PO-fC- | | mU-PO-fG-PO-mA-PO-fU- | |
| | PO-mA-PO-fA-PO-mU-PO-fU- | | PO-mC-PO-fA-PO-mA-PO- | |
| | PO-mC-PO-fA-PO-invdT-Hy | | fU-PO-mU-PO-dT-PO-dT-Hy | |
| 165 | Hy-mC-PO-mC-PO-mA-PO-fA- | 593 | Hy-fA-PO-fU-PO-mG-PO- | 807 |
| | PO-mU-PO-fU-PO-mG-PO-fA- | | fA-PO-mA-PO-fU-PO-mU- | |
| | PO-mU-PO-fC-PO-mA-PO-fA- | | PO-fG-PO-mU-PO-fC-PO- | |
| | PO-mG-PO-fA-PO-mC-PO-fA- | | mU-PO-fU-PO-mG-PO-fA- | |
| | PO-mA-PO-fU-PO-mU-PO-fC- | | PO-mU-PO-fC-PO-mA-PO- | |
| | PO-mA-PO-fU-PO-invdT-Hy | | fA-PO-mU-PO-dT-PO-dT-Hy | |
| 166 | Hy-mC-PO-mC-PO-mA-PO-fU- | 594 | Hy-fG-PO-fA-PO-mU-PO- | 808 |
| | PO-mU-PO-fG-PO-mA-PO-fU- | | fG-PO-mA-PO-fA-PO-mU- | |
| | PO-mC-PO-fA-PO-mA-PO-fG- | | PO-fU-PO-mG-PO-fU-PO- | |
| | PO-mA-PO-fC-PO-mA-PO-fA- | | mC-PO-fU-PO-mU-PO-fG- | |
| | PO-mU-PO-fU-PO-mC-PO-fA- | | PO-mA-PO-fU-PO-mC-PO- | |
| | PO-mU-PO-fC-PO-invdT-Hy | | fA-PO-mA-PO-dT-PO-dT-Hy | |
| 167 | Hy-mC-PO-mC-PO-mA-PO-fU- | 595 | Hy-fU-PO-fG-PO-mA-PO- | 809 |
| | PO-mG-PO-fA-PO-mU-PO-fC- | | fU-PO-mG-PO-fA-PO-mA- | |
| | PO-mA-PO-fA-PO-mG-PO-fA- | | PO-fU-PO-mU-PO-fG-PO- | |
| | PO-mC-PO-fA-PO-mA-PO-fU- | | mU-PO-fC-PO-mU-PO-fU- | |
| | PO-mU-PO-fC-PO-mA-PO-fU- | | PO-mG-PO-fA-PO-mU-PO- | |
| | PO-mC-PO-fA-PO-invdT-Hy | | fC-PO-mA-PO-dT-PO-dT-Hy | |
| 168 | Hy-mC-PO-mC-PO-mA-PO-fG- | 596 | Hy-fA-PO-fU-PO-mG-PO- | 810 |
| | PO-mA-PO-fU-PO-mC-PO-fA- | | fA-PO-mU-PO-fG-PO-mA- | |
| | PO-mA-PO-fG-PO-mA-PO-fC- | | PO-fA-PO-mU-PO-fU-PO- | |
| | PO-mA-PO-fA-PO-mU-PO-fU- | | mG-PO-fU-PO-mC-PO-fU- | |
| | PO-mC-PO-fA-PO-mU-PO-fC- | | PO-mU-PO-fG-PO-mA-PO- | |
| | PO-mA-PO-fU-PO-invdT-Hy | | fU-PO-mC-PO-dT-PO-dT-Hy | |
| 169 | Hy-mC-PO-mC-PO-mA-PO-fA- | 597 | Hy-fA-PO-fA-PO-mU-PO- | 811 |
| | PO-mU-PO-fC-PO-mA-PO-fA- | | fG-PO-mA-PO-fU-PO-mG- | |
| | PO-mG-PO-fA-PO-mC-PO-fA- | | PO-fA-PO-mA-PO-fU-PO- | |
| | PO-mA-PO-fU-PO-mU-PO-fC- | | mU-PO-fG-PO-mU-PO-fC- | |
| | PO-mA-PO-fU-PO-mC-PO-fA- | | PO-mU-PO-fU-PO-mG-PO- | |
| | PO-mU-PO-fU-PO-invdT-Hy | | fA-PO-mU-PO-dT-PO-dT-Hy | |
| 170 | Hy-mC-PO-mC-PO-mA-PO-fU- | 598 | Hy-fA-PO-fA-PO-mA-PO- | 812 |
| | PO-mC-PO-fA-PO-mA-PO-fG- | | fU-PO-mG-PO-fA-PO-mU- | |
| | PO-mA-PO-fC-PO-mA-PO-fA- | | PO-fG-PO-mA-PO-fA-PO- | |
| | PO-mU-PO-fU-PO-mC-PO-fA- | | mU-PO-fU-PO-mG-PO-fU- | |
| | PO-mU-PO-fC-PO-mA-PO-fU- | | PO-mC-PO-fU-PO-mU-PO- | |
| | PO-mU-PO-fU-PO-invdT-Hy | | fG-PO-mA-PO-dT-PO-dT-Hy | |
| 171 | Hy-mC-PO-mC-PO-mA-PO-fU- | 599 | Hy-fA-PO-fU-PO-mA-PO- | 813 |
| | PO-mC-PO-fA-PO-mU-PO-fC- | | fG-PO-mA-PO-fG-PO-mA- | |
| | PO-mA-PO-fU-PO-mU-PO-fU- | | PO-fA-PO-mU-PO-fC-PO- | |
| | PO-mG-PO-fA-PO-mU-PO-fU- | | mA-PO-fA-PO-mA-PO-fU- | |
| | PO-mC-PO-fU-PO-mC-PO-fU- | | PO-mG-PO-fA-PO-mU-PO- | |
| | PO-mA-PO-fU-PO-invdT-Hy | | fG-PO-mA-PO-dT-PO-dT-Hy | |
| 172 | Hy-mC-PO-mC-PO-mA-PO-fC- | 600 | Hy-fG-PO-fA-PO-mU-PO- | 814 |
| | PO-mA-PO-fU-PO-mC-PO-fA- | | fA-PO-mG-PO-fA-PO-mG- | |
| | PO-mU-PO-fU-PO-mU-PO-fG- | | PO-fA-PO-mA-PO-fU-PO- | |
| | PO-mA-PO-fU-PO-mU-PO-fC- | | mC-PO-fA-PO-mA-PO-fA- | |
| | PO-mU-PO-fC-PO-mU-PO-fA- | | PO-mU-PO-fG-PO-mA-PO- | |
| | PO-mU-PO-fC-PO-invdT-Hy | | fU-PO-mG-PO-dT-PO-dT-Hy | |
| 173 | Hy-mC-PO-mC-PO-mA-PO-fA- | 601 | Hy-fA-PO-fG-PO-mA-PO- | 815 |
| | PO-mU-PO-fC-PO-mA-PO-fU- | | fU-PO-mA-PO-fG-PO-mA- | |
| | PO-mU-PO-fU-PO-mG-PO-fA- | | PO-fG-PO-mA-PO-fA-PO- | |
| | PO-mU-PO-fU-PO-mC-PO-fU- | | mU-PO-fC-PO-mA-PO-fA- | |
| | PO-mC-PO-fU-PO-mA-PO-fU- | | PO-mA-PO-fU-PO-mG-PO- | |
| | PO-mC-PO-fU-PO-invdT-Hy | | fA-PO-mU-PO-dT-PO-dT-Hy | |
| 174 | Hy-mC-PO-mC-PO-mA-PO-fU- | 602 | Hy-fC-PO-fU-PO-mU-PO- | 816 |
| | PO-mA-PO-fG-PO-mA-PO-fA- | | fC-PO-mU-PO-fG-PO-mA- | |
| | PO-mA-PO-fA-PO-mU-PO-fC- | | PO-fG-PO-mC-PO-fU-PO- | |
| | PO-mA-PO-fG-PO-mC-PO-fU- | | mG-PO-fA-PO-mU-PO-fU- | |
| | PO-mC-PO-fA-PO-mG-PO-fA- | | PO-mU-PO-fU-PO-mC-PO- | |
| | PO-mA-PO-fG-PO-invdT-Hy | | fU-PO-mA-PO-dT-PO-dT-Hy | |
| 175 | Hy-mC-PO-mC-PO-mA-PO-fG- | 603 | Hy-fU-PO-fC-PO-mC-PO- | 817 |
| | PO-mA-PO-fA-PO-mA-PO-fA- | | fU-PO-mU-PO-fC-PO-mU- | |
| | PO-mU-PO-fC-PO-mA-PO-fG- | | PO-fG-PO-mA-PO-fG-PO- | |
| | PO-mC-PO-fU-PO-mC-PO-fA- | | mC-PO-fU-PO-mG-PO-fA- | |
| | PO-mG-PO-fA-PO-mA-PO-fG- | | PO-mU-PO-fU-PO-mU-PO- | |
| | PO-mG-PO-fA-PO-invdT-Hy | | fU-PO-mC-PO-dT-PO-dT-Hy | |
| 176 | Hy-mC-PO-mC-PO-mA-PO-fA- | 604 | Hy-fG-PO-fU-PO-mC-PO- | 818 |
| | PO-mA-PO-fA-PO-mA-PO-fU- | | fC-PO-mU-PO-fU-PO-mC- | |
| | PO-mC-PO-fA-PO-mG-PO-fC- | | PO-fU-PO-mG-PO-fA-PO- | |
| | PO-mU-PO-fC-PO-mA-PO-fG- | | mG-PO-fC-PO-mU-PO-fG- | |
| | PO-mA-PO-fA-PO-mG-PO-fG- | | PO-mA-PO-fU-PO-mU-PO- | |
| | PO-mA-PO-fC-PO-invdT-Hy | | fU-PO-mU-PO-dT-PO-dT-Hy | |
| 177 | Hy-mC-PO-mC-PO-mA-PO-fA- | 605 | Hy-fA-PO-fG-PO-mU-PO- | 819 |
| | PO-mA-PO-fA-PO-mU-PO-fC- | | fC-PO-mC-PO-fU-PO-mU- | |
| | PO-mA-PO-fG-PO-mC-PO-fU- | | PO-fC-PO-mU-PO-fG-PO- | |
| | PO-mC-PO-fA-PO-mG-PO-fA- | | mA-PO-fG-PO-mC-PO-fU- | |
| | PO-mA-PO-fG-PO-mG-PO-fA- | | PO-mG-PO-fA-PO-mU-PO- | |
| | PO-mC-PO-fU-PO-invdT-Hy | | fU-PO-mU-PO-dT-PO-dT-Hy | |
| 178 | Hy-mC-PO-mC-PO-mA-PO-fA- | 606 | Hy-fU-PO-fA-PO-mG-PO- | 820 |
| | PO-mA-PO-fU-PO-mC-PO-fA- | | fU-PO-mC-PO-fC-PO-mU- | |
| | PO-mG-PO-fC-PO-mU-PO-fC- | | PO-fU-PO-mC-PO-fU-PO- | |
| | PO-mA-PO-fG-PO-mA-PO-fA- | | mG-PO-fA-PO-mG-PO-fC- | |
| | PO-mG-PO-fG-PO-mA-PO-fC- | | PO-mU-PO-fG-PO-mA-PO- | |
| | PO-mU-PO-fA-PO-invdT-Hy | | fU-PO-mU-PO-dT-PO-dT-Hy | |
| 179 | Hy-mC-PO-mC-PO-mA-PO-fG- | 607 | Hy-fA-PO-fU-PO-mU-PO- | 821 |
| | PO-mA-PO-fU-PO-mG-PO-fG- | | fC-PO-mA-PO-fG-PO-mC- | |
| | PO-mC-PO-fA-PO-mU-PO-fU- | | PO-fA-PO-mG-PO-fG-PO- | |
| | PO-mC-PO-fC-PO-mU-PO-fG- | | mA-PO-fA-PO-mU-PO-fG- | |
| | PO-mC-PO-fU-PO-mG-PO-fA- | | PO-mC-PO-fC-PO-mA-PO- | |
| | PO-mA-PO-fU-PO-invdT-Hy | | fU-PO-mC-PO-dT-PO-dT-Hy | |
| 180 | Hy-mC-PO-mC-PO-mA-PO-fC- | 608 | Hy-fG-PO-fG-PO-mU-PO- | 822 |
| | PO-mA-PO-fU-PO-mU-PO-fC- | | fA-PO-mC-PO-fA-PO-mU- | |
| | PO-mC-PO-fU-PO-mG-PO-fC- | | PO-fU-PO-mC-PO-fA-PO- | |
| | PO-mU-PO-fG-PO-mA-PO-fA- | | mG-PO-fC-PO-mA-PO-fG- | |
| | PO-mU-PO-fG-PO-mU-PO-fA- | | PO-mG-PO-fA-PO-mA-PO- | |
| | PO-mC-PO-fC-PO-invdT-Hy | | fU-PO-mG-PO-dT-PO-dT-Hy | |
| 181 | Hy-mC-PO-mC-PO-mA-PO-fU- | 609 | Hy-fG-PO-fG-PO-mU-PO- | 823 |
| | PO-mC-PO-fC-PO-mU-PO-fG- | | fG-PO-mG-PO-fU-PO-mA- | |
| | PO-mC-PO-fU-PO-mG-PO-fA- | | PO-fC-PO-mA-PO-fU-PO- | |
| | PO-mA-PO-fU-PO-mG-PO-fU- | | mU-PO-fC-PO-mA-PO-fG- | |
| | PO-mA-PO-fC-PO-mC-PO-fA- | | PO-mC-PO-fA-PO-mG-PO- | |
| | PO-mC-PO-fC-PO-invdT-Hy | | fG-PO-mA-PO-dT-PO-dT-Hy | |
| 182 | Hy-mC-PO-mC-PO-mA-PO-fC- | 610 | Hy-fA-PO-fU-PO-mG-PO- | 824 |
| | PO-mU-PO-fG-PO-mC-PO-fU- | | fG-PO-mU-PO-fG-PO-mG- | |
| | PO-mG-PO-fA-PO-mA-PO-fU- | | PO-fU-PO-mA-PO-fC-PO- | |
| | PO-mG-PO-fU-PO-mA-PO-fC- | | mA-PO-fU-PO-mU-PO-fC- | |
| | PO-mC-PO-fA-PO-mC-PO-fC- | | PO-mA-PO-fG-PO-mC-PO- | |
| | PO-mA-PO-fU-PO-invdT-Hy | | fA-PO-mG-PO-dT-PO-dT-Hy | |
| 183 | Hy-mC-PO-mC-PO-mA-PO-fU- | 611 | Hy-fA-PO-fA-PO-mU-PO- | 825 |
| | PO-mG-PO-fC-PO-mU-PO-fG- | | fG-PO-mG-PO-fU-PO-mG- | |
| | PO-mA-PO-fA-PO-mU-PO-fG- | | PO-fG-PO-mU-PO-fA-PO- | |
| | PO-mU-PO-fA-PO-mC-PO-fC- | | mC-PO-fA-PO-mU-PO-fU- | |
| | PO-mA-PO-fC-PO-mC-PO-fA- | | PO-mC-PO-fA-PO-mG-PO- | |
| | PO-mU-PO-fU-PO-invdT-Hy | | fC-PO-mA-PO-dT-PO-dT-Hy | |
| 184 | Hy-mC-PO-mC-PO-mA-PO-fG- | 612 | Hy-fA-PO-fA-PO-mA-PO- | 826 |
| | PO-mC-PO-fU-PO-mG-PO-fA- | | fU-PO-mG-PO-fG-PO-mU- | |
| | PO-mA-PO-fU-PO-mG-PO-fU- | | PO-fG-PO-mG-PO-fU-PO- | |
| | PO-mA-PO-fC-PO-mC-PO-fA- | | mA-PO-fC-PO-mA-PO-fU- | |
| | PO-mC-PO-fC-PO-mA-PO-fU- | | PO-mU-PO-fC-PO-mA-PO- | |
| | PO-mU-PO-fU-PO-invdT-Hy | | fG-PO-mC-PO-dT-PO-dT-Hy | |
| 185 | Hy-mC-PO-mC-PO-mA-PO-fC- | 613 | Hy-fU-PO-fA-PO-mA-PO- | 827 |
| | PO-mU-PO-fG-PO-mA-PO-fA- | | fA-PO-mU-PO-fG-PO-mG- | |
| | PO-mU-PO-fG-PO-mU-PO-fA- | | PO-fU-PO-mG-PO-fG-PO- | |
| | PO-mC-PO-fC-PO-mA-PO-fC- | | mU-PO-fA-PO-mC-PO-fA- | |
| | PO-mC-PO-fA-PO-mU-PO-fU- | | PO-mU-PO-fU-PO-mC-PO- | |
| | PO-mU-PO-fA-PO-invdT-Hy | | fA-PO-mG-PO-dT-PO-dT-Hy | |
| 186 | Hy-mC-PO-mC-PO-mA-PO-fA- | 614 | Hy-fC-PO-fA-PO-mC-PO- | 828 |
| | PO-mG-PO-fA-PO-mG-PO-fG- | | fU-PO-mU-PO-fG-PO-mU- | |
| | PO-mU-PO-fG-PO-mA-PO-fA- | | PO-fA-PO-mU-PO-fG-PO- | |
| | PO-mC-PO-fA-PO-mU-PO-fA- | | mU-PO-fU-PO-mC-PO-fA- | |
| | PO-mC-PO-fA-PO-mA-PO-fG- | | PO-mC-PO-fC-PO-mU-PO- | |
| | PO-mU-PO-fG-PO-invdT-Hy | | fC-PO-mU-PO-dT-PO-dT-Hy | |
| 187 | Hy-mC-PO-mC-PO-mA-PO-fG- | 615 | Hy-fC-PO-fC-PO-mA-PO- | 829 |
| | PO-mA-PO-fG-PO-mG-PO-fU- | | fC-PO-mU-PO-fU-PO-mG- | |
| | PO-mG-PO-fA-PO-mA-PO-fC- | | PO-fU-PO-mA-PO-fU-PO- | |
| | PO-mA-PO-fU-PO-mA-PO-fC- | | mG-PO-fU-PO-mU-PO-fC- | |
| | PO-mA-PO-fA-PO-mG-PO-fU- | | PO-mA-PO-fC-PO-mC-PO- | |
| | PO-mG-PO-fG-PO-invdT-Hy | | fU-PO-mC-PO-dT-PO-dT-Hy | |
| 188 | Hy-mC-PO-mC-PO-mA-PO-fA- | 616 | Hy-fG-PO-fC-PO-mC-PO- | 830 |
| | PO-mG-PO-fG-PO-mU-PO-fG- | | fA-PO-mC-PO-fU-PO-mU- | |
| | PO-mA-PO-fA-PO-mC-PO-fA- | | PO-fG-PO-mU-PO-fA-PO- | |
| | PO-mU-PO-fA-PO-mC-PO-fA- | | mU-PO-fG-PO-mU-PO-fU- | |
| | PO-mA-PO-fG-PO-mU-PO-fG- | | PO-mC-PO-fA-PO-mC-PO- | |
| | PO-mG-PO-fC-PO-invdT-Hy | | fC-PO-mU-PO-dT-PO-dT-Hy | |
| 189 | Hy-mC-PO-mC-PO-mA-PO-fG- | 617 | Hy-fU-PO-fG-PO-mC-PO- | 831 |
| | PO-mG-PO-fU-PO-mG-PO-fA- | | fC-PO-mA-PO-fC-PO-mU- | |
| | PO-mA-PO-fC-PO-mA-PO-fU- | | PO-fU-PO-mG-PO-fU-PO- | |
| | PO-mA-PO-fC-PO-mA-PO-fA- | | mA-PO-fU-PO-mG-PO-fU- | |
| | PO-mG-PO-fU-PO-mG-PO-fG- | | PO-mU-PO-fC-PO-mA-PO- | |
| | PO-mC-PO-fA-PO-invdT-Hy | | fC-PO-mC-PO-dT-PO-dT-Hy | |
| 190 | Hy-mC-PO-mC-PO-mA-PO-fA- | 618 | Hy-fG-PO-fA-PO-mU-PO- | 832 |
| | PO-mA-PO-fG-PO-mU-PO-fG- | | fG-PO-mG-PO-fC-PO-mA- | |
| | PO-mG-PO-fC-PO-mA-PO-fU- | | PO-fU-PO-mA-PO-fC-PO- | |
| | PO-mG-PO-fU-PO-mA-PO-fU- | | mA-PO-fU-PO-mG-PO-fC- | |
| | PO-mG-PO-fC-PO-mC-PO-fA- | | PO-mC-PO-fA-PO-mC-PO- | |
| | PO-mU-PO-fC-PO-invdT-Hy | | fU-PO-mU-PO-dT-PO-dT-Hy | |
| 191 | Hy-mC-PO-mC-PO-mA-PO-fA- | 619 | Hy-fU-PO-fG-PO-mA-PO- | 833 |
| | PO-mG-PO-fU-PO-mG-PO-fG- | | fU-PO-mG-PO-fG-PO-mC- | |
| | PO-mC-PO-fA-PO-mU-PO-fG- | | PO-fA-PO-mU-PO-fA-PO- | |
| | PO-mU-PO-fA-PO-mU-PO-fG- | | mC-PO-fA-PO-mU-PO-fG- | |
| | PO-mC-PO-fC-PO-mA-PO-fU- | | PO-mC-PO-fC-PO-mA-PO- | |
| | PO-mC-PO-fA-PO-invdT-Hy | | fC-PO-mU-PO-dT-PO-dT-Hy | |
| 192 | Hy-mC-PO-mC-PO-mA-PO-fG- | 620 | Hy-fC-PO-fU-PO-mG-PO- | 834 |
| | PO-mU-PO-fG-PO-mG-PO-fC- | | fA-PO-mU-PO-fG-PO-mG- | |
| | PO-mA-PO-fU-PO-mG-PO-fU- | | PO-fC-PO-mA-PO-fU-PO- | |
| | PO-mA-PO-fU-PO-mG-PO-fC- | | mA-PO-fC-PO-mA-PO-fU- | |
| | PO-mC-PO-fA-PO-mU-PO-fC- | | PO-mG-PO-fC-PO-mC-PO- | |
| | PO-mA-PO-fG-PO-invdT-Hy | | fA-PO-mC-PO-dT-PO-dT-Hy | |
| 193 | Hy-mC-PO-mC-PO-mA-PO-fU- | 621 | Hy-fU-PO-fC-PO-mU-PO- | 835 |
| | PO-mG-PO-fG-PO-mC-PO-fA- | | fG-PO-mA-PO-fU-PO-mG- | |
| | PO-mU-PO-fG-PO-mU-PO-fA- | | PO-fG-PO-mC-PO-fA-PO- | |
| | PO-mU-PO-fG-PO-mC-PO-fC- | | mU-PO-fA-PO-mC-PO-fA- | |
| | PO-mA-PO-fU-PO-mC-PO-fA- | | PO-mU-PO-fG-PO-mC-PO- | |
| | PO-mG-PO-fA-PO-invdT-Hy | | fC-PO-mA-PO-dT-PO-dT-Hy | |
| 194 | Hy-mC-PO-mC-PO-mA-PO-fC- | 622 | Hy-fA-PO-fA-PO-mC-PO- | 836 |
| | PO-mU-PO-fA-PO-mU-PO-fA- | | fU-PO-mA-PO-fG-PO-mA- | |
| | PO-mC-PO-fG-PO-mC-PO-fU- | | PO-fU-PO-mG-PO-fU-PO- | |
| | PO-mA-PO-fC-PO-mA-PO-fU- | | mA-PO-fG-PO-mC-PO-fG- | |
| | PO-mC-PO-fU-PO-mA-PO-fG- | | PO-mU-PO-fA-PO-mU-PO- | |
| | PO-mU-PO-fU-PO-invdT-Hy | | fA-PO-mG-PO-dT-PO-dT-Hy | |
| 195 | Hy-mC-PO-mC-PO-mA-PO-fC- | 623 | Hy-fC-PO-fA-PO-mG-PO- | 837 |
| | PO-mA-PO-fA-PO-mA-PO-fA- | | fU-PO-mU-PO-fG-PO-mA- | |
| | PO-mG-PO-fG-PO-mA-PO-fC- | | PO-fA-PO-mG-PO-fU-PO- | |
| | PO-mA-PO-fC-PO-mU-PO-fU- | | mG-PO-fU-PO-mC-PO-fC- | |
| | PO-mC-PO-fA-PO-mA-PO-fC- | | PO-mU-PO-fU-PO-mU-PO- | |
| | PO-mU-PO-fG-PO-invdT-Hy | | fU-PO-mG-PO-dT-PO-dT-Hy | |
| 196 | Hy-mC-PO-mC-PO-mA-PO-fA- | 624 | Hy-fA-PO-fC-PO-mA-PO- | 838 |
| | PO-mA-PO-fA-PO-mA-PO-fG- | | fG-PO-mU-PO-fU-PO-mG- | |
| | PO-mG-PO-fA-PO-mC-PO-fA- | | PO-fA-PO-mA-PO-fG-PO- | |
| | PO-mC-PO-fU-PO-mU-PO-fC- | | mU-PO-fG-PO-mU-PO-fC- | |
| | PO-mA-PO-fA-PO-mC-PO-fU- | | PO-mC-PO-fU-PO-mU-PO- | |
| | PO-mG-PO-fU-PO-invdT-Hy | | fU-PO-mU-PO-dT-PO-dT-Hy | |
| 197 | Hy-mC-PO-mC-PO-mA-PO-fA- | 625 | Hy-fG-PO-fA-PO-mC-PO- | 839 |
| | PO-mA-PO-fA-PO-mG-PO-fG- | | fA-PO-mG-PO-fU-PO-mU- | |
| | PO-mA-PO-fC-PO-mA-PO-fC- | | PO-fG-PO-mA-PO-fA-PO- | |
| | PO-mU-PO-fU-PO-mC-PO-fA- | | mG-PO-fU-PO-mG-PO-fU- | |
| | PO-mA-PO-fC-PO-mU-PO-fG- | | PO-mC-PO-fC-PO-mU-PO- | |
| | PO-mU-PO-fC-PO-invdT-Hy | | fU-PO-mU-PO-dT-PO-dT-Hy | |
| 198 | Hy-mC-PO-mC-PO-mA-PO-fA- | 626 | Hy-fG-PO-fG-PO-mA-PO- | 840 |
| | PO-mA-PO-fG-PO-mG-PO-fA- | | fC-PO-mA-PO-fG-PO-mU- | |
| | PO-mC-PO-fA-PO-mC-PO-fU- | | PO-fU-PO-mG-PO-fA-PO- | |
| | PO-mU-PO-fC-PO-mA-PO-fA- | | mA-PO-fG-PO-mU-PO-fG- | |
| | PO-mC-PO-fU-PO-mG-PO-fU- | | PO-mU-PO-fC-PO-mC-PO- | |
| | PO-mC-PO-fC-PO-invdT-Hy | | fU-PO-mU-PO-dT-PO-dT-Hy | |
| 199 | Hy-mC-PO-mC-PO-mA-PO-fA- | 627 | Hy-fU-PO-fG-PO-mG-PO- | 841 |
| | PO-mG-PO-fG-PO-mA-PO-fC- | | fA-PO-mC-PO-fA-PO-mG- | |
| | PO-mA-PO-fC-PO-mU-PO-fU- | | PO-fU-PO-mU-PO-fG-PO- | |
| | PO-mC-PO-fA-PO-mA-PO-fC- | | mA-PO-fA-PO-mG-PO-fU- | |
| | PO-mU-PO-fG-PO-mU-PO-fC- | | PO-mG-PO-fU-PO-mC-PO- | |
| | PO-mC-PO-fA-PO-invdT-Hy | | fC-PO-mU-PO-dT-PO-dT-Hy | |
| 200 | Hy-mC-PO-mC-PO-mA-PO-fG- | 628 | Hy-fC-PO-fU-PO-mG-PO- | 842 |
| | PO-mG-PO-fA-PO-mC-PO-fA- | | fG-PO-mA-PO-fC-PO-mA- | |
| | PO-mC-PO-fU-PO-mU-PO-fC- | | PO-fG-PO-mU-PO-fU-PO- | |
| | PO-mA-PO-fA-PO-mC-PO-fU- | | mG-PO-fA-PO-mA-PO-fG- | |
| | PO-mG-PO-fU-PO-mC-PO-fC- | | PO-mU-PO-fG-PO-mU-PO- | |
| | PO-mA-PO-fG-PO-invdT-Hy | | fC-PO-mC-PO-dT-PO-dT-Hy | |
| 201 | Hy-mC-PO-mC-PO-mA-PO-fG- | 629 | Hy-fC-PO-fC-PO-mU-PO- | 843 |
| | PO-mU-PO-fC-PO-mC-PO-fA- | | fG-PO-mA-PO-fA-PO-mU- | |
| | PO-mG-PO-fA-PO-mG-PO-fG- | | PO-fA-PO-mA-PO-fC-PO- | |
| | PO-mG-PO-fU-PO-mU-PO-fA- | | mC-PO-fC-PO-mU-PO-fC- | |
| | PO-mU-PO-fU-PO-mC-PO-fA- | | PO-mU-PO-fG-PO-mG-PO- | |
| | PO-mG-PO-fG-PO-invdT-Hy | | fA-PO-mC-PO-dT-PO-dT-Hy | |
| 202 | Hy-mC-PO-mC-PO-mA-PO-fU- | 630 | Hy-fU-PO-fC-PO-mC-PO- | 844 |
| | PO-mC-PO-fC-PO-mA-PO-fG- | | fU-PO-mG-PO-fA-PO-mA- | |
| | PO-mA-PO-fG-PO-mG-PO-fG- | | PO-fU-PO-mA-PO-fA-PO- | |
| | PO-mU-PO-fU-PO-mA-PO-fU- | | mC-PO-fC-PO-mC-PO-fU- | |
| | PO-mU-PO-fC-PO-mA-PO-fG- | | PO-mC-PO-fU-PO-mG-PO- | |
| | PO-mG-PO-fA-PO-invdT-Hy | | fG-PO-mA-PO-dT-PO-dT-Hy | |
| 203 | Hy-mC-PO-mC-PO-mA-PO-fC- | 631 | Hy-fC-PO-fU-PO-mC-PO- | 845 |
| | PO-mC-PO-fA-PO-mG-PO-fA- | | fC-PO-mU-PO-fG-PO-mA- | |
| | PO-mG-PO-fG-PO-mG-PO-fU- | | PO-fA-PO-mU-PO-fA-PO- | |
| | PO-mU-PO-fA-PO-mU-PO-fU- | | mA-PO-fC-PO-mC-PO-fC- | |
| | PO-mC-PO-fA-PO-mG-PO-fG- | | PO-mU-PO-fC-PO-mU-PO- | |
| | PO-mA-PO-fG-PO-invdT-Hy | | fG-PO-mG-PO-dT-PO-dT-Hy | |
| 204 | Hy-mC-PO-mC-PO-mA-PO-fC- | 632 | Hy-fC-PO-fC-PO-mU-PO- | 846 |
| | PO-mA-PO-fG-PO-mA-PO-fG- | | fC-PO-mC-PO-fU-PO-mG- | |
| | PO-mG-PO-fG-PO-mU-PO-fU- | | PO-fA-PO-mA-PO-fU-PO- | |
| | PO-mA-PO-fU-PO-mU-PO-fC- | | mA-PO-fA-PO-mC-PO-fC- | |
| | PO-mA-PO-fG-PO-mG-PO-fA- | | PO-mC-PO-fU-PO-mC-PO- | |
| | PO-mG-PO-fG-PO-invdT-Hy | | fU-PO-mG-PO-dT-PO-dT-Hy | |
| 205 | Hy-mC-PO-mC-PO-mA-PO-fA- | 633 | Hy-fG-PO-fC-PO-mC-PO- | 847 |
| | PO-mG-PO-fA-PO-mG-PO-fG- | | fU-PO-mC-PO-fC-PO-mU- | |
| | PO-mG-PO-fU-PO-mU-PO-fA- | | PO-fG-PO-mA-PO-fA-PO- | |
| | PO-mU-PO-fU-PO-mC-PO-fA- | | mU-PO-fA-PO-mA-PO-fC- | |
| | PO-mG-PO-fG-PO-mA-PO-fG- | | PO-mC-PO-fC-PO-mU-PO- | |
| | PO-mG-PO-fC-PO-invdT-Hy | | fC-PO-mU-PO-dT-PO-dT-Hy | |
| 206 | Hy-mC-PO-mC-PO-mA-PO-fG- | 634 | Hy-fA-PO-fG-PO-mC-PO- | 848 |
| | PO-mA-PO-fG-PO-mG-PO-fG- | | fC-PO-mU-PO-fC-PO-mC- | |
| | PO-mU-PO-fU-PO-mA-PO-fU- | | PO-fU-PO-mG-PO-fA-PO- | |
| | PO-mU-PO-fC-PO-mA-PO-fG- | | mA-PO-fU-PO-mA-PO-fA- | |
| | PO-mG-PO-fA-PO-mG-PO-fG- | | PO-mC-PO-fC-PO-mC-PO- | |
| | PO-mC-PO-fU-PO-invdT-Hy | | fU-PO-mC-PO-dT-PO-dT-Hy | |
| 207 | Hy-mC-PO-mC-PO-mA-PO-fA- | 635 | Hy-fC-PO-fA-PO-mG-PO- | 849 |
| | PO-mG-PO-fG-PO-mG-PO-fU- | | fC-PO-mC-PO-fU-PO-mC- | |
| | PO-mU-PO-fA-PO-mU-PO-fU- | | PO-fC-PO-mU-PO-fG-PO- | |
| | PO-mC-PO-fA-PO-mG-PO-fG- | | mA-PO-fA-PO-mU-PO-fA- | |
| | PO-mA-PO-fG-PO-mG-PO-fC- | | PO-mA-PO-fC-PO-mC-PO- | |
| | PO-mU-PO-fG-PO-invdT-Hy | | fC-PO-mU-PO-dT-PO-dT-Hy | |
| 208 | Hy-mC-PO-mC-PO-mA-PO-fG- | 636 | Hy-fA-PO-fC-PO-mC-PO- | 850 |
| | PO-mG-PO-fU-PO-mU-PO-fA- | | fA-PO-mG-PO-fC-PO-mC- | |
| | PO-mU-PO-fU-PO-mC-PO-fA- | | PO-fU-PO-mC-PO-fC-PO- | |
| | PO-mG-PO-fG-PO-mA-PO-fG- | | mU-PO-fG-PO-mA-PO-fA- | |
| | PO-mG-PO-fC-PO-mU-PO-fG- | | PO-mU-PO-fA-PO-mA-PO- | |
| | PO-mG-PO-fU-PO-invdT-Hy | | fC-PO-mC-PO-dT-PO-dT-Hy | |
| 209 | Hy-mC-PO-mC-PO-mA-PO-fA- | 637 | Hy-fC-PO-fU-PO-mU-PO- | 851 |
| | PO-mA-PO-fG-PO-mA-PO-fG- | | fC-PO-mC-PO-fA-PO-mA- | |
| | PO-mG-PO-fA-PO-mU-PO-fU- | | PO-fG-PO-mA-PO-fU-PO- | |
| | PO-mA-PO-fU-PO-mC-PO-fU- | | mA-PO-fA-PO-mU-PO-fC- | |
| | PO-mU-PO-fG-PO-mG-PO-fA- | | PO-mC-PO-fU-PO-mC-PO- | |
| | PO-mA-PO-fG-PO-invdT-Hy | | fU-PO-mU-PO-dT-PO-dT-Hy | |
| 210 | Hy-mC-PO-mC-PO-mA-PO-fA- | 638 | Hy-fA-PO-fC-PO-mU-PO- | 852 |
| | PO-mG-PO-fA-PO-mG-PO-fG- | | fU-PO-mC-PO-fC-PO-mA- | |
| | PO-mA-PO-fU-PO-mU-PO-fA- | | PO-fA-PO-mG-PO-fA-PO- | |
| | PO-mU-PO-fC-PO-mU-PO-fU- | | mU-PO-fA-PO-mA-PO-fU- | |
| | PO-mG-PO-fG-PO-mA-PO-fA- | | PO-mC-PO-fC-PO-mU-PO- | |
| | PO-mG-PO-fU-PO-invdT-Hy | | fC-PO-mU-PO-dT-PO-dT-Hy | |
| 211 | Hy-mC-PO-mC-PO-mA-PO-fG- | 639 | Hy-fG-PO-fA-PO-mC-PO- | 853 |
| | PO-mA-PO-fG-PO-mG-PO-fA- | | fU-PO-mU-PO-fC-PO-mC- | |
| | PO-mU-PO-fU-PO-mA-PO-fU- | | PO-fA-PO-mA-PO-fG-PO- | |
| | PO-mC-PO-fU-PO-mU-PO-fG- | | mA-PO-fU-PO-mA-PO-fA- | |
| | PO-mG-PO-fA-PO-mA-PO-fG- | | PO-mU-PO-fC-PO-mC-PO- | |
| | PO-mU-PO-fC-PO-invdT-Hy | | fU-PO-mC-PO-dT-PO-dT-Hy | |
| 212 | Hy-mC-PO-mC-PO-mA-PO-fA- | 640 | Hy-mG-PO-fA-PO-mA-PO- | 854 |
| | PO-mG-PO-fA-PO-mC-PO-fA- | | fU-PO-mC-PO-fA-PO-mA- | |
| | PO-mA-PO-fU-PO-mU-PO-fC- | | PO-fA-PO-mU-PO-fG-PO- | |
| | PO-mA-PO-fU-PO-mC-PO-fA- | | mA-PO-fU-PO-mG-PO-fA- | |
| | PO-mU-PO-fU-PO-mU-PO-fG- | | PO-mA-PO-fU-PO-mU-PO- | |
| | PO-mA-PO-fU-PO-mU-PO-fC- | | fG-PO-mU-PO-fC-PO-mU- | |
| | PO-invdT-Hy | | PO-dT-PO-dT-Hy | |
| 213 | Hy-mC-PO-mC-PO-mA-PO-fA- | 641 | Hy-fA-PO-mA-PO-fU-PO- | 855 |
| | PO-mG-PO-fA-PO-mC-PO-fA- | | mC-PO-fA-PO-mA-PO-fA- | |
| | PO-mA-PO-fU-PO-mU-PO-fC- | | PO-mU-PO-fG-PO-mA-PO- | |
| | PO-mA-PO-fU-PO-mC-PO-fA- | | fU-PO-mG-PO-fA-PO-mA- | |
| | PO-mU-PO-fU-PO-mU-PO-fG- | | PO-fU-PO-mU-PO-fG-PO- | |
| | PO-mA-PO-fU-PO-mU-PO- | | mU-PO-fC-PO-mU-PO-dT- | |
| | invdT-Hy | | PO-dT-Hy | |
| 214 | Hy-mC-PO-mC-PO-mA-PO-fG- | 642 | Hy-fG-PO-mA-PO-fA-PO- | 856 |
| | PO-mA-PO-fC-PO-mA-PO-fA- | | mU-PO-fC-PO-mA-PO-fA- | |
| | PO-mU-PO-fU-PO-mC-PO-fA- | | PO-mA-PO-fU-PO-mG-PO- | |
| | PO-mU-PO-fC-PO-mA-PO-fU- | | fA-PO-mU-PO-fG-PO-mA- | |
| | PO-mU-PO-fU-PO-mG-PO-fA- | | PO-fA-PO-mU-PO-fU-PO- | |
| | PO-mU-PO-fU-PO-mC-PO- | | mG-PO-fU-PO-mC-PO-dT- | |
| | invdT-Hy | | PO-dT-Hy | |

In some embodiments, the dsRNA comprises one or more modified nucleotides described in PCT Publication WO 2019/170731, the disclosure of which is incorporated herein in its entirety. In such modified nucleotides, the ribose ring has been replaced by a six-membered heterocyclic ring. Such a modified nucleotide has the structure of formula (I):

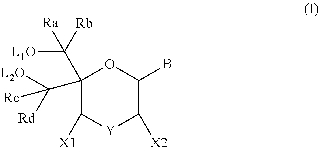

wherein:
   B is a heterocyclic nucleobase;
   one of L1 and L2 is an internucleoside linking group linking the compound of formula (I) to a polynucleotide and the other of L1 and L2 is H, a protecting group, a phosphorus moiety or an internucleoside linking group linking the compound of formula (I) to a polynucleotide,
   Y is O, NH, NR1 or N—C(═O)—R1, wherein R1 is:
a (C1-C20) alkyl group, optionally substituted by one or more groups selected from an halogen atom, a (C1-C6) alkyl group, a (C3-C8) cycloalkyl group, a (C3-C14) heterocycle, a (C6-C14) aryl group, a (C5-C14) heteroaryl group, —O-Z1, —N(Z1)(Z2), —S-Z1, —CN, —C(=J)-O-Z1, —O—C(=J)-Z1, —C(=J)-N(Z1)(Z2), and —N(Z1)-C(=J)-Z2, wherein
J is O or S,
each of Z1 and Z2 is, independently, H, a (C1-C6) alkyl group, optionally substituted by one or more groups selected from a halogen atom and a (C1-C6) alkyl group,
a (C3-C8) cycloalkyl group, optionally substituted by one or more groups selected from a halogen atom and a (C1-C6) alkyl group,
a group —[C(═O)]m-R2-(O—CH₂—CH₂)p-R3, wherein
m is an integer meaning 0 or 1,
p is an integer ranging from 0 to 10, R2 is a (C1-C20) alkylene group optionally substituted by a (C1-C6) alkyl group, —O-Z3, —N(Z3)(Z4), —S-Z3, —CN, —C(═K)—O—Z3, —O—C(═K)—Z3, —C(═K)—N(Z3)(Z4), or —N(Z3)-C(═K)—Z4,
wherein
K is O or S,
each of Z3 and Z4 is, independently, H, a (C1-C6) alkyl group, optionally substituted by one or more groups selected from a halogen atom and a (C1-C6) alkyl group, and
R3 is selected from the group consisting of a hydrogen atom, a (C1-C6) alkyl group, a (C1-C6) alkoxy group, a (C3-C8) cycloalkyl group, a (C3-C14) heterocycle, a (C6-C14) aryl group or a (C5-C14) heteroaryl group, or R3 is a cell targeting moiety,
   X1 and X2 are each, independently, a hydrogen atom, a (C1-C6) alkyl group, and
   each of Ra, Rb, Rc and Rd is, independently, H or a (C1-C6) alkyl group,
or is a pharmaceutically acceptable salt thereof.

In some embodiments, Y is NR1, R1 is a non-substituted (C1-C20) alkyl group, and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a non-substituted (C1-C16) alkyl group, which includes an alkyl group selected from a group comprising methyl, isopropyl, butyl, octyl, hexadecyl, and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a (C3-C8) cycloalkyl group, optionally substituted by one or more groups selected from a halogen atom and a (C1-C6) alkyl group, and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a cyclohexyl group, and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a (C1-C20) alkyl group substituted by a (C6-C14) aryl group and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a methyl group substituted by a phenyl group, and L1, L2, Ra, Rb, Rc, Rd, X1, X2, R2, R3 and B have the same meaning as defined for the general formula (I), or a pharmaceutically acceptable salt thereof.

In some embodiments, Y is N—C(═O)—R1, R1 is an optionally substituted (C1-C20) alkyl group, and L1, L2, Ra, Rb, Rc, Rd, X1, X2, R2, R3 and B have the same meaning as defined for the general formula (I), or a pharmaceutically acceptable salt thereof.

In some embodiments, Y is N—C(═O)—R1, R1 is selected from a group comprising methyl and pentadecyl and L1, L2, Ra, Rb, Rc, Rd, X1, X2, R2, R3 and B have the same meaning as defined for the general formula (I), or a pharmaceutically acceptable salt thereof.

In some embodiments, B is selected from a group comprising a pyrimidine, a substituted pyrimidine, a purine and a substituted purine, or a pharmaceutically acceptable salt thereof.

In some embodiments, the internucleoside linking group in the dsRNA is independently selected from the group consisting of phosphodiester, phosphotriester, phosphorothioate, phosphorodithioate, alkyl-phosphonate and phosphoramidate backbone linking groups, or a pharmaceutically acceptable salt thereof.

In some embodiments, the dsRNA comprises from 2 to 10 compounds of formula (I), or a pharmaceutically acceptable salt thereof.

In further embodiments, the dsRNA comprises one or more targeted nucleotides or a pharmaceutically acceptable salt thereof.

In some embodiments, R3 is of the formula (II):

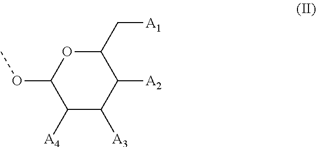

wherein A1, A2 and A3 are OH,
A4 is OH or NHC(═O)—R5, wherein R5 is a (C1-C6) alkyl group, optionally substituted by a halogen atom.

In some embodiments, R3 is N-acetyl-galactosamine.

The precursors that can be used to make modified siRNAs having nucleotides of formula (I) are exemplified in Table A, which shows examples of phosphoramidite nucleotide analogs for oligonucleotide synthesis. In the (2S,6R) diastereomeric series, the phosphoramidites as nucleotide precursors are abbreviated with a “pre-1”, the nucleotide analogs are abbreviated with an “1”, followed by the nucleobase and a number, which specifies the group Y in formula (I). To distinguish both stereochemistries, the analogues (2R,6R)-diastereoisomers are indicated with an additional “b.” Targeted nucleotide precursors, targeted nucleotide analogs and solid supports are abbreviated as described above, but with an “lg” instead of the “l.”

TABLE A

| | | | Name in | |
|---|---|---|---|---|
| | | Precursor | oligo- | |
| N^{o} | Structure | name | sequence | Stereochemistry |
| 1 | 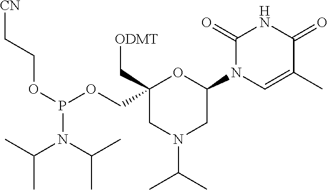 | pre-lT3 | lT3 | (2S,6R) |
| 2 | 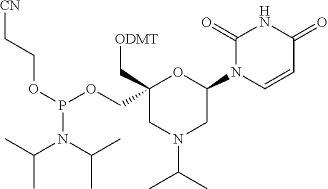 | pre-lU3 | lU3 | (2S,6R) |
| 3 | 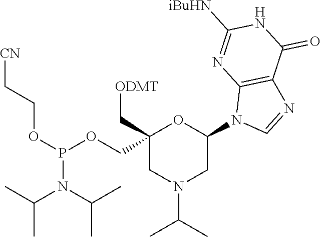 | pre-lG3 | lG3 | (2S,6R) |
| 4 | 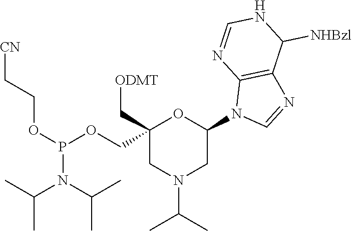 | pre-lA3 | lA3 | (2S,6R) |
| 5 | 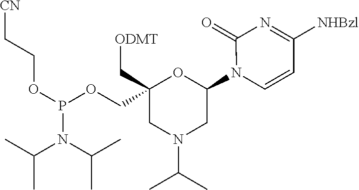 | pre-lC3 | lC3 | (2S,6R) |
| 6 | 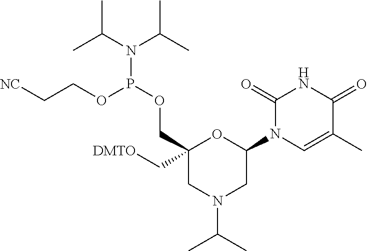 | pre-lT3b | lT3b | (2R,6R) |
| 7 | 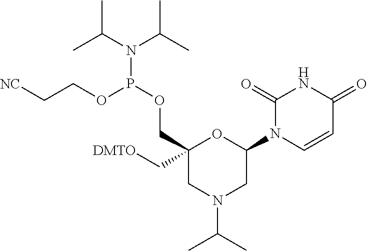 | pre-lU3b | lU3b | (2R,6R) |
| 8 | 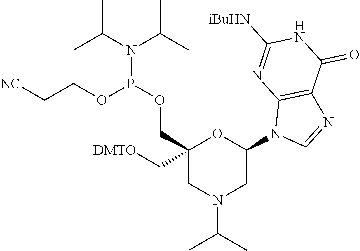 | pre-lG3b | lG3b | (2R,6R) |
| 9 | 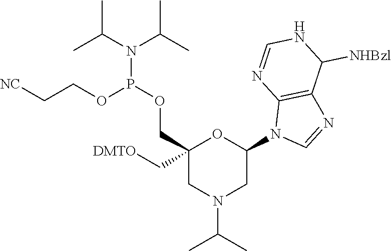 | pre-lA3b | lA3b | (2R,6R) |
| 10 | 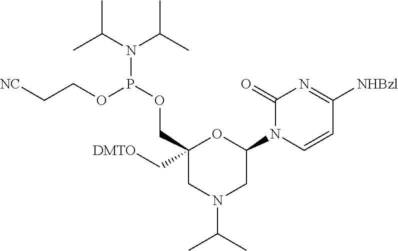 | pre-lC3b | lC3b | (2R,6R) |
| 11 | 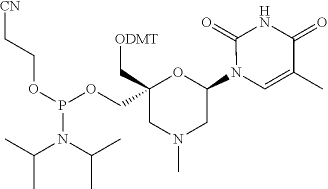 | pre-lT2 | lT2 | (2S,6R) |
| 12 | 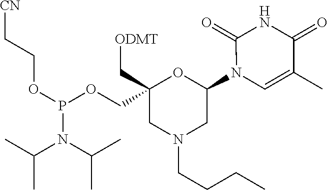 | pre-lT6 | lT6 | (2S,6R) |
| 13 | 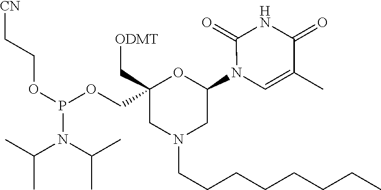 | pre-lT7 | lT7 | (2S,6R) |
| 14 | 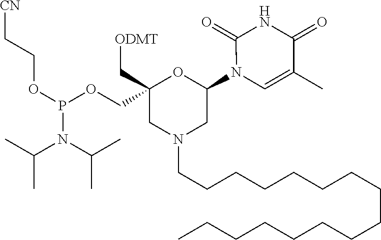 | pre-lT8 | lT8 | (2S,6R) |
| 15 | 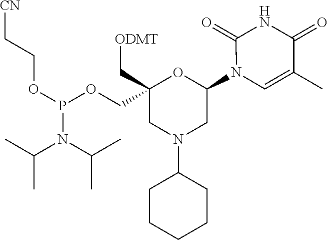 | pre-lT4 | lT4 | (2S,6R) |
| 16 | 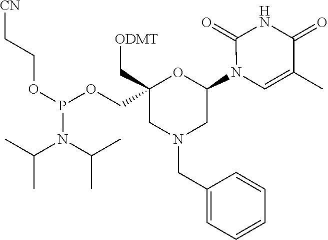 | pre-lT5 | lT5 | (2S,6R) |
| 17 | 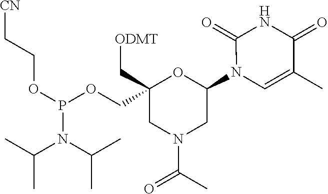 | pre-lT9 | lT9 | (2S,6R) |
| 18 | 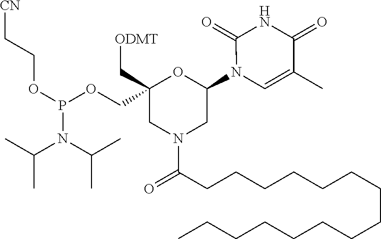 | pre-lT10 | lT10 | (2S,6R) |
| 19 | 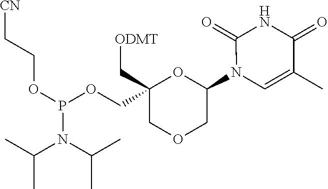 | pre-lT1 | lT1 | (2S,6R) |
| 20 | 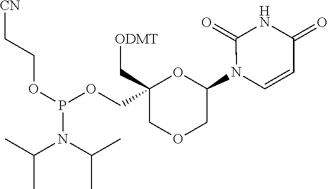 | pre-lU1 | lU1 | (2S,6R) |
| 21 | 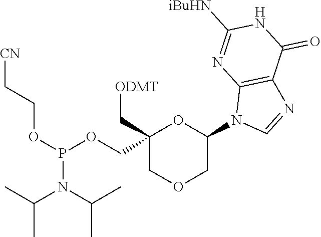 | pre-lG1 | lG1 | (2S,6R) |
| 22 | 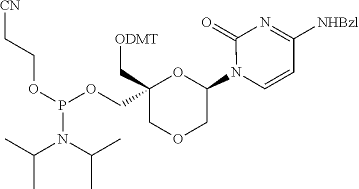 | pre-lC1 | lC1 | (2S,6R) |
| 23 | 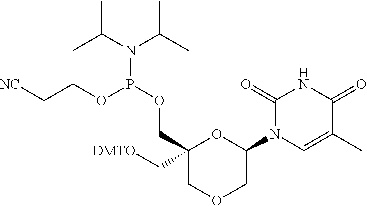 | pre-lT1b | lT1b | (2R,6R) |
| 24 | 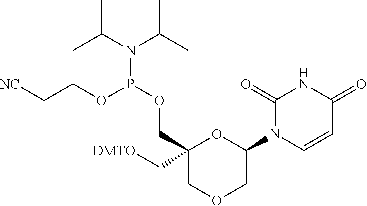 | pre-lU1b | lU1b | (2R,6R) |
| 25 | 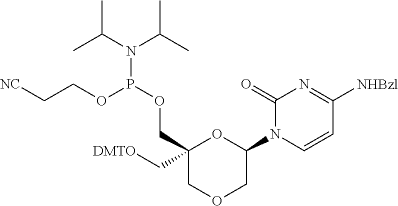 | pre-lC1b | lC1b | (2R,6R) |
| 26 | 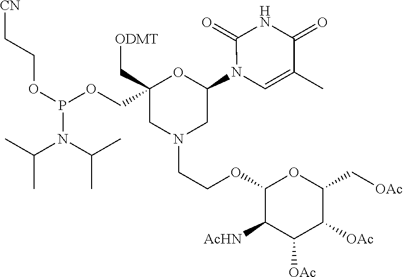 | pre-lgT9 | lgT9 | (2S,6R) |
| 27 | 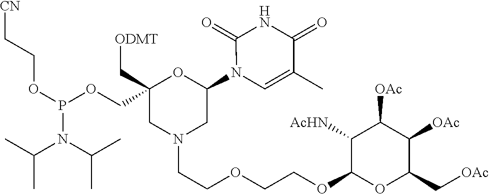 | pre-lgT8 | lgT8 | (2S,6R) |
| 28 | 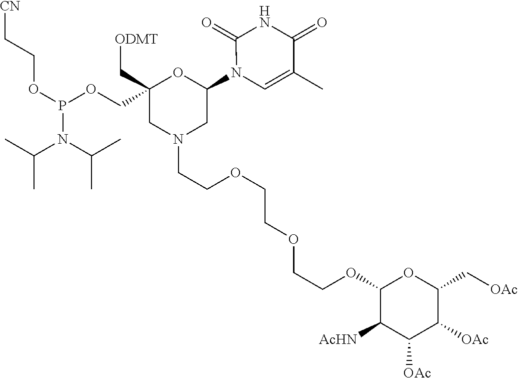 | pre-lgT7 | lgT7 | (2S,6R) |
| 29 | 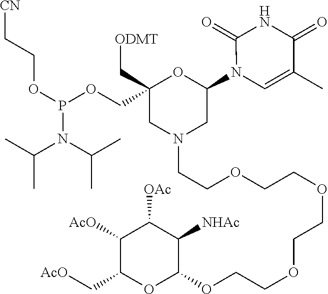 | pre-lgT6 | lgT6 | (2S,6R) |
| 30 | 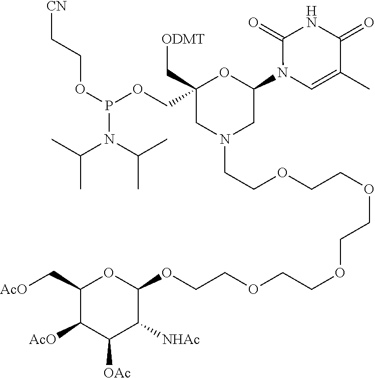 | pre-lgT5 | lgT5 | (2S,6R) |
| 31 | 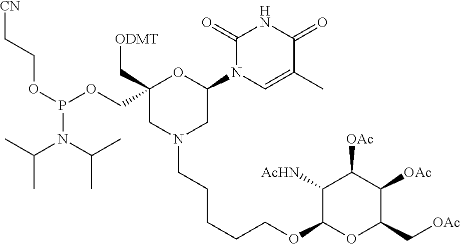 | pre-lgT3 | lgT3 | (2S,6R) |
| 32 | 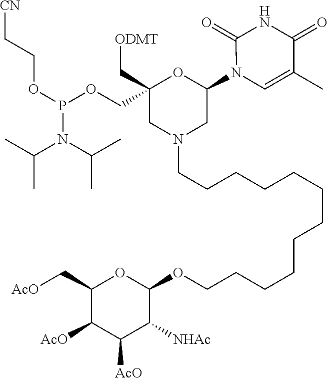 | pre-lgT4 | lgT4 | (2S,6R) |
| 33 | 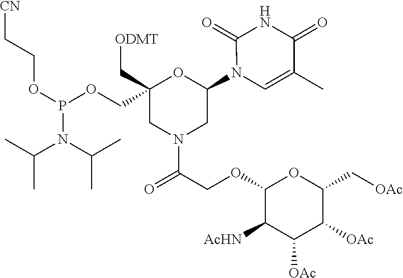 | pre-lgT12 | lgT12 | (2S,6R) |
| 34 | 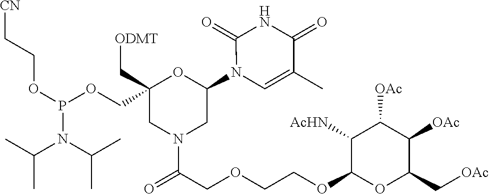 | pre-lgT11 | lgT11 | (2S,6R) |
| 35 | 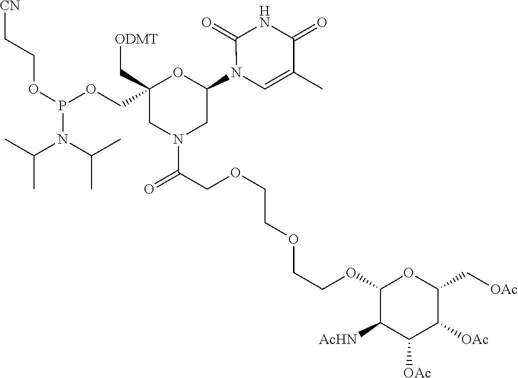 | pre-lgT10 | lgT10 | (2S,6R) |
| 36 | 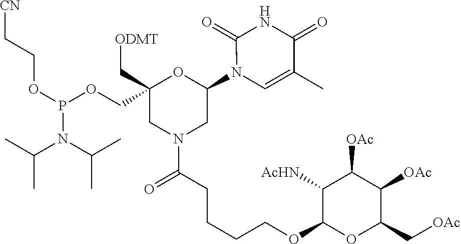 | pre-lgT1 | lgT1 | (2S,6R) |
| 37 | 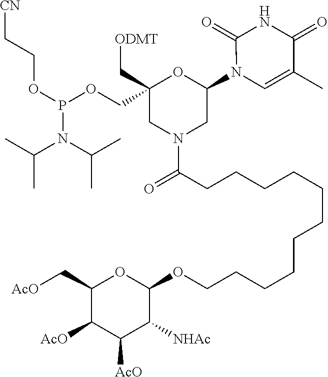 | pre-lgT2 | lgT2 | (2S,6R) |
| 38 | 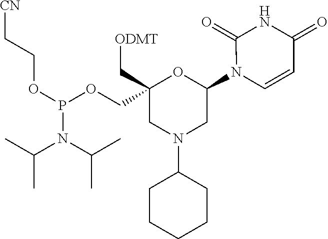 | pre-lU4 | lU4 | (2S,6R) |
| 39 | 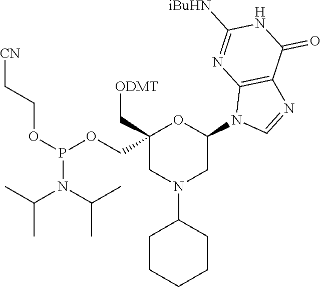 | pre-lG4 | lG4 | (2S,6R) |
| 40 | 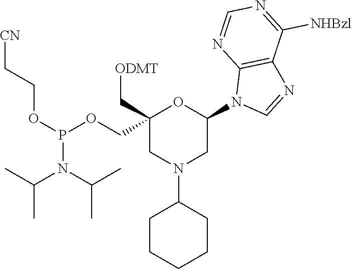 | pre-lA4 | lA4 | (2S,6R) |
| 41 | 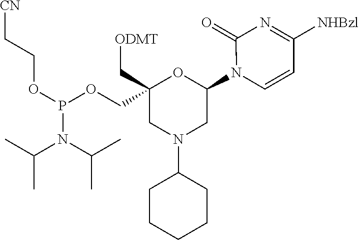 | pre-lC4 | lC4 | (2S,6R) |
| 42 | 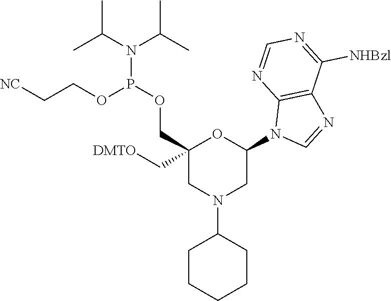 | pre-lA4b | lA4b | (2R,6R) |
| 43 | 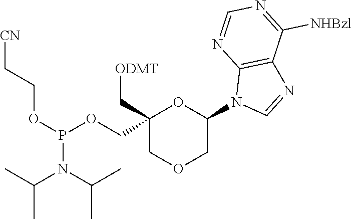 | pre-lA1 | lA1 | (2S,6R) |
| 44 | 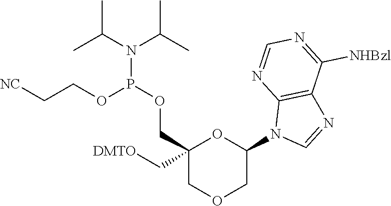 | pre-lA1b | lA1b | (2R,6R) |
| 45 | 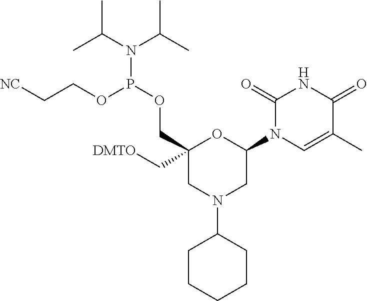 | pre-lT4b | lT4b | (2R,6R) |
| 46 | 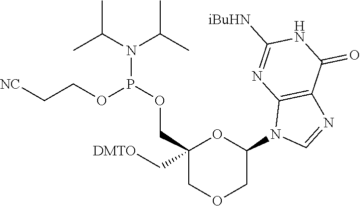 | pre-lG1b | lG1b | (2R,6R) |

The modified nucleotides of formula (I) may be incorporated at the 5′, 3′, or both ends of the sense strand and/or antisense strand of the dsRNA. By way of example, one or more (e.g., 1, 2, 3, 4, or 5 or more) modified nucleotides may be incorporated at the 5′ end of the sense strand of the dsRNA. In some embodiments, one or more (e.g., 1, 2, 3, or more) modified nucleotides are positioned in the 5′ end of the sense strand, where the modified nucleotides do not complement the antisense sequence but may be optionally paired with an equal or smaller number of complementary nucleotides at the corresponding 3′ end of the antisense strand.

In some embodiments, the dsRNA may comprise a sense strand having a sense sequence of 17, 18, or 19 nucleotides in length, where three to five nucleotides of formula (I) (e.g., three consecutive lgT3 or lgT7 with or without additional nucleotides of formula (I)) are placed in the 5′ end of the sense sequence, making the sense strand 20, 21, or 22 nucleotides in length. In such embodiments, the sense strand may additionally comprise two consecutive nucleotides of formula (I) (e.g., 1T4 or lT3) at the 3′ of the sense sequence, making the sense strand 22, 23, or 24 nucleotides in length. The dsRNA may comprise an antisense sequence of 19 nucleotides in length, where the antisense sequence may additionally be linked to 2 modified nucleotides or deoxyribonucleotides (e.g., dT) at its 3′ end, making the antisense strand 21 nucleotides in length. In further embodiments, the sense strand of the dsRNA contains only naturally occurring internucleotide bonds (phosphodiester bond), where the antisense strand may optionally contain non-naturally occurring internucleotide bonds. For example, the antisense strand may contain phosphoro-thioate bonds in the backbone near or at its 5′ and/or 3′ ends.

In some embodiments, the use of modified nucleotides of formula (I) circumvents the need for other RNA modifications such as the use of non-naturally occurring internucleotide bonds, thereby simplifying the chemical synthesis of dsRNAs. Moreover, the modified nucleotides of formula (I) can be readily made to contain cell targeted moieties such as GalNAc derivatives (which include GalNAc itself), enhancing the delivery efficiency of dsRNAs incorporating such nucleotides. Further, it has been shown that dsRNAs incorporating modified nucleotides of formula (I), e.g., at the sense strand, significantly improve the stability and therapeutic potency of the dsRNAs.

Table 3 below lists the sequences of exemplary modified GalNAc-siRNA constructs derived from constructs siRNA #013, siRNA #051, and siRNA #165 listed in Table 2. In the table, mX=2′-O-Me nucleotide; fX=2′-F nucleotide; dX=DNA nucleotide; lx=locked nucleic acid (LNA); PO=phosphodiester linkage; PS=phosphorothioate linkage; and Hy=hydroxyl group.

TABLE 3
Sequences of Exemplary Modified GalNAc-siRNA Constructs from siRNA#013,
siRNA#051, and siRNA#165

| SIRNA | Sense strand sequence | | Antisense strand sequence | |
|---|---|---|---|---|
| # | (5′-3′) | SEQ | (5′-3′) | SEQ |
| 013-c-01 | Hy-lgT7-PO-lgT7-PO-lgT7- | 857 | Hy-mA-PS-fU-PS-mC-PO- | 1019 |
| | PO-mA-PO-mG-PO-mA-PO-mC- | | fA-PO-mA-PO-mA-PO-mU- | |
| | PO-lA-PO-mA-PO-fU-PO-fU- | | PO-mG-PO-mA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-02 | Hy-lgT7-PO-lgT7-PO-lgT7- | 858 | Hy-mA-PS-fU-PS-mC-PO- | 1020 |
| | PO-mA-PO-mG-PO-mA-PO-mC- | | mA-PO-mA-PO-fA-PO-mU- | |
| | PO-fA-PO-mA-PO-fU-PO-fU- | | PO-mG-PO-mA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-03 | Hy-lgT7-PO-lgT7-PO-lgT7- | 859 | Hy-mA-PS-fU-PS-mC-PO- | 1021 |
| | PO-mA-PO-mG-PO-mA-PO-mC- | | fA-PO-mA-PO-mA-PO-mU- | |
| | PO-fA-PO-mA-PO-fU-PO-fU- | | PO-mG-PO-fA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-04 | Hy-lgT7-PO-lgT7-PO-lgT7- | 860 | Hy-fA-PS-fU-PS-mC-PO- | 1022 |
| | PO-mA-PO-mG-PO-mA-PO-mC- | | fA-PO-mA-PO-fA-PO-mU- | |
| | PO-fA-PO-mA-PO-fU-PO-fU- | | PO-fG-PO-mA-PO-fU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | fC-PO-mU-PS-dT-PS-dT- | |
| | | | Hy | |
| 013-c-05 | Hy-lgT7-PO-lgT7-PO-lgT7- | 861 | Hy-mA-PS-fU-PS-mC-PO- | 1023 |
| | PO-lA-PO-lG-PO-mA-PO-mC- | | fA-PO-mA-PO-mA-PO-mU- | |
| | PO-fA-PO-mA-PO-fU-PO-fU- | | PO-mG-PO-mA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-06 | Hy-lgT7-PO-lgT7-PO-lgT7- | 862 | Hy-mA-PS-fU-PS-mC-PO- | 1024 |
| | PO-lA-PO-lG-PO-mA-PO-mC- | | mA-PO-mA-PO-fA-PO-mU- | |
| | PO-fA-PO-mA-PO-fU-PO-fU- | | PO-mG-PO-mA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-07 | Hy-lgT7-PO-lgT7-PO-lgT7- | 863 | Hy-mA-PS-fU-PS-mC-PO- | 1025 |
| | PO-lA-PO-lG-PO-mA-PO-mC- | | fA-PO-mA-PO-mA-PO-mU- | |
| | PO-lA-PO-mA-PO-fU-PO-fU- | | PO-mG-PO-fA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-08 | Hy-lgT7-PO-lgT7-PO-lgT7- | 864 | Hy-fA-PS-fU-PS-mC-PO- | 1026 |
| | PO-lA-PO-lG-PO-mA-PO-mC- | | fA-PO-mA-PO-fA-PO-mU- | |
| | PO-lA-PO-mA-PO-fU-PO-fU- | | PO-fG-PO-mA-PO-fU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | fC-PO-mU-PS-dT-PS-dT- | |
| | | | Hy | |
| 013-c-09 | Hy-lgT7-PO-lgT7-PO-lgT7- | 865 | Hy-mA-PS-fU-PS-mC-PO- | 1027 |
| | PO-mA-PO-mG-PO-mA-PO-mC- | | fA-PO-mA-PO-mA-PO-mU- | |
| | PO-fA-PO-mA-PO-fU-PO-mU- | | PO-mG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-10 | Hy-lgT7-PO-lgT7-PO-lgT7- | 866 | Hy-mA-PS-fU-PS-mC-PO- | 1028 |
| | PO-mA-PO-mG-PO-mA-PO-mC- | | mA-PO-mA-PO-fA-PO-mU- | |
| | PO-fA-PO-mA-PO-fU-PO-mU- | | PO-mG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-11 | Hy-lgT7-PO-lgT7-PO-lgT7- | 867 | Hy-mA-PS-fU-PS-mC-PO- | 1029 |
| | PO-mA-PO-mG-PO-mA-PO-mC- | | fA-PO-mA-PO-mA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-12 | Hy-lgT7-PO-lgT7-PO-lgT7- | 868 | Hy-mA-PS-fU-PS-mC-PO- | 1030 |
| | PO-mA-PO-mG-PO-mA-PO-mC- | | mA-PO-mA-PO-mA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-13 | Hy-lgT7-PO-lgT7-PO-lgT7- | 869 | Hy-fA-PS-fU-PS-mC-PO- | 1031 |
| | PO-mA-PO-mG-PO-mA-PO-mC- | | fA-PO-mA-PO-fA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-fA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | fC-PO-mU-PS-dT-PS-dT- | |
| | | | Hy | |
| 013-c-14 | Hy-lgT7-PO-lgT7-PO-lgT7- | 870 | Hy-mA-PS-fU-PS-mC-PO- | 1032 |
| | PO-lA-PO-lG-PO-mA-PO-mC- | | fA-PO-mA-PO-mA-PO-mU- | |
| | PO-fA-PO-mA-PO-fU-PO-mU- | | PO-mG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-15 | Hy-lgT7-PO-lgT7-PO-lgT7- | 871 | Hy-mA-PS-fU-PS-mC-PO- | 1033 |
| | PO-lA-PO-lG-PO-mA-PO-mC- | | mA-PO-mA-PO-lA-PO-mU- | |
| | PO-fA-PO-mA-PO-fU-PO-mU- | | PO-mG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| 013-c-16 | Hy-lgT7-PO-lgT7-PO-lgT7- | 872 | Hy-mA-PS-fU-PS-mC-PO- | 1034 |
| | PO-lA-PO-lG-PO-mA-PO-mC- | | fA-PO-mA-PO-mA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-17 | Hy-lgT7-PO-lgT7-PO-lgT7- | 873 | Hy-mA-PS-fU-PS-mC-PO- | 1035 |
| | PO-lA-PO-lG-PO-mA-PO-mC- | | mA-PO-mA-PO-mA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | mC-PO-mU-PS-mA-PS-mA- | |
| | | | Hy | |
| 013-c-18 | Hy-lgT7-PO-lgT7-PO-lgT7- | 874 | Hy-fA-PS-fU-PS-mC-PO- | 1036 |
| | PO-lA-PO-lG-PO-mA-PO-mC- | | fA-PO-mA-PO-fA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | ImG-PO-fA-PO-mA-PO-fU- | |
| | PO-mA-PO-mU-PO-mU-PO-mU- | | PO-mU-PO-fG-PO-mU-PO- | |
| | PO-mG-PS-mA-PS-mU-Hy | | fC-PO-mU-PS-dT-PS-dT- | |
| | | | Hy | |
| 013-c-19 | Hy-lgT7-PO-lgT7-PO-lgT7- | 875 | Hy-mA-PS-fU-PS-mC-PO- | 1037 |
| | PO-mA-PO-mG-PO-mA-PO-mC- | | fA-PO-mA-PO-mA-PO-mU- | |
| | PO-fA-PO-mA-PO-fU-PO-fU- | | PO-mG-PO-mA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-20 | Hy-lgT7-PO-lgT7-PO-lgT7- | 876 | Hy-mA-PS-fU-PS-mC-PO- | 1038 |
| | PO-mA-PO-mG-PO-mA-PO-mC- | | mA-PO-mA-PO-lA-PO-mU- | |
| | PO-fA-PO-mA-PO-fU-PO-fU- | | PO-mG-PO-mA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-21 | Hy-lgT7-PO-lgT7-PO-lgT7- | 877 | Hy-mA-PS-fU-PS-mC-PO- | 1039 |
| | PO-mA-PO-mG-PO-mA-PO-mC- | | fA-PO-mA-PO-mA-PO-mU- | |
| | PO-fA-PO-mA-PO-fU-PO-fU- | | PO-mG-PO-fA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-22 | Hy-lgT7-PO-lgT7-PO-lgT7- | 878 | Hy-fA-PS-fU-PS-mC-PO- | 1040 |
| | PO-mA-PO-mG-PO-mA-PO-mC- | | fA-PO-mA-PO-fA-PO-mU- | |
| | PO-fA-PO-mA-PO-fU-PO-fU- | | PO-fG-PO-mA-PO-fU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | fC-PO-mU-PS-dT-PS-dT- | |
| | | | Hy | |
| 013-c-23 | Hy-lgT7-PO-lgT7-PO-lgT7- | 879 | Hy-mA-PS-fU-PS-mC-PO- | 1041 |
| | PO-lA-PO-lG-PO-mA-PO-mC- | | fA-PO-mA-PO-mA-PO-mU- | |
| | PO-lA-PO-mA-PO-fU-PO-fU- | | PO-mG-PO-mA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-24 | Hy-lgT7-PO-lgT7-PO-lgT7- | 880 | Hy-mA-PS-fU-PS-mC-PO- | 1042 |
| | PO-lA-PO-lG-PO-mA-PO-mC- | | mA-PO-mA-PO-fA-PO-mU- | |
| | PO-fA-PO-mA-PO-fU-PO-fU- | | PO-mG-PO-mA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-25 | Hy-lgT7-PO-lgT7-PO-lgT7- | 881 | Hy-mA-PS-fU-PS-mC-PO- | 1043 |
| | PO-lA-PO-lG-PO-mA-PO-mC- | | fA-PO-mA-PO-mA-PO-mU- | |
| | PO-lA-PO-mA-PO-fU-PO-fU- | | PO-mG-PO-fA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-26 | Hy-lgT7-PO-lgT7-PO-lgT7- | 882 | Hy-fA-PS-fU-PS-mC-PO- | 1044 |
| | PO-lA-PO-lG-PO-mA-PO-mC- | | fA-PO-mA-PO-fA-PO-mU- | |
| | PO-fA-PO-mA-PO-fU-PO-fU- | | PO-fG-PO-mA-PO-fU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | fC-PO-mU-PS-dT-PS-dT- | |
| | | | Hy | |
| 013-c-27 | Hy-lgT7-PO-lgT7-PO-lgT7- | 883 | Hy-mA-PS-fU-PS-mC-PO- | 1045 |
| | PO-mA-PO-mG-PO-mA-PO-mC- | | fA-PO-mA-PO-mA-PO-mU- | |
| | PO-fA-PO-mA-PO-fU-PO-mU- | | PO-mG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-28 | Hy-lgT7-PO-lgT7-PO-lgT7- | 884 | Hy-mA-PS-fU-PS-mC-PO- | 1046 |
| | PO-mA-PO-mG-PO-mA-PO-mC- | | mA-PO-mA-PO-fA-PO-mU- | |
| | PO-fA-PO-mA-PO-fU-PO-mU- | | PO-mG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-29 | Hy-lgT7-PO-lgT7-PO-lgT7- | 885 | Hy-mA-PS-fU-PS-mC-PO- | 1047 |
| | PO-mA-PO-mG-PO-mA-PO-mC- | | fA-PO-mA-PO-mA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-30 | Hy-lgT7-PO-lgT7-PO-lgT7- | 886 | Hy-mA-PS-fU-PS-mC-PO- | 1048 |
| | PO-mA-PO-mG-PO-mA-PO-mC- | | mA-PO-mA-PO-mA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| 013-c-31 | Hy-lgT7-PO-lgT7-PO-lgT7- | 887 | Hy-fA-PS-fU-PS-mC-PO- | 1049 |
| | PO-mA-PO-mG-PO-mA-PO-mC- | | fA-PO-mA-PO-fA-PO-mU- | |
| | PO-lA-PO-mA-PO-fU-PO-mU- | | PO-fG-PO-mA-PO-mU-PO- | |
| | PO-fC-PO-fA-PO-fU-PO-mC- | | mG-PO-lA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | fC-PO-mU-PS-dT-PS-dT- | |
| | | | Hy | |
| 013-c-32 | Hy-lgT7-PO-lgT7-PO-lgT7- | 888 | Hy-mA-PS-fU-PS-mC-PO- | 1050 |
| | PO-lA-PO-lG-PO-mA-PO-mC- | | fA-PO-mA-PO-mA-PO-mU- | |
| | PO-fA-PO-mA-PO-fU-PO-mU- | | PO-mG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-33 | Hy-lgT7-PO-lgT7-PO-lgT7- | 889 | Hy-mA-PS-fU-PS-mC-PO- | 1051 |
| | PO-lA-PO-lG-PO-mA-PO-mC- | | mA-PO-mA-PO-fA-PO-mU- | |
| | PO-fA-PO-mA-PO-fU-PO-mU- | | PO-mG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-34 | Hy-lgT7-PO-lgT7-PO-lgT7- | 890 | Hy-mA-PS-fU-PS-mC-PO- | 1052 |
| | PO-lA-PO-lG-PO-mA-PO-mC- | | fA-PO-mA-PO-mA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-35 | Hy-lgT7-PO-lgT7-PO-lgT7- | 891 | Hy-mA-PS-fU-PS-mC-PO- | 1053 |
| | PO-lA-PO-lG-PO-mA-PO-mC- | | mA-PO-mA-PO-mA-PO-mU- | |
| | PO-lA-PO-mA-PO-fU-PO-mU- | | PO-fG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | mC-PO-mU-PS-mA-PS-mA- | |
| | | | Hy | |
| 013-c-36 | Hy-lgT7-PO-lgT7-PO-lgT7- | 892 | Hy-fA-PS-fU-PS-mC-PO- | 1054 |
| | PO-lA-PO-lG-PO-mA-PO-mC- | | fA-PO-mA-PO-lA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-fA-PO-mA-PO-fU- | |
| | PO-mA-PO-mU-PO-mU-PO-mU- | | PO-mU-PO-fG-PO-mU-PO- | |
| | PO-mG-PO-lT4-PO-lT4-Hy | | fC-PO-mU-PS-dT-PS-dT- | |
| | | | Hy | |
| 013-c-37 | Hy-lgT7-PO-lgT7-PO-lgT7- | 893 | Hy-mA-PS-fU-PS-mC-PO- | 1055 |
| | PO-mA-PO-mG-PO-mA-PO-mC- | | fA-PO-mA-PO-mA-PO-mU- | |
| | PO-fA-PO-mA-PO-fU-PO-fU- | | PO-mG-PO-mA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-38 | Hy-lgT7-PO-lgT7-PO-lgT7- | 894 | Hy-mA-PS-fU-PS-mC-PO- | 1056 |
| | PO-mA-PO-mG-PO-mA-PO-mC- | | mA-PO-mA-PO-lA-PO-mU- | |
| | PO-fA-PO-mA-PO-fU-PO-fU- | | PO-mG-PO-mA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-39 | Hy-lgT7-PO-lgT7-PO-lgT7- | 895 | Hy-mA-PS-fU-PS-mC-PO- | 1057 |
| | PO-mA-PO-mG-PO-mA-PO-mC- | | fA-PO-mA-PO-mA-PO-mU- | |
| | PO-fA-PO-mA-PO-fU-PO-fU- | | PO-mG-PO-fA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-40 | Hy-lgT7-PO-lgT7-PO-lgT7- | 896 | Hy-fA-PS-fU-PS-mC-PO- | 1058 |
| | PO-mA-PO-mG-PO-mA-PO-mC- | | fA-PO-mA-PO-fA-PO-mU- | |
| | PO-fA-PO-mA-PO-fU-PO-fU- | | PO-fG-PO-mA-PO-fU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | fC-PO-mU-PS-dT-PS-dT- | |
| | PO-lT4-Hy | | Hy | |
| 013-c-41 | Hy-lgT7-PO-lgT7-PO-lgT7- | 897 | Hy-mA-PS-fU-PS-mC-PO- | 1059 |
| | PO-lA-PO-lG-PO-mA-PO-mC- | | fA-PO-mA-PO-mA-PO-mU- | |
| | PO-fA-PO-mA-PO-fU-PO-fU- | | PO-mG-PO-mA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-42 | Hy-lgT7-PO-lgT7-PO-lgT7- | 898 | Hy-mA-PS-fU-PS-mC-PO- | 1060 |
| | PO-lA-PO-lG-PO-mA-PO-mC- | | mA-PO-mA-PO-lA-PO-mU- | |
| | PO-fA-PO-mA-PO-fU-PO-fU- | | PO-mG-PO-mA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-43 | Hy-lgT7-PO-lgT7-PO-lgT7- | 899 | Hy-mA-PS-fU-PS-mC-PO- | 1061 |
| | PO-lA-PO-lG-PO-mA-PO-mC- | | fA-PO-mA-PO-mA-PO-mU- | |
| | PO-fA-PO-mA-PO-fU-PO-fU- | | PO-mG-PO-fA-PO-mU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-44 | Hy-lgT7-PO-lgT7-PO-lgT7- | 900 | Hy-fA-PS-fU-PS-mC-PO- | 1062 |
| | PO-lA-PO-lG-PO-mA-PO-mC- | | fA-PO-mA-PO-fA-PO-mU- | |
| | PO-lA-PO-mA-PO-fU-PO-fU- | | PO-fG-PO-mA-PO-fU-PO- | |
| | PO-fC-PO-mA-PO-mU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | fC-PO-mU-PS-dT-PS-dT- | |
| | PO-lT4-Hy | | Hy | |
| 013-c-45 | Hy-lgT7-PO-lgT7-PO-lgT7- | 901 | Hy-mA-PS-fU-PS-mC-PO- | 1063 |
| | PO-mA-PO-mG-PO-mA-PO-mC- | | fA-PO-mA-PO-mA-PO-mU- | |
| | PO-fA-PO-mA-PO-fU-PO-mU- | | PO-mG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-46 | Hy-lgT7-PO-lgT7-PO-lgT7- | 902 | Hy-mA-PS-fU-PS-mC-PO- | 1064 |
| | PO-mA-PO-mG-PO-mA-PO-mC- | | mA-PO-mA-PO-fA-PO-mU- | |
| | PO-fA-PO-mA-PO-fU-PO-mU- | | PO-mG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-47 | Hy-lgT7-PO-lgT7-PO-lgT7- | 903 | Hy-mA-PS-fU-PS-mC-PO- | 1065 |
| | PO-mA-PO-mG-PO-mA-PO-mC- | | fA-PO-mA-PO-mA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-48 | Hy-lgT7-PO-lgT7-PO-lgT7- | 904 | Hy-mA-PS-fU-PS-mC-PO- | 1066 |
| | PO-mA-PO-mG-PO-mA-PO-mC- | | mA-PO-mA-PO-mA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-49 | Hy-lgT7-PO-lgT7-PO-lgT7- | 905 | Hy-fA-PS-fU-PS-mC-PO- | 1067 |
| | PO-mA-PO-mG-PO-mA-PO-mC- | | fA-PO-mA-PO-fA-PO-mU- | |
| | PO-lA-PO-mA-PO-fU-PO-mU- | | PO-fG-PO-mA-PO-mU-PO- | |
| | PO-fC-PO-fA-PO-fU-PO-mC- | | mG-PO-fA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | fC-PO-mU-PS-dT-PS-dT- | |
| | PO-lT4-Hy | | Hy | |
| 013-c-50 | Hy-lgT7-PO-lgT7-PO-lgT7- | 906 | Hy-mA-PS-fU-PS-mC-PO- | 1068 |
| | PO-lA-PO-lG-PO-mA-PO-mC- | | fA-PO-mA-PO-mA-PO-mU- | |
| | PO-fA-PO-mA-PO-fU-PO-mU- | | PO-mG-PO-mA-PO-mU-PO- | |
| | PO-fC-PO-lA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-51 | Hy-lgT7-PO-lgT7-PO-lgT7- | 907 | Hy-mA-PS-fU-PS-mC-PO- | 1069 |
| | PO-lA-PO-lG-PO-mA-PO-mC- | | mA-PO-mA-PO-fA-PO-mU- | |
| | PO-fA-PO-mA-PO-fU-PO-mU- | | PO-mG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-52 | Hy-lgT7-PO-lgT7-PO-lgT7- | 908 | Hy-mA-PS-fU-PS-mC-PO- | 1070 |
| | PO-lA-PO-lG-PO-mA-PO-mC- | | fA-PO-mA-PO-mA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-53 | Hy-lgT7-PO-lgT7-PO-lgT7- | 909 | Hy-mA-PS-fU-PS-mC-PO- | 1071 |
| | PO-lA-PO-lG-PO-mA-PO-mC- | | mA-PO-mA-PO-mA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-mA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | mC-PO-mU-PS-mA-PS-mA- | |
| | PO-lT4-Hy | | Hy | |
| 013-c-54 | Hy-lgT7-PO-lgT7-PO-lgT7- | 910 | Hy-fA-PS-fU-PS-mC-PO- | 1072 |
| | PO-lA-PO-lG-PO-mA-PO-mC- | | fA-PO-mA-PO-fA-PO-mU- | |
| | PO-fA-PO-mA-PO-fU-PO-mU- | | PO-fG-PO-mA-PO-mU-PO- | |
| | PO-fC-PO-fA-PO-fU-PO-mC- | | mG-PO-fA-PO-mA-PO-fU- | |
| | PO-mA-PO-mU-PO-mU-PO-mU- | | PO-mU-PO-fG-PO-mU-PO- | |
| | PO-mG-PO-mA-PO-mU-PO-lT4- | | fC-PO-mU-PS-dT-PS-dT- | |
| | PO-lT4-Hy | | Hy | |
| 051-c-01 | Hy-lgT7-PO-lgT7-PO-lgT7- | 911 | Hy-mU-PS-fC-PS-mU-PO- | 1073 |
| | PO-mG-PO-mU-PO-mA-PO-mU- | | fC-PO-mU-PO-mU-PO-mA- | |
| | PO-fU-PO-mA-PO-fA-PO-fA- | | PO-mA-PO-mG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-lA-PO-mU-PO- | |
| | PO-mA-PS-mG-PS-mA-Hy | | mA-PO-mC-PS-mA-PS-mA- | |
| | | | Hy | |
| 051-c-02 | Hy-lgT7-PO-lgT7-PO-lgT7- | 912 | Hy-mU-PS-fC-PS-mU-PO- | 1074 |
| | PO-mG-PO-mU-PO-mA-PO-mU- | | mC-PO-mU-PO-fU-PO-mA- | |
| | PO-fU-PO-mA-PO-fA-PO-fA- | | PO-mA-PO-mG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PS-mG-PS-mA-Hy | | mA-PO-mC-PS-mA-PS-mA- | |
| | | | Hy | |
| 051-c-03 | Hy-lgT7-PO-lgT7-PO-lgT7- | 913 | Hy-mU-PS-fC-PS-mU-PO- | 1075 |
| | PO-mG-PO-mU-PO-mA-PO-mU- | | fC-PO-mU-PO-mU-PO-mA- | |
| | PO-fU-PO-mA-PO-fA-PO-fA- | | PO-mA-PO-fG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PS-mG-PS-mA-Hy | | mA-PO-mC-PS-mA-PS-mA- | |
| | | | Hy | |
| 051-c-04 | Hy-lgT7-PO-lgT7-PO-lgT7- | 914 | Hy-fU-PS-fC-PS-mU-PO- | 1076 |
| | PO-mG-PO-mU-PO-mA-PO-mU- | | fC-PO-mU-PO-fU-PO-mA- | |
| | PO-fU-PO-mA-PO-fA-PO-fA- | | PO-fA-PO-mG-PO-fG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-lA-PO-mU-PO- | |
| | PO-mA-PS-mG-PS-mA-Hy | | fA-PO-mC-PS-dT-PS-dT- | |
| | | | Hy | |
| 051-c-05 | Hy-lgT7-PO-lgT7-PO-lgT7- | 915 | Hy-mU-PS-fC-PS-mU-PO- | 1077 |
| | PO-lG-PO-lU-PO-mA-PO-mU- | | fC-PO-mU-PO-mU-PO-mA- | |
| | PO-fU-PO-mA-PO-lA-PO-fA- | | PO-mA-PO-mG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-lA-PO-mU-PO- | |
| | PO-mA-PS-mG-PS-mA-Hy | | mA-PO-mC-PS-mA-PS-mA- | |
| | | | Hy | |
| 051-c-06 | Hy-lgT7-PO-lgT7-PO-lgT7- | 916 | Hy-mU-PS-fC-PS-mU-PO- | 1078 |
| | PO-lG-PO-lU-PO-mA-PO-mU- | | mC-PO-mU-PO-fU-PO-mA- | |
| | PO-fU-PO-mA-PO-fA-PO-fA- | | PO-mA-PO-mG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-lA-PO-mU-PO- | |
| | PO-mA-PS-mG-PS-mA-Hy | | mA-PO-mC-PS-mA-PS-mA- | |
| | | | Hy | |
| 051-c-07 | Hy-lgT7-PO-lgT7-PO-lgT7- | 917 | Hy-mU-PS-fC-PS-mU-PO- | 1079 |
| | PO-lG-PO-lU-PO-mA-PO-mU- | | fC-PO-mU-PO-mU-PO-mA- | |
| | PO-fU-PO-mA-PO-fA-PO-lA- | | PO-mA-PO-fG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PS-mG-PS-mA-Hy | | mA-PO-mC-PS-mA-PS-mA- | |
| | | | Hy | |
| 051-c-08 | Hy-lgT7-PO-lgT7-PO-lgT7- | 918 | Hy-fU-PS-fC-PS-mU-PO- | 1080 |
| | PO-lG-PO-lU-PO-mA-PO-mU- | | fC-PO-mU-PO-fU-PO-mA- | |
| | PO-fU-PO-mA-PO-fA-PO-fA- | | PO-fA-PO-mG-PO-fG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PS-mG-PS-mA-Hy | | fA-PO-mC-PS-dT-PS-dT- | |
| | | | Hy | |
| 051-c-09 | Hy-lgT7-PO-lgT7-PO-lgT7- | 919 | Hy-mU-PS-fC-PS-mU-PO- | 1081 |
| | PO-mG-PO-mU-PO-mA-PO-mU- | | fC-PO-mU-PO-mU-PO-mA- | |
| | PO-fU-PO-mA-PO-lA-PO-mA- | | PO-mA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-lA-PO-mU-PO- | |
| | PO-mA-PS-mG-PS-mA-Hy | | mA-PO-mC-PS-mA-PS-mA- | |
| | | | Hy | |
| 051-c-10 | Hy-lgT7-PO-lgT7-PO-lgT7- | 920 | Hy-mU-PS-fC-PS-mU-PO- | 1082 |
| | PO-mG-PO-mU-PO-mA-PO-mU- | | mC-PO-mU-PO-fU-PO-mA- | |
| | PO-fU-PO-mA-PO-fA-PO-mA- | | PO-mA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PS-mG-PS-mA-Hy | | mA-PO-mC-PS-mA-PS-mA- | |
| | | | Hy | |
| 051-c-11 | Hy-lgT7-PO-lgT7-PO-lgT7- | 921 | Hy-mU-PS-fC-PS-mU-PO- | 1083 |
| | PO-mG-PO-mU-PO-mA-PO-mU- | | fC-PO-mU-PO-mU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PS-mG-PS-mA-Hy | | mA-PO-mC-PS-mA-PS-mA- | |
| | | | Hy | |
| 051-c-12 | Hy-lgT7-PO-lgT7-PO-lgT7- | 922 | Hy-mU-PS-fC-PS-mU-PO- | 1084 |
| | PO-mG-PO-mU-PO-mA-PO-mU- | | mC-PO-mU-PO-mU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PS-mG-PS-mA-Hy | | mA-PO-mC-PS-mA-PS-mA- | |
| | | | Hy | |
| 051-c-13 | Hy-lgT7-PO-lgT7-PO-lgT7- | 923 | Hy-fU-PS-fC-PS-mU-PO- | 1085 |
| | PO-mG-PO-mU-PO-mA-PO-mU- | | fC-PO-mU-PO-fU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-fU-PO-mU-PO-fU- | |
| | PO-mU-PO-mA-PO-mA-PO-mG- | | PO-mA-PO-lA-PO-mU-PO- | |
| | PO-mA-PS-mG-PS-mA-Hy | | fA-PO-mC-PS-dT-PS-dT- | |
| | | | Hy | |
| 051-c-14 | Hy-lgT7-PO-lgT7-PO-lgT7- | 924 | Hy-mU-PS-fC-PS-mU-PO- | 1086 |
| | PO-lG-PO-lU-PO-mA-PO-mU- | | fC-PO-mU-PO-mU-PO-mA- | |
| | PO-fU-PO-mA-PO-fA-PO-mA- | | PO-mA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PS-mG-PS-mA-Hy | | mA-PO-mC-PS-mA-PS-mA- | |
| | | | Hy | |
| 051-c-15 | Hy-lgT7-PO-lgT7-PO-lgT7- | 925 | Hy-mU-PS-fC-PS-mU-PO- | 1087 |
| | PO-lG-PO-lU-PO-mA-PO-mU- | | mC-PO-mU-PO-fU-PO-mA- | |
| | PO-fU-PO-mA-PO-fA-PO-mA- | | PO-mA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PS-mG-PS-mA-Hy | | mA-PO-mC-PS-mA-PS-mA- | |
| 051-c-16 | Hy-lgT7-PO-lgT7-PO-lgT7- | 926 | Hy-mU-PS-fC-PS-mU-PO- | 1088 |
| | PO-lG-PO-lU-PO-mA-PO-mU- | | fC-PO-mU-PO-mU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PS-mG-PS-mA-Hy | | mA-PO-mC-PS-mA-PS-mA- | |
| | | | Hy | |
| 051-c-17 | Hy-lgT7-PO-lgT7-PO-lgT7- | 927 | Hy-mU-PS-fC-PS-mU-PO- | 1089 |
| | PO-lG-PO-lU-PO-mA-PO-mU- | | mC-PO-mU-PO-mU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PS-mG-PS-mA-Hy | | mA-PO-mC-PS-mA-PS-mA- | |
| | | | Hy | |
| 051-c-18 | Hy-lgT7-PO-lgT7-PO-lgT7- | 928 | Hy-fU-PS-fC-PS-mU-PO- | 1090 |
| | PO-lG-PO-lU-PO-mA-PO-mU- | | fC-PO-mU-PO-fU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-fU-PO-mU-PO-fU- | |
| | PO-mU-PO-mA-PO-mA-PO-mG- | | PO-mA-PO-fA-PO-mU-PO- | |
| | PO-mA-PS-mG-PS-mA-Hy | | fA-PO-mC-PS-dT-PS-dT- | |
| | | | Hy | |
| 051-c-19 | Hy-lgT7-PO-lgT7-PO-lgT7- | 929 | Hy-mU-PS-fC-PS-mU-PO- | 1091 |
| | PO-mG-PO-mU-PO-mA-PO-mU- | | fC-PO-mU-PO-mU-PO-mA- | |
| | PO-fU-PO-mA-PO-fA-PO-fA- | | PO-mA-PO-mG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-lA-PO-mU-PO- | |
| | PO-mA-PO-lT4-PO-lT4-Hy | | mA-PO-mC-PS-mA-PS-mA- | |
| | | | Hy | |
| 051-c-20 | Hy-lgT7-PO-lgT7-PO-lgT7- | 930 | Hy-mU-PS-fC-PS-mU-PO- | 1092 |
| | PO-mG-PO-mU-PO-mA-PO-mU- | | mC-PO-mU-PO-fU-PO-mA- | |
| | PO-fU-PO-mA-PO-fA-PO-fA- | | PO-mA-PO-mG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| | | | Hy | |
| 051-c-21 | Hy-lgT7-PO-lgT7-PO-lgT7- | 931 | Hy-mU-PS-fC-PS-mU-PO- | 1093 |
| | PO-mG-PO-mU-PO-mA-PO-mU- | | fC-PO-mU-PO-mU-PO-mA- | |
| | PO-fU-PO-mA-PO-fA-PO-fA- | | PO-mA-PO-fG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| | | | Hy | |
| 051-c-22 | Hy-lgT7-PO-lgT7-PO-lgT7- | 932 | Hy-fU-PS-fC-PS-mU-PO- | 1094 |
| | PO-mG-PO-mU-PO-mA-PO-mU- | | fC-PO-mU-PO-fU-PO-mA- | |
| | PO-fU-PO-mA-PO-fA-PO-fA- | | PO-fA-PO-mG-PO-fG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | fA-PO-mC-PS-dT-PS-dT- | |
| | | | Hy | |
| 051-c-23 | Hy-lgT7-PO-lgT7-PO-lgT7- | 933 | Hy-mU-PS-fC-PS-mU-PO- | 1095 |
| | PO-lG-PO-lU-PO-mA-PO-mU- | | fC-PO-mU-PO-mU-PO-mA- | |
| | PO-fU-PO-mA-PO-fA-PO-fA- | | PO-mA-PO-mG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| | | | Hy | |
| 051-c-24 | Hy-lgT7-PO-lgT7-PO-lgT7- | 934 | Hy-mU-PS-fC-PS-mU-PO- | 1096 |
| | PO-lG-PO-lU-PO-mA-PO-mU- | | mC-PO-mU-PO-fU-PO-mA- | |
| | PO-fU-PO-mA-PO-fA-PO-fA- | | PO-mA-PO-mG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| | | | Hy | |
| 051-c-25 | Hy-lgT7-PO-lgT7-PO-lgT7- | 935 | Hy-mU-PS-fC-PS-mU-PO- | 1097 |
| | PO-lG-PO-lU-PO-mA-PO-mU- | | fC-PO-mU-PO-mU-PO-mA- | |
| | PO-fU-PO-mA-PO-fA-PO-fA- | | PO-mA-PO-fG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| | | | Hy | |
| 051-c-26 | Hy-lgT7-PO-lgT7-PO-lgT7- | 936 | Hy-fU-PS-fC-PS-mU-PO- | 1098 |
| | PO-lG-PO-lU-PO-mA-PO-mU- | | fC-PO-mU-PO-fU-PO-mA- | |
| | PO-fU-PO-mA-PO-fA-PO-fA- | | PO-fA-PO-mG-PO-fG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-lA-PO-mU-PO- | |
| | PO-mA-PO-lT4-PO-lT4-Hy | | fA-PO-mC-PS-dT-PS-dT- | |
| | | | Hy | |
| 051-c-27 | Hy-lgT7-PO-lgT7-PO-lgT7- | 937 | Hy-mU-PS-fC-PS-mU-PO- | 1099 |
| | PO-mG-PO-mU-PO-mA-PO-mU- | | fC-PO-mU-PO-mU-PO-mA- | |
| | PO-fU-PO-mA-PO-lA-PO-mA- | | PO-mA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| 051-c-28 | Hy-lgT7-PO-lgT7-PO-lgT7- | 938 | Hy-mU-PS-fC-PS-mU-PO- | 1100 |
| | PO-mG-PO-mU-PO-mA-PO-mU- | | mC-PO-mU-PO-fU-PO-mA- | |
| | PO-fU-PO-mA-PO-fA-PO-mA- | | PO-mA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| | | | Hy | |
| 051-c-29 | Hy-lgT7-PO-lgT7-PO-lgT7- | 939 | Hy-mU-PS-fC-PS-mU-PO- | 1101 |
| | PO-mG-PO-mU-PO-mA-PO-mU- | | fC-PO-mU-PO-mU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| | | | Hy | |
| 051-c-30 | Hy-lgT7-PO-lgT7-PO-lgT7- | 940 | Hy-mU-PS-fC-PS-mU-PO- | 1102 |
| | PO-mG-PO-mU-PO-mA-PO-mU- | | mC-PO-mU-PO-mU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| | | | Hy | |
| 051-c-31 | Hy-lgT7-PO-lgT7-PO-lgT7- | 941 | Hy-fU-PS-fC-PS-mU-PO- | 1103 |
| | PO-mG-PO-mU-PO-mA-PO-mU- | | fC-PO-mU-PO-fU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-fU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | fA-PO-mC-PS-dT-PS-dT- | |
| | | | Hy | |
| 051-c-32 | Hy-lgT7-PO-lgT7-PO-lgT7- | 942 | Hy-mU-PS-fC-PS-mU-PO- | 1104 |
| | PO-lG-PO-lU-PO-mA-PO-mU- | | fC-PO-mU-PO-mU-PO-mA- | |
| | PO-fU-PO-mA-PO-fA-PO-mA- | | PO-mA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| | | | Hy | |
| 051-c-33 | Hy-lgT7-PO-lgT7-PO-lgT7- | 943 | Hy-mU-PS-fC-PS-mU-PO- | 1105 |
| | PO-lG-PO-lU-PO-mA-PO-mU- | | mC-PO-mU-PO-fU-PO-mA- | |
| | PO-fU-PO-mA-PO-fA-PO-mA- | | PO-mA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| | | | Hy | |
| 051-c-34 | Hy-lgT7-PO-lgT7-PO-lgT7- | 944 | Hy-mU-PS-fC-PS-mU-PO- | 1106 |
| | PO-lG-PO-lU-PO-mA-PO-mU- | | fC-PO-mU-PO-mU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| | | | Hy | |
| 051-c-35 | Hy-lgT7-PO-lgT7-PO-lgT7- | 945 | Hy-mU-PS-fC-PS-mU-PO- | 1107 |
| | PO-lG-PO-lU-PO-mA-PO-mU- | | mC-PO-mU-PO-mU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | mA-PO-mC-PS-mA-PS-mA- | |
| | | | Hy | |
| 051-c-36 | Hy-lgT7-PO-lgT7-PO-lgT7- | 946 | Hy-fU-PS-fC-PS-mU-PO- | 1108 |
| | PO-lG-PO-lU-PO-mA-PO-mU- | | fC-PO-mU-PO-fU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-fU-PO-mU-PO-fU- | |
| | PO-mU-PO-mA-PO-mA-PO-mG- | | PO-mA-PO-fA-PO-mU-PO- | |
| | PO-mA-PO-lT4-PO-lT4-Hy | | fA-PO-mC-PS-dT-PS-dT- | |
| | | | Hy | |
| 051-c-37 | Hy-lgT7-PO-lgT7-PO-lgT7- | 947 | Hy-mU-PS-fC-PS-mU-PO- | 1109 |
| | PO-mG-PO-mU-PO-mA-PO-mU- | | fC-PO-mU-PO-mU-PO-mA- | |
| | PO-fU-PO-mA-PO-fA-PO-fA- | | PO-mA-PO-mG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-38 | Hy-lgT7-PO-lgT7-PO-lgT7- | 948 | Hy-mU-PS-fC-PS-mU-PO- | 1110 |
| | PO-mG-PO-mU-PO-mA-PO-mU- | | mC-PO-mU-PO-fU-PO-mA- | |
| | PO-fU-PO-mA-PO-fA-PO-fA- | | PO-mA-PO-mG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-39 | Hy-lgT7-PO-lgT7-PO-lgT7- | 949 | Hy-mU-PS-fC-PS-mU-PO- | 1111 |
| | PO-mG-PO-mU-PO-mA-PO-mU- | | fC-PO-mU-PO-mU-PO-mA- | |
| | PO-fU-PO-mA-PO-fA-PO-fA- | | PO-mA-PO-fG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-40 | Hy-lgT7-PO-lgT7-PO-lgT7- | 950 | Hy-fU-PS-fC-PS-mU-PO- | 1112 |
| | PO-mG-PO-mU-PO-mA-PO-mU- | | fC-PO-mU-PO-fU-PO-mA- | |
| | PO-fU-PO-mA-PO-fA-PO-fA- | | PO-fA-PO-mG-PO-fG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | fA-PO-mC-PS-dT-PS-dT- | |
| | PO-lT4-Hy | | | |
| 051-c-41 | Hy-lgT7-PO-lgT7-PO-lgT7- | 951 | Hy-mU-PS-fC-PS-mU-PO- | 1113 |
| | PO-lG-PO-lU-PO-mA-PO-mU- | | fC-PO-mU-PO-mU-PO-mA- | |
| | PO-fU-PO-mA-PO-fA-PO-fA- | | PO-mA-PO-mG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-42 | Hy-lgT7-PO-lgT7-PO-lgT7- | 952 | Hy-mU-PS-fC-PS-mU-PO- | 1114 |
| | PO-lG-PO-lU-PO-mA-PO-mU- | | mC-PO-mU-PO-fU-PO-mA- | |
| | PO-fU-PO-mA-PO-fA-PO-fA- | | PO-mA-PO-mG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-43 | Hy-lgT7-PO-lgT7-PO-lgT7- | 953 | Hy-mU-PS-fC-PS-mU-PO- | 1115 |
| | PO-lG-PO-lU-PO-mA-PO-mU- | | fC-PO-mU-PO-mU-PO-mA- | |
| | PO-fU-PO-mA-PO-fA-PO-fA- | | PO-mA-PO-fG-PO-mG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-44 | Hy-lgT7-PO-lgT7-PO-lgT7- | 954 | Hy-fU-PS-fC-PS-mU-PO- | 1116 |
| | PO-lG-PO-lU-PO-mA-PO-mU- | | fC-PO-mU-PO-fU-PO-mA- | |
| | PO-fU-PO-mA-PO-fA-PO-fA- | | PO-fA-PO-mG-PO-fG-PO- | |
| | PO-fU-PO-mC-PO-mC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | fA-PO-mC-PS-dT-PS-dT- | |
| | PO-lT4-Hy | | Hy | |
| 051-c-45 | Hy-lgT7-PO-lgT7-PO-lgT7- | 955 | Hy-mU-PS-fC-PS-mU-PO- | 1117 |
| | PO-mG-PO-mU-PO-mA-PO-mU- | | fC-PO-mU-PO-mU-PO-mA- | |
| | PO-fU-PO-mA-PO-fA-PO-mA- | | PO-mA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-46 | Hy-lgT7-PO-lgT7-PO-lgT7- | 956 | Hy-mU-PS-fC-PS-mU-PO- | 1118 |
| | PO-mG-PO-mU-PO-mA-PO-mU- | | mC-PO-mU-PO-fU-PO-mA- | |
| | PO-fU-PO-mA-PO-fA-PO-mA- | | PO-mA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-lA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-47 | Hy-lgT7-PO-lgT7-PO-lgT7- | 957 | Hy-mU-PS-fC-PS-mU-PO- | 1119 |
| | PO-mG-PO-mU-PO-mA-PO-mU- | | fC-PO-mU-PO-mU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-48 | Hy-lgT7-PO-lgT7-PO-lgT7- | 958 | Hy-mU-PS-fC-PS-mU-PO- | 1120 |
| | PO-mG-PO-mU-PO-mA-PO-mU- | | mC-PO-mU-PO-mU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-49 | Hy-lgT7-PO-lgT7-PO-lgT7- | 959 | Hy-fU-PS-fC-PS-mU-PO- | 1121 |
| | PO-mG-PO-mU-PO-mA-PO-mU- | | fC-PO-mU-PO-fU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-fU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | fA-PO-mC-PS-dT-PS-dT- | |
| | PO-lT4-Hy | | Hy | |
| 051-c-50 | Hy-lgT7-PO-lgT7-PO-lgT7- | 960 | Hy-mU-PS-fC-PS-mU-PO- | 1122 |
| | PO-lG-PO-lU-PO-mA-PO-mU- | | fC-PO-mU-PO-mU-PO-mA- | |
| | PO-fU-PO-mA-PO-fA-PO-mA- | | PO-mA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-51 | Hy-lgT7-PO-lgT7-PO-lgT7- | 961 | Hy-mU-PS-fC-PS-mU-PO- | 1123 |
| | PO-lG-PO-lU-PO-mA-PO-mU- | | mC-PO-mU-PO-fU-PO-mA- | |
| | PO-fU-PO-mA-PO-fA-PO-mA- | | PO-mA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-lA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-52 | Hy-lgT7-PO-lgT7-PO-lgT7- | 962 | Hy-mU-PS-fC-PS-mU-PO- | 1124 |
| | PO-lG-PO-lU-PO-mA-PO-mU- | | fC-PO-mU-PO-mU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-53 | Hy-lgT7-PO-lgT7-PO-lgT7- | 963 | Hy-mU-PS-fC-PS-mU-PO- | 1125 |
| | PO-lG-PO-lU-PO-mA-PO-mU- | | mC-PO-mU-PO-mU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-mU-PO-mU-PO-fU- | |
| | PO-mU-PO-mA-PO-mA-PO-mG- | | PO-mA-PO-fA-PO-mU-PO- | |
| | PO-mA-PO-mG-PO-mA-PO-lT4- | | mA-PO-mC-PS-mA-PS-mA- | |
| | PO-lT4-Hy | | Hy | |
| 051-c-54 | Hy-lgT7-PO-lgT7-PO-lgT7- | 964 | Hy-fU-PS-fC-PS-mU-PO- | 1126 |
| | PO-lG-PO-lU-PO-mA-PO-mU- | | fC-PO-mU-PO-fU-PO-mA- | |
| | PO-fU-PO-mA-PO-fA-PO-mA- | | PO-fA-PO-mG-PO-mG-PO- | |
| | PO-fU-PO-fC-PO-fC-PO-mU- | | mA-PO-fU-PO-mU-PO-fU- | |
| | PO-mU-PO-mA-PO-mA-PO-mG- | | PO-mA-PO-lA-PO-mU-PO- | |
| | PO-mA-PO-mG-PO-mA-PO-lT4- | | fA-PO-mC-PS-dT-PS-dT- | |
| | PO-lT4-Hy | | Hy | |
| 165-c-01 | Hy-lgT7-PO-lgT7-PO-lgT7- | 965 | Hy-mA-PS-fU-PS-mG-PO- | 1127 |
| | PO-mA-PO-mU-PO-mU-PO-mG- | | fA-PO-mA-PO-mU-PO-mU- | |
| | PO-fA-PO-mU-PO-fC-PO-fA- | | PO-mG-PO-mU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-02 | Hy-lgT7-PO-lgT7-PO-lgT7- | 966 | Hy-mA-PS-fU-PS-mG-PO- | 1128 |
| | PO-mA-PO-mU-PO-mU-PO-mG- | | mA-PO-mA-PO-fU-PO-mU- | |
| | PO-fA-PO-mU-PO-fC-PO-fA- | | PO-mG-PO-mU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-03 | Hy-lgT7-PO-lgT7-PO-lgT7- | 967 | Hy-mA-PS-fU-PS-mG-PO- | 1129 |
| | PO-mA-PO-mU-PO-mU-PO-mG- | | fA-PO-mA-PO-mU-PO-mU- | |
| | PO-fA-PO-mU-PO-fC-PO-fA- | | PO-mG-PO-fU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-04 | Hy-lgT7-PO-lgT7-PO-lgT7- | 968 | Hy-fA-PS-fU-PS-mG-PO- | 1130 |
| | PO-mA-PO-mU-PO-mU-PO-mG- | | fA-PO-mA-PO-fU-PO-mU- | |
| | PO-fA-PO-mU-PO-fC-PO-fA- | | PO-fG-PO-mU-PO-fC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | fA-PO-mU-PS-dT-PS-dT- | |
| | | | Hy | |
| 165-c-05 | Hy-lgT7-PO-lgT7-PO-lgT7- | 969 | Hy-mA-PS-fU-PS-mG-PO- | 1131 |
| | PO-lA-PO-lU-PO-mU-PO-mG- | | fA-PO-mA-PO-mU-PO-mU- | |
| | PO-fA-PO-mU-PO-fC-PO-fA- | | PO-mG-PO-mU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-06 | Hy-lgT7-PO-lgT7-PO-lgT7- | 970 | Hy-mA-PS-fU-PS-mG-PO- | 1132 |
| | PO-lA-PO-lU-PO-mU-PO-mG- | | mA-PO-mA-PO-fU-PO-mU- | |
| | PO-lA-PO-mU-PO-fC-PO-fA- | | PO-mG-PO-mU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-07 | Hy-lgT7-PO-lgT7-PO-lgT7- | 971 | Hy-mA-PS-fU-PS-mG-PO- | 1133 |
| | PO-lA-PO-lU-PO-mU-PO-mG- | | fA-PO-mA-PO-mU-PO-mU- | |
| | PO-fA-PO-mU-PO-fC-PO-fA- | | PO-mG-PO-fU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-08 | Hy-lgT7-PO-lgT7-PO-lgT7- | 972 | Hy-fA-PS-fU-PS-mG-PO- | 1134 |
| | PO-lA-PO-lU-PO-mU-PO-mG- | | fA-PO-mA-PO-fU-PO-mU- | |
| | PO-fA-PO-mU-PO-fC-PO-fA- | | PO-fG-PO-mU-PO-fC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | fA-PO-mU-PS-dT-PS-dT- | |
| | | | Hy | |
| 165-c-09 | Hy-lgT7-PO-lgT7-PO-lgT7- | 973 | Hy-mA-PS-fU-PS-mG-PO- | 1135 |
| | PO-mA-PO-mU-PO-mU-PO-mG- | | fA-PO-mA-PO-mU-PO-mU- | |
| | PO-fA-PO-mU-PO-fC-PO-mA- | | PO-mG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-10 | Hy-lgT7-PO-lgT7-PO-lgT7- | 974 | Hy-mA-PS-fU-PS-mG-PO- | 1136 |
| | PO-mA-PO-mU-PO-mU-PO-mG- | | mA-PO-mA-PO-fU-PO-mU- | |
| | PO-fA-PO-mU-PO-fC-PO-mA- | | PO-mG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-11 | Hy-lgT7-PO-lgT7-PO-lgT7- | 975 | Hy-mA-PS-fU-PS-mG-PO- | 1137 |
| | PO-mA-PO-mU-PO-mU-PO-mG- | | fA-PO-mA-PO-mU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-12 | Hy-lgT7-PO-lgT7-PO-lgT7- | 976 | Hy-mA-PS-fU-PS-mG-PO- | 1138 |
| | PO-mA-PO-mU-PO-mU-PO-mG- | | mA-PO-mA-PO-mU-PO-mU- | |
| | PO-fA-PO-mU-PO-fC-PO-mA- | | PO-fG-PO-mU-PO-mC-PO- | |
| | PO-fA-PO-fG-PO-lA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-13 | Hy-lgT7-PO-lgT7-PO-lgT7- | 977 | Hy-fA-PS-fU-PS-mG-PO- | 1139 |
| | PO-mA-PO-mU-PO-mU-PO-mG- | | fA-PO-mA-PO-fU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-fU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | fA-PO-mU-PS-dT-PS-dT- | |
| | | | Hy | |
| 165-c-14 | Hy-lgT7-PO-lgT7-PO-lgT7- | 978 | Hy-mA-PS-fU-PS-mG-PO- | 1140 |
| | PO-lA-PO-lU-PO-mU-PO-mG- | | fA-PO-mA-PO-mU-PO-mU- | |
| | PO-fA-PO-mU-PO-fC-PO-mA- | | PO-mG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-15 | Hy-lgT7-PO-lgT7-PO-lgT7- | 979 | Hy-mA-PS-fU-PS-mG-PO- | 1141 |
| | PO-lA-PO-lU-PO-mU-PO-mG- | | mA-PO-mA-PO-fU-PO-mU- | |
| | PO-fA-PO-mU-PO-fC-PO-mA- | | PO-mG-PO-mU-PO-mC-PO- | |
| | PO-fA-PO-fG-PO-lA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-16 | Hy-lgT7-PO-lgT7-PO-lgT7- | 980 | Hy-mA-PS-fU-PS-mG-PO- | 1142 |
| | PO-lA-PO-lU-PO-mU-PO-mG- | | fA-PO-mA-PO-mU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-17 | Hy-lgT7-PO-lgT7-PO-lgT7- | 981 | Hy-mA-PS-fU-PS-mG-PO- | 1143 |
| | PO-lA-PO-lU-PO-mU-PO-mG- | | mA-PO-mA-PO-mU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | mA-PO-mU-PS-mA-PS-mA- | |
| | | | Hy | |
| 165-c-18 | Hy-lgT7-PO-lgT7-PO-lgT7- | 982 | Hy-fA-PS-fU-PS-mG-PO- | 1144 |
| | PO-lA-PO-lU-PO-mU-PO-mG- | | fA-PO-mA-PO-fU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-fU-PO-mG-PO-fA- | |
| | PO-mA-PO-mA-PO-mU-PO-mU- | | PO-mU-PO-fC-PO-mA-PO- | |
| | PO-mC-PS-mA-PS-mU-Hy | | fA-PO-mU-PS-dT-PS-dT- | |
| | | | Hy | |
| 165-c-19 | Hy-lgT7-PO-lgT7-PO-lgT7- | 983 | Hy-mA-PS-fU-PS-mG-PO- | 1145 |
| | PO-mA-PO-mU-PO-mU-PO-mG- | | fA-PO-mA-PO-mU-PO-mU- | |
| | PO-fA-PO-mU-PO-fC-PO-fA- | | PO-mG-PO-mU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-20 | Hy-lgT7-PO-lgT7-PO-lgT7- | 984 | Hy-mA-PS-fU-PS-mG-PO- | 1146 |
| | PO-mA-PO-mU-PO-mU-PO-mG- | | mA-PO-mA-PO-fU-PO-mU- | |
| | PO-fA-PO-mU-PO-fC-PO-fA- | | PO-mG-PO-mU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-21 | Hy-lgT7-PO-lgT7-PO-lgT7- | 985 | Hy-mA-PS-fU-PS-mG-PO- | 1147 |
| | PO-mA-PO-mU-PO-mU-PO-mG- | | fA-PO-mA-PO-mU-PO-mU- | |
| | PO-fA-PO-mU-PO-fC-PO-fA- | | PO-mG-PO-fU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-22 | Hy-lgT7-PO-lgT7-PO-lgT7- | 986 | Hy-fA-PS-fU-PS-mG-PO- | 1148 |
| | PO-mA-PO-mU-PO-mU-PO-mG- | | fA-PO-mA-PO-fU-PO-mU- | |
| | PO-fA-PO-mU-PO-fC-PO-fA- | | PO-fG-PO-mU-PO-fC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | fA-PO-mU-PS-dT-PS-dT- | |
| | | | Hy | |
| 165-c-23 | Hy-lgT7-PO-lgT7-PO-lgT7- | 987 | Hy-mA-PS-fU-PS-mG-PO- | 1149 |
| | PO-lA-PO-lU-PO-mU-PO-mG- | | fA-PO-mA-PO-mU-PO-mU- | |
| | PO-fA-PO-mU-PO-fC-PO-fA- | | PO-mG-PO-mU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-24 | Hy-lgT7-PO-lgT7-PO-lgT7- | 988 | Hy-mA-PS-fU-PS-mG-PO- | 1150 |
| | PO-lA-PO-lU-PO-mU-PO-mG- | | mA-PO-mA-PO-fU-PO-mU- | |
| | PO-fA-PO-mU-PO-fC-PO-fA- | | PO-mG-PO-mU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-25 | Hy-lgT7-PO-lgT7-PO-lgT7- | 989 | Hy-mA-PS-fU-PS-mG-PO- | 1151 |
| | PO-lA-PO-lU-PO-mU-PO-mG- | | fA-PO-mA-PO-mU-PO-mU- | |
| | PO-fA-PO-mU-PO-fC-PO-fA- | | PO-mG-PO-fU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-26 | Hy-lgT7-PO-lgT7-PO-lgT7- | 990 | Hy-fA-PS-fU-PS-mG-PO- | 1152 |
| | PO-lA-PO-lU-PO-mU-PO-mG- | | fA-PO-mA-PO-fU-PO-mU- | |
| | PO-fA-PO-mU-PO-fC-PO-fA- | | PO-fG-PO-mU-PO-fC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | fA-PO-mU-PS-dT-PS-dT- | |
| | | | Hy | |
| 165-c-27 | Hy-lgT7-PO-lgT7-PO-lgT7- | 991 | Hy-mA-PS-fU-PS-mG-PO- | 1153 |
| | PO-mA-PO-mU-PO-mU-PO-mG- | | fA-PO-mA-PO-mU-PO-mU- | |
| | PO-fA-PO-mU-PO-fC-PO-mA- | | PO-mG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-28 | Hy-lgT7-PO-lgT7-PO-lgT7- | 992 | Hy-mA-PS-fU-PS-mG-PO- | 1154 |
| | PO-mA-PO-mU-PO-mU-PO-mG- | | mA-PO-mA-PO-fU-PO-mU- | |
| | PO-fA-PO-mU-PO-fC-PO-mA- | | PO-mG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-29 | Hy-lgT7-PO-lgT7-PO-lgT7- | 993 | Hy-mA-PS-fU-PS-mG-PO- | 1155 |
| | PO-mA-PO-mU-PO-mU-PO-mG- | | fA-PO-mA-PO-mU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-30 | Hy-lgT7-PO-lgT7-PO-lgT7- | 994 | Hy-mA-PS-fU-PS-mG-PO- | 1156 |
| | PO-mA-PO-mU-PO-mU-PO-mG- | | mA-PO-mA-PO-mU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-31 | Hy-lgT7-PO-lgT7-PO-lgT7- | 995 | Hy-fA-PS-fU-PS-mG-PO- | 1157 |
| | PO-mA-PO-mU-PO-mU-PO-mG- | | fA-PO-mA-PO-fU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-fU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | fA-PO-mU-PS-dT-PS-dT- | |
| | | | Hy | |
| 165-c-32 | Hy-lgT7-PO-lgT7-PO-lgT7- | 996 | Hy-mA-PS-fU-PS-mG-PO- | 1158 |
| | PO-lA-PO-lU-PO-mU-PO-mG- | | fA-PO-mA-PO-mU-PO-mU- | |
| | PO-fA-PO-mU-PO-fC-PO-mA- | | PO-mG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-33 | Hy-lgT7-PO-lgT7-PO-lgT7- | 997 | Hy-mA-PS-fU-PS-mG-PO- | 1159 |
| | PO-lA-PO-lU-PO-mU-PO-mG- | | mA-PO-mA-PO-fU-PO-mU- | |
| | PO-fA-PO-mU-PO-fC-PO-mA- | | PO-mG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-34 | Hy-lgT7-PO-lgT7-PO-lgT7- | 998 | Hy-mA-PS-fU-PS-mG-PO- | 1160 |
| | PO-lA-PO-lU-PO-mU-PO-mG- | | fA-PO-mA-PO-mU-PO-mU- | |
| | PO-fA-PO-mU-PO-fC-PO-mA- | | PO-fG-PO-mU-PO-mC-PO- | |
| | PO-fA-PO-fG-PO-lA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-35 | Hy-lgT7-PO-lgT7-PO-lgT7- | 999 | Hy-mA-PS-fU-PS-mG-PO- | 1161 |
| | PO-lA-PO-lU-PO-mU-PO-mG- | | mA-PO-mA-PO-mU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | mA-PO-mU-PS-mA-PS-mA- | |
| | | | Hy | |
| 165-c-36 | Hy-lgT7-PO-lgT7-PO-lgT7- | 1000 | Hy-fA-PS-fU-PS-mG-PO- | 1162 |
| | PO-lA-PO-lU-PO-mU-PO-mG- | | fA-PO-mA-PO-fU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-fU-PO-mG-PO-fA- | |
| | PO-mA-PO-mA-PO-mU-PO-mU- | | PO-mU-PO-fC-PO-mA-PO- | |
| | PO-mC-PO-lT4-PO-lT4-Hy | | fA-PO-mU-PS-dT-PS-dT- | |
| 165-c-37 | Hy-lgT7-PO-lgT7-PO-lgT7- | 1001 | Hy-mA-PS-fU-PS-mG-PO- | 1163 |
| | PO-mA-PO-mU-PO-mU-PO-mG- | | fA-PO-mA-PO-mU-PO-mU- | |
| | PO-fA-PO-mU-PO-fC-PO-fA- | | PO-mG-PO-mU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | Hy | |
| 165-c-38 | Hy-lgT7-PO-lgT7-PO-lgT7- | 1002 | Hy-mA-PS-fU-PS-mG-PO- | 1164 |
| | PO-mA-PO-mU-PO-mU-PO-mG- | | mA-PO-mA-PO-fU-PO-mU- | |
| | PO-fA-PO-mU-PO-fC-PO-fA- | | PO-mG-PO-mU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | Hy | |
| 165-c-39 | Hy-lgT7-PO-lgT7-PO-lgT7- | 1003 | Hy-mA-PS-fU-PS-mG-PO- | 1165 |
| | PO-mA-PO-mU-PO-mU-PO-mG- | | fA-PO-mA-PO-mU-PO-mU- | |
| | PO-fA-PO-mU-PO-fC-PO-fA- | | PO-mG-PO-fU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | Hy | |
| 165-c-40 | Hy-lgT7-PO-lgT7-PO-lgT7- | 1004 | Hy-fA-PS-fU-PS-mG-PO- | 1166 |
| | PO-mA-PO-mU-PO-mU-PO-mG- | | fA-PO-mA-PO-fU-PO-mU- | |
| | PO-fA-PO-mU-PO-fC-PO-fA- | | PO-fG-PO-mU-PO-fC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | fA-PO-mU-PS-dT-PS-dT- | |
| | PO-lT4-Hy | | Hy | |
| 165-c-41 | Hy-lgT7-PO-lgT7-PO-lgT7- | 1005 | Hy-mA-PS-fU-PS-mG-PO- | 1167 |
| | PO-lA-PO-lU-PO-mU-PO-mG- | | fA-PO-mA-PO-mU-PO-mU- | |
| | PO-fA-PO-mU-PO-fC-PO-fA- | | PO-mG-PO-mU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | Hy | |
| 165-c-42 | Hy-lgT7-PO-lgT7-PO-lgT7- | 1006 | Hy-mA-PS-fU-PS-mG-PO- | 1168 |
| | PO-lA-PO-lU-PO-mU-PO-mG- | | mA-PO-mA-PO-fU-PO-mU- | |
| | PO-fA-PO-mU-PO-fC-PO-fA- | | PO-mG-PO-mU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | Hy | |
| 165-c-43 | Hy-lgT7-PO-lgT7-PO-lgT7- | 1007 | Hy-mA-PS-fU-PS-mG-PO- | 1169 |
| | PO-lA-PO-lU-PO-mU-PO-mG- | | fA-PO-mA-PO-mU-PO-mU- | |
| | PO-fA-PO-mU-PO-fC-PO-fA- | | PO-mG-PO-fU-PO-mC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | | |
| 165-c-44 | Hy-lgT7-PO-lgT7-PO-lgT7- | 1008 | Hy-fA-PS-fU-PS-mG-PO- | 1170 |
| | PO-lA-PO-lU-PO-mU-PO-mG- | | fA-PO-mA-PO-fU-PO-mU- | |
| | PO-lA-PO-mU-PO-fC-PO-fA- | | PO-fG-PO-mU-PO-fC-PO- | |
| | PO-fA-PO-mG-PO-mA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | fA-PO-mU-PS-dT-PS-dT- | |
| | PO-lT4-Hy | | Hy | |
| 165-c-45 | Hy-lgT7-PO-lgT7-PO-lgT7- | 1009 | Hy-mA-PS-fU-PS-mG-PO- | 1171 |
| | PO-mA-PO-mU-PO-mU-PO-mG- | | fA-PO-mA-PO-mU-PO-mU- | |
| | PO-fA-PO-mU-PO-fC-PO-mA- | | PO-mG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | Hy | |
| 165-c-46 | Hy-lgT7-PO-lgT7-PO-lgT7- | 1010 | Hy-mA-PS-fU-PS-mG-PO- | 1172 |
| | PO-mA-PO-mU-PO-mU-PO-mG- | | mA-PO-mA-PO-fU-PO-mU- | |
| | PO-fA-PO-mU-PO-fC-PO-mA- | | PO-mG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | Hy | |
| 165-c-47 | Hy-lgT7-PO-lgT7-PO-lgT7- | 1011 | Hy-mA-PS-fU-PS-mG-PO- | 1173 |
| | PO-mA-PO-mU-PO-mU-PO-mG- | | fA-PO-mA-PO-mU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | Hy | |
| 165-c-48 | Hy-lgT7-PO-lgT7-PO-lgT7- | 1012 | Hy-mA-PS-fU-PS-mG-PO- | 1174 |
| | PO-mA-PO-mU-PO-mU-PO-mG- | | mA-PO-mA-PO-mU-PO-mU- | |
| | PO-fA-PO-mU-PO-fC-PO-mA- | | PO-fG-PO-mU-PO-mC-PO- | |
| | PO-fA-PO-fG-PO-lA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | | |
| 165-c-49 | Hy-lgT7-PO-lgT7-PO-lgT7- | 1013 | Hy-fA-PS-fU-PS-mG-PO- | 1175 |
| | PO-mA-PO-mU-PO-mU-PO-mG- | | fA-PO-mA-PO-fU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-fU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | fA-PO-mU-PS-dT-PS-dT- | |
| | PO-lT4-Hy | | Hy | |
| 165-c-50 | Hy-lgT7-PO-lgT7-PO-lgT7- | 1014 | Hy-mA-PS-fU-PS-mG-PO- | 1176 |
| | PO-lA-PO-lU-PO-mU-PO-mG- | | fA-PO-mA-PO-mU-PO-mU- | |
| | PO-fA-PO-mU-PO-fC-PO-mA- | | PO-mG-PO-mU-PO-mC-PO- | |
| | PO-fA-PO-fG-PO-lA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | Hy | |
| 165-c-51 | Hy-lgT7-PO-lgT7-PO-lgT7- | 1015 | Hy-mA-PS-fU-PS-mG-PO- | 1177 |
| | PO-lA-PO-lU-PO-mU-PO-mG- | | mA-PO-mA-PO-fU-PO-mU- | |
| | PO-fA-PO-mU-PO-fC-PO-mA- | | PO-mG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | Hy | |
| 165-c-52 | Hy-lgT7-PO-lgT7-PO-lgT7- | 1016 | Hy-mA-PS-fU-PS-mG-PO- | 1178 |
| | PO-lA-PO-lU-PO-mU-PO-mG- | | fA-PO-mA-PO-mU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | Hy | |
| 165-c-53 | Hy-lgT7-PO-lgT7-PO-lgT7- | 1017 | Hy-mA-PS-fU-PS-mG-PO- | 1179 |
| | PO-lA-PO-lU-PO-mU-PO-mG- | | mA-PO-mA-PO-mU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-mU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | mA-PO-mU-PS-mA-PS-mA- | |
| | PO-lT4-Hy | | Hy | |
| 165-c-54 | Hy-lgT7-PO-lgT7-PO-lgT7- | 1018 | Hy-fA-PS-fU-PS-mG-PO- | 1180 |
| | PO-lA-PO-lU-PO-mU-PO-mG- | | fA-PO-mA-PO-fU-PO-mU- | |
| | PO-fA-PO-mU-PO-fC-PO-mA- | | PO-fG-PO-mU-PO-mC-PO- | |
| | PO-fA-PO-fG-PO-fA-PO-mC- | | mU-PO-fU-PO-mG-PO-fA- | |
| | PO-mA-PO-mA-PO-mU-PO-mU- | | PO-mU-PO-fC-PO-mA-PO- | |
| | PO-mC-PO-mA-PO-mU-PO-lT4- | | fA-PO-mU-PS-dT-PS-dT- | |
| | PO-lT4-Hy | | Hy | |

While the exemplary siRNAs shown in Tables 2 and 3 include nucleotide modifications, siRNAs having the same or substantially the same sequences but different numbers, patterns, and/or types of modifications, are also contemplated.

In some embodiments, a dsRNA comprises a sense strand shown in Table 1 with the addition of nucleotides (or modified versions thereof) at either or both of its termini. For example, the dsRNA comprises a sense strand shown in Table 1 with the addition of a 5′ CCA and/or a 3′ invdT. In some embodiments, a dsRNA comprises an antisense strand shown in Table 1 with the addition of nucleotides (or modified versions thereof) at either or both of its termini. For example, the dsRNA comprises an antisense strand shown in Table 1 with the addition of a 3′ dTdT. In certain embodiments, a dsRNA comprises a pair of sense and antisense strands as shown in Table 1, with the addition of a 5′ CCA and a 3′ invdT to the sense strand and with the addition of a 3′ dTdT to the antisense strand. In certain embodiments, a dsRNA comprises a pair of sense and antisense strands as shown in Table 2, with the addition of a 5′ lgT7-lgT7-lgT7 and a 3′ lT4-lT4 to the sense strand.

In some embodiments, a dsRNA of the present disclosure comprises a sense sequence that is at least 70%, 75%, 80%, 85%, 90%, 95%, 96%, 97%, 98%, or 99% identical in sequence to a sense sequence shown in Table 1. In some embodiments, a dsRNA of the present disclosure comprises an antisense sequence that is at least 70%, 75%, 80%, 85%, 90%, 95%, 96%, 97%, 98%, or 99% identical in sequence to an antisense sequence shown in Table 1. In some embodiments, a dsRNA of the present disclosure comprises sense and antisense sequences that are at least 70%, 75%, 80%, 85%, 90%, 95%, 96%, 97%, 98%, or 99% identical in sequence to sense and antisense sequences, respectively, shown in Table 1. In certain embodiments, the dsRNA comprises sense and antisense strands having the sequences shown in Table 2. In certain embodiments, the dsRNA comprises sense and antisense strands having the sequences shown in Table 3.

The “percentage identity” between two nucleotide sequences is determined by comparing the two optimally-aligned sequences in which the nucleic acid sequence to compare can have additions or deletions compared to the reference sequence for optimal alignment between the two sequences. “Percentage identity” is calculated by determining the number of positions at which the nucleotide residue is identical between the two sequences, preferably between the two complete sequences, dividing the number of identical positions by the total number of positions in the alignment window and multiplying the result by 100 to obtain the percentage identity between the two sequences. For purposes herein, when determining “percentage identity” between two nucleotide sequences, modifications to the nucleotides are not considered. For example, a sequence of 5′-mC-fU-mA-fG-3′ is considered having 100% sequence identity as a sequence of 5′-CUAG-3′.

I.5 dsRNA Conjugates

The present dsRNAs may be covalently or noncovalently linked to one or more ligands or moieties. Examples of such ligands and moieties may be found, e.g., in Jeong et al., Bioconjugate Chem. (2009) 20:5-14 and Sebestydn et al., Methods Mol Biol. (2015) 1218:163-86. In some embodiments, the dsRNA is conjugated/attached to one or more ligands via a linker. Any linker known in the art may be used, including, for example, multivalent (e.g., bivalent, trivalent, or tetravalent) branched linkers. The linker may be cleavable or non-cleavable. Conjugating a ligand to a dsRNA may alter its distribution, enhance its cellular absorption and/or targeting to a particular tissue and/or uptake by one or more specific cell types (e.g., liver cells), and/or enhance the lifetime or half-life of the dsRNA. In some embodiments, a hydrophobic ligand is conjugated to the dsRNA to facilitate direct permeation of the cellular membrane and/or uptake across cells (e.g., liver cells). For ANGPTL3-targeting dsRNAs (e.g., siRNAs), the target tissue may be the liver, including parenchymal cells of the liver (e.g., hepatocytes).

In some embodiments, the dsRNA of the present disclosure is conjugated to a cell-targeting ligand. A cell-targeting ligand refers to a molecular moiety that facilitates delivery of the dsRNA to the target cell, which encompasses (i) increased specificity of the dsRNA to bind to cells expressing the selected target receptors (e.g., target proteins); (ii) increased uptake of the dsRNA by the target cells; and (iii) increased ability of the dsRNA to be appropriately processed once it has entered into a target cell, such as increased intracellular release of an siRNA, e.g., by facilitating the translocation of the siRNA from transport vesicles into the cytoplasm. The ligand may be, for example, a protein (e.g., a glycoprotein), a peptide, a lipid, a carbohydrate, or a molecule having a specific affinity for a co-ligand.

Specific examples of ligands include, without limitation, an antibody or antigen-binding fragment thereof that binds to a specific receptor on a liver cell, thyrotropin, melanotropin, surfactant protein A, mucin carbohydrate, multivalent lactose, multivalent galactose, multivalent mannose, multivalent fucose, N-acetylgalactosamine, N-acetylglucosamine, transferrin, bisphosphonate, a steroid, bile acid, lipopolysaccharide, a recombinant or synthetic molecule such as a synthetic polymer, polyamino acids, an alpha helical peptide, polyglutamate, polyaspartate, lectins, and cofactors. In some embodiments, the ligand is one or more dyes, crosslinkers, polycyclic aromatic hydrocarbons, peptide conjugates (e.g., antennapedia peptide, Tat peptide), polyethylene glycol (PEG), enzymes, haptens, transport/absorption facilitators, synthetic ribonucleases (e.g., imidazole, bisimidazole, histamine, or imidazole clusters), human serum albumin (HSA), or LDL.

In some embodiments, the dsRNA is conjugated to one or more cholesterol derivatives or lipophilic moieties such as cholesterol or a cholesterol derivative; cholic acid; a vitamin (such as folate, vitamin A, vitamin E (tocopherol), biotin, or pyridoxal); bile or fatty acid conjugates, including both saturated and non-saturated (such as lauroyl (C12), myristoyl (C14), palmitoyl (C16), stearoyl (C18) and docosanyl (C22), lithocholic acid and/or lithocholic acid oleylamine conjugate (lithocholic-oleyl, C43)); polymeric backbones or scaffolds (such as PEG, triethylene glycol (TEG), hexaethylene glycol (HEG), poly(lactic-co-glycolic acid) (PLGA), poly(lactide-co-glycolide) (PLG), hydrodynamic polymers); steroids (such as dihydrotestosterone); terpene (such as triterpene); cationic lipids or peptides; and/or a lipid or lipid-based molecule. Such a lipid or lipid-based molecule may bind a serum protein, e.g., human serum albumin (HSA). A lipid-based ligand may be used to modulate (e.g., control) the binding of the conjugate to a target tissue. For example, a lipid or lipid-based ligand that binds to HSA more strongly will be less likely to be targeted to the kidney and therefore less likely to be cleared from the body.

In some embodiments, the cell-targeting moiety or ligand is a N-acetylgalactosamine (GalNAc) derivative. In some embodiments, the dsRNA is attached to one or more (e.g., two, three, four, or more) GalNAc derivatives. The attachment may be via one or more linkers (e.g., two, three, four, or more linkers). In some embodiments, a linker described herein is a multivalent (e.g., bivalent, trivalent, or tetravalent) branched linker. In some embodiments, the dsRNA is attached to two or more GalNAc derivatives via a bivalent branched linker. In some embodiments, the dsRNA is attached to three or more GalNAc derivatives via a trivalent branched linker. In some embodiments, the dsRNA is attached to three or more GalNAc derivatives with or without linkers. In some embodiments, the dsRNA is attached to four or more GalNAc derivatives via four separate linkers. In some embodiments, the dsRNA is attached to four or more GalNAc derivatives via a tetravalent branched linker. In some embodiments, the one or more GalNAc derivatives is attached to the 3′-end of the sense strand, the 3′-end of the antisense strand, the 5′-end of the sense strand, and/or the 5′-end of the antisense strand of the dsRNA. Exemplary and non-limiting conjugates and linkers are described, e.g., in Biessen et al., Bioconjugate Chem. (2002) 13(2):295-302; Cedillo et al., Molecules (2017) 22(8):E1356; Grijalvo et al., Genes (2018) 9(2):E74; Huang et al., Molecular Therapy: Nucleic Acids (2017) 6:116-32; Nair et al., J Am Chem Soc. (2014) 136:16958-61; Ostergaard et al., Bioconjugate Chem. (2015) 26:1451-5; Springer et al., Nucleic Acid Therapeutics (2018) 28(3):109-18; and U.S. Pat. Nos. 8,106,022, 9,127,276, and 8,927,705. GalNAc conjugation can be readily performed by methods well known in the art (e.g., as described in the above documents)

II. Methods of Making dsRNAs

A dsRNA of the present disclosure may be synthesized by any method known in the art. For example, a dsRNA may be synthesized by use of an automated synthesizer, by in vitro transcription and purification (e.g., using commercially available in vitro RNA synthesis kits), by transcription and purification from cells (e.g., cells comprising an expression cassette/vector encoding the dsRNA), and the like. In some embodiments, the sense and antisense strands of the dsRNA are synthesized separately and then annealed to form the dsRNA. In some embodiments, the dsRNA comprising modified nucleotides of formula (I) optionally conjugated to a cell targeting moiety (e.g., GalNAc) may be prepared according to the disclosure of PCT Publication WO 2019/170731.

Ligand-conjugated dsRNAs and ligand molecules bearing sequence-specific linked nucleosides of the present disclosure may be assembled by any method known in the art, including, for example, assembly on a suitable polynucleotide synthesizer utilizing standard nucleotide or nucleoside precursors, or nucleotide or nucleoside conjugate precursors that already bear the linking moiety, ligand-nucleotide, or nucleoside-conjugated precursors that already bear the ligand molecule, or non-nucleoside ligand-bearing building blocks.

Ligand-conjugated dsRNAs of the present disclosure may be synthesized by any method known in the art, including, for example, by the use of a dsRNA bearing a pendant reactive functionality such as that derived from the attachment of a linking molecule onto the dsRNA. In some embodiments, this reactive oligonucleotide may be reacted directly with commercially-available ligands, ligands that are synthesized bearing any of a variety of protecting groups, or ligands that have a linking moiety attached thereto. In some embodiments, the methods facilitate the synthesis of ligand-conjugated dsRNA by the use of nucleoside monomers that have been appropriately conjugated with ligands and that may further be attached to a solid support material. In some embodiments, a dsRNA bearing an aralkyl ligand attached to the 3′-end of the dsRNA is prepared by first covalently attaching a monomer building block to a controlled-pore-glass support via a long-chain aminoalkyl group; then, nucleotides are bonded via standard solid-phase synthesis techniques to the monomer building-block bound to the solid support. The monomer building-block may be a nucleoside or other organic compound that is compatible with solid-phase synthesis.

In some embodiments, functionalized nucleoside sequences of the present disclosure possessing an amino group at the 5′-terminus are prepared using a polynucleotide synthesizer, and then reacted with an active ester derivative of a selected ligand. Active ester derivatives are well known to one of ordinary skill in the art. The reaction of the amino group and the active ester produces an oligonucleotide in which the selected ligand is attached to the 5′-position through a linking group. The amino group at the 5′-terminus can be prepared utilizing a 5′-amino-modifier C6 reagent. In some embodiments, ligand molecules are conjugated to oligonucleotides at the 5′-position by the use of a ligand-nucleoside phosphoramidite wherein the ligand is linked to the 5′-hydroxy group directly or indirectly via a linker. Such ligand-nucleoside phosphoramidites are typically used at the end of an automated synthesis procedure to provide a ligand-conjugated oligonucleotide bearing the ligand at the 5′-terminus.

In some embodiments, click chemistry is used to synthesize siRNA conjugates. See, e.g., Astakhova et al., Mol Pharm. (2018) 15(8):2892-9; Mercier et al., Bioconjugate Chem. (2011) 22(1):108-14.

III. Compositions and Delivery of dsRNAs

Certain aspects of the present disclosure relate to compositions (e.g., pharmaceutical compositions) comprising a dsRNA as described herein. In some embodiments, the composition further comprises a pharmaceutically acceptable excipient. In some embodiments, the composition is useful for treating patient having or at risk of having a disease or disorder associated with the expression or activity of the ANGPTL3 gene. In some embodiments, the disease or disorder associated with the expression of the ANGPTL3 gene is a lipid metabolism disorder (e.g., hypertriglyceridemia and hyperlipidemia (such as familial combined hyperlipidemia, familial hypercholesterolemia (e.g., HoFH), and polygenic hypercholesterolemia) and conditions and diseases associated with elevated TGs and/or LDL-c (e.g., atherosclerosis, arteriosclerosis, heart disease, heart attack, stroke, and pancreatitis), and/or any other associated condition and disease described herein and in the art. Compositions of the present disclosure may be formulated based upon the mode of delivery, including, for example, compositions formulated for delivery to the liver via parenteral administration.

The present dsRNAs can be formulated with a pharmaceutically acceptable excipient. Pharmaceutically acceptable excipients can be liquid or solid, and may be selected with the planned manner of administration in mind so as to provide for the desired bulk, consistency, and other pertinent transport and chemical properties. Any known pharmaceutically acceptable excipient may be used, including, for example, water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), calcium salts (e.g., calcium sulfate, calcium chloride, calcium phosphate, and hydroxyapatite), and wetting agents (e.g., sodium lauryl sulfate).

The present dsRNAs can be formulated into compositions (e.g., pharmaceutical compositions) containing the dsRNA admixed, encapsulated, conjugated, or otherwise associated with other molecules, molecular structures, or mixtures of nucleic acids. For example, a composition comprising one or more dsRNAs as described herein can contain other therapeutic agents such as other lipid lowering agents (e.g., statins). In some embodiments, the composition (e.g., pharmaceutical composition) further comprises a delivery vehicle as described herein.

A dsRNA of the present disclosure may be delivered directly or indirectly. In some embodiments, the dsRNA is delivered directly by administering a pharmaceutical composition comprising the dsRNA to a subject. In some embodiments, the dsRNA is delivered indirectly by administering one or more vectors described below.

A dsRNA of the present disclosure may be delivered by any method known in the art, including, for example, by adapting a method of delivering a nucleic acid molecule for use with a dsRNA (see, e.g., Akhtar et al., Trends Cell. Biol. (1992) 2(5):139-44; PCT Patent Publication No. WO 94/02595), or via additional methods known in the art (see, e.g., Kanasty et al., Nature Materials (2013) 12:967-77; Wittrup and Lieberman, Nature Reviews Genetics (2015) 16:543-52; Whitehead et al., (2009) Nature Reviews Drug Discovery 8:129-38; Gary et al., J Control Release (2007) 121(1-2):64-73; Wang et al., AAPS J. (2010) 12(4):492-503; Draz et al., Theranostics (2014) 4(9):872-92; Wan et al., Drug Deliv Transl Res. (2013) 4(1):74-83; Erdmann and Barciszewski (eds.) (2010) “RNA Technologies and Their Applications,” Springer-Verlag Berlin Heidelberg, DOI 10.1007/978-3-642-12168-5; Xu and Wang, Asian Journal of Pharmaceutical Sciences (2015) 10(1):1-12). For in vivo delivery, dsRNA can be injected into a tissue site or administered systemically (e.g., in nanoparticle form via inhalation). In vivo delivery can also be mediated by a beta-glucan delivery system (see, e.g., Tesz et al., Biochem J. (2011) 436(2):351-62). In vitro introduction into a cell includes methods known in the art such as electroporation and lipofection.

In some embodiments, a dsRNA of the present disclosure is delivered by a delivery vehicle comprising the dsRNA. In some embodiments, the delivery vehicle is a liposome, lipoplex, complex, or nanoparticle.

### III.1 Liposomal Formulations

Liposomes are unilamellar or multilamellar vesicles which have a membrane formed from a lipophilic material and an aqueous interior. In some embodiments, a liposome is a vesicle composed of amphiphilic lipids arranged in a spherical bilayer or bilayers. The aqueous portion contains the composition to be delivered. Cationic liposomes possess the advantage of being able to fuse to the cell wall. Advantages of liposomes include, e.g., that liposomes obtained from natural phospholipids are biocompatible and biodegradable; that liposomes can incorporate a wide range of water and lipid soluble drugs; and that liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes. For example, engineered cationic liposomes and sterically stabilized liposomes can be used to deliver the dsRNA. See, e.g., Podesta et al., Methods Enzymol. (2009) 464:343-54; U.S. Pat. No. 5,665,710.

### III.2 Nucleic Acid-Lipid Particles

In some embodiments, a dsRNA of the present disclosure is fully encapsulated in a lipid formulation, e.g., to form a nucleic acid-lipid particle such as, without limitation, a SPLP, pSPLP, or SNALP. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. Nucleic acid-lipid particles, e.g., SNALPs, typically contain a cationic lipid, a non-cationic lipid, and a lipid that prevents aggregation of the particle (e.g., a PEG-lipid conjugate). SNALPs and SPLPs are useful for systemic applications, as they exhibit extended circulation lifetimes following intravenous (i.v.) injection and accumulate at distal sites (e.g., sites physically separated from the administration site). SPLPs include “pSPLPs,” which include an encapsulated condensing agent-nucleic acid complex as set forth in PCT Publication No. WO 00/03683.

In some embodiments, dsRNAs when present in nucleic acid-lipid particles are resistant in aqueous solution to degradation with a nuclease. Nucleic acid-lipid particles and their methods of preparation are disclosed in, e.g., U.S. Pat. Nos. 5,976,567; 5,981,501; 6,534,484; 6,586,410; and 6,815,432; and PCT Publication WO 96/40964.

In some embodiments, the nucleic acid-lipid particles comprise a cationic lipid. Any cationic lipid or mixture thereof known in the art may be used. In some embodiments, the nucleic acid-lipid particles comprise a non-cationic lipid. Any non-cationic lipid or mixture thereof known in the art may be used. In some embodiments, the nucleic acid-lipid particle comprises a conjugated lipid (e.g., to prevent aggregation). Any conjugated lipid known in the art may be used.

### III.3 Additional Formulations

Factors that are important to consider in order to successfully deliver a dsRNA molecule in vivo include: (1) biological stability of the delivered molecule, (2) preventing nonspecific effects, and (3) accumulation of the delivered molecule in the target tissue. The nonspecific effects of a dsRNA can be minimized by local administration, for example by direct injection or implantation into a tissue or topically administering the preparation. For administering a dsRNA systemically for the treatment of a disease, the dsRNA may be modified or alternatively delivered using a drug delivery system; both methods act to prevent the rapid degradation of the dsRNA by endo- and exonucleases in vivo. Modification of the RNA or the pharmaceutical excipient may also permit targeting of the dsRNA composition to the target tissue and avoid undesirable off-target effects. As described above, dsRNA molecules may be modified by chemical conjugation to lipophilic groups such as cholesterol to enhance cellular uptake and prevent degradation. In some embodiments, the dsRNA is delivered using drug delivery systems such as a nanoparticle (e.g., a calcium phosphate nanoparticle), a dendrimer, a polymer, liposomes, or a cationic delivery system. Positively charged cationic delivery systems facilitate binding of a dsRNA molecule (negatively charged) and also enhance interactions at the negatively charged cell membrane to permit efficient uptake of a dsRNA by the cell. Cationic lipids, dendrimers, or polymers can either be bound to a dsRNA, or induced to form a vesicle or micelle (See, e.g., Kim et al., Journal of Controlled Release (2008) 129(2):107-16) that encases a dsRNA. The formation of vesicles or micelles further prevents degradation of the dsRNA when administered systemically. Methods for making and administering cationic-dsRNA complexes are known in the art. In some embodiments, a dsRNA may form a complex with cyclodextrin for systemic administration.

III.4 Vector-Encoded dsRNAs

A dsRNA of the present disclosure may be delivered to the target cell indirectly by introducing into the target cell a recombinant vector (DNA or RNA vector) encoding the dsRNA. The dsRNA will be expressed from the vector inside the cell, e.g., in the form of shRNA, where the shRNA is subsequently processed into siRNA intracellularly. In some embodiments, the vector is a plasmid, cosmid, or viral vector. In some embodiments, the vector is compatible with expression in prokaryotic cells. In some embodiments, the vector is compatible with expression in E. coli. In some embodiments, the vector is compatible with expression in eukaryotic cells. In some embodiments, the vector is compatible with expression in yeast cells. In some embodiments, the vector is compatible with expression in vertebrate cells. Any expression vector capable of encoding dsRNA known in the art may be used, including, for example, vectors derived from adenovirus (AV), adeno-associated virus (AAV), retroviruses (e.g., lentiviruses (LV), Rhabdoviruses, murine leukemia virus, etc.), herpes virus, SV40 virus, polyoma virus, papilloma virus, picornavirus, pox virus (e.g., orthopox or avipox), and the like. The tropism of viral vectors or viral-derived vectors may be modified by pseudotyping the vectors with envelope proteins or other surface antigens from one or more other viruses, or by substituting different viral capsid proteins, as appropriate. For example, lentiviral vectors may be pseudotypes with surface proteins from vesicular stomatitis virus (VSV), rabies, Ebola, Mokola, and the like. AAV vectors may be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes have been described previously (see, e.g., Rabinowitz et al., J. Virol. (2002) 76:791-801).

Selection of recombinant vectors, methods for inserting nucleic acid sequences into the vector for expressing a dsRNA, and methods of delivering vectors into one or more cells of interest are known in the art. See, e.g., Domburg, Gene Therap. (1995) 2:301-10; Eglitis, Biotechniques (1998) 6:608-14; Miller, Hum Gene Therap. (1990) 1:5-14; Anderson, Nature (1998) 392:25-30; Xia et al., Nat Biotech. (2002) 20:1006-10; Robinson et al., Nat Genet. (2003) 33:401-6; Samulski et al., J Virol. (1987) 61:3096-101; Fisher et al., J Virol. (1996) 70:520-32; Samulski et al., J Virol. (1989) 63:3822-6; U.S. Pat. Nos. 5,252,479 and 5,139,941; and PCT Publications WO 94/13788 and WO 93/24641.

Vectors useful for the delivery of a dsRNA as described herein may include regulatory elements (e.g., heterologous promoter, enhancer, etc.) sufficient for expression of the dsRNA in the desired target cell or tissue. In some embodiments, the vector comprises one or more sequences encoding the dsRNA linked to one or more heterologous promoters. Any heterologous promoter known in the art capable of expressing a dsRNA may be used, including, for example, the U6 or H1 RNA pol III promoters, the T7 promoter, and the cytomegalovirus promoter. The one or more heterologous promoters may be an inducible promoter, a repressible promoter, a regulatable promoter, and/or a tissue-specific promoter. Selection of additional promoters is within the abilities of one of ordinary skill in the art. In some embodiments, the regulatory elements are selected to provide constitutive expression. In some embodiments, the regulatory elements are selected to provide regulated/inducible/repressible expression. In some embodiments, the regulatory elements are selected to provide tissue-specific expression. In some embodiments, the regulatory elements and sequence encoding the dsRNA form a transcription unit.

A dsRNA of the present disclosure may be expressed from transcription units inserted into DNA or RNA vectors (see, e.g., Couture et al., TIG (1996) 12:5-10; PCT Patent Publications WO 00/22113 and WO 00/22114; and U.S. Pat. No. 6,054,299). Expression may be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann et al., PNAS (1995) 92:1292).

In some embodiments, the sense and antisense strands of a dsRNA are encoded on separate expression vectors. In some embodiments, the sense and antisense strands are expressed on two separate expression vectors that are co-introduced (e.g., by transfection or infection) into the same target cell. In some embodiments, the sense and antisense strands are encoded on the same expression vector. In some embodiments, the sense and antisense strands are transcribed from separate promoters which are located on the same expression vector. In some embodiments, the sense and antisense strands are transcribed from the same promoter on the same expression vector. In some embodiments, the sense and antisense strands are transcribed from the same promoter as an inverted repeat joined by a linker polynucleotide sequence such that the dsRNA has a stem and loop structure.

IV. dsRNA Therapy

Certain aspects of the present disclosure relate to methods for inhibiting the expression of the ANGPTL3 gene in a subject (e.g., a primate subject such as a human) comprising administering a therapeutically effective amount of one or more dsRNAs of the present disclosure, one or more vectors of the present disclosure, or one or more pharmaceutical compositions of the present disclosure. Certain aspects of the present disclosure relate to methods of treating and/or preventing one or more conditions described herein (e.g., an ANGPTL3-associated condition) comprising administering one or more dsRNAs of the present disclosure and/or one or more vectors of the present disclosure and/or one or more pharmaceutical compositions comprising one or more dsRNAs as described herein. In some embodiments, downregulating ANGPTL3 expression in a subject alleviates one or more symptoms of a lipid metabolism disorder such as hyperlipidemia, familial combined hyperlipidemia, familial hypercholesterolemia (e.g., HoFH), and polygenic hypercholesterolemia; or a disease or condition associated with elevated TGs and LDL-c (e.g., atherosclerosis, arteriosclerosis, coronary heart disease, heart attack, stroke, cachexia, pancreatitis, and diseases in the central nervous system such as Alzheimer's disease and multiple sclerosis), in the subject.

The pharmaceutical composition of the present disclosure may be administered in dosages sufficient to inhibit expression of the ANGPTL3 gene. In some embodiments, a suitable dose of a dsRNA described herein is in the range of 0.001 mg/kg-200 mg/kg body weight of the recipient. In certain embodiments, a suitable dose is in the range of 0.001 mg/kg-50 mg/kg body weight of the recipient, e.g., in the range of 0.001 mg/kg-20 mg/kg body weight of the recipient. Treatment of a subject with a therapeutically effective amount of a pharmaceutical composition can include a single treatment or a series of treatments.

As used herein, the terms “therapeutically effective amount” and “prophylactically effective amount” refer to an amount that provides a therapeutic benefit in the treatment, prevention, or management of pathological processes mediated by ANGPTL3 expression, or an overt symptom of pathological processes mediated by ANGPTL3 expression.

As used herein, the term “ANGPTL3-associated condition” is intended to include any condition in which inhibiting the activity of ANGPTL3 is beneficial. Such a condition may be caused, for example, by excess production of the ANGPTL3 protein, by ANGPTL3 gene mutations that increase ANGPTL3 activity or expression, by abnormal cleavage of the ANGPTL3 protein that increases activity or decreases degradation, and/or by abnormal interactions between ANGPTL3 and other proteins or other endogenous or exogenous substances such that ANGPTL3 activity is increased or degradation is decreased. An ANGPTL3-associated condition may be selected from hypertriglyceridemia and associated diseases and conditions such as atherosclerosis, pancreatitis, and hyperlipidemia such as familial combined hyperlipidemia, familial hypercholesterolemia (e.g., HoFH), and polygenic hypercholesterolemia. An ANGPTL3-associated condition may be, e.g., a lipid metabolism disorder, such as hypertriglyceridemia.

In some embodiments, a dsRNA described herein is used to treat a subject with a lipid metabolism disorder such as hypertriglyceridemia or any symptoms or conditions associated with hypertriglyceridemia. In certain embodiments, a dsRNA described herein is used to treat a patient with drug-induced hypertriglyceridemia, diuretic-induced hypertriglyceridemia, alcohol-induced hypertriglyceridemia, β-adrenergic blocking agent-induced hypertriglyceridemia, estrogen-induced hypertriglyceridemia, glucocorticoid-induced hypertriglyceridemia, retinoid-induced hypertriglyceridemia, cimetidine-induced hypertriglyceridemia, familial hypertriglyceridemia, acute pancreatitis associated with hypertriglyceridemia, and/or hepatosplenomegaly associated with hypertriglyceridemia.

In some embodiments, a dsRNA described herein is used to treat a subject having one or more conditions selected from: lipidemia (e.g., hyperlipidemia), dyslipidemia (e.g., atherogenic dyslipidemia, diabetic dyslipidemia, or mixed dyslipidemia), hyperlipoproteinemia, hypercholesterolemia (e.g., HoFH caused by, for example, a loss-of-function genetic mutation in the LDL receptor (LDLR), rendering a deficient or inactive LDLR), gout associated with hypercholesterolemia, chylomicronemia, lipodystrophy, lipoatrophy, metabolic syndrome, diabetes (Type I or Type II), pre-diabetes, Cushing's syndrome, acromegaly, systemic lupus erythematosus, dysglobulinemia, polycystic ovary syndrome, Addison's disease, glycogen storage disease type 1, hypothyroidism, uremia, adriamycin cardiomyopathy, lipoprotein lipase deficiency, lysosomal acid lipase deficiency, xanthomatosis, eruptive xanthoma, and lipemia retinalis.

Additionally or alternatively, a dsRNA described herein may be used to treat a subject with one or more pathological conditions associated with any of the disorders described herein, such as heart and circulatory conditions (e.g., atherosclerosis, angina, hypertension, congestive heart failure, coronary artery disease, restenosis, myocardial infarction, stroke, aneurysm, cerebrovascular diseases, and peripheral vascular diseases), liver disease, kidney disease, nephrotic syndrome, and chronic renal disease (e.g., uremia, nephrotic syndrome, maintenance dialysis, and renal transplantation).

In some embodiments, a dsRNA described herein may be used to treat a subject with one or more conditions associated with any genetic profile (e.g., familial hypertriglyceridemia, familial combined lipidemia, familial hypobetalipoproteinemia, or familial dysbetalipoproteinemia), treatment (e.g., use of thiazide diuretics, oral contraceptives and other estrogens, certain beta-adrenergic blocking drugs, propofol, HIV medications, isotretinoin, or protease inhibitors), or lifestyle (e.g., cigarette smoking, excessive alcohol consumption, high carbohydrate diet, or high fat diet) that results in or results from elevated blood triglycerides or lipids. Triglyceride levels (e.g., serum triglyceride levels) of over 150 mg/dL are considered elevated for risk of cardiovascular conditions. Triglyceride levels (e.g., serum triglyceride levels) of 500 mg/dL or higher are considered elevated for risk of pancreatitis.

In some embodiments, a dsRNA described herein may be used to manage body weight or reduce fat mass in a subject.

In some embodiments, a dsRNA as described herein inhibits expression of the human ANGPTL3 gene, or both human and cynomolgus ANGPTL3 genes. The expression of the ANGPTL3 gene in a subject may be inhibited, and/or the ANGPTL3 protein levels in the subject may be reduced, by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or about 100% after treatment as compared to pretreatment levels. In some embodiments, expression of the ANGPTL3 gene is inhibited, and/or the ANGPTL3 protein levels in the subject may be reduced, by at least about 2, at least about 5, at least about 10, at least about 15, at least about 20, at least about 25, at least about 50, at least about 75, or at least about 100 fold after treatment as compared to pretreatment levels. In some embodiments, the ANGPTL3 gene is inhibited, or the ANGPTL3 protein levels are reduced, in the liver of the subject.

In some embodiments, expression of the ANGPTL3 gene is decreased by the dsRNA for about 12 or more, 24 or more, or 36 or more hours. In some embodiments, expression of the ANGPTL3 gene is decreased for an extended duration, e.g., at least about two, three, four, five, or six days or more, e.g., about one week, two weeks, three weeks, four weeks, one month, two months, or longer.

As used herein, the terms “inhibit the expression of” or “inhibiting expression of,” insofar as they refer to the ANGPTL3 gene, refer to the at least partial suppression of the expression of the ANGPTL3 gene, as manifested by a reduction in the amount of mRNA transcribed from the ANGPTL3 gene in a first cell or group of cells treated such that the expression of the ANGPTL3 gene is inhibited, as compared to a second cell or group of cells substantially identical to the first cell or group of cells but which has or have not been so treated (control cells). Such inhibition can be assessed, e.g., by Northern analysis, in situ hybridization, B-DNA analysis, expression profiling, transcription of reporter constructs, and other techniques known in the art. As used herein, the term “inhibiting” is used interchangeably with “reducing,” “silencing,” “downregulating,” “suppressing,” and other similar terms, and include any level of inhibition. The degree of inhibition is usually expressed in terms of (((mRNA in control cells)−(mRNA in treated cells))/(mRNA in control cells))×100%.

Alternatively, the degree of inhibition may be given in terms of a reduction of a parameter that is functionally linked to ANGPTL3 gene transcription, e.g., the amount of protein encoded by the ANGPTL3 gene in a cell (as assessed, e.g., by Western analysis, expression of a reporter protein, ELISA, immunoprecipitation, or other techniques known in the art), or the number of cells displaying a certain phenotype, e.g., apoptosis. In principle, ANGPTL3 gene silencing may be determined in any cell expressing the target, either constitutively or by genomic engineering, and by any appropriate assay. However, when a reference is needed in order to determine whether a given dsRNA inhibits the expression of the ANGPTL3 gene by a certain degree and therefore is encompassed by the present disclosure, the assays provided in the Examples below shall serve as such a reference.

In some embodiments, the effect of inhibiting ANGPTL3 gene expression by any of the methods described herein results in a decrease in triglyceride levels in a subject (e.g., in the blood and/or serum of the subject). In some embodiments, triglyceride levels are decreased to below one of the following levels: 500, 450, 400, 350, 300, 250, 200, 190, 180, 170, 160, 150, 140, 130, 120, 110, 100, 90, 80, 70, 60, or 50 mg/dL. In some embodiments, LDL levels are decreased to below one of the following levels: 190, 180, 170, 160, 150, 140, 130, 120, 110, 100, or 70 mg/dL.

A subject's triglyceride levels may be determined in any of numerous ways known in the art. In some embodiments, a subject's triglyceride levels are determined using a sample from the subject such as blood, serum, or plasma.

A dsRNA or pharmaceutical composition described herein may be administered by any means known in the art, including, without limitation, oral or parenteral routes, including intravenous, intramuscular, subcutaneous, pulmonary, transdermal, and airway (aerosol) administration. Typically, when treating a patient with hypertriglyceridemia, the dsRNA molecules are administered systemically via parenteral means. In some embodiments, the dsRNAs and/or compositions are administered by subcutaneous administration. In some embodiments, the dsRNAs and/or compositions are administered by intravenous administration. In some embodiments, the dsRNAs and/or compositions are administered by pulmonary administration.

As used herein, in the context of ANGPTL3 expression, the terms “treat,” “treatment” and the like refer to relief from or alleviation of pathological processes mediated by target gene expression. In the context of the present disclosure, insofar as it relates to any of the conditions recited herein, the terms “treat,” “treatment,” and the like refer to relieving or alleviating one or more symptoms associated with said condition. For example, in the context of hypertriglyceridemia, treatment may involve a decrease in serum triglyceride levels. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

As used herein, the terms “prevent” or “delay progression of” (and grammatical variants thereof), with respect to a condition relate to prophylactic treatment of a condition, e.g., in an individual suspected to have or be at risk for developing the condition. Prevention may include, but is not limited to, preventing or delaying onset or progression of the condition and/or maintaining one or more symptoms of the disease at a desired or sub-pathological level. For example, in the context of hypertriglyceridemia, prevention may involve maintaining serum triglyceride levels at a desired level in an individual suspected to have or be at risk for developing hypertriglyceridemia.

It is understood that the dsRNAs of the present disclosure may be for use in a treatment as described herein, may be used in a method of treatment as described herein, and/or may be for use in the manufacture of a medicament for a treatment as described herein.

In some embodiments, a dsRNA of the present disclosure is administered in combination with one or more additional therapeutic agents, such as other siRNA therapeutic agents, monoclonal antibodies, and small molecules, to provide a greater improvement to the condition of the patient than administration of the dsRNA alone. In certain embodiments, the additional therapeutic agent provides an anti-inflammatory effect. In certain embodiments, the additional therapeutic agent is an agent that treats hypertriglyceridemia, such as a lipid-lowering agent.

In some embodiments, the additional agent may be one or more of a HMG-CoA reductase inhibitor (e.g., a statin), a fibrate, a bile acid sequestrant, nicotinic acid, an antiplatelet agent, an angiotensin converting enzyme inhibitor, an angiotensin II receptor antagonist (e.g., losartan potassium), an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, an omega-3 fatty acid (e.g., fish oil or flaxseed oil), and insulin or an insulin analog. Particular examples include, without limitation, atorvastatin, pravastatin, simvastatin, lovastatin, fluvastatin, cerivastatin, rosuvastatin, pitavastatin, ezetimibe, bezafibrate, clofibrate, fenofibrate, gemfibrozil, ciprofibrate, cholestyramine, colestipol, colesevelam, and niacin.

In certain embodiments, a dsRNA as described herein may be administered in combination with another therapeutic intervention such as lipid lowering, weight loss, dietary modification, and/or moderate exercise.

Genetic predisposition plays a role in the development of target gene associated diseases, e.g., hypertriglyceridemia. Therefore, a subject in need of treatment with one or more dsRNAs of the present disclosure may be identified by taking a family history, or, for example, screening for one or more genetic markers or variants. Examples of genes involved in hypertriglyceridemia may include, without limitation, LPL, APOB, APOC2, APOA5, APOE, LMF1, GCKR, GPIHBP1, and GPD1. In certain embodiments, a subject in need of treatment with one or more dsRNAs of the present disclosure may be identified by screening for variants of or loss-of-function mutations in any of these genes or any combination thereof.

A healthcare provider, such as a doctor, nurse, or family member, can take a family history before prescribing or administering a dsRNA of the present disclosure. In addition, a test may be performed to determine a genotype or phenotype. For example, a DNA test may be performed on a sample from the subject, e.g., a blood sample, to identify the ANGPTL3 genotype and/or phenotype before the dsRNA is administered to the subject.

### V. Kits and Articles of Manufacture

Certain aspects of the present disclosure relate to an article of manufacture or a kit comprising one or more of the dsRNAs, vectors, or compositions (e.g., pharmaceutical compositions) as described herein useful for the treatment and/or prevention of an ANGPTL3-associated condition (e.g., a lipid metabolism disorder such as hypertriglyceridemia). The article of manufacture or kit may further comprise a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating or preventing the disease and may have a sterile access port (for example the container may be an intravenous solution bag or a vial having a stopper pierceable by a hypodermic injection needle). At least one active agent in the composition is a dsRNA as described herein. The label or package insert indicates that the composition is used for treating an ANGPTL3-associated condition. In some embodiments, the condition is a lipid metabolism disorder such as hypertriglyceridemia and/or another condition described herein. Moreover, the article of manufacture or kit may comprise (a) a first container with a composition contained therein, wherein the composition comprises a dsRNA as described herein; and (b) a second container with a composition contained therein, wherein the composition comprises a second therapeutic agent (e.g., an additional agent as described herein). The article of manufacture or kit in this aspect of the present disclosure may further comprise a package insert indicating that the compositions can be used to treat a particular disease. Alternatively, or additionally, the article of manufacture or kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and/or user standpoint, including other buffers, diluents, filters, needles, and syringes.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control.

Generally, nomenclature used in connection with, and techniques of, cell and tissue culture, molecular biology, cardiology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to the manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

All publications and other references mentioned herein are incorporated by reference in their entirety. Although a number of documents are cited herein, this citation does not constitute an admission that any of these documents forms part of the common general knowledge in the art.

### EXAMPLES

In order for the present disclosure to be better understood, the following examples are set forth. These examples are for illustration only and are not to be construed as limiting the scope of the present disclosure in any manner.

### Example 1: siRNA Synthesis and Purification

siRNAs, including non-targeting control siRNAs (NT control), were produced using solid phase oligonucleotide synthesis.

### Methods

siRNA Production

RNA oligonucleotides were synthesized at a scale of 1 μmol (in vitro) or 10 μmol (in vivo) on a ABI 394 DNA/RNA or BioAutomation MerMade 12 synthesizer using commercially available 5′-O-DMT-3′-O-(2-cyanoethyl-N,N-diisopropyl) phosphoramidite monomers (SAFC) of uridine, 4-N-acetylcytidine (C^{Ac}), 6-N-benzoyladenosine (A^{Bz}) and 2-N-isobutyrylguanosine (G^{iBu}) with 2′-OMe or 2′-F modification, and the solid supports 5′-O-DMT-thymidine-CPG and 3′-O-DMT-thymidine-CPG (invdT, Link) following standard protocols for solid phase synthesis and deprotection (Beaucage, Curr Opin Drug Discov Devel. (2008) 11:203-16; Mueller et al., Curr Org Synth (2004) 1:293-307).

Phosphoramidite building blocks were used as 0.1 M solutions in acetonitrile and activated with 5-(bis-3,5-trifluoromethylphenyl)-1H-tetrazole (activator 42, 0.25 M in acetonitrile, Sigma Aldrich). Reaction times of 300 s were used for the phosphoramidite couplings. As capping reagents, acetic anhydride in THF (CapA for ABI, Sigma Aldrich) and N-methylimidazole in THF (CapB for ABI, Sigma Aldrich) were used. As oxidizing reagent, iodine in THF/pyridine/water (0.02 M; oxidizer for ABI, Sigma Aldrich) was used. Deprotection of the DMT-protecting group was done using dichloroacetic acid in DCM (DCA deblock, Sigma Aldrich). Final cleavage from solid support and deprotection (acyl- and cyanoethyl-protecting groups) was achieved with NH₃ (32% aqueous solution/ethanol, v/v 3:1).

The crude oligonucleotides were analyzed by IEX and LC-MS and purified by anion-exchange high-performance liquid chromatography (IEX) using a linear gradient of 10-65% buffer B in 30 min. ÄKTA purifier (Thermo Fisher Scientific DNAPac PA200 semi prep ion exchange column, 8 μm particles, width 22 mm×length 250 mm).
Buffer A: 1.50 l H₂O, 2.107 g NaClO₄, 438 mg EDTA, 1.818 g TRIS, 540.54 g urea, pH 7.4.
Buffer B: 1.50 l H₂O, 105.34 g NaClO₄, 438 mg EDTA, 1.818 g TRIS, 540.54 g urea, pH 7.4.

Isolation of the oligonucleotides was achieved by precipitation, induced by the addition of 4 volumes of ethanol and storing at −20° C.

To ensure high fidelity of the data, all single strands were HPLC purified to >85% purity. The purity and identity of the oligonucleotides was confirmed by ion exchange chromatography and LC-MS, respectively.

### Duplex Annealing

For the in vitro experiments (100 μM solutions) and in vivo experiments (10 mg/ml), stock solutions of siRNAs in PBS were prepared by mixing equimolar amounts of complementary sense and antisense strands in 1×PBS buffer. The solutions were heated to 90° C. for 10 min and allowed to slowly cool to room temperature to complete the annealing process. siRNAs were further characterized by HPLC and were stored frozen until use.

siRNA Sequences

The sequences of each siRNA, and sequences including nucleotide modifications, are shown in Tables 1 and 2, supra.

siRNA Stability in Mouse Serum

Modified siRNAs listed in Table 2 were tested for nuclease stability in 50% mouse serum. 160 μL of 2.5 μM siRNA in 1×DPBS (Life Technologies, cat. no. 14190-094) and 160 μL mouse serum (Sigma, cat. no. M5905) were incubated at 37° C. for up to 72 h. At each time point (0 h, 8 h, 24 h, 32 h, 48 h, 56 h, and 72 h), 21 μL of the reaction was taken out and quenched with 23 μL stop solution (Tissue & Cell Lysis Solution (Epicentre, cat. no. MTC096H), 183 μL 20 mg/mL Proteinase K (Sigma, cat. no. P2308), 1694 μL water) at 65° C. for 30 min. Prior to HPLC analysis on a Waters 2695 Separation Module and a 2487 Dual Absorbance Detector, 33 μL RNase-free water was added to each sample. 50 μL of the solution was analyzed by HPLC using a DNAPac PA200 analytical column (Thermo Scientific, cat. no. 063000) and the following gradient:

| Time | Flow | % Buffer | % Buffer |
|---|---|---|---|
| (min) | (mL/min) | A* | B** |
| 0 | 1 | 75 | 25 |
| 20 | 1 | 35 | 65 |

Buffer A: 20 mM sodium phosphate (Sigma, Cat. No. 342483), pH 11;
Buffer B: 20 mM sodium phosphate (Sigma, Cat. No. 342483), 1 M sodium bromide (Sigma, Cat. No. 02119), pH 11.

Serum half-lives were estimated for both strands of the siRNA.

### Example 2: Identification of siRNAs for Inhibition of Human ANGPTL3 Expression

### Methods

### Cells and Tissue Culture

Human Hep3B cells were grown at 37° C., 5% CO₂ and 95% relative humidity (RH), and cultivated in EMEM medium (ATCC, cat. no. 30-2003) supplemented with 10% FBS.

siRNA Transfections

For knock-down experiments in Hep3B cells, 20,000 cells/well were used in 96-well plates (Greiner, cat. no. 655180). The cells were transfected with ANGPTL3 siRNAs at 0.1 nM and 1 nM using 0.2 μL/well of Lipofectamine RNAiMAX transfection reagent (ThermoFisher) according to the manufacturer's protocol in a reverse transfection setup, and incubated for 48 h without medium change. Usually, N=4 technical replicates were carried out per test sample.

mRNA Expression Analysis

48 or 72 hours after siRNA transfection or free siRNA uptake, the cellular RNA was harvested by usage of Promega's SV96 total RNA isolation system (cat. no. Z3500) according to the manufacturer's protocol, including a DNase step during the procedure.

For cDNA synthesis, the ThermoFisher Reverse Transcriptase kit (cat. no. N8080234) was used. cDNA was synthesized from 30 ng RNA using 1.2 μL 10×RT buffer, 2.64 μL MgCl₂ (25 mM), 2.4 μL dNTPs (10 mM), 0.6 μL random hexamers (50 μM), 0.6 μL Oligo(dT) 16 (SEQ ID NO: 1185) (50 μM), 0.24 μL RNase inhibitor (20 U/μL) and 0.3 μL Multiscribe (50 U/μL) in a total volume of 12 μL. Samples were incubated at 25° C. for 10 minutes and 42° C. for 60 minutes. The reaction was stopped by heating to 95° C. for 5 minutes.

Human and cynomolgus ANGPTL3 mRNA levels were quantified by qPCR using the ThermoFisher TaqMan Universal PCR Master Mix (cat. no. 4305719) and the following TaqMan Gene Expression assays:

| | |
|---|---|
| Human | Hs00205581_m1 |
| Cynomolgus | Mf04384789_m1 |

PCR was performed in technical duplicates with an ABI Prism 7900 system under the following PCR conditions: 2 minutes at 50° C., 10 minutes at 95° C., 40 cycles with 95° C. for 15 seconds and 1 minute at 60° C. PCR was set up as a simplex PCR detecting the target gene in one reaction and the housekeeping gene (human/cynomolgus RPL37A) for normalization in a parallel reaction. The final volume for the PCR reaction was 12.5 μL in a 1×PCR master mix; RPL37A primers were used at a final concentration of 50 nM and the probe was used at a final concentration of 200 nM. The ΔΔCt method was applied to calculate relative expression levels of the target transcripts. Percentage of target gene expression was calculated by normalization based on the levels of non-targeting siRNA control treated cells.

### IC₅₀ Measurements

For IC₅₀ measurements, 20,000 human Hep3B cells in 96-well plates were transfected with Lipofectamine RNAiMAX for 48 hours with the indicated ANGPTL3 siRNAs at 7 concentrations starting from 25 nM using 5-8-fold dilution steps. The half maximal inhibitory concentration (IC₅₀) for each siRNA was calculated by applying a Biostat-Speed statistical calculation tool. Results were obtained using the 4-parameter logistic model according to Ratkovsky and Reedy (Biometrics 42(3):575-582 (1986)). The adjustment was obtained by non-linear regression using the Levenberg-Marquardt algorithm in SAS v9.1.3 software.

### Cytotoxicity

Cytotoxicity was measured 72 hours after 5 nM and 50 nM siRNA transfections of a culture of 10,000 Hep3B cells per well of a 96-well plate by determining the ratio of cellular viability/toxicity in each sample. Cell viability was measured by determination of the intracellular ATP content using the CellTiter-Glo assay (Promega, cat. no. G7570) according to the manufacturer's protocol. Cell toxicity was measured in the supernatant using the ToxiLight assay (Lonza, cat. no. LT07-217) according to the manufacturer's protocol. 10 nM AllStars Hs Cell Death siRNA (Qiagen, cat. no. SI04381048), 25 μM Ketoconazole (Calbiochem, cat. no. 420600) and 1% Triton X-100 (Sigma, cat. no. T9284) were used as toxic positive controls.

### Results

In order to identify siRNAs useful in targeting human ANGPTL3, the following criteria were applied for in silico library generation: first, 19mers from the human ANGPTL3 mRNA sequence as set forth in NM_014495.3 were identified in silico with an overlap of 18 nucleotides. From this list of 2933 sequences, molecules were further removed if they had a G/C content of greater than 55% or one or more mismatches with the ANGPTL3 mRNA sequence of Macaca fascicularis (cynomolgus monkey).

For the remaining sequences, an in silico analysis was then carried out to identify any potential off-target transcripts matching either siRNA strand (sense/antisense) in the human transcriptome (RefSeq RNA version 2015-11-24). Human off-target sequences with RNAseq expression (Illumina Body Atlas) FPKM<0.5 in liver tissue were not considered. All siRNA sequences of interest had either greater than three mismatches to any human transcript expressed in liver other than ANGPTL3, or had two mismatches with four or fewer human genes; sequences that did not meet one of these two criteria were filtered out. After this filtration, 162 potential siRNAs were left (see Table 1, constructs 001-162).

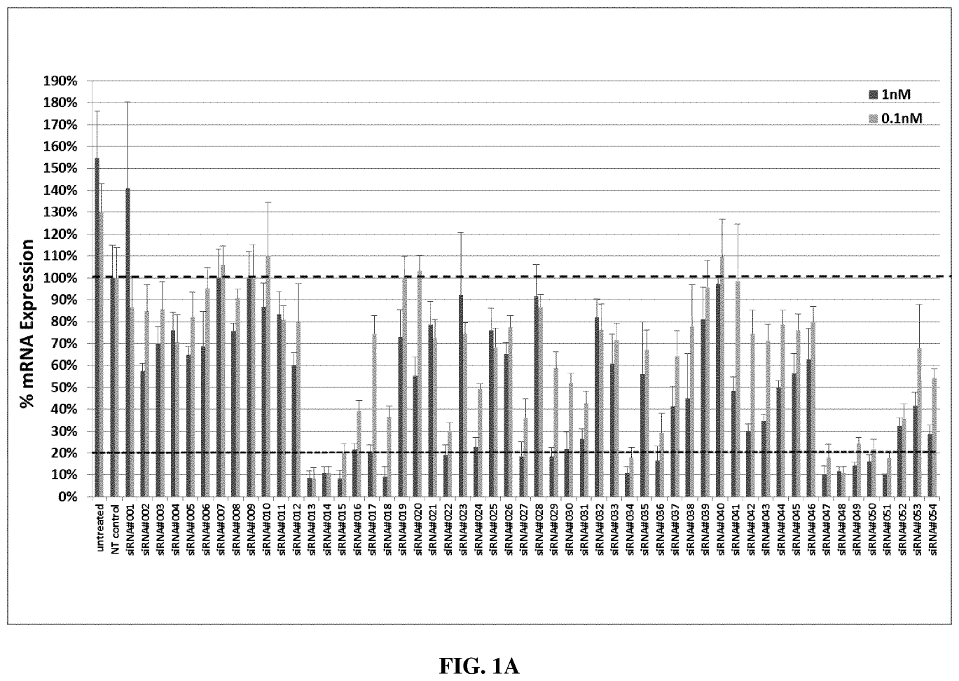

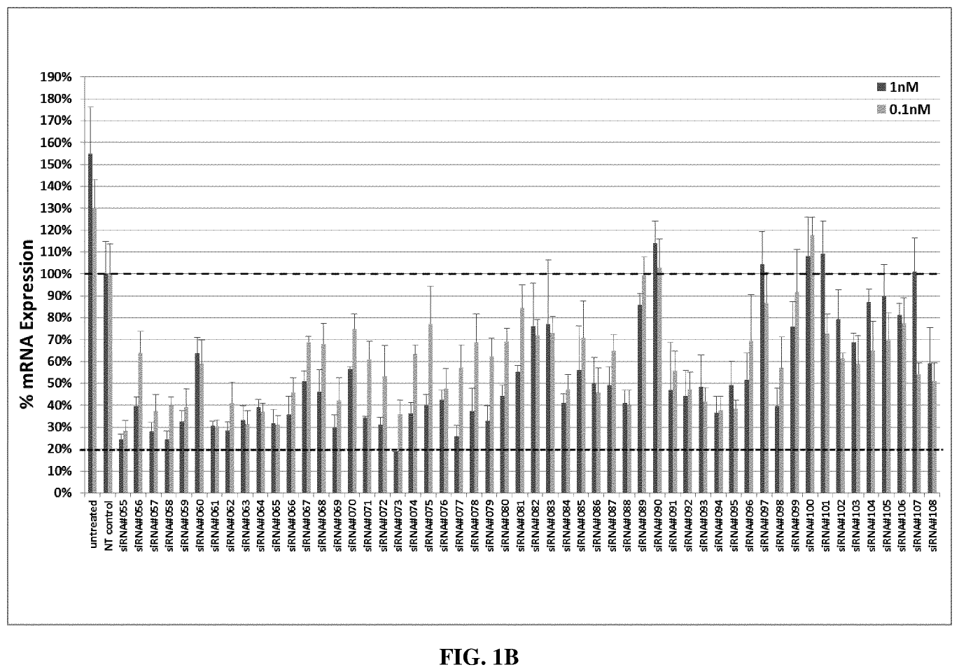

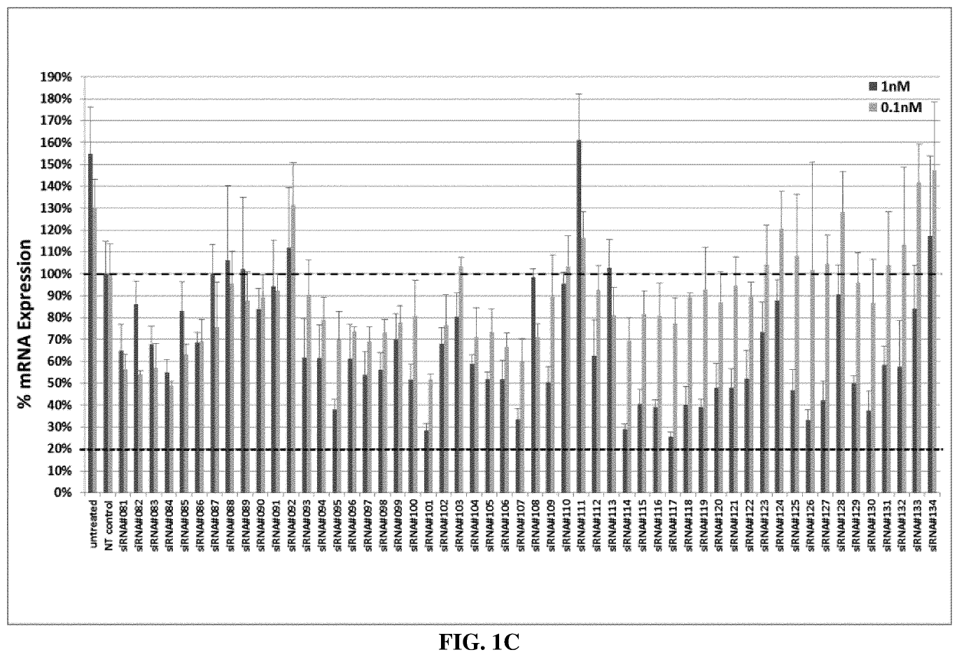

As described above, the 162 siRNAs were produced with nucleotides having a fixed pattern (see Table 2, constructs 001-162). To test the ability of these 162 siRNAs to reduce expression of ANGPTL3, human Hep3B cells were transfected with 0.1 nM or 1.0 nM of each siRNA and incubated for 48 hours. After incubation, mRNA expression of ANGPTL3 was measured in each sample and compared to negative controls treated with non-targeting siRNA (FIGS. 1A-1C). 15 siRNAs that showed reduction of mRNA expression by at least 80% at a concentration of 1.0 nM, or by at least 70% at a concentration of 0.1 nM, plus three siRNAs binding to a distant sequence region, were selected for further characterization.

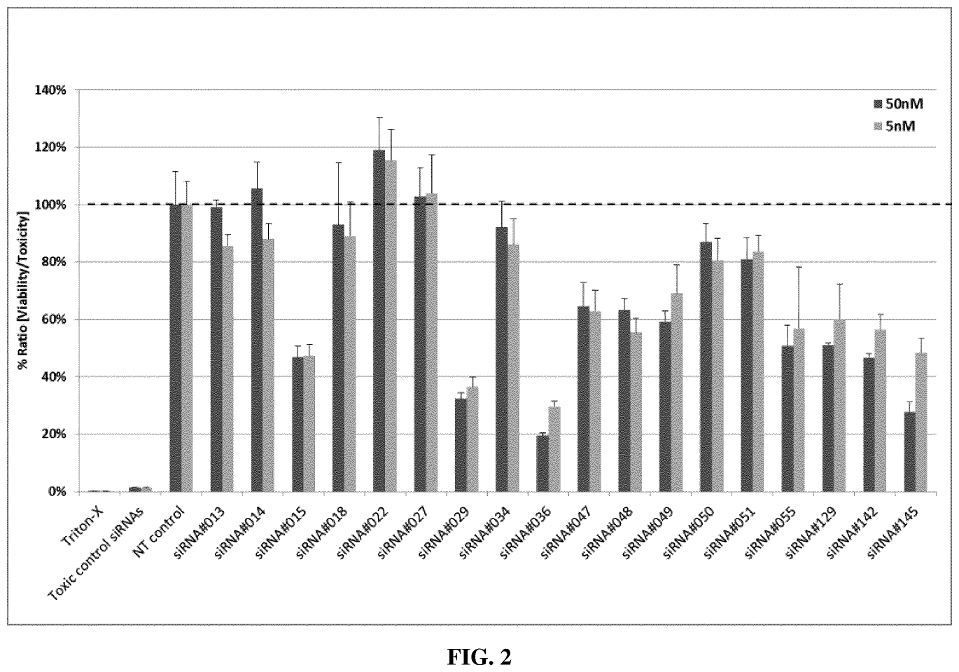

IC₅₀ measurements (Table 4) and a cytotoxicity assay (FIG. 2) were carried out for the selected 18 siRNAs in human Hep3B cells. After removal of 3 siRNAs (siRNA #029, #036, and #145) that showed <40% of NT control Viability/Toxicity ratio (at 50 nM), 11 siRNAs were selected based on their IC₅₀ values for conjugation to GalNAc (Table 4).

TABLE 4
Activity of selected siRNAs

| | | | Selected for |
|---|---|---|---|
| Compound | Iₘₐₓ | IC₅₀ [nM] | GalNAc conjugation |
| siRNA#013 | 0.945 | 9.83E−03 | X |
| siRNA#014 | 0.937 | 3.34E−04 | X |
| siRNA#015 | 0.923 | 7.22E−03 | X |
| siRNA#018 | 0.980 | 5.28E−02 | |
| siRNA#022 | 0.882 | 2.07E−02 | |
| siRNA#027 | 0.818 | 4.75E−02 | |
| siRNA#029 | 0.943 | 2.19E−01 | |
| siRNA#034 | 0.910 | 3.34E−02 | |
| siRNA#036 | 0.872 | 6.52E−02 | |
| siRNA#047 | 0.894 | 2.72E−02 | X |
| siRNA#048 | 0.879 | 1.17E−02 | X |
| siRNA#049 | 0.867 | 9.67E−03 | X |
| siRNA#050 | 0.890 | 5.32E−02 | X |
| siRNA#051 | 0.890 | 1.51E−02 | X |
| siRNA#055 | 0.704 | 1.98E−02 | X |
| siRNA#129 | 0.730 | 2.19E−02 | X |
| siRNA#142 | 0.689 | 1.75E−02 | X |
| siRNA#145 | 0.733 | 2.66E−01 | |

Taken together, these results demonstrate the identification of siRNAs capable of potent inhibition of human ANGPTL3 expression without significant cytotoxicity in human cells.

### Example 3: Identification of Active GalNAc-Conjugated siRNAs for Inhibition of Human and Cynomolgus ANGPTL3 Expression

### Methods

GalNAc-siRNAs, including non-targeting control siRNAs (NT control), were generated based on the sequences as indicated (see sequence listings above).

### Cells Culture and Assays

Human (BioreclamationIVT, cat. no. M00995-P) and cynomolgus (Primacyt, cat. no. CHCP-I-T) primary hepatocytes were cultured as follows: cryopreserved cells were thawed and plated using a plating and thawing kit (Primacyt, cat. no. PTK-1), and were incubated at 37° C., 5% C02 and 95% RH. 6 hours after plating, the medium was changed to maintenance medium (KaLy-Cell, cat. no. KLC-MM) supplemented with 1% FBS.

mRNA expression analysis was performed as described above in Example 2.

### IC₅₀ Measurements

For demonstration of dose-activity relationships and IC₅₀ measurements in human and cynomolgus primary hepatocytes under free uptake conditions, 50,000-70,000 cells in 96-well plates were incubated for 72 hours without medium change with the siRNAs at concentrations ranging from 10 μM-0.01 nM using 10-fold dilution steps. The half maximal inhibitory concentration (IC₅₀) for each siRNA was calculated by applying a Biostat-Speed statistical calculation tool. Results were obtained using the 4-parameter logistic model according to Ratkovsky and Reedy (Biometrics (1986) 42(3):575-82). The adjustment was obtained by non-linear regression using the Levenberg-Marquardt algorithm in SAS v9.1.3 software.

### Results

Following selection of potent siRNAs as described above, the inventors went on to demonstrate whether the selected molecules retain their activity in the context of a GalNAc-conjugate suitable for liver specific siRNA delivery in vivo. They also assessed whether this activity holds up in cells from M. fascicularis (cynomolgus monkey).

The results of the IC₅₀ measurements show that all tested siRNA conjugates except for two retain activity when delivered by free uptake to human primary hepatocytes (Table 5), with IC₅₀ values ranging from 1.95 to 9.2 nM. Surprisingly, however, the performance ranking following free uptake of GalNAc-siRNA differs significantly from that obtained after transfection assisted uptake of unconjugated siRNA (Table 4), including complete failure of two molecules to produce measurable knock-down activity. This indicates that siRNAs seem to have inherent properties based on their sequence that makes them differentially suited for application in the context of GalNAc conjugates with regard to resulting knock-down potency.

TABLE 5
Iₘₐₓ and IC₅₀ of selected GalNAc-conjugated
siRNAs in human primary hepatocytes

| Compound | Iₘₐₓ | IC₅₀ [nM] |
|---|---|---|
| siRNA#013-c | 0.891 | 2.32 |
| siRNA#014-c | 0.912 | 1.95 |
| siRNA#015-c | 0.850 | 3.07 |
| siRNA#047-c | 0.782 | 5.03 |
| siRNA#048-c | 0.768 | 4.39 |
| siRNA#049-c | 0.711 | 4.36 |
| siRNA#050-c | 0.670 | 9.20 |
| siRNA#051-c | 0.735 | 5.50 |
| siRNA#055-c | 0.502 | 2.06 |
| siRNA#129-c | N/A | N/A |
| siRNA#142-c | N/A | N/A |

N/A: No measurable activity.

Even more surprisingly, some of the tested siRNAs show absence of activity in cynomolgus hepatocytes despite predicted sequence homology to the M. fascicularis sequence XM_005543185.1 (Table 6). This unexpected observation highlights the requirement of a functional assay for activity detection and that the efficacy of siRNAs cannot be predicted purely based on bioinformatical information.

TABLE 6
Iₘₐₓ and IC₅₀ of selected GalNAc-conjugated
siRNAs in cynomolgus primary hepatocytes

| Compound | Iₘₐₓ | IC₅₀ [nM] |
|---|---|---|
| siRNA#013-c | 0.897 | 2.31 |
| siRNA#014-c | 0.893 | 4.15 |
| siRNA#015-c | 0.738 | 2.89 |
| siRNA#047-c | N/A | N/A |
| siRNA#048-c | N/A | N/A |
| siRNA#049-c | N/A | N/A |
| siRNA#050-c | N/A | N/A |
| siRNA#051-c | N/A | N/A |
| siRNA#055-c | N/A | N/A |
| siRNA#129-c | N/A | N/A |
| siRNA#142-c | N/A | N/A |

N/A: No measurable activity.

### Example 4: In Vitro and In Vivo Characterization of GalNAc-Conjugated siRNAs for Inhibition of Human ANGPTL3 Expression

### Methods

### Cells and Tissue Culture

Human Hep3B cells were grown at 37° C., 5% CO₂ and 95% RH, and cultivated in EMEM medium (ATCC, cat. no. 30-2003) supplemented with 10% FBS.

Human (BioreclamationIVT, cat. no. M00995-P) and cynomolgus (Primacyt, cat. no. CHCP-T-T) primary hepatocytes were cultured as follows: cryopreserved cells were thawed and plated using a plating and thawing kit (Primacyt, cat. no. PTK-1), and were incubated at 37° C., 5% C02 and 95% RH. 6 hours after plating, the medium was changed to maintenance medium (KaLy-Cell, cat. no. KLC-MM) supplemented with 1% FBS.

Human peripheral blood mononuclear cells (PBMCs) were isolated from approximately 16 mL of blood from three healthy donors that were collected in Vacutainer tubes coated with sodium heparin (BD, Heidelberg Germany) according to the manufacturer's instructions.

For transfection of human PBMCs, 100 nM of the siRNAs were reverse transfected into 1×10⁵ PBMCs with 0.3 μL Lipofectamine 2000 per well of a 96-well plate (N=2) in a total volume of 150 μL serum-free RPMI medium (ThermoFisher, cat. no. 11875) for 24 hours. Single-stranded RNA (“R-0006”) and DNA (“CpG ODN”) oligonucleotides, as well as double-stranded unmodified and 2′-O-methyl modified siRNA (“132/161”), were applied as controls.

### ANGPTL3 ELISA Assay

ANGPTL3 protein concentration was quantified in the supernatant from IC₅₀ experiments for selected siRNA concentrations by applying R&D Systems' human ANGPTL3 Quantikine ELISA kit (cat. no. DANL30). The ELISA assay was performed using 50 μl of 1:2-1:8 pre-diluted supernatant from human Hep3B cells, human primary hepatocytes, or cynomolgus primary hepatocytes according to the manufacturer's protocol. The percentage of ANGPTL3 protein expression was calculated by normalization based on the mean ANGPTL3 levels of cells treated with non-targeting siRNA control sequences.

### IFNα Determination

IFNα protein concentration was quantified in the supernatant of human PBMCs as follows: 25 μL of the cell culture supernatant was used for measurement of IFNα concentration applying a self-established electrochemiluminescence assay based on MesoScale Discovery's technology, and using a pan IFNα monoclonal capture antibody (MT1/3/5, Mabtech). Alternatively, a human IFNα2a isoform-specific assay (cat. no. K151VHK) was applied based on MesoScale's U-PLEX platform and according to the supplier's protocol.

### Cytotoxicity

siRNA cytotoxicity in human primary hepatocytes was measured 72 hours after incubation of 45,000-50,000 cells per well of a 96-well plate with 1 μM, 5 μM and 25 μM siRNA under free uptake conditions by determining the ratio of cellular viability/toxicity in each sample. Cell viability was measured by determination of the intracellular ATP content using the CellTiter-Glo assay (Promega, cat. no. G7570), and cell toxicity was measured in the supernatant using the LDH assay (Sigma, cat. no. 11644793001) according to the manufacturer's protocols. 25 μM Ketoconazole and 1% Triton X-100 were used as positive controls.

### Nuclease Stability

The GalNAc-conjugated siRNAs were tested for nuclease stability using the method described in Example 1.

### In Vivo Assay

To assess the effect of GalNAc-siRNAs targeting human ANGPTL3 in vivo, a transgene expression system based on adeno-associated viral vectors was applied in mice. To this end, an AAV8 vector with liver specific expression of mRNA, encoding human ANGPTL3 from an ApoA2 promoter (Vectalys, Toulouse, France), was administered intravenously to female C57BL/6 mice (Charles River, Germany) before siRNA dosing. GalNAc-conjugated siRNAs (including non-targeting control) were administered subcutaneously at 12 mg/kg (n=8) after serum levels of human ANGPTL3 expressed from the AAV vector reached sufficiently high serum levels. Activity of siRNAs was quantified by measuring human ANGPTL3 protein serum using ELISA.

### ANGPTL3 ELISA Assay

Serum ANGPTL3 protein levels in mice treated with siRNAs were quantified by applying R&D Systems' human ANGPTL3 Quantikine ELISA kit (cat. no. DANL30). ANGPTL3 serum levels were calculated relative to the group treated with non-targeting control siRNA.

### Results

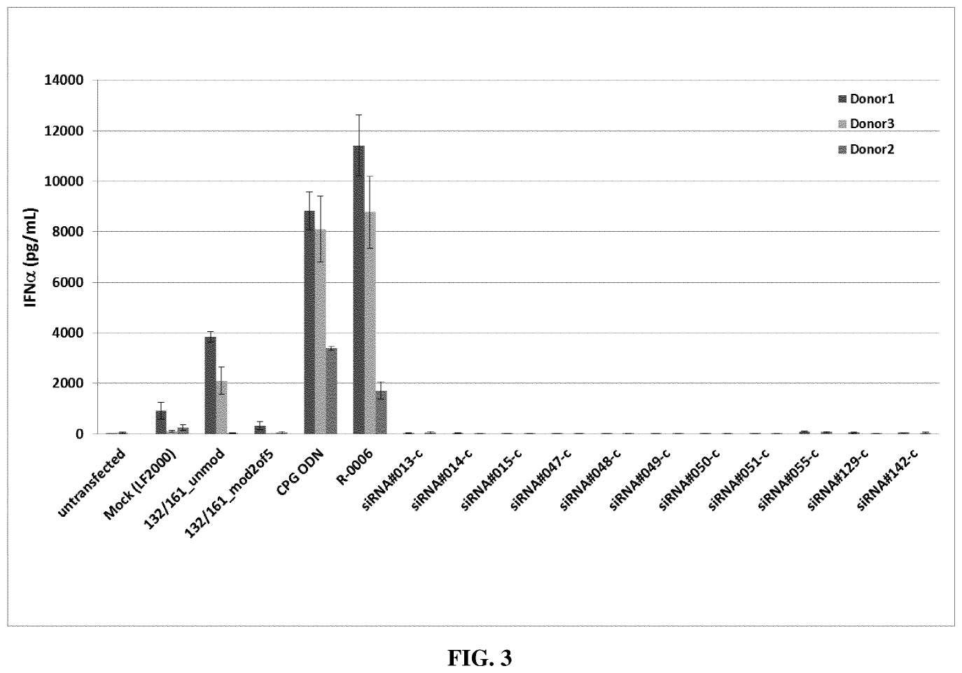

The immune response to 11 GalNAc-siRNAs targeting ANGPTL3 (selected as described above) was measured in vitro in human primary cells by examining the production of interferon α secreted from human primary PMBCs isolated from three different healthy donors (FIG. 3) in response to transfection of the siRNAs. No signs of immune stimulation in human PBMCs were observed for any of the tested siRNAs.

The ANGPTL3 GalNAc-siRNAs were also tested for their in vitro nuclease stability in 50% murine serum by determining their relative stability and half-lives (Table 7). Half-lives ranged between <32 h and 72 h.

TABLE 7
In vitro Serum Stability

| Compound | t_{1/2} | |
|---|---|---|
| siRNA#013-c | 72 | h |
| siRNA#014-c | <32 | h |
| siRNA#015-c | 32 | h |
| siRNA#047-c | <32 | h |
| siRNA#048-c | 32 | h |
| siRNA#049-c | 32 | h |
| siRNA#050-c | 48 | h |
| siRNA#051-c | 72 | h |
| siRNA#055-c | 32 | h |
| siRNA#129-c | 48 | h |
| siRNA#142-c | 48 | h |

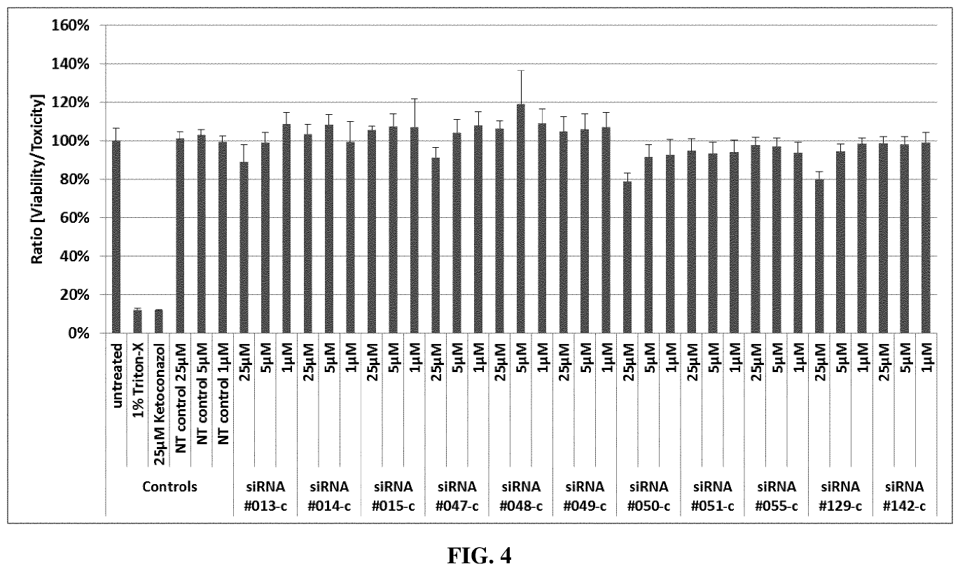

A cytotoxicity assay was carried out in human primary hepatocytes to exclude GalNAc-siRNAs with any toxic potential from further selection (FIG. 4). No obvious toxic effects were observed for any molecules.

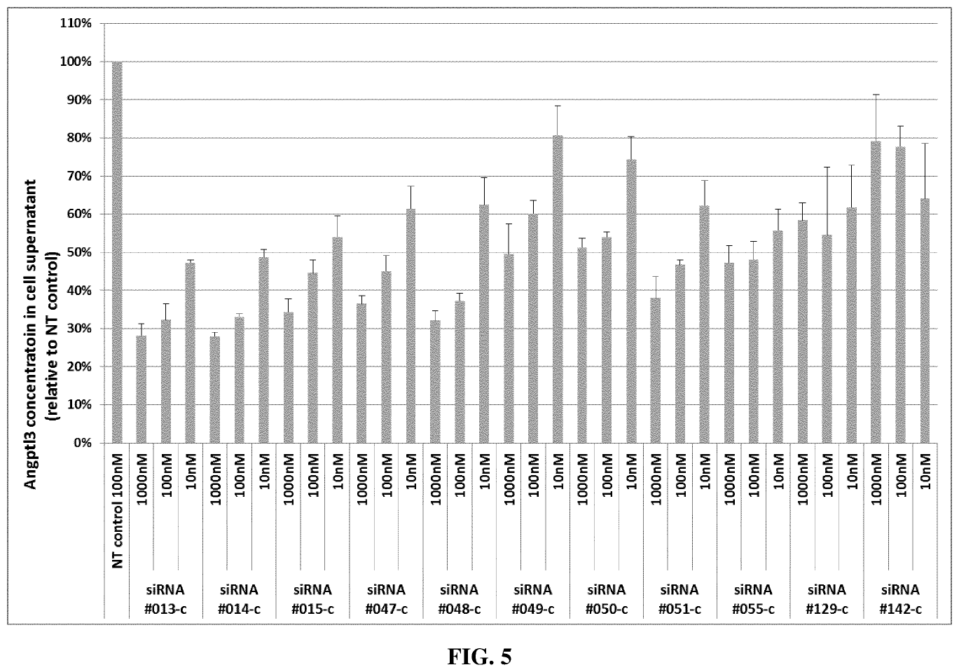

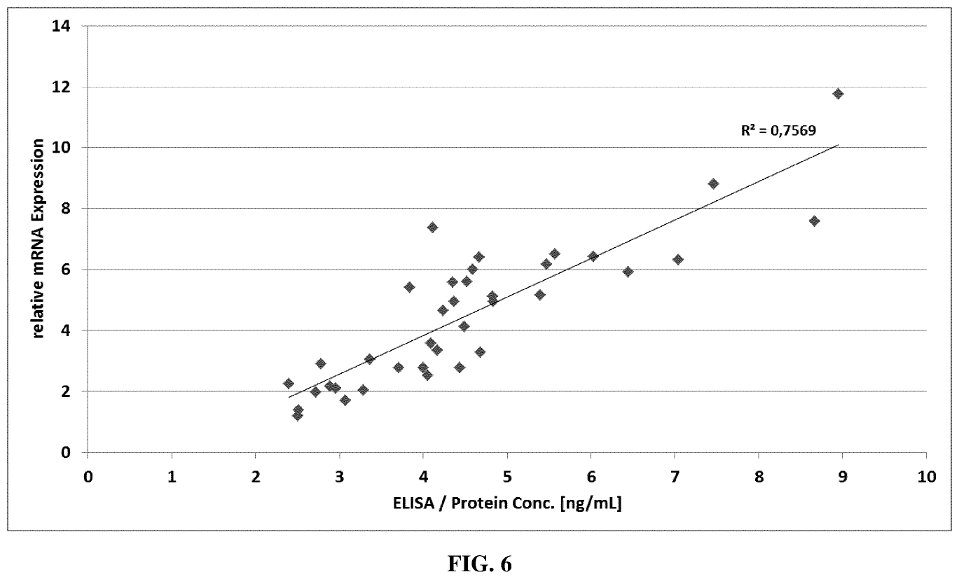

Dose dependent ANGPTL3 protein knockdown was confirmed by quantification of ANGPTL3 levels in the supernatants of human primary hepatocytes treated with three different concentrations (10, 100, and 1000 nM) of the GalNAc-siRNAs (FIG. 5). Target protein reduction showed a good correlation with mRNA knock-down as quantified by qPCR (FIG. 6). This included the two GalNAc-siRNAs that did not demonstrate mRNA knock-down activity observed by qPCR, which translated to the protein level. These data confirm that successful mRNA knock-down obtained with our siRNAs reliably translates to reduction of the corresponding target protein.

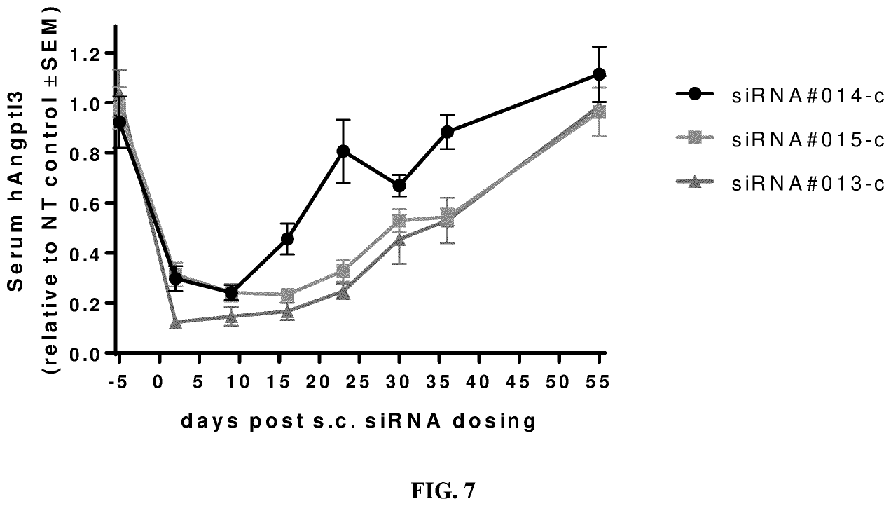

Finally, three selected GalNAc-siRNA molecules were tested in vivo using the above-described humanized mouse model expressing human ANGPTL3 mRNA (FIG. 7). After subcutaneous administration of the selected compounds at 12 mg/kg, target protein levels were reduced between 75% and 90% (KDₘₐₓ) compared to animals treated with a non-targeting control. Depending on the compound, the levels returned to 50% of the maximum knock-down (KD₅₀) between ˜d20 and ˜d35 post treatment. All groups had returned to baseline by day 55.

In summary, the inventors have demonstrated successful identification of siRNAs that strongly reduce expression of human ANGPTL3 mRNA and protein translated from it in the context of GalNAc conjugates in vivo and in vitro.

### Example 5: Identification of Additional siRNAs for Inhibition of Human ANGPTL3 Expression

### Methods

The methods used were the same as those used in Example 2.

### Results

In order to identify additional siRNAs useful in targeting human ANGPTL3, the design and selection criteria as described in Example 2 were adjusted to allow 1 mismatch to M. fascicularis (cynomolgus monkey). Additionally, all siRNA sequences of interest had either greater than three mismatches to any human transcript expressed in liver other than ANGPTL3, or had two mismatches in a maximum of one human gene; sequences that did not meet one of these two criteria were filtered out. This resulted in a list of 49 additional siRNAs (see Table 1, constructs 163-211). In addition, three siRNAs were included in the analyses, which represent extended variants of siRNA #013, siRNA #014 and siRNA #015 (see Table 1, constructs 212-214).

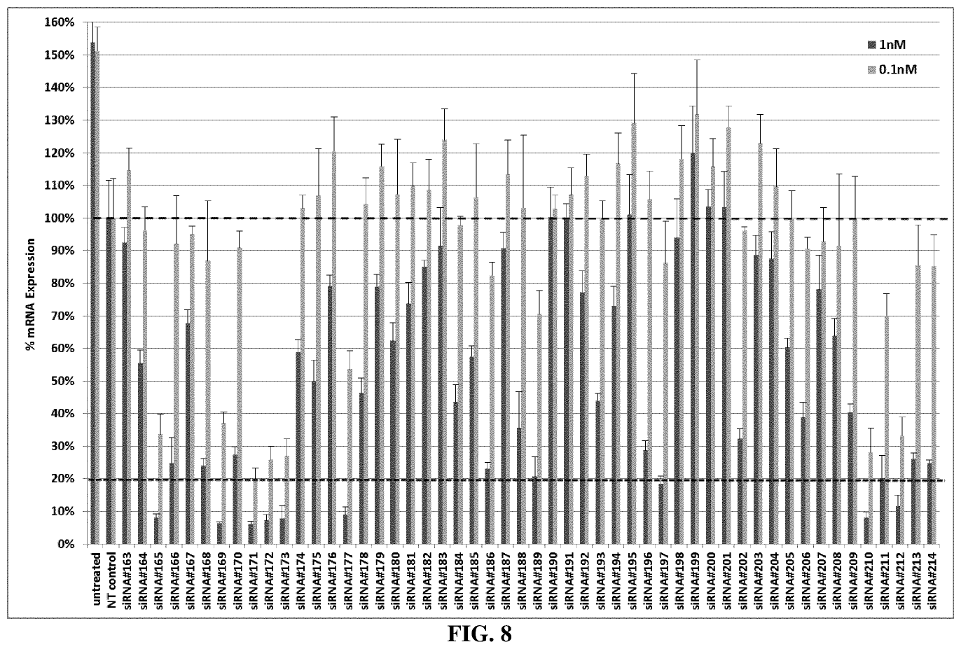

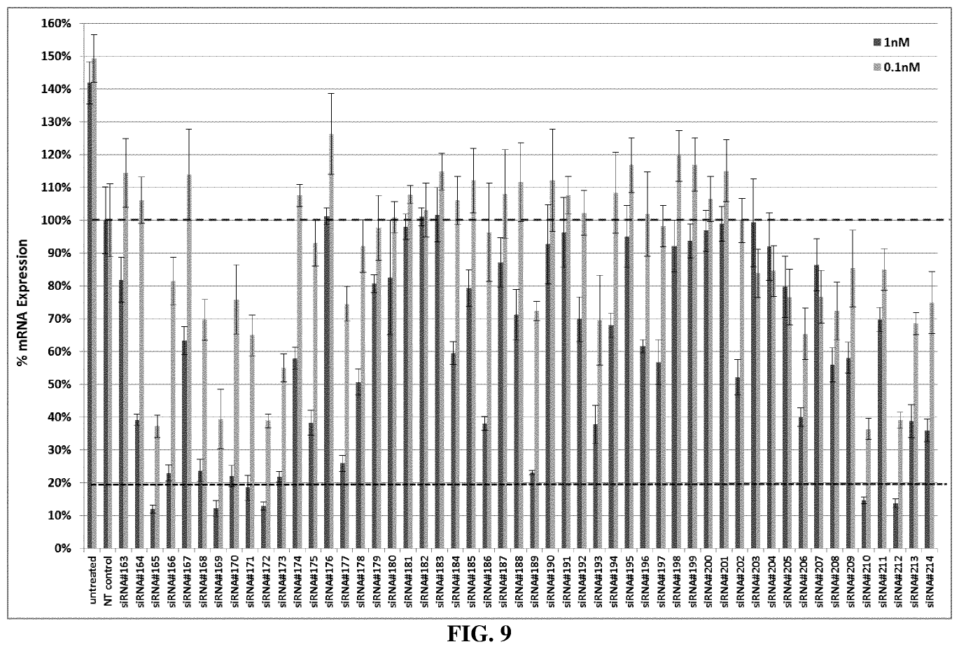

As described above, the 52 siRNAs were produced with nucleotides having a fixed pattern (see Table 2, constructs 163-214). To test the ability of these 52 siRNAs to reduce expression of ANGPTL3, human Hep3B cells and cynomolgus primary hepatocytes were transfected with 0.1 nM or 1.0 nM of each siRNA and incubated for 48 hours. After incubation, mRNA expression of ANGPTL3 was measured in each sample and compared to cells treated with non-targeting control siRNA (FIGS. 8 and 9). 11 siRNAs that showed reduction of mRNA expression at a concentration of 1.0 nM by at least 75% in human Hep3B, or by at least 70% in cynomolgus hepatocytes, were selected for further characterization. Surprisingly, the majority of siRNAs which are active in human cells also work in cynomolgus hepatocytes, despite a single nucleotide mismatch.

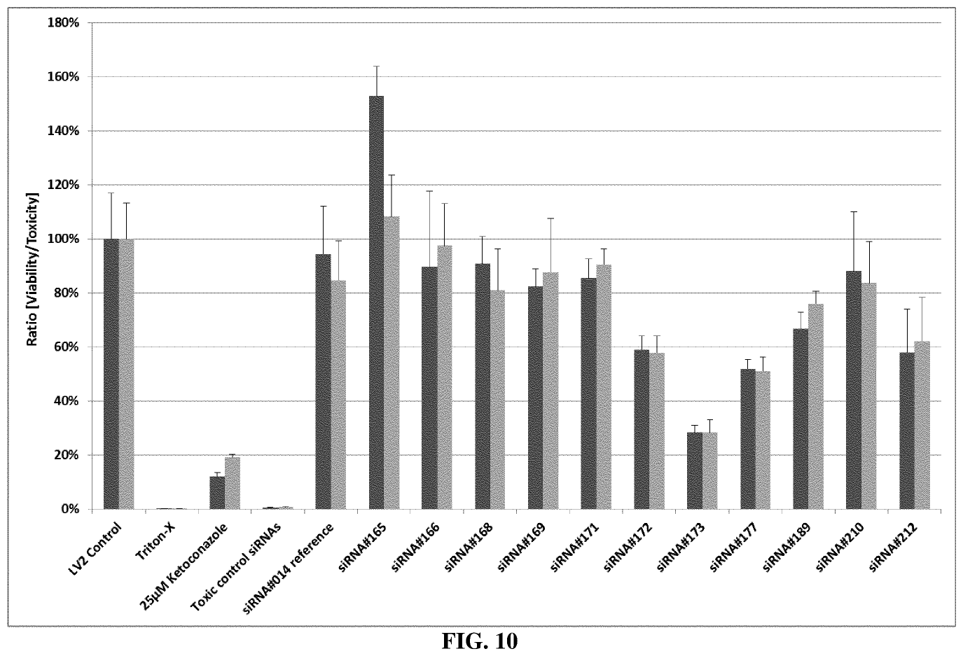

IC₅₀ measurements (Table 8) and a cytotoxicity assay (FIG. 10) were carried out for the selected 11 siRNAs in human Hep3B cells. After removal of one siRNA (siRNA #173) that showed <30% of NT control Viability/Toxicity ratio (at 50 nM), four siRNAs were selected based on their human IC₅₀ values for conjugation to GalNAc (Table 8).

TABLE 8
Activity of additional selected siRNAs

| | | | Selected for |
|---|---|---|---|
| Compound | Iₘₐₓ | IC₅₀ [nM] | GalNAc conjugation |
| siRNA#165 | 0.919 | 4.19E−02 | X |
| siRNA#166 | 0.875 | 5.58E−01 | |
| siRNA#168 | 0.866 | 1.15E−01 | |
| siRNA#169 | 0.949 | 6.47E−02 | |
| siRNA#171 | 0.940 | 5.21E−03 | X |
| siRNA#172 | 0.942 | 3.99E−02 | X |
| siRNA#173 | 0.961 | 6.07E−02 | |
| siRNA#177 | 0.967 | 1.30E−01 | |
| siRNA#189 | 0.902 | 2.67E−01 | |
| siRNA#210 | 0.947 | 6.43E−02 | |
| siRNA#212 | 0.915 | 2.84E−02 | X |

Taken together, these results demonstrate the identification of siRNAs capable of potent inhibition of human and M. fascicularis ANGPTL3 mRNA expression despite a single nucleotide mismatch in M. fascicularis.

### Example 6: Identification of Active GalNAc-Conjugated siRNAs for Inhibition of Human and Cynomolgus ANGPTL3 Expression

### Methods

The methods used were the same as those used in Example 3.

### Results

Following selection of additional potent siRNAs as described in Example 5, the inventors went on to demonstrate whether the selected molecules retain their activity in the context of a GalNAc-conjugate suitable for liver specific siRNA delivery in vivo. They also assessed whether this activity holds up in cells from M. fascicularis (cynomolgus monkey), a critical pre-clinical species.

The results of the IC₅₀ measurements show that all tested siRNA conjugates retain activity when delivered by free uptake to human primary hepatocytes (Table 9; two siRNAs resulting from the first round of screening were included as references), with IC₅₀ values ranging from 1.91 to 9.68 nM. However, surprisingly, the performance ranking following free uptake of GalNAc-siRNA differs from that obtained after transfection assisted uptake of unconjugated siRNA (Table 8). This indicates again that siRNAs seem to have inherent properties based on their sequence that make them differentially suited for application in the context of GalNAc conjugates with regard to resulting knock-down potency.

TABLE 9
Iₘₐₓ and IC₅₀ of additional selected GalNAc-conjugated
siRNAs in human primary hepatocytes

| Compound | Iₘₐₓ | IC₅₀ [nM] |
|---|---|---|
| siRNA#165-c | 0.786 | 3.45 |
| siRNA#171-c | 0.904 | 1.91 |
| siRNA#172-c | 0.620 | 8.15 |
| siRNA#212-c | 0.741 | 9.68 |
| siRNA#013-c | 0.873 | 2.09 |
| siRNA#015-c | 0.846 | 1.97 |

Measured IC₅₀ activity in cynomolgus hepatocytes (Table 10) was less heterogeneous than observed in human hepatocytes (0.406 to 0.987 nM), while Iₘₐₓ was similarly variable (0.605 to 0.892 in cynomolgus vs 0.620 to 0.904 in human) but with different siRNAs showing the best Iₘₐₓ (siRNA #171-c in human, siRNA #013-c in cynomolgus).

TABLE 10
Iₘₐₓ and IC₅₀ of additional selected GalNAc-conjugated
siRNAs in cynomolgus primary hepatocytes

| Compound | Iₘₐₓ | IC₅₀ [nM] |
|---|---|---|
| siRNA#165-c | 0.719 | 9.87E−01 |
| siRNA#171-c | 0.605 | 8.48E−01 |
| siRNA#172-c | N/A | N/A |
| siRNA#212-c | 0.752 | 9.12E−01 |
| siRNA#013-c | 0.892 | 4.06E−01 |
| siRNA#015-c | 0.864 | 8.22E−01 |

N/A: No measurable activity.

These unexpected observations again highlight the requirement to use functional in vitro assays for activity quantification and molecule selection.

### Example 7: In Vitro and In Vivo Characterization of Additional GalNAc-Conjugated siRNAs for Inhibition of Human ANGPTL3 Expression

### Methods

The methods used were the same as those used in Example 4.

### Results

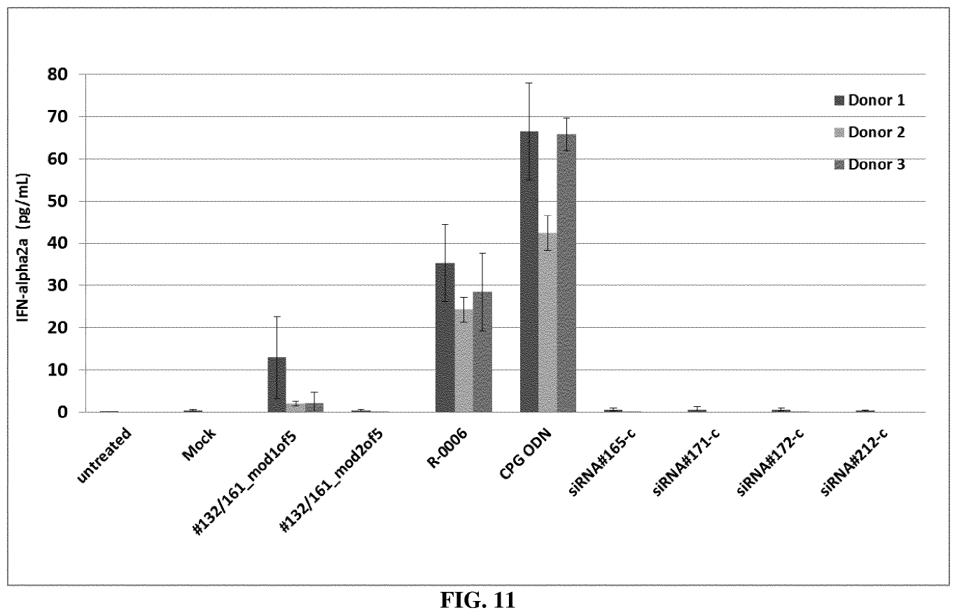

The immune response to four additional GalNAc-siRNAs targeting ANGPTL3 (selected as described in Example 5) was measured in vitro in human cells by examining the production of interferon α2a secreted from human primary PMBCs isolated from three different healthy donors (FIG. 11) in response to transfection of the siRNAs. No signs of immune stimulation in human PBMCs were observed for any of the tested GalNAc-siRNAs.

The additional ANGPTL3 GalNAc-siRNAs were also tested for their in vitro nuclease stability in 50% murine serum by determining their relative stability and half-lives (Table 11). Half-lives ranged between 24 h and 72 h.

TABLE 11
In vitro Serum Stability

| Compound | t_{1/2} |
|---|---|
| siRNA#165-c | 24 h |
| siRNA#171-c | 72 h |
| siRNA#172-c | 48 h |
| siRNA#212-c | 72 h |

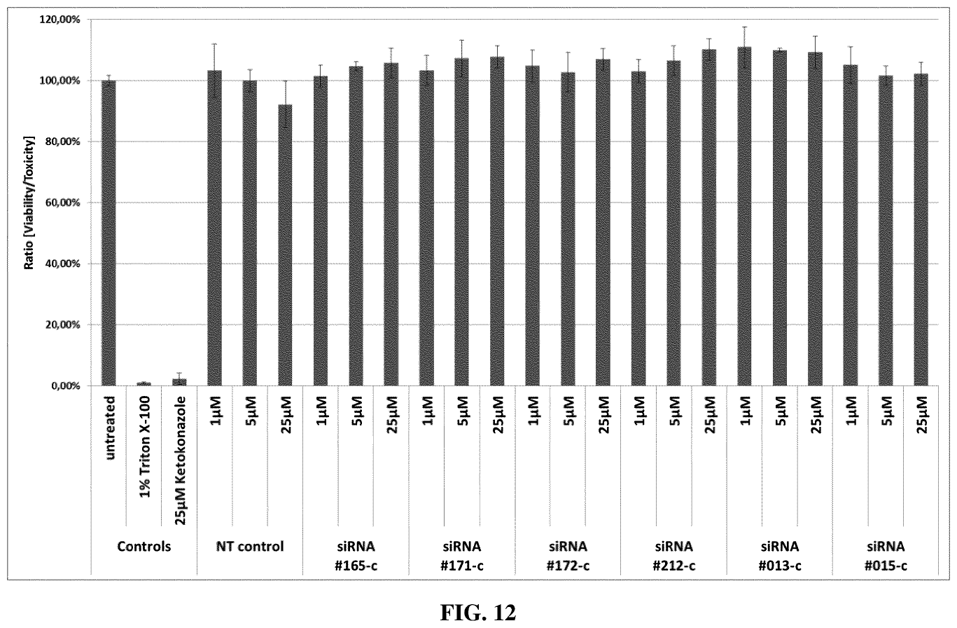

A cytotoxicity assay was carried out in human primary hepatocytes to exclude GalNAc-siRNAs with toxic potential from further selection (FIG. 12). No obvious dose-dependent toxic effects were observed for any molecules. These results demonstrate that application of our selected siRNAs in the context of GalNAc conjugates generally does not confer cytotoxicity.

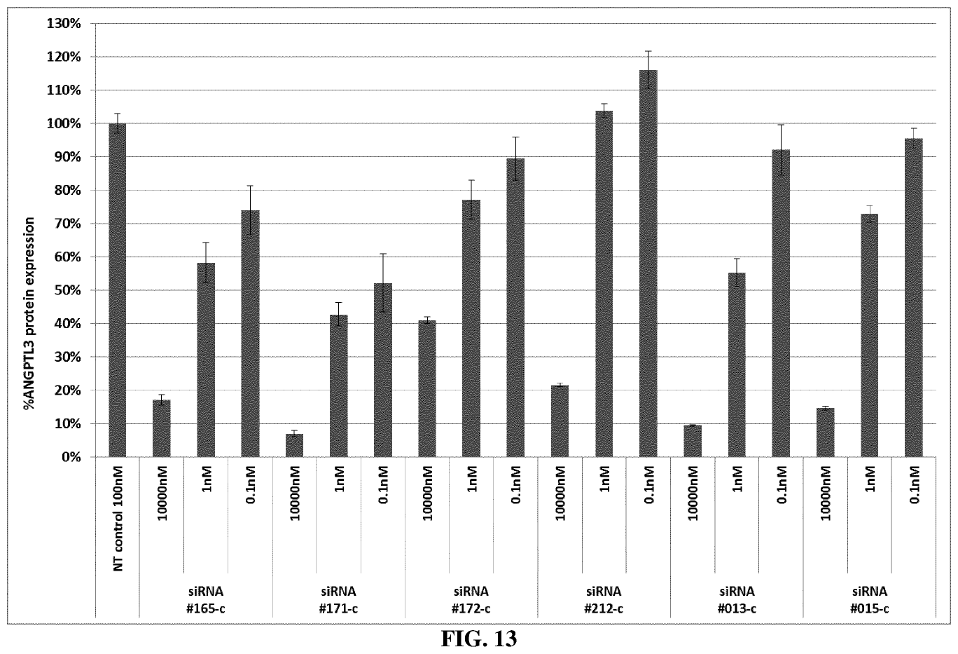

Dose dependent ANGPTL3 protein knockdown was confirmed by quantification of ANGPTL3 levels in the supernatants of human primary hepatocytes treated with three different concentrations (0.1, 1, and 1000 nM) of the GalNAc-siRNAs (FIG. 13). These data confirm that successful mRNA knock-down obtained with our GalNAc-siRNAs reliably translates to reduction of the corresponding target protein.

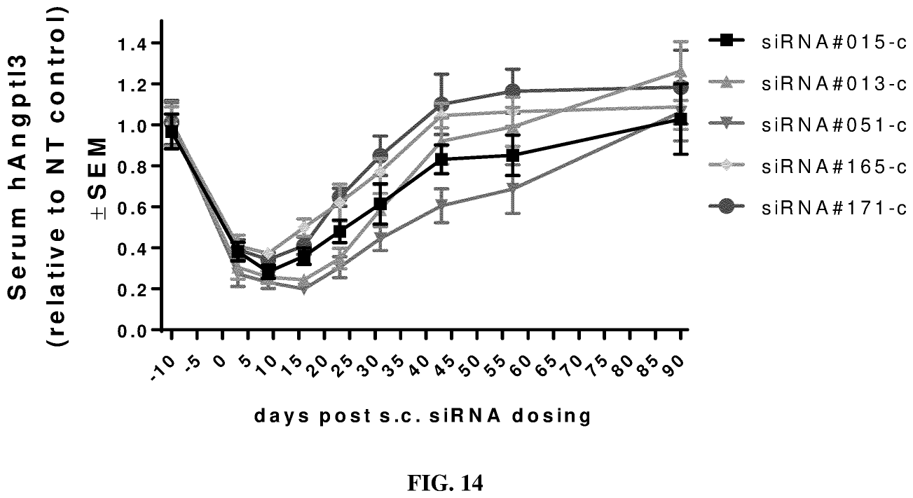

Finally, two additionally selected GalNAc-siRNAs were tested side-by-side with three GalNAc-siRNAs obtained in the first screening campaign (Examples 2-4) in an in vivo mouse model expressing human ANGPTL3 (FIG. 14). After subcutaneous administration of the selected compounds at 10 mg/kg, target protein levels were reduced between 60% and 80% (KDₘₐₓ) compared to animals treated with a non-targeting control. Depending on the compound, the levels returned to 50% of the maximum knock-down (KD₅₀) between ˜d20 and ˜d45 post treatment. All groups had returned to baseline by day 90.

In summary, the inventors have demonstrated the successful identification of additional siRNAs that strongly reduce expression of human ANGPTL3 mRNA and protein translated from it in the context of GalNAc conjugates in vivo and in vitro.

### Example 8: Optimization of GalNAc-Conjugated ANGPTL3 siRNA Sequences

### Methods

Production of Modified GalNAc siRNA Sequences

GalNAc siRNA sequences further optimized with modified nucleotides of formula (I) were synthesized as described in PCT Patent Publication WO 2019/170731. All oligonucleotides were synthesized on an ABI 394 synthesizer. Commercially available (Sigma Aldrich) DNA-, RNA-, 2′-OMe-RNA, and 2′-deoxy-F-RNA-phosphoramidites with standard protecting groups, e.g., 5′-O-dimethoxytrityl-thymidine-3′-O-(N,N-diisopropyl-2-cyanoethyl-phosphoramidite, 5′-O-dimethoxytrityl-2′-O-tert-butyldimethylsilyl-uracile-3′-O-(N,N-diisopropyl-2-cyanoethyl)-phosphoramidite, 5′-O-dimethoxytrityl-2′-O-tert-butyldimethylsilyl-N4-cytidine-3′-O-(N,N-diisopropyl-2-cyanoethyl)-phosphoramidite, 5′-O-dimethoxytrityl-2′-O-tert-butyldimethylsilyl-N6-benzoyl-adenosine-3′-O-(N,N-diisopropyl-2-cyanoethyl)-phosphoramidite,5′-O-dimethoxytrityl-2′-O-tert-butyldimethylsilyl-N2-isobutyryl-guanosine-3′-O-(N,N-diisopropyl-2-cyanoethyl)-phosphoramidite,5′-O-dimethoxytrityl-2′-O-methyl-uracile-3′-O-(N,N-diisopropyl-2-cyanoethyl)-phosphoramidite, 5′-O-dimethoxytrityl-2′-O-methyl-N4-cytidine-3′-O-(N,N-diisopropyl-2-cyanoethyl)-phosphoramidite, 5′-O-dimethoxytrityl-2′-O-methyl-N6-benzoyl-adenosine-3′-O-(N,N-diisopropyl-2-cyanoethyl)-phosphoramidite,5′-O-dimethoxytrityl-2′-O-methyl-N2-isobutyryl-guanosine-3′-O-(N,N-diisopropyl-2-cyanoethyl)-phosphoramidite,5′-O-dimethoxytrityl-2′-desoxy-fluoro-uracile-3′-O-(N,N-diisopropyl-2-cyanoethyl)-phosphoramidite, 5′-O-dimethoxytrityl-2′-deoxy-fluoro-N4-cytidine-3′-O-(N,N-diisopropyl-2-cyanoethyl)-phosphoramidite, 5′-O-dimethoxytrityl-2′-deoxy-fluoro-N6-benzoyl-adenosine-3′-O-(N,N-diisopropyl-2-cyanoethyl)-phosphoramidite, and 5′-O-dimethoxytrityl-2′-deoxy-fluoro-N2-isobutyryl-guanosine-3′-O-(N,N-diisopropyl-2-cyanoethyl)-phosphoramidite as well as the corresponding solid support materials (CPG-500 Å, loading 40 μmol/g, ChemGenes) were used for automated oligonucleotide synthesis.

Phosphoramidite building blocks were used as 0.1 M solutions in acetonitrile and activated with 5-(bis-3,5-trifluoromethylphenyl)-1H-tetrazole (activator 42, 0.25 M in acetonitrile, Sigma Aldrich). Reaction times of 200 s were used for standard phosphoramidite couplings. In case of phosphoramidites described herein, coupling times of 300 s were applied. As capping reagents, acetic anhydride in THF (capA for ABI, Sigma Aldrich) and N-methylimidazole in THF (capB for ABI, Sigma Aldrich) were used. As oxidizing reagent, iodine in THF/pyridine/water (0.02 M; oxidizer for ABI, Sigma Aldrich) was used. Alternatively, PS-oxidation was achieved with a 0.05 M solution of 3-((N,N-dimethyl-aminomethylidene)amino)-3H-1,2,4-dithiazole-5-thione (DDTT) in pyridine/acetonitrile (1:1). Deprotection of the DMT-protecting group was done using dichloroacetic acid in DCM (DCA deblock, Sigma Aldrich). Final cleavage from solid support and deprotection (acyl- and cyanoethyl-protecting groups) was achieved with NH₃ (32% aqueous solution/ethanol, v/v 3:1). Treatment with NMP/NEt₃/HF (3:1.5:2) was applied for TBDMS-deprotection.

Oligonucleotides with herein described building blocks at the 3′-end were synthesized on solid support materials or on universal linker-solid support (CPG-500 Å, loading 39 μmol/g, AM Chemicals LLC) and the corresponding phosphoramidites shown in Table A.

Crude products were analyzed by HPLC and single strand purification was performed using ion exchange or preparative HPLC-methods.
Ion exchange: ÄKTA purifier, (Thermo Fisher Scientific DNAPac PA200 semi prep ion exchange column, 8 μm particles, width 22 mm×length 250 mm).
Buffer A: 1.5 L H₂O, 2.107 g NaClO₄, 438 mg EDTA, 1.818 g TRIS, 540.54 g urea, pH 7.4.
Buffer B: 1.5 L H₂O, 105.34 g NaClO₄, 438 mg EDTA, 1.818 g TRIS, 40.54 g urea, pH 7.4.
Isolation of the oligonucleotides was achieved by precipitation induced by the addition of 4 volumes of ethanol and storing at −20° C.

Preparative HPLC: Agilent 1100 series prep HPLC (Waters XBridge®BEH C18 OBD™ Prep Column 130 Å, 5 μm, 10 mm×100 mm); Eluent: Triethylammonium acetate (0.1 M in acetonitrile/water). After lyophilization, the products were dissolved in 1.0 mL 2.5 M NaCl solution and 4.0 mL H₂O. The corresponding Na⁺-salts were isolated after precipitation by adding 20 mL ethanol and storing at −20° C. for 18 h.

Final analysis of the single strands was done by LC/MS-TOF methods. For double strand formation, equimolar amounts of sense strands and antisense strands were mixed in 1×PBS buffer and heated to 85° C. for 10 min. Then it was slowly cooled down to room temperature. Final analysis of the siRNA-double strands was done by LC/MS-TOF methods.

Annealing of siRNA duplexes was performed as described in Example 1. The sequences of each siRNA, including nucleotide modifications, are shown in Table 3.

siRNA Stability in Mouse Serum

Stability of optimized ANGPTL3 siRNAs listed in Table 3 was determined as described in Example 1 with the following exceptions: siRNAs were incubated at 37° C. for 0 h, 24 h, 48 h, 72 h, 96 h, and 168 h. Proteinase K was purchased from Qiagen (cat. no. 19133) and HPLC analysis was done on an Agilent Technologies 1260 Infinity II instrument using a 1260 DAD detector.

### Cell Culture and Cell-Based Assays

Human Hep3B cells, primary human hepatocytes, and primary human PBMCs were isolated and cultivated as described in Examples 2-7. Analysis of mRNA was performed as described in Example 2. Cytotoxicity was measured 72 hours after 5 nM and 50 nM siRNA transfections of human Hep3B cells as described in Example 2. IFNα protein concentration was quantified in the supernatant of human PBMCs as described in Example 4.

### In Vivo Assay

In vivo activity of modified GalNAc-ANGPTL3 siRNAs was measured in mice transduced with an AAV8 vector encoding for human ANGPTL3 mRNA from an ApoA2 promoter as described in Example 4. In contrast with Example 4, a single siRNA dose of 5 mg/kg was injected subcutaneously into 5 male C57BL/6 mice per treatment group.

### ANGPTL3 ELISA Assay

Serum ANGPTL3 protein levels in mice treated with modified GalNAc-siRNAs were quantified as described in Example 4.

### Results

54 different siRNA modification patterns were designed and applied to three pre-selected siRNA sequences (siRNA #013, siRNA #051, and siRNA #165). Libraries of 3×54 siRNA molecules (siRNA #013-c-01 to siRNA #013-c-54, siRNA #051-c-01 to siRNA #051-c-54, and siRNA #165-c-01 to siRNA #165-c-54, Table 3) were synthesized using three consecutive modified GalNAc conjugated nucleotides at the 5′-end of respective siRNA sense strands.

All 162 modified ANGPTL3 siRNAs were tested for their nuclease stability in 50% mouse serum. As depicted in Table 12, several molecules were identified with significantly improved stability as compared to respective parent sequences with a fixed pattern of 2′O-methyl and 2′-fluoro modified nucleotides. For the constructs derived from siRNA #013-c and siRNA #051-c, the serum half-lives improved from approximately 72 h for the parental construct pattern to 168 h or more for the modified constructs. For the constructs derived from siRNA #165-c, serum half-lives improved from approximately 24 h to 96 h or more.

TABLE 12
In vitro Serum Stability

| siRNA Construct | t_{1/2} | | siRNA Construct | t1/2 | | siRNA Construct | t_{1/2} | |
|---|---|---|---|---|---|---|---|---|
| siRNA#013-c | =72 | h | siRNA#051-c | =72 | h | siRNA#165-c | =24 | h |
| siRNA#013-c-01 | >96 | h | siRNA#051-c-01 | >168 | h | siRNA#165-c-01 | >96 | h |
| siRNA#013-c-02 | >96 | h | siRNA#051-c-02 | >168 | h | siRNA#165-c-02 | >96 | h |
| siRNA#013-c-03 | >96 | h | siRNA#051-c-03 | >168 | h | siRNA#165-c-03 | >96 | h |
| siRNA#013-c-04 | >72 | h | siRNA#051-c-04 | >96 | h | siRNA#165-c-04 | >48 | h |
| siRNA#013-c-05 | >96 | h | siRNA#051-c-05 | >168 | h | siRNA#165-c-05 | >96 | h |
| siRNA#013-c-06 | >96 | h | siRNA#051-c-06 | >168 | h | siRNA#165-c-06 | >96 | h |
| siRNA#013-c-07 | >96 | h | siRNA#051-c-07 | >168 | h | siRNA#165-c-07 | >96 | h |
| siRNA#013-c-08 | >48 | h | siRNA#051-c-08 | >96 | h | siRNA#165-c-08 | >72 | h |
| siRNA#013-c-09 | >96 | h | siRNA#051-c-09 | >168 | h | siRNA#165-c-09 | =168 | h |
| siRNA#013-c-10 | >96 | h | siRNA#051-c-10 | >168 | h | siRNA#165-c-10 | =96 | h |
| siRNA#013-c-11 | >96 | h | siRNA#051-c-11 | >168 | h | siRNA#165-c-11 | >96 | h |
| siRNA#013-c-12 | >96 | h | siRNA#051-c-12 | >168 | h | siRNA#165-c-12 | >96 | h |
| siRNA#013-c-13 | >96 | h | siRNA#051-c-13 | >96 | h | siRNA#165-c-13 | =48 | h |
| siRNA#013-c-14 | >96 | h | siRNA#051-c-14 | >168 | h | siRNA#165-c-14 | >96 | h |
| siRNA#013-c-15 | >72 | h | siRNA#051-c-15 | >168 | h | siRNA#165-c-15 | >96 | h |
| siRNA#013-c-16 | >96 | h | siRNA#051-c-16 | >168 | h | siRNA#165-c-16 | >96 | h |
| siRNA#013-c-17 | =168 | h | siRNA#051-c-17 | >168 | h | siRNA#165-c-17 | =168 | h |
| siRNA#013-c-18 | >96 | h | siRNA#051-c-18 | >96 | h | siRNA#165-c-18 | >72 | h |
| siRNA#013-c-19 | >96 | h | siRNA#051-c-19 | >96 | h | siRNA#165-c-19 | >96 | h |
| siRNA#013-c-20 | >96 | h | siRNA#051-c-20 | >96 | h | siRNA#165-c-20 | >96 | h |
| siRNA#013-c-21 | =168 | h | siRNA#051-c-21 | =168 | h | siRNA#165-c-21 | >96 | h |
| siRNA#013-c-22 | =96 | h | siRNA#051-c-22 | >96 | h | siRNA#165-c-22 | =48 | h |
| siRNA#013-c-23 | >168 | h | siRNA#051-c-23 | =168 | h | siRNA#165-c-23 | =168 | h |
| siRNA#013-c-24 | >96 | h | siRNA#051-c-24 | >168 | h | siRNA#165-c-24 | >96 | h |
| siRNA#013-c-25 | >96 | h | siRNA#051-c-25 | =168 | h | siRNA#165-c-25 | >96 | h |
| siRNA#013-c-26 | =96 | h | siRNA#051-c-26 | >96 | h | siRNA#165-c-26 | =96 | h |
| siRNA#013-c-27 | >96 | h | siRNA#051-c-27 | =168 | h | siRNA#165-c-27 | >96 | h |
| siRNA#013-c-28 | >96 | h | siRNA#051-c-28 | >96 | h | siRNA#165-c-28 | >96 | h |
| siRNA#013-c-29 | >96 | h | siRNA#051-c-29 | =168 | h | siRNA#165-c-29 | >96 | h |
| siRNA#013-c-30 | >96 | h | siRNA#051-c-30 | =168 | h | siRNA#165-c-30 | >96 | h |
| siRNA#013-c-31 | =96 | h | siRNA#051-c-31 | >96 | h | siRNA#165-c-31 | >48 | h |
| siRNA#013-c-32 | >96 | h | siRNA#051-c-32 | >168 | h | siRNA#165-c-32 | >96 | h |
| siRNA#013-c-33 | >96 | h | siRNA#051-c-33 | =168 | h | siRNA#165-c-33 | =96 | h |
| siRNA#013-c-34 | =168 | h | siRNA#051-c-34 | >168 | h | siRNA#165-c-34 | >96 | h |
| siRNA#013-c-35 | =168 | h | siRNA#051-c-35 | >168 | h | siRNA#165-c-35 | >96 | h |
| siRNA#013-c-36 | =96 | h | siRNA#051-c-36 | =96 | h | siRNA#165-c-36 | =96 | h |
| siRNA#013-c-37 | >168 | h | siRNA#051-c-37 | =168 | h | siRNA#165-c-37 | >96 | h |
| siRNA#013-c-38 | >96 | h | siRNA#051-c-38 | >168 | h | siRNA#165-c-38 | >96 | h |
| siRNA#013-c-39 | =96 | h | siRNA#051-c-39 | >168 | h | siRNA#165-c-39 | >96 | h |
| siRNA#013-c-40 | =72 | h | siRNA#051-c-40 | >96 | h | siRNA#165-c-40 | =48 | h |
| siRNA#013-c-41 | >96 | h | siRNA#051-c-41 | =168 | h | siRNA#165-c-41 | >96 | h |
| siRNA#013-c-42 | =168 | h | siRNA#051-c-42 | =168 | h | siRNA#165-c-42 | >96 | h |
| siRNA#013-c-43 | >96 | h | siRNA#051-c-43 | >96 | h | siRNA#165-c-43 | >96 | h |
| siRNA#013-c-44 | =96 | h | siRNA#051-c-44 | >96 | h | siRNA#165-c-44 | =72 | h |
| siRNA#013-c-45 | >96 | h | siRNA#051-c-45 | >168 | h | siRNA#165-c-45 | >96 | h |
| siRNA#013-c-46 | >168 | h | siRNA#051-c-46 | >96 | h | siRNA#165-c-46 | >96 | h |
| siRNA#013-c-47 | >96 | h | siRNA#051-c-47 | =168 | h | siRNA#165-c-47 | >96 | h |
| siRNA#013-c-48 | >168 | h | siRNA#051-c-48 | >168 | h | siRNA#165-c-48 | >96 | h |
| siRNA#013-c-49 | =72 | h | siRNA#051-c-49 | >96 | h | siRNA#165-c-49 | >48 | h |
| siRNA#013-c-50 | =168 | h | siRNA#051-c-50 | >168 | h | siRNA#165-c-50 | >96 | h |
| siRNA#013-c-51 | >168 | h | siRNA#051-c-51 | >168 | h | siRNA#165-c-51 | >96 | h |
| siRNA#013-c-52 | >168 | h | siRNA#051-c-52 | >168 | h | siRNA#165-c-52 | >96 | h |
| siRNA#013-c-53 | >168 | h | siRNA#051-c-53 | >168 | h | siRNA#165-c-53 | >96 | h |
| siRNA#013-c-54 | >168 | h | siRNA#051-c-54 | >96 | h | siRNA#165-c-54 | >96 | h |

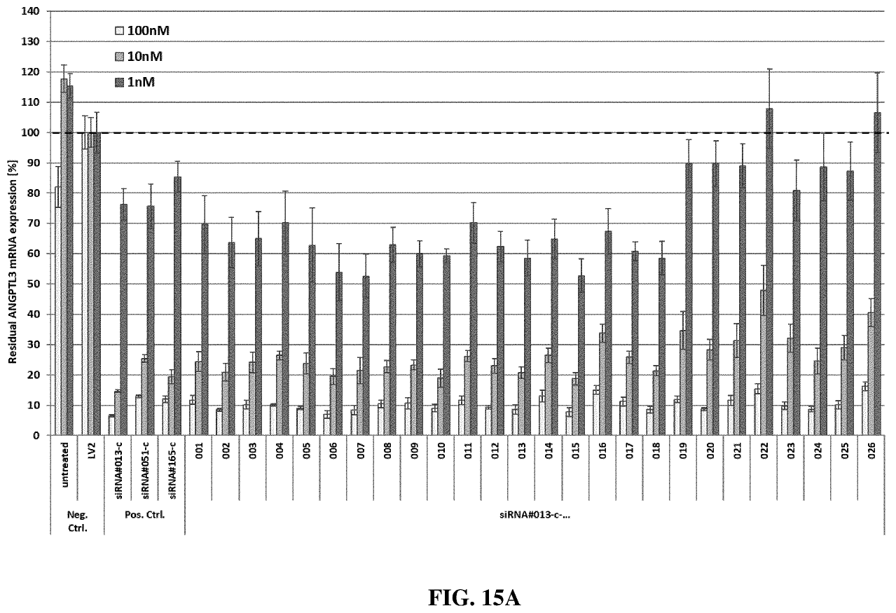

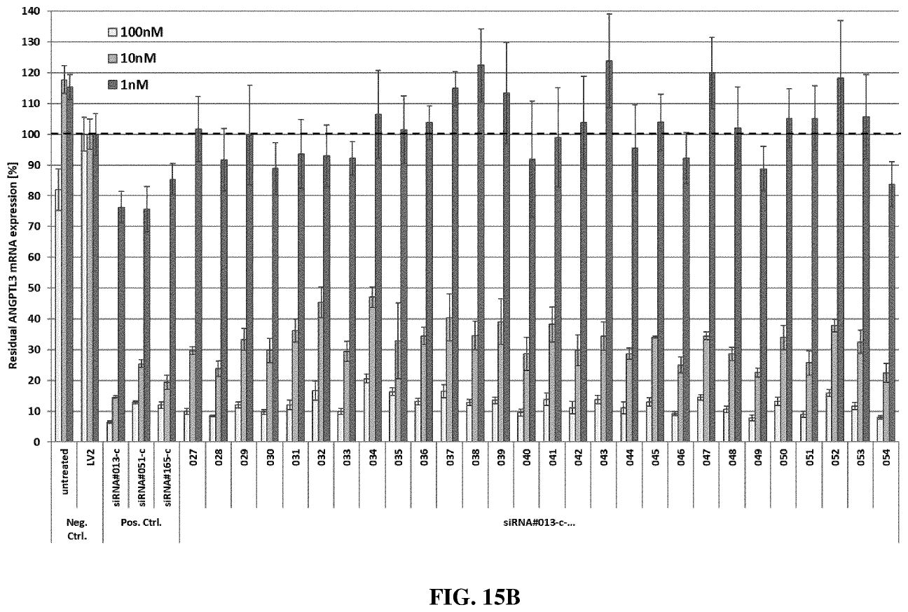

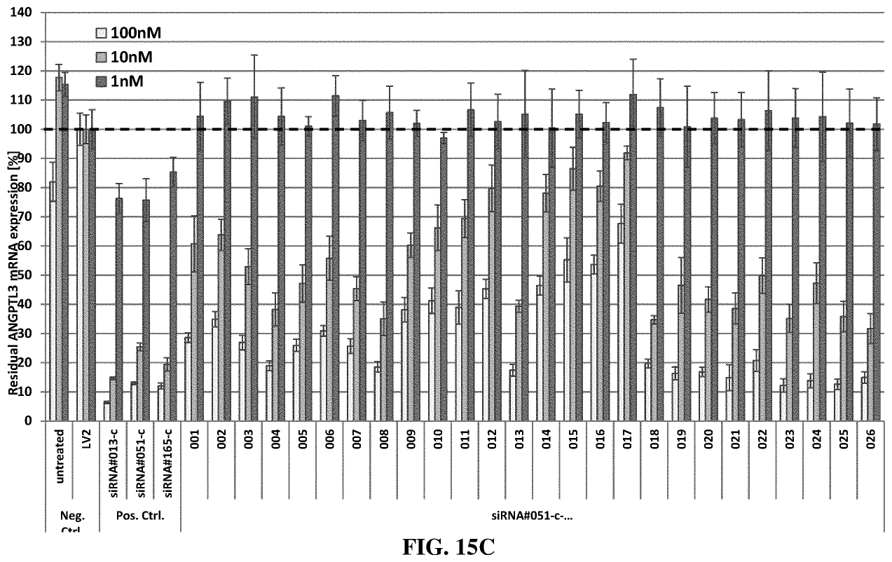

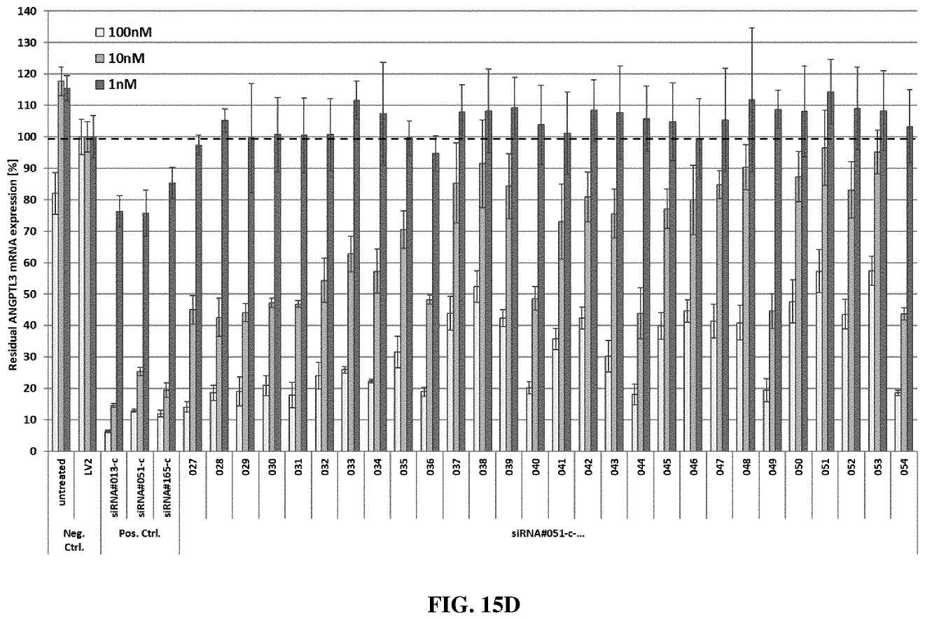

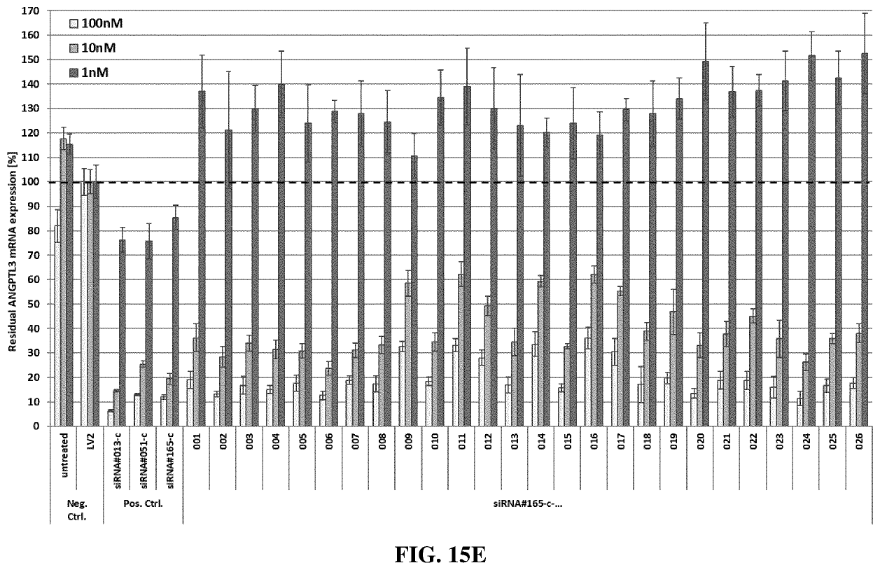

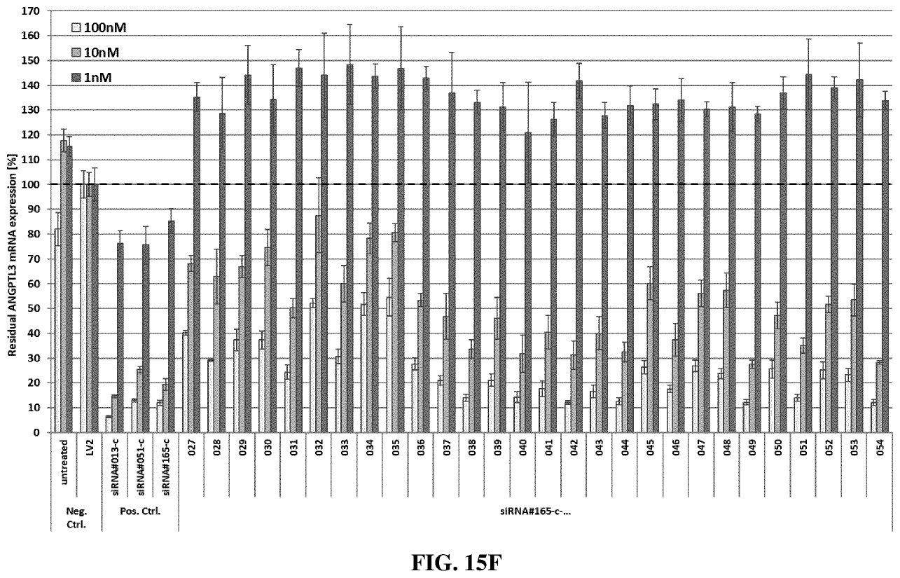

Next, all of the 162 modified GalNAc-siRNAs were evaluated for their knock-down potency in primary human hepatocytes under free uptake conditions and using 1 nM, 10 nM and 100 nM concentrations of the modified siRNAs. The parent constructs siRNA #013-c, siRNA #051-c, and siRNA #165-c were used as positive controls. Data are shown in FIGS. 15A-F.

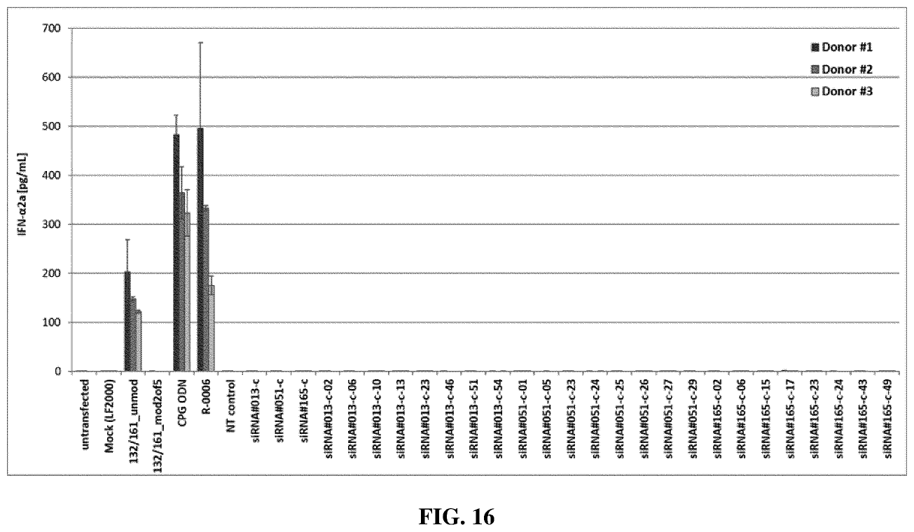

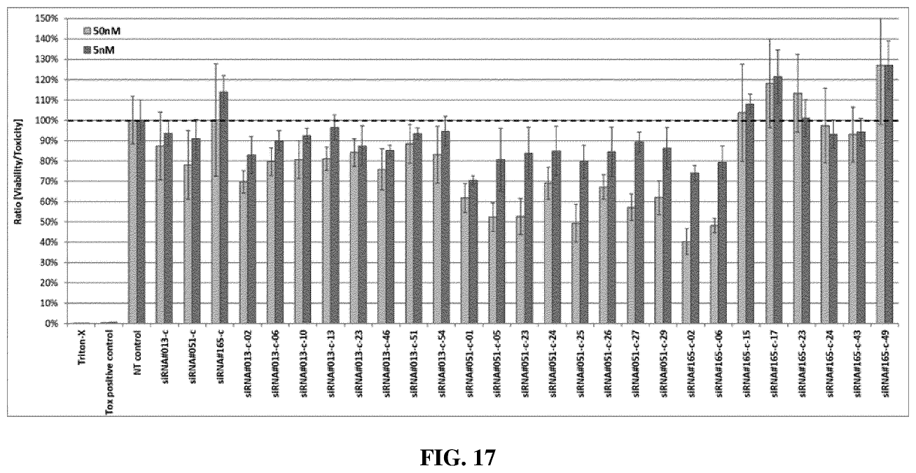

Based on the in vitro knock-down activities and nuclease stability data, eight modified variants were selected for each of the three parent constructs. Prior to in vivo activity testing, the 3×8 modified constructs were investigated for their ability to stimulate innate immunity in human PBMCs (FIG. 16) and for their general cytotoxicity in human Hep3B cells (FIG. 17). In both assays, no apparent adverse effects were observed.

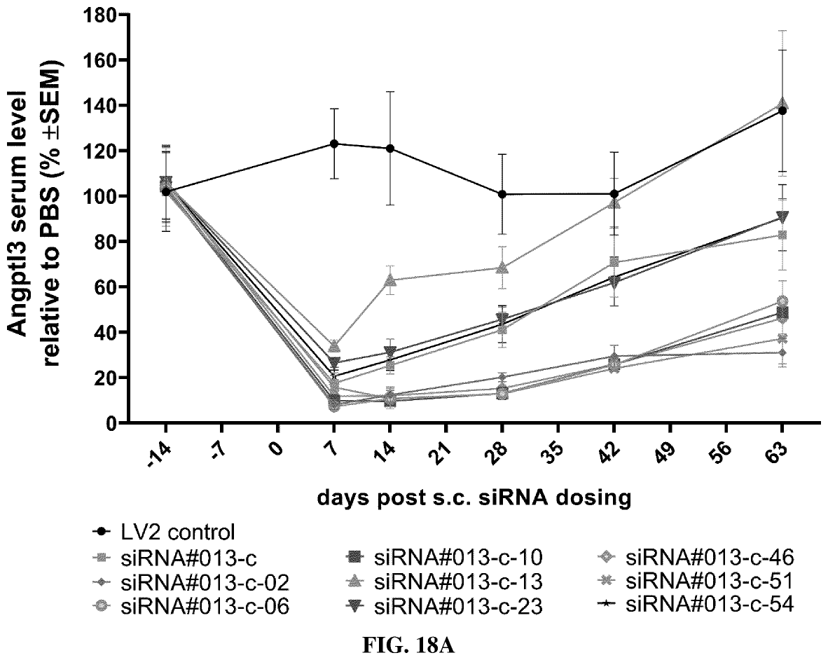

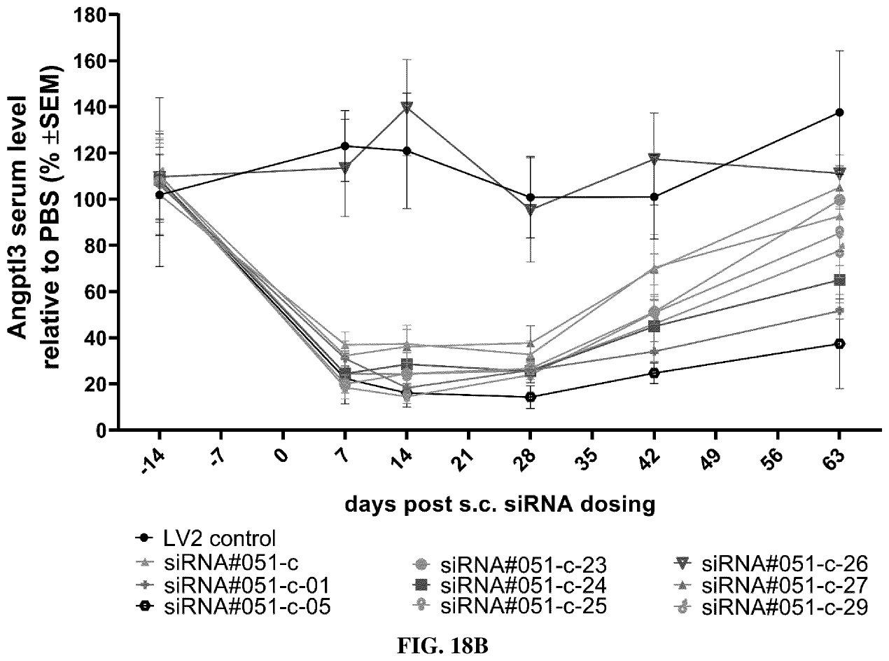

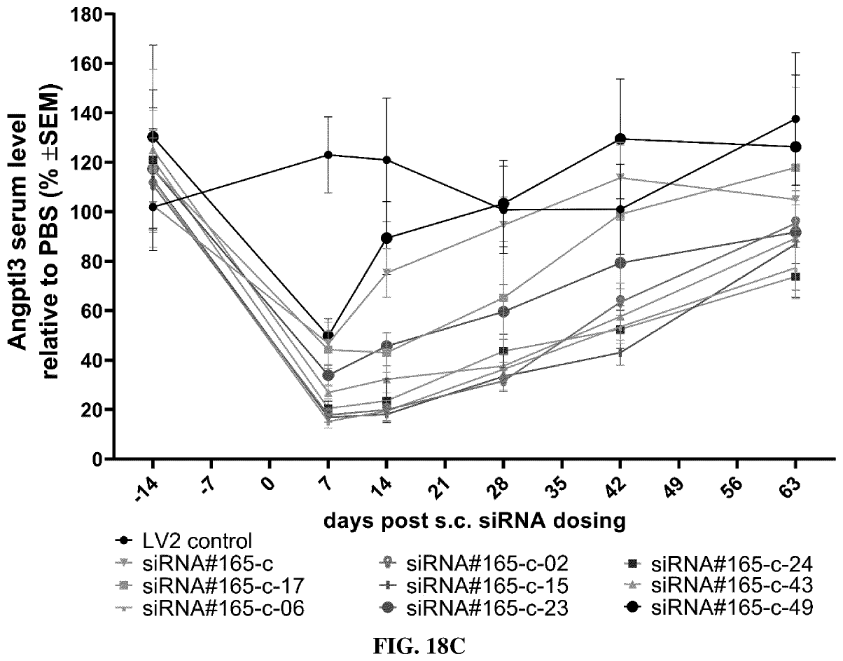

Finally, the 3×8 selected modified GalNAc-siRNA constructs were tested in vivo using the above-described humanized mouse model expressing human ANGPTL3 mRNA (FIG. 18 A-C). After single subcutaneous administrations of the selected compounds at a dose of 5 mg/kg, target protein levels were reduced by up to ˜95% (KDₘₐₓ) compared to animals treated with PBS. The most long-lasting optimized molecules did not yet return to 50% % of the maximum knock-down (KD₅₀) at day 63, whereas all three parent constructs exhibited <15% residual activity at that point.

In summary, the inventors have demonstrated successful identification of siRNAs that strongly reduce expression of human ANGPTL3 mRNA and protein translated from it in the context of GalNAc conjugates in vivo and in vitro. They have also demonstrated unexpectedly strong improvement of in vivo efficacy of siRNAs by introduction of optimized modification patterns using modified nucleotides. Despite a loose correlation between stability and in vitro performance, the in vivo potency of certain modified siRNAs could not be systematically predicted based on non-in vivo data.

### ANGPTL3 Sequences

| human ANGPTL3 mRNA sequence | |
|---|---|
| (SEQ ID NO: 1181) | |
| 1 | atatatagag ttaagaagtc taggtctgct tccagaagaa aacagttcca cgttgcttga |
| 61 | aattgaaaat caagataaaa atgttcacaa ttaagctcct tctttttatt gttcctctag |
| 121 | ttatttcctc cagaattgat caagacaatt catcatttga ttctctatct ccagagccaa |
| 181 | aatcaagatt tgctatgtta gacgatgtaa aaattttagc caatggcctc cttcagttgg |
| 241 | gacatggtct taaagacttt gtccataaga cgaagggcca aattaatgac atatttcaaa |
| 301 | aactcaacat atttgatcag tctttttatg atctatcgct gcaaaccagt gaaatcaaag |
| 361 | aagaagaaaa ggaactgaga agaactacat ataaactaca agtcaaaaat gaagaggtaa |
| 421 | agaatatgtc acttgaactc aactcaaaac ttgaaagcct cctagaagaa aaaattctac |
| 481 | ttcaacaaaa agtgaaatat ttagaagagc aactaactaa cttaattcaa aatcaacctg |
| 541 | aaactccaga acacccagaa gtaacttcac ttaaaacttt tgtagaaaaa caagataata |
| 601 | gcatcaaaga ccttctccag accgtggaag accaatataa acaattaaac caacagcata |
| 661 | gtcaaataaa agaaatagaa aatcagctca gaaggactag tattcaagaa cccacagaaa |
| 721 | tttctctatc ttccaagcca agagcaccaa gaactactcc ctttcttcag ttgaatgaaa |
| 781 | taagaaatgt aaaacatgat ggcattcctg ctgaatgtac caccatttat aacagaggtg |
| 841 | aacatacaag tggcatgtat gccatcagac ccagcaactc tcaagttttt catgtctact |
| 901 | gtgatgttat atcaggtagt ccatggacat taattcaaca tcgaatagat ggatcacaaa |
| 961 | acttcaatga aacgtgggag aactacaaat atggttttgg gaggcttgat ggagaatttt |
| 1021 | ggttgggcct agagaagata tactccatag tgaagcaatc taattatgtt ttacgaattg |
| 1081 | agttggaaga ctggaaagac aacaaacatt atattgaata ttctttttac ttgggaaatc |
| 1141 | acgaaaccaa ctatacgcta catctagttg cgattactgg caatgtcccc aatgcaatcc |
| 1201 | cggaaaacaa agatttggtg ttttctactt gggatcacaa agcaaaagga cacttcaact |
| 1261 | gtccagaggg ttattcagga ggctggtggt ggcatgatga gtgtggagaa aacaacctaa |
| 1321 | atggtaaata taacaaacca agagcaaaat ctaagccaga gaggagaaga ggattatctt |
| 1381 | ggaagtctca aaatggaagg ttatactcta taaaatcaac caaaatgttg atccatccaa |
| 1441 | cagattcaga aagctttgaa tgaactgagg caaatttaaa aggcaataat ttaaacatta |
| 1501 | acctcattcc aagttaatgt ggtctaataa tctggtatta aatccttaag agaaagcttg |
| 1561 | agaaatagat tttttttatc ttaaagtcac tgtctattta agattaaaca tacaatcaca |
| 1621 | taaccttaaa gaataccgtt tacatttctc aatcaaaatt cttataatac tatttgtttt |
| 1681 | aaattttgtg atgtgggaat caattttaga tggtcacaat ctagattata atcaataggt |
| 1741 | gaacttatta aataactttt ctaaataaaa aatttagaga cttttatttt aaaaggcatc |
| 1801 | atatgagcta atatcacaac tttcccagtt taaaaaacta gtactcttgt taaaactcta |
| 1861 | aacttgacta aatacagagg actggtaatt gtacagttct taaatgttgt agtattaatt |
| 1921 | tcaaaactaa aaatcgtcag cacagagtat gtgtaaaaat ctgtaataca aatttttaaa |
| 1981 | ctgatgcttc attttgctac aaaataattt ggagtaaatg tttgatatga tttatttatg |
| 2041 | aaacctaatg aagcagaatt aaatactgta ttaaaataag ttcgctgtct ttaaacaaat |
| 2101 | ggagatgact actaagtcac attgacttta acatgaggta tcactatacc ttatttgtta |
| 2161 | aaatatatac tgtatacatt ttatatattt taacacttaa tactatgaaa acaaataatt |
| 2221 | gtaaaggaat cttgtcagat tacagtaaga atgaacatat ttgtggcatc gagttaaagt |
| 2281 | ttatatttcc cctaaatatg ctgtgattct aatacattcg tgtaggtttt caagtagaaa |
| 2341 | taaacctcgt aacaagttac tgaacgttta aacagcctga caagcatgta tatatgttta |
| 2401 | aaattcaata aacaaagacc cagtccctaa attatagaaa tttaaattat tcttgcatgt |
| 2461 | ttatcgacat cacaacagat ccctaaatcc ctaaatccct aaagattaga tacaaatttt |
| 2521 | ttaccacagt atcacttgtc agaatttatt tttaaatatg attttttaaa actgccagta |
| 2581 | agaaatttta aattaaaccc atttgttaaa ggatatagtg cccaagttat atggtgacct |
| 2641 | acctttgtca atacttagca ttatgtattt caaattatcc aatatacatg tcatatatat |
| 2701 | ttttatatgt cacatatata aaagatatgt atgatctatg tgaatcctaa gtaaatattt |
| 2761 | tgttccagaa aagtacaaaa taataaaggt aaaaataatc tataattttc aggaccacag |
| 2821 | actaagctgt cgaaattaac gctgattttt ttagggccag aataccaaaa tggctcctct |
| 2881 | cttcccccaa aattggacaa tttcaaatgc aaaataattc attatttaat atatgagttg |
| 2941 | cttcctctat t |
| human ANGPTL3 polypeptide sequence | |
| (SEQ ID NO: 1182) | |
| MFTIKLLLFI VPLVISSRID QDNSSFDSLS PEPKSRFAML DDVKILANGL LQLGHGLKDF | |
| VHKTKGQIND IFQKLNIFDQ SFYDLSLQTS EIKEEEKELR RTTYKLQVKN EEVKNMSLEL | |
| NSKLESLLEE KILLQQKVKY LEEQLTNLIQ NQPETPEHPE VTSLKTFVEK QDNSIKDLLQ | |
| TVEDQYKQLN QQHSQIKEIE NQLRRTSIQE PTEISLSSKP RAPRTTPFLQ LNEIRNVKHD | |
| GIPAECTTIY NRGEHTSGMY AIRPSNSQVF HVYCDVISGS PWTLIQHRID GSQNFNETWE | |
| NYKYGFGRLD GEFWLGLEKI YSIVKQSNYV LRIELEDWKD NKHYIEYSFY LGNHETNYTL | |
| HLVAITGNVP NAIPENKDLV FSTWDHKAKG HFNCPEGYSG GWWWHDECGE NNLNGKYNKP | |
| RAKSKPERRR GLSWKSQNGR LYSIKSTKML IHPTDSESFE | |
| cynomolgus ANGPTL3 mRNA sequence | |
| (SEQ ID NO: 1183) | |
| 1 | tagagttaag aagtctaggt ctgcttccag aagaacacag ttccacgctg cttgaaattg |
| 61 | aaaatcagga taaaaatgtt cacaattaag ctccttcttt ttattgttcc tctagttatt |
| 121 | tcctccagaa ttgaccaaga caattcatca tttgattctg tatctccaga gccaaaatca |
| 181 | agatttgcta tgttagacga tgtaaaaatt ttagccaatg gcctccttca gttgggacat |
| 241 | ggtcttaaag actttgtcca taagactaag ggccaaatta atgacatatt tcaaaaactc |
| 301 | aacatatttg atcagtcttt ttatgatcta tcactgcaaa ccagtgaaat caaagaagaa |
| 361 | gaaaaggaac tgagaagaac tacatataaa ctacaagtca aaaatgaaga ggtaaagaat |
| 421 | atgtcacttg aactcaactc aaaacttgaa agcctcctag aagaaaaaat tctacttcaa |
| 481 | caaaaagtga aatatttaga agagcaacta actaacttaa ttcaaaatca acctgcaact |
| 541 | ccagaacatc cagaagtaac ttcacttaaa agttttgtag aaaaacaaga taatagcatc |
| 601 | aaagaccttc tccagactgt ggaagaacaa tataagcaat taaaccaaca gcatagtcaa |
| 661 | ataaaagaaa tagaaaatca gctcagaatg actaatattc aagaacccac agaaatttct |
| 721 | ctatcttcca agccaagagc accaagaact actccctttc ttcagctgaa tgaaataaga |
| 781 | aatgtaaaac atgatggcat tcctgctgat tgtaccacca tttacaatag aggtgaacat |
| 841 | ataagtggca cgtatgccat cagacccagc aactctcaag tttttcatgt ctactgtgat |
| 901 | gttgtatcag gtagtccatg gacattaatt caacatcgaa tagatggatc acaaaacttc |
| 961 | aatgaaacgt gggagaacta caaatatggt ttcgggaggc ttgatggaga attctggttg |
| 1021 | ggcctagaga agatatactc catagtgaag caatctaatt acgttttacg aattgagttg |
| 1081 | gaagactgga aagacaacaa acattatatt gaatattctt tttacttggg aaatcacgaa |
| 1141 | accaactata cgctacatgt agttaagatt actggcaatg tccccaatgc aatcccggaa |
| 1201 | aacaaagatt tggtgttttc tacttgggat cacaaagcaa aaggacactt cagctgtcca |
| 1261 | gagagttatt caggaggctg gtggtggcat gatgagtgtg gagaaaacaa cctaaatggt |
| 1321 | aaatataaca aaccaagaac aaaatctaag ccagagcgga gaagaggatt atcctggaag |
| 1381 | tctcaaaatg gaaggttata ctctataaaa tcaaccaaaa tgttgatcca tccaacagat |
| 1441 | tcagaaagct ttgaatgaac tgaggcaaat ttaaaaggca ataaattaaa cattaaactc |
| 1501 | attccaagtt aatgtggttt aataatctgg tattaaatcc ttaagagaag gcttgagaaa |
| 1561 | tagatttttt tatcttaaag tcactgtcaa tttaagatta aacatacaat cacataacct |
| 1621 | taaagaatac catttacatt tctcaatcaa aattcttaca acactatttg ttttatattt |
| 1681 | tgtgatgtgg gaatcaattt tagatggtcg caatctaaat tataatcaac aggtgaactt |
| 1741 | actaaataac ttttctaaat aaaaaactta gagactttaa ttttaaaagt catcatatga |
| 1801 | gctaatatca caattttccc agtttaaaaa actagttttc ttgttaaaac tctaaacttg |
| 1861 | actaaataaa gaggactgat aattatacag ttcttaaatt tgttgtaata ttaatttcaa |
| 1921 | aactaaaaat tgtcagcaca gagtatgtgt aaaaatctgt aatataaatt tttaaactga |
| 1981 | tgcctcattt tgctacaaaa taatctggag taaatttttg ataggattta tttatgaaac |
| 2041 | ctaatgaagc aggattaaat actgtattaa aataggttcg ctgtctttta aacaaatgga |
| 2101 | gatgatgatt actaagtcac attgacttta atatgaggta tcactatacc ttaacatatt |
| 2161 | tgttaaaacg tatactgtat acattttgtg tattttaata cttaatacta tgaaaacaag |
| 2221 | taattgtaaa cgtatcttgt cagattacaa taggaatgaa catattggtg acatcgagtt |
| 2281 | aaagtttata tttcccctaa atatgctgcg attccaatat attcatgtag gttttcaagc |
| 2341 | agaaataaac cttgtaacaa gttactgact aaaca |

cynomolgus ANGPTL3 polypeptide sequence
(SEQ ID NO: 1184)
MFTIKLLLFI VPLVISSRID QDNSSFDSVS PEPKSRFAML DDVKILANGL LQLGHGLKDE
VHKTKGQIND IFQKLNIFDQ SFYDLSLQTS EIKEEEKELR RTTYKLQVKN EEVKNMSLEL
NSKLESLLEE KILLQQKVKY LEEQLTNLIQ NQPATPEHPE VISLKSFVEK QDNSIKDLLQ
TVEEQYKQLN QQHSQIKEIE NQLRMTNIQE PTEISLSSKP RAPRITPFLQ LNEIRNVKHD
GIPADCITIY NRGEHISGTY AIRPSNSQVF HVYCDVVSGS PWTLIQHRID GSQNFNETWE
NYKYGFGRLD GEFWLGLEKI YSIVKQSNYV LRIELEDWKD NKHYIEYSFY LGNHETNYTL
HVVKITGNVP NAIPENKDLV FSTWDHKAKG HFSCPESYSG GWWWHDECGE NNLNGKYNKP
RTKSKPERRR GLSWKSQNGR LYSIKSTKML IHPTDSESFE

What is claimed is:

1. A double-stranded ribonucleic acid (dsRNA) that inhibits expression of a human angiopoietin-like protein 3 (ANGPTL3) gene by targeting a target sequence on an RNA transcript of the ANGPTL3 gene, wherein the dsRNA comprises a sense strand comprising a sense sequence, and an antisense strand comprising an antisense sequence, wherein the sense sequence is at least 90% identical to the target sequence, and wherein the target sequence is nucleotides 143-161, 135-153, 1535-1553, 143-163, 144-162, 145-163, 150-168, 151-169, 1528-1546, 1530-1548, 1532-1550, 1533-1551, 1602-1620, 2612-2630, or 2773-2791 of SEQ ID NO: 1181.

2. The dsRNA of claim 1, wherein the sense strand and antisense strand are complementary to each other over a region of 15-25 contiguous nucleotides.

3. The dsRNA of any one of claim 1 or 2, wherein the sense strand and the antisense strand are no more than 30 nucleotides in length.

4. The dsRNA of any one of claims 1 to 3, wherein the target sequence is nucleotides 143-161, 135-153, 1535-1553, 143-163, 144-162, 145-163, or 150-168, of SEQ ID NO: 1181.

5. The dsRNA of any one of claims 1 to 4, wherein the dsRNA comprises an antisense sequence that is at least 90% identical to a nucleotide sequence selected from the group consisting of SEQ ID NOs: 227-229, 261-265, 269, 343, 356, 379, 385, 386, and 426.

6. The dsRNA of claim 1, wherein the sense sequence and the antisense sequence are complementary, wherein:
a) the sense sequence comprises a nucleotide sequence selected from the group consisting of SEQ ID NOs: 13-15, 47-51, 55, 129, 142, 165, 171, 172, and 212; or
b) the antisense sequence comprises a nucleotide sequence selected from the group consisting of SEQ ID NOs: 227-229, 261-265, 269, 343, 356, 379, 385, 386, and 426.

7. The dsRNA of claim 6, wherein the sense strand and antisense strand of the dsRNA respectively comprise the nucleotide sequences of:
a) SEQ ID NOs: 13 and 227;
b) SEQ ID NOs: 51 and 265;
c) SEQ ID NOs: 165 and 379;
d) SEQ ID NOs: 14 and 228;
e) SEQ ID NOs: 15 and 229;
f) SEQ ID NOs: 47 and 261;
g) SEQ ID NOs: 48 and 262;
h) SEQ ID NOs: 49 and 263;
i) SEQ ID NOs: 50 and 264;
j) SEQ ID NOs: 55 and 269;
k) SEQ ID NOs: 129 and 343;
l) SEQ ID NOs: 142 and 356;
m) SEQ ID NOs: 171 and 385;
n) SEQ ID NOs: 172 and 386; or
o) SEQ ID NOs: 212 and 426.

8. The dsRNA of claim 6, wherein the sense strand and antisense strand of the dsRNA respectively comprise the nucleotide sequences of:
a) SEQ ID NOs: 13 and 227;
b) SEQ ID NOs: 51 and 265;
c) SEQ ID NOs: 165 and 379;
d) SEQ ID NOs: 14 and 228;
e) SEQ ID NOs: 15 and 229; or
f) SEQ ID NOs: 171 and 385.

9. The dsRNA of any one of claims 1 to 8, wherein the dsRNA comprises one or more modified nucleotides, wherein at least one of the one or more modified nucleotides is 2′-deoxy-2′-fluoro-ribonucleotide, 2′-deoxyribonucleotide, or 2′-O-methyl-ribonucleotide.

10. The dsRNA of any one of claims 1 to 9, wherein the dsRNA comprises an inverted 2′-deoxyribonucleotide at the 3′-end of its sense or antisense strand.

11. The dsRNA of any one of claims 1 to 10, wherein one or both of the sense strand and the antisense strand further comprise:
(a) a 5′ overhang comprising one or more nucleotides; and/or
(b) a 3′ overhang comprising one or more nucleotides.

12. The dsRNA of claim 11, wherein an overhang in the dsRNA comprises two or three nucleotides.

13. The dsRNA of claim 11 or 12, wherein an overhang in the dsRNA comprises one or more thymines.

14. The dsRNA of any one of claims 1 to 13, wherein the sense sequence and the antisense sequence comprise alternating 2′-O-methyl ribonucleotides and 2′-deoxy-2′-fluoro ribonucleotides.

15. The dsRNA of claim 1, wherein:
a) the sense strand comprises a nucleotide sequence selected from the group consisting of SEQ ID NOs: 441-443, 475-479, 483, 557, 570, 593, 599, 600, and 640; and/or
b) the antisense strand comprises a nucleotide sequence selected from the group consisting of SEQ ID NOs: 655-657, 689-693, 697, 771, 784, 807, 813, 814, and 854.

16. The dsRNA of claim 15, wherein the sense strand and antisense strand of the dsRNA respectively comprise the nucleotide sequences of:
a) SEQ ID NOs: 441 and 655;
b) SEQ ID NOs: 479 and 693;
c) SEQ ID NOs: 593 and 807;
d) SEQ ID NOs: 442 and 656;
e) SEQ ID NOs: 443 and 657;
f) SEQ ID NOs: 475 and 689;
g) SEQ ID NOs: 476 and 690;
h) SEQ ID NOs: 477 and 691;
i) SEQ ID NOs: 478 and 692;
j) SEQ ID NOs: 483 and 697;
k) SEQ ID NOs: 557 and 771;
l) SEQ ID NOs: 570 and 784;
m) SEQ ID NOs: 599 and 813;
n) SEQ ID NOs: 600 and 814; or
o) SEQ ID NOs: 640 and 854.

17. The dsRNA of any one of claims 1 to 16, wherein the dsRNA is conjugated to one or more ligands with or without a linker.

18. The dsRNA of claim 18, wherein the ligand is N-acetylgalactosamine (GalNAc) and the dsRNA is conjugated to one or more GalNAc.

19. The dsRNA of any one of claims 1 to 18, wherein the dsRNA is a small interfering RNA (siRNA).

20. The dsRNA of any one of the preceding claims, wherein one or both strands of the dsRNA comprise one or more compounds having the structure of 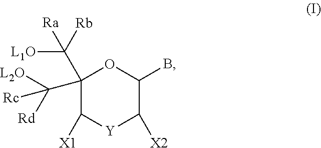
wherein:
B is a heterocyclic nucleobase,
one of L1 and L2 is an internucleoside linking group linking the compound of formula (I) to said strand(s) and the other of L1 and L2 is H, a protecting group, a phosphorus moiety or an internucleoside linking group linking the compound of formula (I) to said strand(s),
Y is O, NH, NR1 or N—C(═O)—R1, wherein R1 is:
a (C1-C20) alkyl group, optionally substituted by one or more groups selected from an halogen atom, a (C1-C6) alkyl group, a (C3-C8) cycloalkyl group, a (C3-C14) heterocycle, a (C6-C14) aryl group, a (C5-C14) heteroaryl group, —O-Z1, —N(Z1)(Z2), —S-Z1, —CN, —C(=J)-O-Z1, —O—C(=J)-Z1, —C(=J)-N(Z1)(Z2), and —N(Z1)-C(=J)-Z2, wherein
J is O or S,
each of Z1 and Z2 is, independently, H, a (C1-C6) alkyl group, optionally substituted by one or more groups selected from a halogen atom and a (C1-C6) alkyl group,
a (C3-C8) cycloalkyl group, optionally substituted by one or more groups selected from a halogen atom and a (C1-C6) alkyl group,
a group —[C(═O)]m-R2-(O—CH₂—CH₂)p-R3, wherein
m is an integer meaning 0 or 1,
p is an integer ranging from 0 to 10,
R2 is a (C1-C20) alkylene group optionally substituted by a (C1-C6) alkyl group, —O-Z3, —N(Z3)(Z4), —S-Z3, —CN, —C(═K)—O—Z3, —O—C(═K)—Z3, —C(═K)—N(Z3)(Z4), or —N(Z3)-C(═K)—Z4,
wherein
K is O or S,
each of Z3 and Z4 is, independently, H, a (C1-C6) alkyl group, optionally substituted by one or more groups selected from a halogen atom and a (C1-C6) alkyl group,
and
R3 is selected from the group consisting of a hydrogen atom, a (C1-C6) alkyl group, a (C1-C6) alkoxy group, a (C3-C8) cycloalkyl group, a (C3-C14) heterocycle, a (C6-C14) aryl group or a (C5-C14) heteroaryl group,
or R3 is a cell targeting moiety,
X1 and X2 are each, independently, a hydrogen atom, a (C1-C6) alkyl group, and
each of Ra, Rb, Rc and Rd is, independently, H or a (C1-C6) alkyl group,
or a pharmaceutically acceptable salt thereof.

21. The dsRNA of claim 20, comprising one or more compounds of formula (I) wherein Y is
a) NR1, R1 is a non-substituted (C1-C20) alkyl group;
b) NR1, R1 is a non-substituted (C1-C16) alkyl group, which includes an alkyl group selected from a group comprising methyl, isopropyl, butyl, octyl, and hexadecyl;
c) NR1, R1 is a (C3-C8) cycloalkyl group, optionally substituted by one or more groups selected from a halogen atom and a (C1-C6) alkyl group;
d) NR1, R1 is a cyclohexyl group;
e) NR1, R1 is a (C1-C20) alkyl group substituted by a (C6-C14) aryl group;
f) NR1, R1 is a methyl group substituted by a phenyl group;
g) N—C(═O)—R1, R1 is an optionally substituted (C1-C20) alkyl group; or
h) N—C(═O)—R1, R1 is methyl or pentadecyl.

22. The dsRNA of claim 20 or 21, comprising one or more compounds of formula (I) wherein B is selected from a group consisting of a pyrimidine, a substituted pyrimidine, a purine and a substituted purine, or a pharmaceutically acceptable salt thereof.

23. The dsRNA of any one of claims 20 to 22, wherein R3 is of the formula (II): 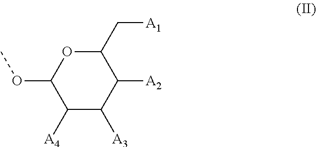
wherein A1, A2 an A3 are H,
A4 is OH or NHC(═O)—R5, wherein R5 is a (C1-C6) alkyl group, optionally substituted by a halogen atom, or a pharmaceutically acceptable salt thereof.

24. The dsRNA of any one of claims 20 to 23, wherein R3 is N-acetyl-galactosamine, or a pharmaceutically acceptable salt thereof.

25. The dsRNA of any one of claims 20 to 24, comprising one or more nucleotides from Tables A.

26. The dsRNA of claims 20 to 25, comprising from 2 to 10 compounds of formula (I), or a pharmaceutically acceptable salt thereof.

27. The dsRNA of claim 26, wherein the 2 to 10 compounds of formula (I) are on the sense strand.

28. The dsRNA of any one of claims 20 to 27, wherein the sense strand comprises two to five compounds of formula (I) at the 5′ end, and/or comprises one to three compounds of formula (I) at the 3′ end.

29. The dsRNA of claim 28, wherein
a) the two to five compounds of formula (I) at the 5′ end of the sense strand comprise lgT3 and/or lgT7, optionally comprising three consecutive lgT3 nucleotides; and/or
b) the one to three compounds of formula (I) at the 3′ end of the sense strand comprise lT4 or lT3;
optionally comprising two consecutive lT4.

30. The dsRNA of any one of claims 1 to 29, comprising one or more internucleoside linking groups independently selected from the group consisting of phosphodiester, phosphotriester, phosphorothioate, phosphorodithioate, alkyl-phosphonate and phosphoramidate backbone linking groups, or a pharmaceutically acceptable salt thereof.

31. The dsRNA of claim 1, selected from the dsRNAs in Tables 1-3.

32. The dsRNA of claim 1, wherein:
a) the sense strand comprises a nucleotide sequence selected from the group consisting of SEQ ID NOs: 858, 902, 907, 911, 915, 934, 970, 979, and 988; or
b) the antisense strand comprises a nucleotide sequence selected from the group consisting of SEQ ID NOs: 1020, 1064, 1069, 1073, 1077, 1096, 1132, 1141, and 1150.

33. The dsRNA of claim 32, wherein the sense strand and antisense strand of the dsRNA respectively comprise the nucleotide sequences of:
a) SEQ ID NOs: 858 and 1020;
b) SEQ ID NOs: 902 and 1064;
c) SEQ ID NOs: 907 and 1069;
d) SEQ ID NOs: 911 and 1073;
e) SEQ ID NOs: 915 and 1077;
f) SEQ ID NOs: 934 and 1096;
g) SEQ ID NOs: 970 and 1132;
h) SEQ ID NOs: 979 and 1141; or
i) SEQ ID NOs: 988 and 1150.

34. A pharmaceutical composition comprising the dsRNA of any one of claims 1 to 33 and a pharmaceutically acceptable excipient.

35. The dsRNA of any one of claims 1 to 33 or the composition of claim 34 for use in inhibiting ANGPTL3 expression in a human in need thereof.

36. The dsRNA or composition for use of claim 35, wherein expression of the ANGPTL3 gene in the liver of the human is inhibited by the dsRNA.

37. The dsRNA of any one of claims 1 to 36 or the composition of claim 34 for use in treating or preventing an ANGPTL3-associated condition in a human in need thereof.

38. The dsRNA or composition for use of claim 37, wherein the ANGPTL3-associated condition is a lipid metabolism disorder.

39. The dsRNA or composition for use of claim 38, wherein the lipid metabolism disorder is hypertriglyceridemia.

40. A method of treating and/or preventing one or more ANGPTL3-associated conditions comprising administering one or more dsRNAs as defined in any one of claims 1 to 33 and/or one or more pharmaceutical compositions as defined in claim 34.
